(19) United States
(12) Patent Application Publication
(10) Pub. No.: US 2023/0322885 A1
(43) Pub. Date: Oct. 12, 2023

(54) COMPOSITIONS AND METHODS FOR SIMULTANEOUSLY MODULATING EXPRESSION OF GENES

(71) Applicant: Versameb AG, Basel (CH)

(72) Inventors: Justin Antony SELVARAJ, Weil am Rhein (DE); Friedrich METZGER, Freiburg (DE); Kiaas Pieter ZUIDEVELD, Riehen (CH); Hervé SCHAFFHAUSER, Habsheim (FR); Petra HILLMANN-WÜLLNER, Zurich (CH)

(21) Appl. No.: 18/192,717

(22) Filed: Mar. 30, 2023

Related U.S. Application Data
(63) Continuation of application No. PCT/IB21/00682, filed on Oct. 4, 2021.
(60) Provisional application No. 63/087,643, filed on Oct. 5, 2020.
(60) Provisional application No. 63/213,841, filed on Jun. 23, 2021.

(51) Int. Cl. C07K 14/55; C12N 15/113; A61P 35/00

(52) U.S. Cl. CPC C07K 14/55; C12N 15/113; A61P 35/00; C12N 2310/11

(57) ABSTRACT

The present invention relates to compositions of recombinant polynucleic acid constructs comprising at least one nucleic acid sequence encoding an siRNA capable of binding to a target mRNA and at least one nucleic acid sequence encoding a gene of interest. Also disclosed herein is use of the compositions in treating cancers and in simultaneously modulating expression of two or more genes.

### CROSS-REFERENCE TO RELATED APPLICATIONS

This application is a continuation of International Application No. PCT/IB2021/000682, filed Oct. 4, 2021, which claims the benefit of U.S. Provisional Application No. 63/087,643, filed Oct. 5, 2020 and U.S. Provisional Application No. 63/213,841, filed Jun. 23, 2021, each of which is incorporated by reference herein in its entirety.

### SEQUENCE LISTING

The instant application contains a Sequence Listing which has been submitted electronically in XML format and is hereby incorporated by reference in its entirety. Said XML copy, created on Mar. 17, 2023, is named 57623-707_301_SL.xml and is 330,489 bytes in size.

### BACKGROUND

Many aberrant human conditions are caused by or associated with shifts in gene expression level relative to those protein expression levels in subjects without such aberrant human conditions. This is particularly so in the case of cancer. For example, cancer cells are known to benefit from increasing expression of proteins involved in cell proliferation or angiogenesis and reducing expression of proteins involved in immune response to tumors. Thus, there is a need for therapies that decrease production of one or more target gene products involved in cell proliferation or angiogenesis and concomitantly increase production of others such as proteins involved in immune response to tumors needed to prevent or treat incidents of cancer in a subject.

### BRIEF SUMMARY

Provided herein are compositions and methods for simultaneously modulating expression of two or more proteins or nucleic acid sequences using one recombinant polynucleic acid or RNA construct. In some aspects, provided herein, is a composition comprising a first RNA linked to a second RNA, wherein the first RNA encodes for a cytokine, and wherein the second RNA encodes for a genetic element that modulates expression of a gene associated with tumor proliferation. In some aspects, provided herein, is a composition comprising a first RNA linked to a second RNA, wherein the first RNA encodes for a cytokine, and wherein the second RNA encodes for a genetic element that modulates expression of a gene associated with recognition by the immune system. In some aspects, provided herein, is a pharmaceutical composition comprising any of the compositions described herein and a pharmaceutically acceptable excipient.

In some aspects, provided herein, is a composition comprising a first RNA encoding for interleukin-2 (IL-2), IL-15, a fragment thereof, or a functional variant thereof linked to a second RNA encoding for a genetic element that modulates expression of vascular endothelial growth factor A (VEGFA), an isoform of VEGFA, placental growth factor (PIGF), cluster of differentiation 155 (CD155), programmed cell death-ligand 1 (PD-L1), myc proto-oncogene (c-Myc), a fragment thereof, or a functional variant thereof. In some aspects, provided herein, is a composition comprising a first RNA encoding for interleukin-2 (IL-2), a fragment thereof, or a functional variant thereof linked to a second RNA encoding for a genetic element that modulates expression of MHC class I chain-related sequence A (MICA), MHC class I chain-related sequence B (MICB), endoplasmic reticulum protein (ERp5), a disintegrin and metalloproteinase (ADAM), matrix metalloproteinase (MMP), a fragment thereof, or a functional variant thereof. In some embodiments, the ADAM is ADAM17. In some aspects, provided herein, is a composition comprising a first RNA encoding for interleukin-12 (IL-12), IL-7, a fragment thereof, or a functional variant thereof linked to a second RNA encoding for a genetic element that modulates expression of isocitrate dehydrogenase (IDH1), cyclin-dependent kinase 4 (CDK4), CDK6, epidermal growth factor receptor (EGFR), mechanistic target of rapamycin (mTOR), Kirsten rat sarcoma viral oncogene (KRAS), programmed cell death-ligand 1 (PD-L1), a fragment thereof, or a functional variant thereof. In some aspects, provided herein, is a pharmaceutical composition comprising any of the compositions described herein and a pharmaceutically acceptable excipient.

In some aspects, provided herein, is a method of treating cancer, comprising administering any of the compositions or the pharmaceutical composition described herein to a subject having a cancer. In some embodiments, the cancer is a solid tumor. In some embodiments, the cancer is melanoma. In some embodiments, the cancer is renal cell carcinoma. In some embodiments, the cancer is a head and neck cancer. In some embodiments, the head and neck cancer is head and neck squamous cell carcinoma. In some embodiments, the head and neck cancer is laryngeal cancer, hypopharyngeal cancer, tonsil cancer, nasal cavity cancer, paranasal sinus cancer, nasopharyngeal cancer, metastatic squamous neck cancer with occult primary, lip cancer, oral cancer, oral cancer, oropharyngeal cancer, salivary gland cancer, brain tumors, esophageal cancer, eye cancer, parathyroid cancer, sarcoma of the head and neck, or thyroid cancer. In some embodiments, the subject is a human.

In some aspects, provided herein, is a composition comprising a recombinant polynucleic acid construct comprising a nucleic acid sequence selected from the group consisting of SEQ ID NOs: 1-17 and 125-141.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a schematic representation of construct design. A polynucleic acid construct may comprise a T7 promoter sequence upstream of the gene of interest sequence (IL-2 given as an example) for T7 RNA polymerase binding and successful in vitro transcription of both the gene of interest and siRNA in a single transcript. Signal peptide of IL-2 is highlighted in a grey box. Linkers to connect mRNA to siRNA or siRNA to siRNA are indicated with boxes with horizontal stripes or boxes with checkered stripes, respectively. T7: T7 promoter, siRNA: small interfering RNA.

FIG. 2A is a plot for induction of IL-2 secretion from human embryonic kidney cells (HEK-293). The X-axis indicates mRNAs used for transfection into HEK-293 cells: Compound (Cpd.) 1, Cpd.2, Cpd.3, or Cpd.4. The Y-axis is a measurement of IL-2 protein secretion fold change compared to IL-2 protein secretion by Cpd.1 using ELISA. Data represent means±standard error of the mean of 3 replicates per Cpd. Significance (**, p<0.01) was assessed by one way ANOVA followed by Dunnet's multiple comparing test using Cpd.1 as control.

FIG. 2B is a plot for induction of IL-2 secretion from human adult keratinocytes (HaCaT). The X-axis indicates mRNAs used for transfection into HaCaT cells: Compound (Cpd.) 1, Cpd.2, Cpd.3, or Cpd.4. The Y-axis is a measurement of IL-2 protein secretion fold change compared to IL-2 protein secretion by Cpd.1 using ELISA. Data represent means±standard error of the mean of 3 replicates per Cpd. Significance (**, p<0.01) was assessed by one way ANOVA followed by Dunnet's multiple comparing test using Cpd.1 as control.

FIG. 2C is a plot for induction of IL-2 secretion from human lung epithelial cells (A549). The X-axis indicates mRNAs used for transfection into A549 cells: Compound (Cpd.) 1, Cpd.2, Cpd.3, or Cpd.4. The Y-axis is a measurement of IL-2 protein secretion fold change compared to IL-2 protein secretion by Cpd.1 using ELISA. Data represent means±standard error of the mean of 3 replicates per Cpd. Significance (**, p<0.01) was assessed by one way ANOVA followed by Dunnet's multiple comparing test using Cpd.1 as control.

FIG. 3 is a plot for dose-dependent secretion of IL-2 protein and simultaneous interference of VEGFA expression by Compound 5 (Cpd.5) in lung epithelial cells (A549 cells) which overexpresses VEGFA (0.3 μg VEGFA mRNA). The X-axis indicates concentrations of Cpd.5 (4.4, 8.8, 17.6, 26.4, 35.2 and 44.02 nM that correspond to 0, 150, 300, 600, 900, or 1200 ng/well, respectively) used for transfection into A549 cells. The Y-axis is a measurement of VEGFA (left) and IL-2 (right) protein levels (ng/ml) in the same cell culture supernatant by ELISA, 24 hours after transfection with Cpd.5. Data represent means±standard error of the mean of 4 replicates.

FIG. 4A is a plot for interference of VEGFA expression by Compound 5 (Cpd.5) in human tongue cell carcinoma cells (SCC-4) transfected with VEGFA mRNA to overexpress VEGFA. The X-axis indicates SCC-4 cells transfected with 9.5 nM (300 ng) of VEGFA mRNA only (VEGFA mRNA) or co-transfected with 9.5 nM (300 ng) of VEGFA mRNA and 26.4 nM (900 ng) of Cpd.5 (Cpd.5). The Y-axis is a measurement of VEGFA protein level (ng/ml) in cell culture supernatant by ELISA, 24 hours after transfection. Data represent means±standard error of the mean of 4 replicates.

FIG. 4B is a plot for IL-2 protein level (ng/ml) in the same cell culture supernatant as in FIG. 4A, measured by ELISA. Data represent means±standard error of the mean of 4 replicates.

FIG. 5A is a plot for interference of VEGFA expression by Compound 5 (Cpd.5) in human tongue cell carcinoma cells (SCC-4) that endogenously overexpress VEGFA. The X-axis indicates SCC-4 cells before (Endogenous) and after transfection (Cpd.5) with 26.4 nM (900 ng) of Cpd.5. The Y-axis is a measurement for VEGFA protein level (ng/ml) in cell culture supernatant by ELISA, 24 hours after transfection. Data represent means±standard error of the mean of two replicates.

FIG. 5B is a plot for IL-2 protein level (ng/ml) in the same cell culture supernatant as in FIG. 5A, measured by ELISA. Data represent means±standard error of the mean of two replicates.

FIG. 6A is a plot for interference of VEGFA expression by Compound 5 (Cpd.5) and commercial siRNA in human tongue cell carcinoma cells (SCC-4) transfected with VEGFA mRNA to overexpress VEGFA (9.5 nM or 0.3 μg VEGFA mRNA). The X-axis indicates SCC-4 cells transfected with increasing concentration of Cpd.5 (4.4 nM to 44.02 nM) or commercial siRNA (0.05 mM to 2.5 mM). The Y-axis indicates a measurement of VEGFA protein level (pg/ml) in cell culture supernatant by ELISA, 24 hours after transfection. Data represent means±standard error of the mean of 4 replicates.

FIG. 6B is a plot for interference of VEGFA expression by Compound 5 (Cpd.5) and commercial siRNA in human lung epithelial cells (A549) transfected with VEGFA mRNA to overexpress VEGFA (9.5 nM or 0.3 μg VEGFA mRNA). The X-axis indicates A549 cells transfected with increasing concentration of Cpd.5 (4.4 nM to 44.02 nM) or commercial siRNA (0.05 mM to 2.5 mM). The Y-axis indicates a measurement of VEGFA protein level (pg/ml) in cell culture supernatant by ELISA, 24 hours after transfection. Data represent means±standard error of the mean of 4 replicates.

FIG. 6C is a table for comparison of IC50 values of Cpd. 5 and commercial siRNAs in SCC-4 and A549 cells.

FIG. 7A is a plot for interference of MICB expression by Compound 6 (Cpd.6) in human tongue cell carcinoma cells (SCC-4) that constitutively express soluble and membrane MICB. The X-axis indicates SCC-4 cells before (Endogenous) and after transfection (Cpd.6) with 35.11 nM (900 ng) of Cpd.6. The Y-axis is a measurement for soluble MICB protein level (pg/ml) in cell culture supernatant by ELISA, 24 hours after transfection. Data represent means±standard error of the mean of 4 replicates.

FIG. 7B is a plot for interference of MICB expression by Compound 6 (Cpd.6) in human tongue cell carcinoma cells (SCC-4) that constitutively express soluble and membrane MICB. The X-axis indicates SCC-4 cells before (Endogenous) and after transfection (Cpd.6) with 35.11 nM (900 ng) of Cpd.6. The Y-axis is a measurement for membrane MICB protein level (pg/ml) in cell culture supernatant by ELISA, 24 hours after transfection. Data represent means±standard error of the mean of 4 replicates.

FIG. 7C is a plot for IL-2 protein level (ng/ml) in the same cell culture supernatant as in FIG. 7A and FIG. 7B, measured by ELISA. Data represent means±standard error of the mean of 4 replicates.

FIG. 8A is a plot for dose-dependent secretion of IL-2 protein and simultaneous interference of MICA expression by Compound 6 (Cpd.6) in human tongue cell carcinoma cells (SCC-4) that constitutively express soluble MICA. The X-axis indicates concentrations of Cpd.6 (1.58, 2.93, 5.85, 11.7, 23.41, 35.11 and 46.81 nM) used for transfection into SCC-4 cells. The Y-axis is a measurement for soluble MICA protein level (pg/ml) in cell culture supernatant by ELISA, 24 hours after transfection. Data represent means±standard error of the mean of 4 replicates.

FIG. 8B is a plot for dose-dependent secretion of IL-2 protein and simultaneous interference of MICB expression by Compound 6 (Cpd.6) in the same SCC-4 cells supernatant described in FIG. 8A. SCC-4 cells constitutively express soluble MICB. The X-axis indicates concentrations of Cpd.6 (1.58, 2.93, 5.85, 11.7, 23.41, 35.11 and 46.81 nM) used for transfection into SCC-4 cells. The Y-axis is a measurement for soluble MICB protein level (pg/ml) in cell culture supernatant by ELISA, 24 hours after transfection. Data represent means±standard error of the mean of 4 replicates.

FIG. 9A is a plot for IL-2 expression measured at 12, 24 and 48 hours post transfection with Cpd.3 (100 ng) in three-dimensional (3D) spheroid culture of SK-OV-3-NLR cells seeded at 5000 cells/well into an ultra-low attachment (ULA) plate. IL-2 quantification was performed with TR-FRET assay. Error bars represent mean±SEM of three replicates.

FIGS. 9B-9D shows changes in the total nuclear localized RFP (NLR) integrated intensity of SK-OV-3 NLR spheroids post transfection with Cpd.3 in the presence of peripheral blood mononuclear cells (PBMCs). SK-OV-3 NLR were plated in ULA plates (quadruplicate) at 5000 cells/well and transfected with different doses of Cpd.3 (3 ng, 10 ng, 30 ng and 100 ng) using Lipofectamine 2000. The cells were then centrifuged to form spheroids and cultured for 48 hrs prior to PBMC addition. PBMCs isolated from 3 donors (FIGS. 9B, 9C and 9D) were added at a density of 200,000 cells/well along with anti-CD3. The co-cultures were imaged every 3 hours for 168 hours (7 days). Total NLR integrated intensity was normalized to the 24 hour time point and analysed using the spheroid module within the IncuCyte software. rhIL2: recombinant human IL-2

FIG. 9E shows a set of representative IncuCyte images showing Cpd.3 mediated NLR integrity reduction after PBMC alone control, recombinant human IL-2 (rhIL2) and Cpd.3 treatment (100 ng) in the SK-OV-3 NLR condition at Day-5.

FIG. 10A is a plot showing dose-dependent activation of the JAK3/STATS pathway in HEK-Blue™ IL-2 reporter cells induced by rh-IL-2 (0.001 ng to 300 ng) or IL-2 (0.001 ng-45 ng) derived from supernatant of human embryonic kidney (HEK293) cells that had been transfected with Cpd.5 (0.3 μg/well) and quantified by ELISA. The X-axis indicates different concentration of Cpd.5 derived IL-2 or rh-IL-2. The Y-axis indicates IL-2 signaling activation normalized to rh-IL-2 (lowest SEAP values of rh-IL-2 set to 0 and highest SEAP values of rh-IL-2 set to 100%). Data represent means±standard error of the mean of 4 replicates per dose.

FIG. 10B is a plot showing dose-dependent activation of the JAK3/STATS pathway in HEK-Blue™ IL-2 reporter cells induced by rh-IL-2 (0.001 ng to 300 ng) or IL-2 (0.001 ng-45 ng) derived from supernatant of human embryonic kidney (HEK293) cells that had been transfected with Cpd.6 (0.3 μg/well) and quantified by ELISA. The X-axis indicates different concentration pf Cpd.6 derived IL-2 or rh-IL-2. The Y-axis indicates IL-2 signaling activation normalized to rh-IL-2. Data represent means±standard error of the mean of 4 replicates per dose.

FIG. 10C is a plot showing a NK cell mediated killing assay measured by luminescent cell viability approach (CellTiter-Glo). SCC-4 cells transfected with different doses of Cpd.5, Cpd.6 and two mock control RNAs (0.1 nM to 2.5 nM). 30 minutes after transfection, NK-92 cells were co-cultured with SCC-4 cells at the 10:1 effector to target (E:T) cell ratio and then incubated for 24 hours at 37° C. Cells were then thoroughly washed to remove NK-92 cells, and survived SCC-4 cells were analyzed by cell viability assay using CellTiter-Glo. Untreated SCC-4 cells were used as control and set to 0%. Data represent mean±SEM from 4 replicates per dose.

FIG. 11A is a plot showing dose-dependent downregulation of endogenously expressed VEGFA induced by Compound 7 (Cpd.7) and Compound 8 (Cpd.8) in SCC-4 cells. VEGFA levels in the cell culture supernatant were measured by ELISA, 24 hours after transfection. The X-axis indicates concentrations of Cpd.7 (1.1, 2.2, 4.4, 8.8, 17.6, 26.4, 35.2 and 44.04 nM/well) and Cpd.8 (0.47, 0.94, 1.89, 3.79, 7.58, 15.15, 22.73, 30.31 and 37.88 nM/well) used for transfection into SCC-4 cells. VEGFA levels from untransfected cells were set to 100%. The Y-axis indicates down regulation of VEGFA level normalized to untransfected samples (basal level). Data represent means±standard error of the mean of 4 replicates.

FIG. 11B is a plot showing dose-dependent secretion of IL-2 levels induced by Cpd.7 (3× siRNA) and Cpd.8 (5× siRNA) in SCC-4 cells. IL-2 levels in the cell culture supernatant were measured by ELISA, 24 hours after transfection. The X-axis indicates concentrations of Cpd.7 (1.1, 2.2, 4.4, 8.8, 17.6, 26.4, 35.2 and 44.04 nM/well) and Cpd.8 (0.47, 0.94, 1.89, 3.79, 7.58, 15.15, 22.73, 30.31 and 37.88 nM/well) used for transfection into SCC-4 cells. The Y-axis is a measurement for IL-2 protein level (nM) in cell culture supernatant, 1 nM correspond to dissociation constant (Kd) of IL-2 with its receptor. Data represent means±standard error of the mean of 4 replicates.

FIG. 11C is a plot showing the time-course of IL-2 secretion induced by Compound 9 (Cpd.9) and Compound (Cpd.10) in SCC-4 cells up to 72 hours. IL-2 levels in the cell culture supernatant were measured by ELISA, from 6 to 72 hours after transfection (30 nM). The X-axis indicates hours after transfection and Y-axis is a measurement for IL-2 protein level (nM) in cell culture supernatant. Data represent means±standard error of the mean of 4 replicates.

FIG. 11D is a plot for time-dependent downregulation of constitutively expressed VEGFA level by scrambled siRNA (scr. siRNA), commercial VEGFA siRNA, Cpd.9 and Cpd.10 in SCC-4 cells up to 72 hours. VEGFA levels in the cell culture supernatant were measured by ELISA, from 6 hours to 72 hours after transfection (30 nM). VEGFA levels from untransfected cells were set to 100% and down regulation was normalized to this value. The X-axis indicates hours after transfection and Y-axis indicates down regulation of VEGFA level normalized to untransfected samples (basal level). Data represent means±standard error of the mean of 4 replicates.

FIG. 12A and FIG. 12C are plots showing secretion of IL-12 levels induced by compound 11 (Cpd.11) in SCC-4 cells and A549 cells, respectively. IL-12 levels in the cell culture supernatant were measured by ELISA, 24 hours after transfection. The X-axis indicates concentrations of Cpd.11 (7 (10 nM and 30 nM/well) used for transfection into SCC-4 cells. The Y-axis is an IL-12 protein level (pg/ml) in cell culture supernatant. Data represent means±standard error of the mean of 4 replicates.

FIG. 12B and FIG. 12D are plots showing downregulation of IDH1, CDK4 and CDK6 levels resulting from Cpd.11 treatment in SCC-4 cells and A549 cells, respectively. RNA levels of IDH1, CDK4 and CDK6 were measured from cell lysate by qPCR in technical duplicates, 24 hours after transfection. The X-axis indicates concentrations of Cpd.11 (10 nM and 30 nM/well) used for transfection into SCC-4 cells and A549 cells. The Y-axis indicates down regulation of IDH1, CDK4 and CDK6 level normalized to untransfected samples (basal level). Data represent means±standard error of the mean of 4 replicates.

FIG. 12E and FIG. 12G are plots showing secretion of IL-12 levels induced by compound 12 (Cpd.12) in SCC-4 cells and A549 cells, respectively. IL-12 levels in the cell culture supernatant were measured by ELISA, 24 hours after transfection. The X-axis indicates concentrations of Cpd.12 (10 nM and 30 nM/well) used for transfection into SCC-4 cells and A549 cells. The Y-axis is an IL-12 protein level (pg/ml) in cell culture supernatant. Data represent means±standard error of the mean of 4 replicates.

FIG. 12F and FIG. 12H are plots showing downregulation of EGFR, KRAS and mTOR levels resulting from Cpd.12 treatment in SCC-4 cells and A549 cells, respectively. RNA levels of EGFR, KRAS and mTOR were measured from cell lysate by qPCR in technical duplicates, 24 hours after transfection. The X-axis indicates concentrations of Cpd.12 (10 nM and 30 nM/well) used for transfection into SCC-4 cells and A549 cells. The Y-axis indicates down regulation of EGFR, KRAS and mTOR level normalized to untransfected samples (basal level). BQL=below quantification limit of the assay. Data represent means±standard error of the mean of 4 replicates.

FIG. 13A and FIG. 13B are plots showing secretion of IL-12 levels induced by Compound 13 (Cpd.13) in A549 cells and SCC-4 cells, respectively. IL-12 levels in the cell culture supernatant were measured by ELISA, 24 hours after transfection. The X-axis indicates concentrations of Cpd.13 (10 nM and 30 nM/well) used for transfection into A549 cells and SCC-4 cells. The Y-axis is an IL-12 protein level (pg/ml) in cell culture supernatant. Data represent means±standard error of the mean of 4 replicates.

FIG. 13C is a plot showing secretion of IL-12 levels induced by Compound 14 (Cpd.14) in A549 cells. IL-12 levels in the cell culture supernatant were measured by ELISA, 24 hours after transfection. The X-axis indicates concentrations of Cpd.14 (10 nM and 30 nM/well) used for transfection into A549 cells. The Y-axis is an IL-12 protein level (pg/ml) in cell culture supernatant. Data represent means±standard error of the mean of 4 replicates.

FIG. 13D and FIG. 13E are plots showing downregulation of EGFR expression resulting from Cpd.13 treatment in A549 cells and SCC-4 cells, respectively. RNA levels of EGFR were measured from cell lysate by qPCR in technical duplicates, 24 hours after transfection. The X-axis indicates concentrations of Cpd.13 (10 nM and 30 nM/well) used for transfection into A549 cells and SCC-4 cells. The Y-axis indicates down regulation of EGFR level normalized to untransfected samples (basal level). Data represent means±standard error of the mean of 4 replicates.

FIG. 13F is a plot showing downregulation of mTOR expression resulting from Cpd.14 treatment in A549 cells. RNA levels of mTOR were measured from cell lysate by qPCR in technical duplicates, 24 hours after transfection. The X-axis indicates concentrations of Cpd.14 (10 nM and 30 nM/well) used for transfection into A549 cells. The Y-axis indicates down regulation of mTOR level normalized to untransfected samples (basal level). Data represent means±standard error of the mean of 4 replicates.

FIG. 14A and FIG. 14C are plots showing secretion of IL-15 levels induced by Compound 15 (Cpd.15) in A549 cells and SCC-4 cells, respectively. IL-15 levels in the cell culture supernatant were measured by ELISA, 24 hours after transfection. The X-axis indicates concentrations of Cpd.15 (10 nM and 30 nM/well) used for transfection into A549 cells and SCC-4 cells. The Y-axis is an IL-15 protein level (pg/ml) in cell culture supernatant. Data represent means±standard error of the mean of 4 replicates.

FIG. 14B and FIG. 14D are plots showing downregulation of VEGFA and CD155 expression resulting from Cpd.15 treatment in A549 cells and SCC-4 cells, respectively. RNA levels of VEGFA and CD155 were measured from cell lysate by qPCR in technical duplicates, 24 hours after transfection. The X-axis indicates concentrations of Cpd.15 (10 nM and 30 nM/well) used for transfection into A549 cells and SCC-4 cells. The Y-axis indicates down regulation of VEGFA and CD155 level normalized to untransfected samples (basal level). Data represent means±standard error of the mean of 4 replicates.

FIG. 14E is a plot showing secretion of IL-15 levels induced by Compound 16 (Cpd.16) in human glioblastoma cell line (U251 MG) cells. IL-15 levels in the cell culture supernatant were measured by ELISA, 24 hours after transfection. The X-axis indicates concentrations of Cpd.16 (10 nM and 30 nM/well) used for transfection into U251 MG cells. The Y-axis is an IL-15 protein level (pg/ml) in cell culture supernatant. Data represent means±standard error of the mean of 4 replicates.

FIG. 14F is a plot showing downregulation of VEGFA, PD-L1 and c-Myc expression resulting from Cpd.16 treatment in U251 MG cells. RNA levels of VEGFA, PD-L1 and c-Myc were measured from cell lysate by qPCR in technical duplicates, 24 hours after transfection. The X-axis indicates concentrations of Cpd.16 (10 nM and 30 nM/well) used for transfection into U251 MG cells. The Y-axis indicates down regulation of VEGFA, PD-L1 and c-Myc level normalized to untransfected samples (basal level). Data represent means±standard error of the mean of 4 replicates.

FIG. 14G is a plot showing secretion of IL-7 levels induced by Compound 17 (Cpd.17) in U251 MG cells. IL-7 levels in the cell culture supernatant were measured by ELISA, 24 hours after transfection. The X-axis indicates concentrations of Cpd.17 (10 nM and 30 nM/well) used for transfection into U251 MG cells. The Y-axis is an IL-7 protein level (pg/ml) in cell culture supernatant. Data represent means±standard error of the mean of 4 replicates.

FIG. 14H is a plot showing downregulation of PD-L1 expression resulting from Cpd.17 treatment in U251 MG cells. RNA levels of PD-L1 were measured from cell lysate by qPCR in technical duplicates, 24 hours after transfection. The X-axis indicates concentrations of Cpd.17 (10 nM and 30 nM/well) used for transfection into U251 MG cells. The Y-axis indicates down regulation of PD-L1 level normalized to untransfected samples (basal level). Data represent means±standard error of the mean of 4 replicates.

FIG. 15A is a plot showing downregulation of endogenously expressed VEGFA induced by Compound 5 (Cpd.5) and Compound 10 (Cpd.10) in SCC-4 cells. VEGFA levels in the cell culture supernatant were measured by ELISA, 24 hours after transfection. The X-axis indicates concentrations of Cpd.5 and Cpd.10 (20 and 30 nM) used for transfection into SCC-4 cells. VEGFA levels from untransfected cells represent the endogenous VEGFA secretion levels of SCC-4 cells and were labelled as ‘0’. The Y-axis indicates VEGFA levels measured by ELISA. Data represent means±standard error of the mean of 2 independent measurements.

FIG. 15B is a plot showing the number of branching points induced by VEGFA from different media supernatants in FIG. 15A in the HUVEC in vitro angiogenesis model. Recombinant human VEGFA (VEGF) was used as a control and number of branching points were counted from microscopical pictures at the 6 hours time point. Data represent means±standard error of the mean of 6 independent measurements.

### DETAILED DESCRIPTION

Provided herein are compositions and methods for modulating expression of two or more genes simultaneously, comprising at least one nucleic acid sequence encoding a gene of interest and at least one nucleic acid sequence encoding or comprising a small interfering RNA (siRNA) capable of binding to a target messenger RNA (mRNA). Also provided herein are compositions and methods for treating cancers, comprising recombinant RNA constructs to simultaneously express a cytokine and a genetic element that reduces expression of a gene associated with tumor proliferation, angiogenesis, or recognition by the immune system from a single RNA transcript. Further provided herein are compositions and methods to modulate expression of two or more genes simultaneously. Provided herein are compositions comprising a first RNA linked to a second RNA, wherein the first RNA encodes for a cytokine, and wherein the second RNA encodes for a genetic element that reduces expression of a gene associated with tumor proliferation, angiogenesis, or recognition by the immune system. In one example, the first RNA may be a messenger RNA (mRNA) encoding a cytokine and can increase the protein level of a cytokine. In another example, the second RNA or the genetic element that reduces expression of a gene associated with tumor proliferation, angiogenesis, or recognition by the immune system can include a small interfering RNA (siRNA) capable of binding to a target mRNA and can downregulate the level of protein encoded by the target mRNA. In some embodiments, target mRNAs can include an mRNA of a gene associated with tumor proliferation, angiogenesis, or recognition by the immune system.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

### Definitions

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The term “about” or “approximately” can mean within an acceptable error range for the particular value as determined by one of ordinary skill in the art, which will depend in part on how the value is measured or determined, i.e. the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “embodiments,” “certain embodiments,” “preferred embodiments,” “specific embodiments,” “some embodiments,” “an embodiment,” “one embodiment” or “other embodiments” mean that a particular feature, structure, or characteristic described in connection with the embodiments is included in at least some embodiments, but not necessarily all embodiments, of the present disclosures. To facilitate an understanding of the present disclosure, a number of terms and phrases are defined below.

The term “RNA” as used herein includes RNA which encodes an amino acid sequence (e.g., mRNA, etc.) as well as RNA which does not encode an amino acid sequence (e.g., siRNA, shRNA, miRNA etc.). The RNA as used herein may be a coding RNA, i.e., an RNA which encodes an amino acid sequence. Such RNA molecules are also referred to as mRNA (messenger RNA) and are single-stranded RNA molecules. The RNA as used herein may be a non-coding RNA, i.e., an RNA which does not encode an amino acid sequence or is not translated into a protein. A non-coding RNA can include, but is not limited to, a small interfering RNA (siRNA), a short or small harpin RNA (shRNA), a microRNA (miRNA), a piwi-interacting RNA (piRNA), and a long non-coding RNA (lncRNA). siRNAs as used herein may comprise a double-stranded RNA (dsRNA) region, a hairpin structure, a loop structure, or any combinations thereof. In some embodiments, siRNAs may comprise at least one shRNA, at least one dsRNA region, or at least one loop structure. In some embodiments, siRNAs may be processed from a dsRNA or an shRNA. In some embodiments, siRNAs may be processed or cleaved by an endogenous protein, such as DICER, from an shRNA. In some embodiments, a hairpin structure or a loop structure may be cleaved or removed from an siRNA. For example, a hairpin structure or a loop structure of an shRNA may be cleaved or removed. In some embodiments, RNAs described herein may be made by synthetic, chemical, or enzymatic methodology known to one of ordinary skill in the art, made by recombinant technology known to one of ordinary skill in the art, or isolated from natural sources, or made by any combinations thereof. The RNA may comprise modified or unmodified nucleotides or mixtures thereof, e.g., the RNA may optionally comprise chemical and naturally occurring nucleoside modifications known in the art (e.g., N¹-Methylpseudouridine also referred herein as methylpseudouridine).

The terms “nucleic acid sequence,” “polynucleic acid sequence,” “nucleotide sequence” are used herein interchangeably and have the identical meaning herein and refer to DNA or RNA. In some embodiments, a nucleic acid sequence is a polymer comprising or consisting of nucleotide monomers, which are covalently linked to each other by phosphodiester-bonds of a sugar/phosphate-backbone. The terms “nucleic acid sequence,” “polynucleic acid sequence,” and “nucleotide sequence” may encompass unmodified nucleic acid sequences, i.e., comprise unmodified nucleotides or natural nucleotides. The terms “nucleic acid sequence,” “polynucleic acid sequence,” and “nucleotide sequence” may also encompass modified nucleic acid sequences, such as base-modified, sugar-modified or backbone-modified etc., DNA or RNA.

The terms “natural nucleotide” and “canonical nucleotide” are used herein interchangeably and have the identical meaning herein and refer to the naturally occurring nucleotide bases adenine (A), guanine (G), cytosine (C), uracil (U), thymine (T).

The term “unmodified nucleotide” is used herein to refer to natural nucleotides which are not naturally modified e.g., which are not epigenetically or post-transcriptionally modified in vivo. Preferably the term “unmodified nucleotides” is used herein to refer to natural nucleotides which are not naturally modified e.g., which are not epigenetically or post-transcriptionally modified in vivo and which are not chemically modified e.g. which are not chemically modified in vitro.

The term “modified nucleotide” is used herein to refer to naturally modified nucleotides such as epigenetically or post-transcriptionally modified nucleotides and to chemically modified nucleotides e.g., nucleotides which are chemically modified in vitro.

### Recombinant RNA Constructs

Provided herein are compositions and methods for treating cancers, comprising recombinant polynucleic acid or RNA constructs comprising a gene of interest and a genetic element that reduces expression of another gene by binding to a target RNA. Also provided herein are compositions and methods to modulate expression of two or more genes simultaneously using a single RNA transcript. An example of the genetic element that reduces expression of another gene can include a small interfering RNA (siRNA) capable of binding to a target mRNA.

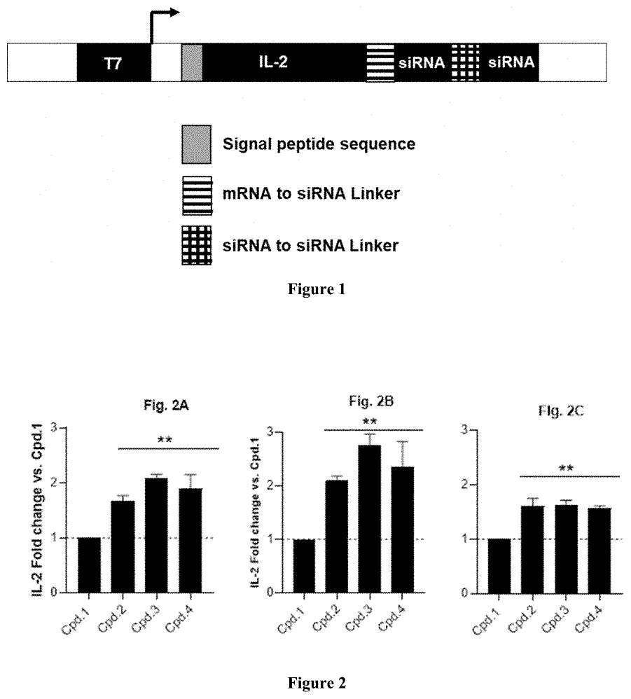

Further provided herein are recombinant polynucleic acid or RNA constructs comprising a gene of interest and a genetic element that reduces expression of another gene such as siRNA, wherein the gene of interest and the genetic element that reduces expression of another gene such as siRNA may be present in a sequential manner from the 5′ to 3′ direction, as illustrated in FIG. 1, or from 3′ to 5′ direction. In one example, the gene of interest can be present 5′ to or upstream of the genetic element that reduces expression of another gene such as siRNA, and the gene of interest can be linked to siRNA by a linker (mRNA to siRNA/shRNA linker, can be also referred s a “spacer”), as illustrated in FIG. 1. In another example, the gene of interest may be present 3′ to or downstream of the genetic element that reduces expression of another gene such as siRNA, and siRNA can be linked to the gene of interest by a linker (siRNA/shRNA to mRNA linker, can be also referred s a “spacer”). Recombinant polynucleic acid or RNA constructs provided herein may comprise more than one species of siRNAs and each of more than one species of siRNAs can be linked by a linker (siRNA to siRNA or shRNA to shRNA linker). In some embodiments, the sequence of mRNA to siRNA (or siRNA to mRNA) linker and the sequence of siRNA to siRNA (or shRNA to shRNA) linker may be different. In some embodiments, the sequence of mRNA to siRNA/shRNA (or siRNA/shRNA to mRNA) linker and the sequence of siRNA to siRNA (or shRNA to shRNA) linker may be the same. Recombinant polynucleic acid or RNA constructs provided herein may comprise more than one gene of interest and each of more than one gene of interest can be linked by a linker (mRNA to mRNA linker). As an example of a gene of interest, interleukin 2 (IL-2) is shown in FIG. 1. IL-2 comprises a signal peptide sequence at the N-terminus. IL-2 may comprise unmodified (WT) signal peptide sequence or modified signal peptide sequence. Recombinant polynucleic acid constructs provided herein may also comprise a promoter sequence for RNA polymerase binding. As an example, T7 promoter for T7 RNA polymerase binding is shown in FIG. 1.

Recombinant RNA constructs provided herein may comprise multiple copies of a gene of interest, wherein each of the multiple copies of a gene of interest encodes the same protein. Also provided herein are compositions comprising recombinant RNA constructs comprising multiple genes of interest, wherein, each of the multiple genes of interest encodes a different protein. Recombinant RNA constructs provided herein may comprise multiple species of siRNAs (e.g., at least two species of siRNAs), wherein each of the multiple species of siRNAs is capable of binding to the same target RNA. In some embodiments, each of the multiple species of siRNAs may bind to the same region of the same target RNA. In some embodiments, each of the multiple species of siRNAs may bind to a different region of the same target RNA. In some embodiments, some of the multiple species of siRNAs may bind to the same target RNA and some of the multiple species of siRNAs may bind to a different region of the same target RNA. Also provided herein are recombinant RNA constructs comprising multiple species of siRNAs, wherein each of the multiple species of siRNAs is capable of binding to a different target RNA. In some embodiments, the target RNA is a messenger (mRNA).

Provided herein are compositions comprising recombinant RNA constructs comprising a first RNA linked to a second RNA, wherein the first RNA encodes for a cytokine, and wherein the second RNA encodes for a genetic element that reduces expression of a gene associated with tumor proliferation, angiogenesis, or recognition by the immune system. In one example, the first RNA may be an mRNA encoding a cytokine and can increase cytokine protein levels. In another example, the second RNA or the genetic element that reduces expression of a gene associated with tumor proliferation, angiogenesis, or recognition by the immune system in compositions described herein can include a small interfering RNA (siRNA) capable of binding to a target mRNA. In some embodiments, a target mRNA may be an mRNA of a gene associated with tumor proliferation, angiogenesis, or recognition by the immune system, and can downregulate protein expression of the target mRNA.

A recombinant polynucleic acid or a recombinant RNA can refer to a polynucleic acid or RNA that is not naturally occurring and is synthesized or manipulated in vitro. A recombinant polynucleic acid or RNA can be synthesized in a laboratory and can be prepared by using recombinant DNA or RNA technology by using enzymatic modification of DNA or RNA, such as enzymatic restriction digestion, ligation, cloning, and/or in vitro transcription. A recombinant polynucleic acid can be transcribed in vitro to produce a messenger RNA (mRNA) and recombinant mRNAs can be isolated, purified, and used for transfection into a cell. A recombinant polynucleic acid or RNA used herein can encode a protein, polypeptide, a target motif, a signal peptide, and/or a non-coding RNA such as small interfering RNA (siRNA). In some embodiments, under suitable conditions, a recombinant polynucleic acid or RNA can be incorporated into a cell and expressed within the cell.

Recombinant RNA constructs provided herein may comprise more than one nucleic acid sequences encoding a gene of interest. For example, recombinant RNA constructs may comprise 2, 3, 4, 5, 6, 7, 8, 9, 10, 11, 12, 13, 14, 15, or more nucleic acid sequences encoding a gene of interest. In some instances, each of the two or more nucleic acid sequences may encode the same gene of interest, wherein the mRNA encoded by the same gene of interest is different from the siRNA target mRNA. In some instances, each of the two or more nucleic acid sequences may encode a different gene of interest, wherein the mRNA encoded by the different gene of interest is not a target of siRNA encoded in the same RNA construct. In some instances, recombinant RNA constructs may comprise three or more nucleic acid sequences encoding a gene of interest, wherein each of the three or more nucleic acid sequences may encode the same gene of interest or a different gene of interest, and wherein mRNAs encoded by the same or the different gene of interest are not a target of siRNA encoded in the same RNA construct. For example, recombinant RNA constructs may comprise four nucleic acid sequences encoding a gene of interest, wherein three of the four nucleic acid sequences encode the same gene of interest and one of the four nucleic acid sequences encodes a different gene of interest, and wherein mRNAs encoded by the same or different gene of interest are not a target of siRNA encoded in the same RNA construct.

Recombinant RNA constructs provided herein may comprise more than one species of siRNA targeting an mRNA of a gene associated with tumor proliferation, angiogenesis, or recognition by the immune system. For example, recombinant RNA constructs provided herein may comprise 1-10 species of siRNA targeting the same mRNA or different mRNAs. In some instances, each of the 1-10 species of siRNA targeting the same mRNA may comprise the same sequence, i.e. each of the 1-10 species of siRNA binds to the same region of the target mRNA. In some instances, each of the 1-10 species of siRNA targeting the same mRNA may comprise different sequences, i.e. each of the 1-10 species of siRNA binds to different regions of the target mRNA. Recombinant RNA constructs provided herein may comprise at least two species of siRNA targeting an mRNA of a gene associated with tumor proliferation, angiogenesis, or recognition by the immune system. For instance, recombinant RNA constructs provided herein, may comprise 3 species of siRNA targeting one mRNA and each of the 3 species of siRNA comprise the same nucleic acid sequence to target the same region of the mRNA. In this example, each of the 3 species of siRNA may comprise the same nucleic acid sequence to target exon 1. In another example, each of the 3 species of siRNA may comprise different nucleic acid sequence to target different regions of the mRNA. In this example, one of the 3 species of siRNA may comprise a nucleic acid sequence targeting exon 1 and another one of the 3 species of siRNA may comprise a nucleic acid sequence targeting exon 2, etc. In yet another example, each of the 3 species of siRNA may comprise different nucleic acid sequence to target different mRNAs. In all aspects, siRNAs in recombinant RNA constructs provided herein may not affect the expression of the gene of interest such as cytokine, expressed by the mRNA in the same RNA construct compositions.

Provided herein are compositions comprising recombinant RNA constructs, comprising a first RNA encoding for a cytokine and a second RNA encoding for a genetic element that reduces expression of a gene associated with tumor proliferation, angiogenesis, or recognition by the immune system. The first RNA and second RNA in compositions described herein may be linked by a linker. In some instances, compositions comprising the first RNA and the second RNA further comprises a nucleic acid sequence encoding for the linker. The linker can be from about 6 to about 50 nucleotides in length. For example, the linker can be at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or at least about 40 nucleotides in length. For example, the linker can be at most about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or at most about 50 nucleotides in length. In some instances, a tRNA linker can be used. The tRNA system is evolutionarily conserved cross living organism and utilizes endogenous RNases P and Z to process multicistronic constructs (Dong et al., 2016). In some instances, the tRNA linker described herein may comprise a nucleic acid sequence comprising AACAAAGCACCAGTGGTCTAGTGGTAGAATAGTACCCTGCCACGGTACAGACCC GGGTTCGATTCCCGGCTGGTGCA (SEQ ID NO: 20). In some instances, a linker comprising a nucleic acid sequence comprising ATAGTGAGTCGTATTAACGTACCAACAA (SEQ ID NO: 21) may be used to link the first RNA and the second RNA.

Recombinant RNA constructs provided herein may further comprise a 5′ cap, a Kozak sequence, and/or internal ribosome entry site (IRES), and/or a poly(A) tail at the 3′ end in a particular in order to improve translation. In some instances, recombinant RNA constructs may further comprise regions promoting translation known to any skilled artisan. Non-limiting examples of the 5′ cap can include an anti-reverse CAP analog, Clean Cap, Cap 0, Cap 1, Cap 2, or Locked Nucleic Acid cap (LNA-cap). In some instances, 5′ cap may comprise m₂^{7,3′-O}G(5)ppp(5′)G, m7G, m7G(5′)G, m7GpppG, or m7GpppGm.

Recombinant RNA constructs provided herein may further comprise a poly(A) tail. In some instances, the poly(A) tail comprises 1 to 220 base pairs of poly(A) (SEQ ID NO: 150). For example, the poly(A) tail comprises 1, 3, 5, 8, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, or 220 base pairs of poly(A) (SEQ ID NO: 150). In some embodiments, the poly(A) tail comprises 1 to 20, 1 to 40, 1 to 60, 1 to 80, 1 to 100, 1 to 120, 1 to 140, 1 to 160, 1 to 180, 1 to 200, 1 to 220, 20 to 40, 20 to 60, 20 to 80, to 100, 20 to 120, 20 to 140, 20 to 160, 20 to 180, 20 to 200, 20 to 220, 40 to 60, 40 to 80, to 100, 40 to 120, 40 to 140, 40 to 160, 40 to 180, 40 to 200, 40 to 220, 60 to 80, 60 to 100, 60 to 120, 60 to 140, 60 to 160, 60 to 180, 60 to 200, 60 to 220, 80 to 100, 80 to 120, 80 to 140, 80 to 160, 80 to 180, 80 to 200, 80 to 220, 100 to 120, 100 to 140, 100 to 160, 100 to 180, 100 to 200, 100 to 220, 120 to 140, 120 to 160, 120 to 180, 120 to 200, 120 to 220, 140 to 160, 140 to 180, 140 to 200, 140 to 220, 160 to 180, 160 to 200, 160 to 220, 180 to 200, 180 to 220, or 200 to 220 base pairs of poly(A) (SEQ ID NO: 150). In some embodiments, the poly(A) tail comprises 1, 20, 40, 60, 80, 100, 120, 140, 160, 180, 200, or 220 base pairs of poly(A) (SEQ ID NO: 150). In some embodiments, the poly(A) tail comprises at least 1, 20, 40, 60, 80, 100, 120, 140, 160, 180, or at least 200 base pairs of poly(A) (SEQ ID NO: 151). In some embodiments, the poly(A) tail comprises at most 20, 40, 60, 80, 100, 120, 140, 160, 180, 200, or at most 220 base pairs of poly(A) (SEQ ID NO: 152). In some embodiments, the poly(A) tail comprises 120 base pairs of poly(A) (SEQ ID NO: 153).

Recombinant RNA constructs provided herein may further comprise a Kozak sequence. A Kozak sequence may refer to a nucleic acid sequence motif that functions as a protein translation initiation site. Kozak sequences are described at length in the literature, e.g., by Kozak, M., Gene 299(1-2):1-34, incorporated herein by reference herein in its entirety. In some embodiments, the Kozak sequence described herein may comprise a sequence comprising GCCACC (SEQ ID NO: 19). In some embodiments, recombinant RNA constructs provided herein may further comprise a nuclear localization signal (NLS).

Recombinant RNA constructs described herein may include one or more nucleotide variants, including nonstandard nucleotide(s), non-natural nucleotide(s), nucleotide analog(s), and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-methyladenosine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine, N1-methylpseudouridine, and the like. In some cases, nucleotides may include modifications in their phosphate moieties, including modifications to a triphosphate moiety. Non-limiting examples of such modifications include phosphate chains of greater length and modifications with thiol moieties. In some embodiments, phosphate chains can comprise 4, 5, 6, 7, 8, 9, 10 or more phosphate moieties. In some embodiments, thiol moieties can include but are not limited to alpha-thiotriphosphate and beta-thiotriphosphates. In some embodiments, a recombinant RNA construct described herein does not comprise 5-methylcytosine and/or N6-methyladenosine.

Recombinant RNA constructs described herein may be modified at the base moiety, sugar moiety, or phosphate backbone. For example, modifications can be at one or more atoms that typically are available to form a hydrogen bond with a complementary nucleotide and/or at one or more atoms that are not typically capable of forming a hydrogen bond with a complementary nucleotide. In some embodiments, backbone modifications include, but are not limited to, a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delay nuclease degradation of oligonucleotides. A phosphorodiamidate linkage (N3′→P5′) allows prevents nuclease recognition and degradation. In some embodiments, backbone modifications include having peptide bonds instead of phosphorous in the backbone structure, or linking groups including carbamate, amides, and linear and cyclic hydrocarbon groups. For example, N-(2-aminoethyl)-glycine units may be linked by peptide bonds in a peptide nucleic acid. Oligonucleotides with modified backbones are reviewed in Micklefield, Backbone modification of nucleic acids: synthesis, structure and therapeutic applications, Curr. Med. Chem., 8 (10): 1157-79, 2001 and Lyer et al., Modified oligonucleotides-synthesis, properties and applications, Curr. Opin. Mol. Ther., 1 (3): 344-358, 1999.

Recombinant RNA constructs provided herein may comprise a combination of modified and unmodified nucleotides. In some instances, the adenosine-, guanosine-, and cytidine-containing nucleotides are unmodified or partially modified. In some instances, for modified RNA constructs, 1%, 5%, 10%, 20%, 30%, 40%, 50%, 60%, 70%, 80%, 90%, or 100% of uridine nucleotides may be modified. In some embodiments, 5% to 25% of uridine nucleotides are modified in recombinant RNA constructs. Non-limiting examples of the modified uridine nucleotides may comprise pseudouridines, N¹-Methylpseudouridines, or N1-methylpseudo-UTP and any modified uridine nucleotides known in the art may be utilized. In some embodiments, recombinant RNA constructs may contain a combination of modified and unmodified nucleotides, wherein 1%, 5%, 10%, 20%, 30%, 40%, 50%, 60%, 70%, 80%, 90%, or 100% of uridine nucleotides may comprise pseudouridines, N¹-Methylpseudouridines, N1-methylpseudo-UTP, or any other modified uridine nucleotide known in the art. In some embodiments, recombinant RNA constructs may contain a combination of modified and unmodified nucleotides, wherein 1%, 5%, 10%, 20%, 30%, 40%, 50%, 60%, 70%, 80%, 90%, or 100% of the uridine nucleotides may comprise N¹-Methylpseudouridines.

Recombinant RNA constructs provided herein may be codon-optimized. In general, codon optimization refers to a process of modifying a nucleic acid sequence for expression in a host cell of interest by replacing at least one codon (e.g., more than 1, 2, 3, 4, 5, 10, 15, 20, 25, 50, or more codons) of a native sequence with codons that are more frequently or most frequently used in the genes of that host cell while maintaining the native amino acid sequence. Codon usage tables are readily available, for example, at the “Codon Usage Database,” and these tables can be adapted in a number of ways. Computer algorithms for codon optimizing a particular sequence for expression in a particular host cell are also available, such as Gene Forge® (Aptagen, PA) and GeneOptimizer® (ThermoFischer, MA) which is preferred. In some embodiments, recombinant RNA constructs may not be codon-optimized.

In some instances, recombinant RNA constructs may comprise a nucleic acid sequence comprising a sequence selected from the group consisting of SEQ ID NOs: 1-17 and 125-141.

RNA Interference and Small Interfering RNA (siRNA)

RNA interference (RNAi) or RNA silencing is a process in which RNA molecules inhibit gene expression or translation, by neutralizing target mRNA molecules. RNAi process is described in Mello & Conte (2004) Nature 431, 338-342, Meister & Tuschl (2004) Nature 431, 343-349, Hannon & Rossi (2004) Nature 431, 371-378, and Fire (2007) Angew. Chem. Int. Ed. 46, 6966-6984. Briefly, in a natural process, the reaction initiates with a cleavage of long double-stranded RNA (dsRNA) into small dsRNA fragments or siRNAs with a hairpin structure (i.e., shRNAs) by a dsRNA-specific endonuclease Dicer. These small dsRNA fragments or siRNAs are then integrated into RNA-induced silencing complex (RISC) and guide the RISC to the target mRNA sequence. During interference, the siRNA duplex unwinds, and the antisense strand remains in complex with RISC to lead RISC to the target mRNA sequence to induce degradation and subsequent suppression of protein translation. Unlike commercially available synthetic siRNAs, siRNAs in the present invention can utilize endogenous Dicer and RISC pathway in the cytoplasm of a cell to get cleaved from recombinant RNA constructs (e.g., recombinant RNA constructs comprising an mRNA and one or more siRNAs) after cellular uptake and follow the natural process detailed above, as siRNAs in the recombinant RNA constructs of the present invention may comprise a hairpin loop structure. In addition, as the rest of the recombinant RNA constructs (i.e., mRNA) is left intact after cleavage of siRNAs by Dicer, the desired protein expression from the gene of interest in the recombinant RNA constructs of the present invention is attained.

Provided herein are compositions comprising recombinant RNA constructs comprising at least one nucleic acid sequence comprising a siRNA capable of binding to a target RNA. In some instances, the target RNA is an mRNA. In some embodiments, the siRNA is capable of binding to a target mRNA in the 5′ untranslated region. In some embodiments, the siRNA is capable of binding to a target mRNA in the 3′ untranslated region. In some embodiments, the siRNA is capable of binding to a target mRNA in an exon. In some instances, the target RNA is a noncoding RNA. In some embodiments, recombinant RNA constructs may comprise a nucleic acid sequence comprising a sense siRNA strand. In some embodiments, recombinant RNA constructs may comprise a nucleic acid sequence comprising an anti-sense siRNA strand. In some embodiments, recombinant RNA constructs may comprise a nucleic acid sequence comprising a sense siRNA strand and a nucleic acid sequence comprising an anti-sense siRNA strand. Details of siRNA comprised in the present invention are described in Cheng, et al. (2018) J. Mater. Chem. B., 6, 4638-4644, which is incorporated by reference herein.

For example, in some instances, recombinant RNA constructs may comprise at least 1 species of siRNA, i.e., a nucleic acid sequence comprising a sense strand of siRNA and a nucleic acid sequence comprising an anti-strand of siRNA. 1 species of siRNA, as described herein, can refer to 1 species of sense strand siRNA and 1 species of anti-sense strand siRNA. In some instances, recombinant RNA constructs may comprise more than 1 species of siRNA, e.g., 2, 3, 4, 5, 6, 7, 8, 9, 10, or more species of siRNA comprising a sense strand of siRNA and an anti-strand of siRNA. In some embodiments, recombinant RNA constructs may comprise 1 to 20 species of siRNA. In some embodiments, recombinant RNA constructs may comprise at least 1, 2, 3, 4, 5, 6, 7, 8, 9, or at least 10 species of siRNA. In some embodiments, recombinant RNA constructs may comprise at most 3, 4, 5, 6, 7, 8, 9, 10, 11, 12, 13, 14, 15, 16, 17, 18, 19, or at most 20 species of siRNA. In a preferred embodiment, recombinant RNA constructs described herein comprise at least 2 species of siRNA. In another preferred embodiment, recombinant RNA constructs described herein comprise at least 3 species of siRNA.

Provided herein are compositions of recombinant RNA constructs comprising 1-20 or more siRNA species, wherein each of the 1-20 or more siRNA species is capable of binding to a target RNA. In some embodiments, a target RNA is an mRNA or a non-coding RNA. In some instances, each of the siRNA species binds to the same target RNA. In one instance, each of the siRNA species may comprise the same sequence and bind to the same region or sequence of the same target RNA. For example, recombinant RNA constructs may comprise 1, 2, 3, 4, 5, or more siRNA species and each of the 1, 2, 3, 4, 5, or more siRNA species comprise the same sequence targeting the same region of a target RNA, i.e. recombinant RNA constructs may comprise 1, 2, 3, 4, 5, or more redundant species of siRNA. In another instance, each of the siRNA species may comprise a different sequence and bind to a different region or sequence of the same target RNA. For example, recombinant RNA constructs may comprise 1, 2, 3, 4, 5, or more siRNA species and each of the 1, 2, 3, 4, 5, or more siRNA species may comprise a different sequence targeting a different region of the same target RNA. In this example, one siRNA of the 1, 2, 3, 4, 5, or more siRNA species may target exon 1 and another siRNA of the 1, 2, 3, 4, 5, or more siRNA species may target exon 2 of the same mRNA, etc. In some instances, recombinant RNA constructs may comprise 1, 2, 3, 4, 5, or more siRNA species and 2 of the 1, 2, 3, 4, 5, or more siRNA species may comprise the same sequence and bind to the same regions of the target RNA and 3 or more of the 1, 2, 3, 4, 5, or more siRNA species may comprise a different sequence and bind to different regions of the same target RNA. In some instances, each of the siRNA species binds to a different target RNA. In some embodiments, a target RNA may be an mRNA or a non-coding RNA, etc.

Provided herein are compositions of recombinant RNA constructs comprising 1-20 or more siRNA species, wherein each of the 1-20 or more siRNA species are connected by a linker. In some instances, the linker may be a non-cleavable linker. In some instances, the linker may be a cleavable linker such as a self-cleavable linker. In some instances, the linker may be a tRNA linker. The tRNA system is evolutionarily conserved across living organism and utilizes endogenous RNases P and Z to process multicistronic constructs (Dong et al., 2016). In some embodiments, the tRNA linker may comprise a nucleic acid sequence comprising AACAAAGCACCAGTGGTCTAGTGGTAGAATAGTACCCTGCCACGGTACAGACCC GGGTTCGATTCCCGGCTGGTGCA (SEQ ID NO: 20). In some embodiments, a linker comprising a nucleic acid sequence comprising TTTATCTTAGAGGCATATCCCTACGTACCAACAA (SEQ ID NO: 22) may be used to connect different siRNA species.

In some instances, specific binding of an siRNA to its mRNA target results in interference with the normal function of the target mRNA to cause a modulation, e.g., downregulation, of function and/or activity, and wherein there is a sufficient degree of complementarity to avoid non-specific binding of the siRNA to non-target nucleic acid sequences under conditions in which specific binding is desired, i.e. under physiological conditions in the case of in vivo assays or therapeutic treatment, and under conditions in which assays are performed in the case of in vitro assays.

A protein as used herein can refer to molecules typically comprising one or more peptides or polypeptides. A peptide or polypeptide is typically a chain of amino acid residues, linked by peptide bonds. A peptide usually comprises between 2 and 50 amino acid residues. A polypeptide usually comprises more than 50 amino acid residues. A protein is typically folded into 3-dimensional form, which may be required for the protein to exert its biological function. A protein as used herein can include a fragment of a protein, a variant of a protein, and fusion proteins. A fragment may be a shorter portion of a full-length sequence of a nucleic acid molecule like DNA, RNA, or a protein. Accordingly, a fragment, typically, comprises a sequence that is identical to the corresponding stretch within the full-length sequence. In some embodiments, a fragment of a sequence may comprise at least 5% to at least 80% of a full-length nucleotide or amino acid sequence from which the fragment is derived. In some embodiments, a protein can be a mammalian protein. In some embodiments, a protein can be a human protein. In some embodiments, a protein may be a protein secreted from a cell. In some embodiments, a protein may be a protein on cell membranes. In some embodiments, a protein as referred to herein can be a protein that is secreted and acts either locally or systemically as a modulator of target cell signaling via receptors on cell surfaces, often involved in immunologic reactions or other host proteins involved in viral infection. Nucleotide and amino acid sequences of proteins useful in the context of the present invention, including proteins that are encoded by a gene of interest, are known in the art and available in the literature. For example, Nucleotide and amino acid sequences of proteins useful in the context of the present invention, including proteins that are encoded by a gene of interest are available in the UniProt database.

Provided herein are compositions of recombinant RNA constructs comprising an siRNA capable of binding to a target mRNA to modulate expression of the target mRNA. In some instances, expression of the target mRNA (e.g., the level of protein encoded by the target mRNA) is downregulated by the siRNA capable of binding to the target mRNA. In some embodiments, expression of the target mRNA is inhibited by the siRNA capable of binding to the target mRNA. Inhibition or downregulation of expression of the target mRNA, as described herein, can refer to, but is not limited to, interference with the target mRNA to interfere with translation of the protein from the target mRNA; thus, inhibition or downregulation of expression of the target mRNA can refer to, but is not limited to, a decreased level of proteins expressed from the target mRNA compared to a level of proteins expressed from the target mRNA in the absence of recombinant RNA constructs comprising siRNA capable of binding to the target mRNA. Levels of protein expression can be measured by using any methods well known in the art and these include, but are not limited to Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Provided herein are compositions comprising recombinant RNA constructs comprising at least one nucleic acid sequence comprising siRNA capable of binding to a target mRNA and at least one nucleic acid sequence encoding a gene of interest wherein the target mRNA is different from an mRNA encoded by the gene of interest. Provided herein are compositions comprising recombinant RNA constructs comprising at least one nucleic acid sequence comprising siRNA capable of binding to a target mRNA and at least one nucleic acid sequence encoding a gene of interest wherein the siRNA does not affect expression of the gene of interest. In some instances, the siRNA is not capable of binding to an mRNA encoded by the gene of interest. In some instances, the siRNA does not inhibit the expression of the gene of interest. In some instances, the siRNA does not downregulate the expression of the gene of interest. Inhibiting or downregulating the expression of the gene of interest, as described herein, can refer to, but is not limited to, interfering with translation of proteins from recombinant RNA constructs; thus, inhibiting or downregulating the expression of the gene of interest can refer to, but is not limited to, a decreased level of protein compared to a level of protein expressed in the absence of recombinant RNA constructs comprising siRNA capable of binding to the target mRNA. Levels of protein expression can be measured by using any methods well known in the art and these include, but are not limited to Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Provided herein are compositions comprising recombinant RNA constructs comprising at least one nucleic acid sequence comprising a siRNA capable of binding to a target mRNA. A list of non-limiting examples of target mRNAs that the siRNA is capable of binding to includes an mRNA of a gene associated with tumor proliferation, angiogenesis, or recognition by the immune system. For example, the target mRNA may be an mRNA encoding vascular endothelial growth factor (VEGF), VEGFA, an isoform of VEGFA, placental growth factor (PIGF), a fragment thereof, or a functional variant thereof. A functional variant as used herein may refer to a full-length molecule, a fragment thereof, or a variant thereof. For example, a variant molecule may comprise a sequence modified by insertion, deletion, and/or substitution of one or more amino acids, in the case of protein sequence, or one or more nucleotides, in the case of nucleic acid sequence. For example, a variant molecule may comprise or encode a mutant protein, including, but not limited to, a gain-of-function or a loss-of-function mutant. A list of non-limiting examples of VEGFA isoforms is shown in Table A.

TABLE A
List of VEGFA Isoforms

| VEGFA Isoforms | UniProt Database # |
|---|---|
| VEGF111 | P15692-10 |
| VEGF121 | P15692-9 |
| VEGF145 | P15692-6 |
| VEGF148 | P15692-5 |
| VEGF165 | P15692-4 |
| VEGF165B | P15692-8 |
| VEGF183 | P15692-3 |
| VEGF189 | P15692-2 |
| VEGF206 | P15692-1 |
| L-VEGF121 | P15692-12 |
| L-VEGF165 | P15692-11 |
| L-VEGF189 | P15692-13 |
| L-VEGF206 | P15692-14 |
| Isoform 15 | P15692-15 |
| Isoform16 | P15692-16 |
| Isoform 17 | P15692-17 |
| Isoform 18 | P15692-18 |

In some embodiments, VEGFA comprises a sequence listed in SEQ ID NO: 34. An exemplary PIGF sequence is shown below:

PIGF NCBI Reference Sequence: NM_001207012.1
(SEQ ID NO: 123)
CCTCGCACGC ACTGCGGGCT CCGGCGCTGC GGGCTGGCCG
GGCGCTGCGG GCTGACCGGG CGCTCCGGGA ACTCGGCTCG
GGAACCTCGT CTGCGGTGGG CGGGGCCGGC CCGGAGCCCC
GCCCCGGCTC AGTCCCTGAA ACCCAGGCGC GGACCGGCTG
CAGTCTCAGA AGGGAGCTGC TGTCTGCGGA GGAAACTGCA
TCGACGGACG GCCGCCCAGC TACGGGAGGA CCTGGAGTGG
CACTGGGCGC CCGACGGACC ATCCCCGGGA CCCGCCTGCC
CCTCGGCGCC CCGCCCCGCC GGGCCGCTCC CCGTCGGGTT
CCCCAGCCAC AGCCTTACCT ACGGGCTCCT GACTCCGCAA
GGCTTCCAGA AGATGCTCGA ACCACCGGCC GGGGCCTCGG
GGCAGCAGTG AGGGAGGCGT CCAGCCCCCC ACTCAGCTCT
TCTCCTCCTG TGCCAGGGGC TCCCCGGGGG ATGAGCATGG
TGGTTTTCCC TCGGAGCCCC CTGGCTCGGG ACGTCTGAGA
AGATGCCGGT CATGAGGCTG TTCCCTTGCT TCCTGCAGCT
CCTGGCCGGG CTGGCGCTGC CTGCTGTGCC CCCCCAGCAG
TGGGCCTTGT CTGCTGGGAA CGGCTCGTCA GAGGTGGAAG
TGGTACCCTT CCAGGAAGTG TGGGGCCGCA GCTACTGCCG
GGCGCTGGAG AGGCTGGTGG ACGTCGTGTC CGAGTACCCC
AGCGAGGTGG AGCACATGTT CAGCCCATCC TGTGTCTCCC
TGCTGCGCTG CACCGGCTGC TGCGGCGATG AGAATCTGCA
CTGTGTGCCG GTGGAGACGG CCAATGTCAC CATGCAGCTC
CTAAAGATCC GTTCTGGGGA CCGGCCCTCC TACGTGGAGC
TGACGTTCTC TCAGCACGTT CGCTGCGAAT GCCGGCCTCT
GCGGGAGAAG ATGAAGCCGG AAAGGTGCGG CGATGCTGTT
CCCCGGAGGT AACCCACCCC TTGGAGGAGA GAGACCCCGC
ACCCGGCTCG TGTATTTATT ACCGTCACAC TCTTCAGTGA
CTCCTGCTGG TACCTGCCCT CTATTTATTA GCCAACTGTT
TCCCTGCTGA ATGCCTCGCT CCCTTCAAGA CGAGGGGCAG
GGAAGGACAG GACCCTCAGG AATTCAGTGC CTTCAACAAC
GTGAGAGAAA GAGAGAAGCC AGCCACAGAC CCCTGGGAGC
TTCCGCTTTG AAAGAAGCAA GACACGTGGC CTCGTGAGGG
GCAAGCTAGG CCCCAGAGGC CCTGGAGGTC TCCAGGGGCC
TGCAGAAGGA AAGAAGGGGG CCCTGCTACC TGTTCTTGGG
CCTCAGGCTC TGCACAGACA AGCAGCCCTT GCTTTCGGAG
CTCCTGTCCA AAGTAGGGAT GCGGATCCTG CTGGGGCCGC
CACGGCCTGG CTGGTGGGAA GGCCGGCAGC GGGCGGAGGG
GATCCAGCCA CTTCCCCCTC TTCTTCTGAA GATCAGAACA
TTCAGCTCTG GAGAACAGTG GTTGCCTGGG GGCTTTTGCC
ACTCCTTGTC CCCCGTGATC TCCCCTCACA CTTTGCCATT
TGCTTGTACT GGGACATTGT TCTTTCCGGC CAAGGTGCCA
CCACCCTGCC CCCCCTAAGA GACACATACA GAGTGGGCCC
CGGGCTGGAG AAAGAGCTGC CTGGATGAGA AACAGCTCAG
CCAGTGGGGA TGAGGTCACC AGGGGAGGAG CCTGTGCGTC
CCAGCTGAAG GCAGTGGCAG GGGAGCAGGT TCCCCAAGGG
CCCTGGCACC CCCACAAGCT GTCCCTGCAG GGCCATCTGA
CTGCCAAGCC AGATTCTCTT GAATAAAGTA TTCTAGTGTG
GAAACGCT

For example, the target mRNA may be an mRNA encoding MHC class I chain-related sequence A (MICA), MHC class I chain-related sequence B (MICB), endoplasmic reticulum protein (ERp5), a disintegrin and metalloproteinase (ADAM), matrix metalloproteinase (MMP), a fragment thereof, or a functional variant thereof. A functional variant as used herein may refer to a full-length molecule, a fragment thereof, or a variant thereof. For example, a variant molecule may comprise a sequence modified by insertion, deletion, and/or substitution of one or more amino acids, in the case of protein sequence, or one or more nucleotides, in the case of nucleic acid sequence. For example, a variant molecule may comprise or encode a mutant protein, including, but not limited to, a gain-of-function or a loss-of-function mutant. In some embodiments, the ADAM is ADAM 17. In some embodiments, the target mRNA may encode a decoy protein. In some embodiments the decoy protein is a soluble form of a cell receptor. In some embodiments, the decoy protein is soluble MICA, MICB, a fragment thereof, or a functional variant thereof. In some embodiments, the target mRNA may encode a protein involved in shedding of MICA and/or MICB from cell membranes. In some embodiments, the protein involved in shedding of MICA and/or MICB from cell membranes comprises ERp5, ADAM, MMP, a fragment thereof, or a functional variant thereof. In some embodiments, the protein involved in shedding of MICA and/or MICB from cell membranes comprises ADAM17, a fragment thereof, or a functional variant thereof. An exemplary sequence of ADAM17 is shown below:

ADAM17 NCBI Reference Sequence: NM_003183.6
(SEQ ID NO: 124)
AGCGGCGGCC GGAAGCTGGC TGAGCCGGCC TTTGGTAACG
CCACCTGCAC TTCTGGGGGC GTCGAGCCTG GCGGTAGAAT
CTTCCCAGTA GGCGGCGCGG GAGGGAAAAG AGGATTGAGG
GGCTAGGCCG GGCGGATCCC GTCCTCCCCC GATGTGAGCA
GTTTTCCGAA ACCCCGTCAG GCGAAGGCTG CCCAGAGAGG
TGGAGTCGGT AGCGGGGCCG GGAACATGAG GCAGTCTCTC
CTATTCCTGA CCAGCGTGGT TCCTTTCGTG CTGGCGCCGC
GACCTCCGGA TGACCCGGGC TTCGGCCCCC ACCAGAGACT
CGAGAAGCTT GATTCTTTGC TCTCAGACTA CGATATTCTC
TCTTTATCTA ATATCCAGCA GCATTCGGTA AGAAAAAGAG
ATCTACAGAC TTCAACACAT GTAGAAACAC TACTAACTTT
TTCAGCTTTG AAAAGGCATT TTAAATTATA CCTGACATCA
AGTACTGAAC GTTTTTCACA AAATTTCAAG GTCGTGGTGG
TGGATGGTAA AAACGAAAGC GAGTACACTG TAAAATGGCA
GGACTTCTTC ACTGGACACG TGGTTGGTGA GCCTGACTCT
AGGGTTCTAG CCCACATAAG AGATGATGAT GTTATAATCA
GAATCAACAC AGATGGGGCC GAATATAACA TAGAGCCACT
TTGGAGATTT GTTAATGATA CCAAAGACAA AAGAATGTTA
GTTTATAAAT CTGAAGATAT CAAGAATGTT TCACGTTTGC
AGTCTCCAAA AGTGTGTGGT TATTTAAAAG TGGATAATGA
AGAGTTGCTC CCAAAAGGGT TAGTAGACAG AGAACCACCT
GAAGAGCTTG TTCATCGAGT GAAAAGAAGA GCTGACCCAG
ATCCCATGAA GAACACGTGT AAATTATTGG TGGTAGCAGA
TCATCGCTTC TACAGATACA TGGGCAGAGG GGAAGAGAGT
ACAACTACAA ATTACTTAAT AGAGCTAATT GACAGAGTTG
ATGACATCTA TCGGAACACT TCATGGGATA ATGCAGGITT
TAAAGGCTAT GGAATACAGA TAGAGCAGAT TCGCATTCTC
AAGTCTCCAC AAGAGGTAAA ACCTGGTGAA AAGCACTACA
ACATGGCAAA AAGTTACCCA AATGAAGAAA AGGATGCTTG
GGATGTGAAG ATGTTGCTAG AGCAATTTAG CTTTGATATA
GCTGAGGAAG CATCTAAAGT TTGCTTGGCA CACCTTTTCA
CATACCAAGA TTTTGATATG GGAACTCTTG GATTAGCTTA
TGTTGGCTCT CCCAGAGCAA ACAGCCATGG AGGTGTTTGT
CCAAAGGCTT ATTATAGCCC AGTTGGGAAG AAAAATATCT
ATTTGAATAG TGGTTTGACG AGCACAAAGA ATTATGGTAA
AACCATCCTT ACAAAGGAAG CTGACCTGGT TACAACTCAT
GAATTGGGAC ATAATTTTGG AGCAGAACAT GATCCGGATG
GTCTAGCAGA ATGTGCCCCG AATGAGGACC AGGGAGGGAA
ATATGTCATG TATCCCATAG CTGTGAGTGG CGATCACGAG
AACAATAAGA TGTTTTCAAA CTGCAGTAAA CAATCAATCT
ATAAGACCAT TGAAAGTAAG GCCCAGGAGT GTTTTCAAGA
ACGCAGCAAT AAAGTTTGTG GGAACTCGAG GGTGGATGAA
GGAGAAGAGT GTGATCCTGG CATCATGTAT CTGAACAACG
ACACCTGCTG CAACAGCGAC TGCACGTTGA AGGAAGGTGT
CCAGTGCAGT GACAGGAACA GTCCTTGCTG TAAAAACTGT
CAGTTTGAGA CTGCCCAGAA GAAGTGCCAG GAGGCGATTA
ATGCTACTTG CAAAGGCGTG TCCTACTGCA CAGGTAATAG
CAGTGAGTGC CCGCCTCCAG GAAATGCTGA AGATGACACT
GTTTGCTTGG ATCTTGGCAA GTGTAAGGAT GGGAAATGCA
TCCCTTTCTG CGAGAGGGAA CAGCAGCTGG AGTCCTGTGC
ATGTAATGAA ACTGACAACT CCTGCAAGGT GTGCTGCAGG
GACCTTTCTG GCCGCTGTGT GCCCTATGTC GATGCTGAAC
AAAAGAACTT ATTTTTGAGG AAAGGAAAGC CCTGTACAGT
AGGATTTTGT GACATGAATG GCAAATGTGA GAAACGAGTA
CAGGATGTAA TTGAACGATT TTGGGATTTC ATTGACCAGC
TGAGCATCAA TACTTTTGGA AAGTTTTTAG CAGACAACAT
CGTTGGGTCT GTCCTGGTTT TCTCCTTGAT ATTTTGGATT
CCTTTCAGCA TTCTTGTCCA TTGTGTGGAT AAGAAATTGG
ATAAACAGTA TGAATCTCTG TCTCTGTTTC ACCCCAGTAA
CGTCGAAATG CTGAGCAGCA TGGATTCTGC ATCGGTTCGC
ATTATCAAAC CCTTTCCTGC GCCCCAGACT CCAGGCCGCC
TGCAGCCTGC CCCTGTGATC CCTTCGGCGC CAGCAGCTCC
AAAACTGGAC CACCAGAGAA TGGACACCAT CCAGGAAGAC
CCCAGCACAG ACTCACATAT GGACGAGGAT GGGTTTGAGA
AGGACCCCTT CCCAAATAGC AGCACAGCTG CCAAGTCATT
TGAGGATCTC ACGGACCATC CGGTCACCAG AAGTGAAAAG
GCTGCCTCCT TTAAACTGCA GCGTCAGAAT CGTGTTGACA
GCAAAGAAAC AGAGTGCTAA TTTAGTTCTC AGCTCTTCTG
ACTTAAGTGT GCAAAATATT TTTATAGATT TGACCTACAA
ATCAATCACA GCTTGTATTT TGTGAAGACT GGGAAGTGAC
TTAGCAGATG CTGGTCATGT GTTTGAACTT CCTGCAGGTA
AACAGTTCTT GTGTGGTTTG GCCCTTCTCC TTTTGAAAAG
GTAAGGTGAA GGTGAATCTA GCTTATTTTG AGGCTTTCAG
GTTTTAGTTT TTAAAATATC TTTTGACCTG TGGTGCAAAA
GCAGAAAATA CAGCTGGATT GGGTTATGAA TATTTACGTT
TTTGTAAATT AATCTTTTAT ATTGATAACA GCACTGACTA
GGGAAATGAT CAGTTTTTTT TTATACACTG TAATGAACCG
CTGAATATGA GGCATTTGGC ATTTATTTGT GATGACAACT
GGAATAGTTT TTTTTTTTTT TTTTTTTTTT TGCCTTCAAC
TAAAAACAAA GGAGATAAAT CTAGTATACA TTGTCTCTAA
ATTGTGGGTC TATTTCTAGT TATTACCCAG AGTTTTTATG
TAGCAGGGAA AATATATATC TAAATTTAGA AATCATTTGG
GTTAATATGG CTCTTCATAA TTCTAAGACT AATGCTCTCT
AGAAACCTAA CCACCTACCT TACAGTGAGG GCTATACATG
GTAGCCAGTT GAATTTATGG AATCTACCAA CTGTTTAGGG
CCCTGATTTG CTGGGCAGTT TTTCTGTATT TTATAAGTAT
CTTCATGTAT CCCTGTTACT GATAGGGATA CATGCTCTTA
GAAAATTCAC TATTGGCTGG GAGTGGTGGC TCATGCCTGT
AATCCCAGCA CTTGGAGAGG CTGAGGTTGC GCCACTACAC
TCCAGCCTGG GTGACAGAGT GAGACTCTGC CTCAAAAAAA
AAAAAAAAAA AAAAAAATTC ACTATCTACA AACCTAGAAT
ATTTAAAATA CAAAGATTGC CTGTTTTCAA ACACTATTGA
ATAAGAGGGT GAGATATTTC TTAACAACAA CAACAACAAA
AAAAACAGGT TGTTTTGAAT GTGATGAGCC AGCCAGGAGA
TAGAATACTA CCTGCCCTTA GGGTTGGGGG CTGTCCCCAC
AAGACTTGAT ACTTCAGAAA CCCTTTTTAT TGACCCACAA
GCAGATATTT GAATTACTTC TTACTTTATT GCTCCAGGAT
TCTGGATGGG CTGCATTTAC TGTGTGAAGG ATAAAAATCA
TTAGCCTGGA TTCTGATTTC TATAAATTGC CATTAAAAGC
TTTTTTTCCC CTAAGAACTG AAATGTGCTC ACCAGCCAAA
ACATTTTAAC TTGTAAATTT TGAGGGCAGT TAACCAAACC
TGTGACTAAT CATATCTCCT CCTACCCCCC ATTTCCAAGG
ACATTTGTTA CTCAGATACT TGTTATACTA ATACTTGAAC
TTGTACCTTA TGGTATTTGC TATCTTTTAA CTAGTCATGA
TATTCTTATA CTTTAGTTAC ACTTTTGGAA TTTGATACAA
GGTTGAGTGG GGTGTGTGGG TGTATGTATG AGTGAAACAG
TTCTCAAAAG AATGTAAGAA AAACCATTTT TATAAAATTG
TGACTTTTTA AAAACATAGT CTTTGTCATT TATAGAATTA
ACAAGCTGCT CAGGGTATAT TTTATAGCTG TAGCACTGAT
ATCTGCATTA ATAAATACTG TCGAAACACA A

For example, the target mRNA may be an mRNA encoding isocitrate dehydrogenase (IDH1), cyclin-dependent kinase 4 (CDK4), CDK6, epidermal growth factor receptor (EGFR), mechanistic target of rapamycin (mTOR), Kirsten rat sarcoma viral oncogene (KRAS), cluster of differentiation (CD155), programmed cell death-ligand 1 (PD-L1), or myc proto-oncogene (c-Myc), a fragment thereof, or a functional variant thereof. A functional variant as used herein may refer to a full-length molecule, a fragment thereof, or a variant thereof. For example, a variant molecule may comprise a sequence modified by insertion, deletion, and/or substitution of one or more amino acids, in the case of protein sequence, or one or more nucleotides, in the case of nucleic acid sequence. For example, a variant molecule may comprise or encode a mutant protein, including, but not limited to, a gain-of-function or a loss-of-function mutant.

In some embodiments, the target mRNA may encode a protein selected from the group consisting of VEGFA, an isoform of VEGFA, PIGF, MICA, MICB, ERp5, ADAM17, MMP, IDH1, CDK4, CDK6, EGFR, mTOR, KRAS, CD155, PD-L1, c-Myc, a fragment thereof, a functional variant thereof, and a combination thereof. In some embodiments, VEGFA mRNA comprises a sequence comprising SEQ ID NO: 36. In some embodiments, MICA mRNA comprises a sequence comprising SEQ ID NO: 39. In some embodiments, MICB mRNA comprises a sequence comprising SEQ ID NO: 42. In some embodiments, IDH1 mRNA comprises a sequence comprising SEQ ID NO: 51. In some embodiments, CDK4 mRNA comprises a sequence comprising SEQ ID NO: 54. In some embodiments, CDK6 mRNA comprises a sequence comprising SEQ ID NO: 57. In some embodiments, EGFR mRNA comprises a sequence comprising SEQ ID NO: 60. In some embodiments, mTOR mRNA comprises a sequence comprising SEQ ID NO: 63. In some embodiments, KRAS mRNA comprises a sequence comprising SEQ ID NO: 66. In some embodiments, CD155 mRNA comprises a sequence comprising SEQ ID NO: 72. In some embodiments, PD-L1 mRNA comprises a sequence comprising SEQ ID NO: 75. In some embodiments, c-Myc mRNA comprises a sequence comprising SEQ ID NO: 78.

### Gene of Interest

Provided herein are recombinant RNA constructs comprising one or more copies of nucleic acid sequence encoding a gene of interest. For example, recombinant RNA constructs may comprise 1, 2, 3, 4, 5, 6, 7, 8, 9, 10, or more copies of nucleic acid sequence encoding a gene of interest. In some instances, each of the 1, 2, 3, 4, 5, 6, 7, 8, 9, 10, or more copies of nucleic acid sequence encoding a gene of interest encodes the same gene of interest. In some instances, recombinant RNA constructs may comprise 1, 2, 3, 4, 5, 6, 7, 8, 9, 10, or more copies of nucleic acid sequence encoding a cytokine.

Also provided herein are recombinant RNA constructs comprising two or more copies of nucleic acid sequence encoding a gene of interest, wherein each of the two or more nucleic acid sequence may encode a different gene of interest. In some cases, each of the two or more nucleic acid sequences encoding different gene of interest may comprise a nucleic acid sequence encoding a secretory protein. In some cases, each of the two or more nucleic acid sequences encoding different gene of interest may comprise a nucleic acid sequence encoding a cytokine. In some embodiments, each of the two or more nucleic acid sequences encoding different gene of interest may encode a different cytokine. Further provided herein are recombinant RNA constructs comprising a linker. In some embodiments, the linker may connect each of the two or more nucleic acid sequences encoding a gene of interest. In some cases, the linker may be a non-cleavable linker. In some cases, the linker may be a cleavable linker. In some cases, the linker may be a self-cleavable linker. Non-limiting examples of the linker comprises a flexible linker, a 2A peptide linker (or 2A self-cleaving peptides) such as T2A, P2A, E2A, or F2A, and a tRNA linker, etc. The tRNA system is evolutionarily conserved across living organism and utilizes endogenous RNases P and Z to process multicistronic constructs (Dong et al., 2016). In some embodiments, the tRNA linker may comprise a nucleic acid sequence comprising

(SEQ ID NO: 20)
AACAAAGCACCAGTGGTCTAGTGGTAGAATAGTACCCTGCCACGGTACAG
ACCCGGGTTCGATTCCCGGCTGGTGCA.

Provided herein are recombinant RNA constructs comprising an RNA encoding for a gene of interest for modulating the expression of the gene of interest. For example, expression of a protein encoded by the mRNA of the gene of interest can be modulated. For example, the expression of the gene of interest is upregulated by expressing a protein encoded by mRNA of the gene of interest in recombinant RNA constructs. For example, the expression of the gene of interest is upregulated by increasing the level of protein encoded by mRNA of the gene of interest in recombinant RNA constructs. The level of protein expression can be measured by using any methods well known in the art and these include, but are not limited to Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Provided herein are recombinant RNA constructs comprising an RNA encoding for a gene of interest wherein the gene of the interest encodes a protein of interest. In some instances, the protein of interest is a therapeutic protein. In some instances, the protein of interest is of human origin i.e., is a human protein. In some instances, the gene of interest encodes a cytokine. In some embodiments, the cytokine comprises an interleukin. In some embodiments, the protein of interest is an interleukin 2 (IL-2), IL-12, IL-15, IL-7, a fragment thereof, or a functional variant thereof. A functional variant as used herein may refer to a full-length molecule, a fragment thereof, or a variant thereof. For example, a variant molecule may comprise a sequence modified by insertion, deletion, and/or substitution of one or more amino acids, in the case of protein sequence, or one or more nucleotides, in the case of nucleic acid sequence.

In some instances, interleukin 2 (IL-2) or IL-2 as used herein may refer to the natural sequence of human IL-2 (Uniprot database: P60568 or Q0GK43 and in the Genbank database: NM 000586.3), a fragment thereof, or a functional variant thereof. The natural DNA sequence encoding human IL-2 may be codon-optimized. The natural sequence of human IL-2 may consist of a signal peptide having 20 amino acids (nucleotides 1-60) and the mature human IL-2 having 133 amino acids (nucleotides 61-459) as shown in SEQ ID NO: 23. In some embodiments, the signal peptide is unmodified IL-2 signal peptide. In some embodiments, the signal peptide is IL-2 signal peptide modified by insertion, deletion, and/or substitution of at least one amino acid. In some embodiments, interleukin 2 (IL-2) or IL-2 as used herein may refer to the mature human IL-2. In some embodiments, a mature protein can refer to a protein synthesized in the endoplasmic reticulum and secreted via the Golgi apparatus in a cell expressing and secreting the protein. In some embodiments, a mature IL-2 may refer to an IL-2 protein synthesized in the endoplasmic reticulum and secreted via the Golgi apparatus in a cell expressing and secreting IL-2. In some embodiments, a mature human IL-2 may refer to an IL-2 protein synthesized in the endoplasmic reticulum and secreted via the Golgi apparatus in a human cell expressing and secreting human IL-2 and normally contains the amino acids encoded by nucleotide as shown in SEQ ID NO: 24. In some embodiments, IL-2 may comprise an IL-2 fragment, an IL-2 variant, an IL-2 mutein, or an IL-2 mutant. In some embodiments, the IL-2 fragment described herein may be at least partially functional, i.e., can perform an IL-2 activity at a similar or lower level compared to a wildtype or a full length IL-2. In some embodiments, the IL-2 fragment described herein may be fully functional, i.e., can perform an IL-2 activity at the same level compared to a wildtype or a full length IL-2. In some embodiments, the IL-2 variant, an IL-2 mutein, or the IL-2 mutant may comprise an IL-2 amino acid sequence modified by insertion, deletion, and/or substitution of at least one amino acid. In some embodiments, the IL-2 variant, an IL-2 mutein, or the IL-2 mutant may be at least partially functional, i.e., can perform an IL-2 activity at a similar or lower level compared to a wildtype IL-2. In some embodiments, the IL-2 variant, an IL-2 mutein, or the IL-2 mutant may be fully functional, i.e., can perform an IL-2 activity at the same level compared to a wildtype IL-2. In some embodiments, the IL-2 variant, an IL-2 mutein, or the IL-2 mutant may perform an IL-2 activity at a higher level compared to a wildtype IL-2.

The mRNA encoding IL-2 may refer to an mRNA comprising a nucleotide sequence encoding the propeptide of human IL-2 having 153 amino acids or a nucleotide sequence encoding the mature human IL-2 having 133 amino acids. The nucleotide sequence encoding the propeptide of human IL-2 and the nucleotide sequence encoding the mature human IL-2 may be codon-optimized. In some instances, recombinant RNA constructs, provided herein, may comprise 1 copy of IL-2 mRNA. In some instances, recombinant RNA constructs, provided herein, may comprise 2 or more copies of IL-2 mRNA.

In some instances, interleukin 12 (IL-12) or IL-12 as used herein may refer to the natural sequence of human IL-12 alpha (Genbank database: NM_000882.4), the natural sequence of human IL-12 beta (Genbank database: NM_002187.2), a fragment thereof, or a functional variant thereof. The natural DNA sequence encoding human IL-12 may be codon-optimized. The natural sequence of human IL-12 alpha may consist of a signal peptide having 22 amino acids and the mature human IL-12 having 197 amino acids as shown in SEQ ID NO: 43. In some embodiments, the signal peptide is unmodified IL-12 alpha signal peptide. In some embodiments, the signal peptide is IL-12 alpha signal peptide modified by insertion, deletion, and/or substitution of at least one amino acid. The natural sequence of human IL-12 beta may consist of a signal peptide having 22 amino acids and the mature human IL-12 having 306 amino acids as shown in SEQ ID NO: 46. In some embodiments, the signal peptide is unmodified IL-12 beta signal peptide. In some embodiments, the signal peptide is IL-12 beta signal peptide modified by insertion, deletion, and/or substitution of at least one amino acid.

In some embodiments, interleukin 12 (IL-12) or IL-12 as used herein may refer to the mature human IL-12 alpha. In some embodiments, interleukin 12 (IL-12) or IL-12 as used herein may refer to the mature human IL-12 beta. In some embodiments, a mature protein can refer to a protein synthesized in the endoplasmic reticulum and secreted via the Golgi apparatus in a cell expressing and secreting the protein. In some embodiments, a mature IL-12 may refer to an IL-12 alpha protein synthesized in the endoplasmic reticulum and secreted via the Golgi apparatus in a cell expressing and secreting IL-12. In some embodiments, a mature IL-12 may refer to an IL-12 beta protein synthesized in the endoplasmic reticulum and secreted via the Golgi apparatus in a cell expressing and secreting IL-12. In some embodiments, a mature human IL-12 may refer to an IL-12 alpha protein synthesized in the endoplasmic reticulum and secreted via the Golgi apparatus in a human cell expressing and secreting human IL-12 and normally contains the amino acids encoded by nucleotide as shown in SEQ ID NO: 44. In some embodiments, a mature human IL-12 may refer to an IL-12 beta protein synthesized in the endoplasmic reticulum and secreted via the Golgi apparatus in a human cell expressing and secreting human IL-12 and normally contains the amino acids encoded by nucleotide as shown in SEQ ID NO: 47.

In some embodiments, IL-12 alpha may comprise an IL-12 alpha fragment, an IL-12 alpha variant, an IL-12 alpha mutein, or an IL-12 alpha mutant. In some embodiments, the IL-12 alpha fragment described herein may be at least partially functional, i.e., can perform an IL-12 alpha activity at a similar or lower level compared to a wildtype or a full-length IL-12 alpha. In some embodiments, the IL-12 alpha fragment described herein may be fully functional, i.e., can perform an IL-12 alpha activity at the same level compared to a wildtype or a full-length IL-12 alpha. In some embodiments, the IL-12 alpha variant, an IL-12 alpha mutein, or the IL-12 alpha mutant may comprise an IL-12 alpha amino acid sequence modified by insertion, deletion, and/or substitution of at least one amino acid. In some embodiments, the IL-12 alpha variant, an IL-12 alpha mutein, or the IL-12 alpha mutant may be at least partially functional, i.e., can perform an IL-12 alpha activity at a similar or lower level compared to a wildtype IL-12 alpha. In some embodiments, the IL-12 alpha variant, an IL-12 alpha mutein, or the IL-12 alpha mutant may be fully functional, i.e., can perform an IL-12 alpha activity at the same level compared to a wildtype IL-12 alpha. In some embodiments, the IL-12 alpha variant, an IL-12 alpha mutein, or the IL-12 alpha mutant may perform an IL-12 alpha activity at a higher level compared to a wildtype IL-12 alpha.

In some embodiments, IL-12 beta may comprise an IL-12 beta fragment, an IL-12 beta variant, an IL-12 beta mutein, or an IL-12 beta mutant. In some embodiments, the IL-12 beta fragment described herein may be at least partially functional, i.e., can perform an IL-12 beta activity at a similar or lower level compared to a wildtype or a full-length IL-12 beta. In some embodiments, the IL-12 beta fragment described herein may be fully functional, i.e., can perform an IL-12 beta activity at the same level compared to a wildtype or a full-length IL-12 beta. In some embodiments, the IL-12 beta variant, an IL-12 beta mutein, or the IL-12 beta mutant may comprise an IL-12 beta amino acid sequence modified by insertion, deletion, and/or substitution of at least one amino acid. In some embodiments, the IL-12 beta variant, an IL-12 beta mutein, or the IL-12 beta mutant may be at least partially functional, i.e., can perform an IL-12 beta activity at a similar or lower level compared to a wildtype IL-12 beta. In some embodiments, the IL-12 beta variant, an IL-12 beta mutein, or the IL-12 beta mutant may be fully functional, i.e., can perform an IL-12 beta activity at the same level compared to a wildtype IL-12 beta. In some embodiments, the IL-12 beta variant, an IL-12 beta mutein, or the IL-12 beta mutant may perform an IL-12 beta activity at a higher level compared to a wildtype IL-12 beta.

The mRNA encoding IL-12 may refer to an mRNA comprising a nucleotide sequence encoding the propeptide of human IL-12 alpha having 219 amino acids or a nucleotide sequence encoding the mature human IL-12 alpha having 197 amino acids. The nucleotide sequence encoding the propeptide of human IL-12 alpha and the nucleotide sequence encoding the mature human IL-12 may be codon-optimized. The mRNA encoding IL-12 may refer to an mRNA comprising a nucleotide sequence encoding the propeptide of human IL-12 beta having 328 amino acids or a nucleotide sequence encoding the mature human IL-12 beta having 306 amino acids. The nucleotide sequence encoding the propeptide of human IL-12 beta and the nucleotide sequence encoding the mature human IL-12 may be codon-optimized. In some instances, recombinant RNA constructs, provided herein, may comprise 1 copy of IL-12 mRNA. In some instances, recombinant RNA constructs, provided herein, may comprise 2 or more copies of IL-12 mRNA.

In some instances, interleukin 15 (IL-15) or IL-15 as used herein may refer to the natural sequence of human IL-15 (Genbank database: NM_000585.4), a fragment thereof, or a functional variant thereof. The natural DNA sequence encoding human IL-15 may be codon-optimized. The natural sequence of human IL-15 may consist of a signal peptide having 29 amino acids and the mature human IL-15 having 133 amino acids as shown in SEQ ID NO: 67. In some embodiments, the signal peptide is unmodified IL-15 signal peptide. In some embodiments, the signal peptide is IL-15 signal peptide modified by insertion, deletion, and/or substitution of at least one amino acid. In some embodiments, interleukin 15 (IL-15) or IL-15 as used herein may refer to the mature human IL-15. In some embodiments, a mature protein can refer to a protein synthesized in the endoplasmic reticulum and secreted via the Golgi apparatus in a cell expressing and secreting the protein. In some embodiments, a mature IL-15 may refer to an IL-15 protein synthesized in the endoplasmic reticulum and secreted via the Golgi apparatus in a cell expressing and secreting IL-15. In some embodiments, a mature human IL-15 may refer to an IL-15 protein synthesized in the endoplasmic reticulum and secreted via the Golgi apparatus in a human cell expressing and secreting human IL-15 and normally contains the amino acids encoded by nucleotide as shown in SEQ ID NO: 68. In some embodiments, IL-15 may comprise an IL-15 fragment, an IL-15 variant, an IL-15 mutein, or an IL-15 mutant. In some embodiments, the IL-15 fragment described herein may be at least partially functional, i.e., can perform an IL-15 activity at a similar or lower level compared to a wildtype or a full-length IL-15. In some embodiments, the IL-15 fragment described herein may be fully functional, i.e., can perform an IL-15 activity at the same level compared to a wildtype or a full-length IL-15. In some embodiments, the IL-15 variant, an IL-mutein, or the IL-15 mutant may comprise an IL-15 amino acid sequence modified by insertion, deletion, and/or substitution of at least one amino acid. In some embodiments, the IL-15 variant, an IL-15 mutein, or the IL-15 mutant may be at least partially functional, i.e., can perform an IL-15 activity at a similar or lower level compared to a wildtype IL-15. In some embodiments, the IL-15 variant, an IL-15 mutein, or the IL-15 mutant may be fully functional, i.e., can perform an IL-15 activity at the same level compared to a wildtype IL-15. In some embodiments, the IL-15 variant, an IL-15 mutein, or the IL-15 mutant may perform an IL-15 activity at a higher level compared to a wildtype IL-15.

The mRNA encoding IL-15 may refer to an mRNA comprising a nucleotide sequence encoding the propeptide of human IL-15 having 162 amino acids or a nucleotide sequence encoding the mature human IL-15 having 133 amino acids. The nucleotide sequence encoding the propeptide of human IL-15 and the nucleotide sequence encoding the mature human IL-15 may be codon-optimized. In some instances, recombinant RNA constructs, provided herein, may comprise 1 copy of IL-15 mRNA. In some instances, recombinant RNA constructs, provided herein, may comprise 2 or more copies of IL-15 mRNA.

In some instances, interleukin 7 (IL-7) or IL-7 as used herein may refer to the natural sequence of human IL-7 (Genbank database: NM_000880.3), a fragment thereof, or a functional variant thereof. The natural DNA sequence encoding human IL-7 may be codon-optimized. The natural sequence of human IL-7 may consist of a signal peptide having 25 amino acids and the mature human IL-7 having 152 amino acids as shown in SEQ ID NO: 79. In some embodiments, the signal peptide is unmodified IL-7 signal peptide. In some embodiments, the signal peptide is IL-7 signal peptide modified by insertion, deletion, and/or substitution of at least one amino acid. In some embodiments, interleukin 7 (IL-7) or IL-7 as used herein may refer to the mature human IL-7. In some embodiments, a mature protein can refer to a protein synthesized in the endoplasmic reticulum and secreted via the Golgi apparatus in a cell expressing and secreting the protein. In some embodiments, a mature IL-7 may refer to an IL-7 protein synthesized in the endoplasmic reticulum and secreted via the Golgi apparatus in a cell expressing and secreting IL-7. In some embodiments, a mature human IL-7 may refer to an IL-7 protein synthesized in the endoplasmic reticulum and secreted via the Golgi apparatus in a human cell expressing and secreting human IL-7 and normally contains the amino acids encoded by nucleotide as shown in SEQ ID NO: 80. In some embodiments, IL-7 may comprise an IL-7 fragment, an IL-7 variant, an IL-7 mutein, or an IL-7 mutant. In some embodiments, the IL-7 fragment described herein may be at least partially functional, i.e., can perform an IL-7 activity at a similar or lower level compared to a wildtype or a full-length IL-7. In some embodiments, the IL-7 fragment described herein may be fully functional, i.e., can perform an IL-7 activity at the same level compared to a wildtype or a full-length IL-7. In some embodiments, the IL-7 variant, an IL-7 mutein, or the IL-7 mutant may comprise an IL-7 amino acid sequence modified by insertion, deletion, and/or substitution of at least one amino acid. In some embodiments, the IL-7 variant, an IL-7 mutein, or the IL-7 mutant may be at least partially functional, i.e., can perform an IL-7 activity at a similar or lower level compared to a wildtype IL-7. In some embodiments, the IL-7 variant, an IL-7 mutein, or the IL-7 mutant may be fully functional, i.e., can perform an IL-7 activity at the same level compared to a wildtype IL-7. In some embodiments, the IL-7 variant, an IL-7 mutein, or the IL-7 mutant may perform an IL-7 activity at a higher level compared to a wildtype IL-7.

The mRNA encoding IL-7 may refer to an mRNA comprising a nucleotide sequence encoding the propeptide of human IL-7 having 177 amino acids or a nucleotide sequence encoding the mature human IL-7 having 152 amino acids. The nucleotide sequence encoding the propeptide of human IL-7 and the nucleotide sequence encoding the mature human IL-7 may be codon-optimized. In some instances, recombinant RNA constructs, provided herein, may comprise 1 copy of IL-7 mRNA. In some instances, recombinant RNA constructs, provided herein, may comprise 2 or more copies of IL-7 mRNA.

### Target Motif

Provided herein are compositions comprising recombinant RNA constructs comprising a target motif. A target motif or a targeting motif as used herein can refer to any short peptide present in the newly synthesized polypeptides or proteins that are destined to any parts of cell membranes, extracellular compartments, or intracellular compartments, except cytoplasm or cytosol. In some embodiments, a peptide may refer to a series of amino acid residues connected one to the other, typically by peptide bonds between the α-amino and carboxyl groups of adjacent amino acid residues. Intracellular compartments include, but are not limited to, intracellular organelles such as nucleus, nucleolus, endosome, proteasome, ribosome, chromatin, nuclear envelope, nuclear pore, exosome, melanosome, Golgi apparatus, peroxisome, endoplasmic reticulum (ER), lysosome, centrosome, microtubule, mitochondria, chloroplast, microfilament, intermediate filament, or plasma membrane. In some embodiments, a signal peptide can be referred to as a signal sequence, a targeting signal, a localization signal, a localization sequence, a transit peptide, a leader sequence, or a leader peptide. In some embodiments, a target motif is operably linked to a nucleic acid sequence encoding a gene of interest. In some embodiments, the term “operably linked” can refer to a functional relationship between two or more nucleic acid sequences, e.g., a functional relationship of a transcriptional regulatory or signal sequence to a transcribed sequence. For example, a target motif or a nucleic acid encoding a target motif is operably linked to a coding sequence if it is expressed as a preprotein that participates in targeting the polypeptide encoded by the coding sequence to a cell membrane, intracellular, or an extracellular compartment. For example, a signal peptide or a nucleic acid encoding a signal peptide is operably linked to a coding sequence if it is expressed as a preprotein that participates in the secretion of the polypeptide encoded by the coding sequence. For example, a promoter is operably linked if it stimulates or modulates the transcription of the coding sequence. Non-limiting examples of a target motif comprise a signal peptide, a nuclear localization signal (NLS), a nucleolar localization signal (NoLS), a lysosomal targeting signal, a mitochondrial targeting signal, a peroxisomal targeting signal, a microtubule tip localization signal (MtLS), an endosomal targeting signal, a chloroplast targeting signal, a Golgi targeting signal, an endoplasmic reticulum (ER) targeting signal, a proteasomal targeting signal, a membrane targeting signal, a transmembrane targeting signal, a centrosomal localization signal (CLS) or any other signal that targets a protein to a certain part of cell membrane, extracellular compartments, or intracellular compartments.

A signal peptide is a short peptide present at the N-terminus of newly synthesized proteins that are destined towards the secretory pathway. The signal peptide of the present invention can be 10-40 amino acids long. A signal peptide can be situated at the N-terminal end of the protein of interest or at the N-terminal end of a pro-protein form of the protein of interest. A signal peptide may be of eukaryotic origin. In some embodiments, a signal peptide may be a mammalian protein. In some embodiments, a signal peptide may be a human protein. In some instances, a signal peptide may be a homologous signal peptide (i.e. from the same protein) or a heterologous signal peptide (i.e. from a different protein or a synthetic signal peptide). In some instances, a signal peptide may be a naturally occurring signal peptide of a protein or a modified signal peptide.

Provided herein are compositions comprising recombinant RNA constructs comprising a target motif, wherein the target motif may be selected from the group consisting of (a) a target motif heterologous to a protein encoded by the gene of interest; (b) a target motif heterologous to a protein encoded by the gene of interest, wherein the target motif heterologous to the protein encoded by the gene of interest is modified by insertion, deletion, and/or substitution of at least one amino acid; (c) a target motif homologous to a protein encoded by the gene of interest; (d) a target motif homologous to a protein encoded by the gene of interest, wherein the target motif homologous to the protein encoded by the gene of interest is modified by insertion, deletion, and/or substitution of at least one amino acid; and (e) a naturally occurring amino acid sequence which does not have the function of a target motif in nature, wherein the naturally occurring amino acid sequence is optionally modified by insertion, deletion, and/or substitution of at least one amino acid.

Provided herein are compositions comprising recombinant RNA constructs comprising a target motif, wherein the target motif is a signal peptide. In some embodiments, the signal peptide is selected from the group consisting of: (a) a signal peptide heterologous to a protein encoded by the gene of interest; (b) a signal peptide heterologous to a protein encoded by the gene of interest, wherein the signal peptide heterologous to the protein encoded by the gene of interest is modified by insertion, deletion, and/or substitution of at least one amino acid, with proviso that the protein is not an oxidoreductase; (c) a signal peptide homologous to a protein encoded by the gene of interest; (d) a signal peptide homologous to a protein encoded by the gene of interest, wherein the signal peptide homologous to the protein encoded by the gene of interest is modified by insertion, deletion, and/or substitution of at least one amino acid; and (e) a naturally occurring amino acid sequence which does not have the function of a signal peptide in nature, wherein the naturally occurring amino acid sequence is optionally modified by insertion, deletion, and/or substitution of at least one amino acid. In some instances, the amino acids 1-9 of the N-terminal end of the signal peptide have an average hydrophobic score of above 2.

In some instances, a target motif heterologous to a protein encoded by the gene of interest or a signal peptide heterologous to a protein encoded by the gene of interest as used herein can refer to a naturally occurring target motif or signal peptide which is different from the naturally occurring target motif or signal peptide of a protein. For example, the target motif or the signal peptide is not derived from the gene of interest. Usually a target motif or a signal peptide heterologous to a given protein is a target motif or a signal peptide from another protein, which is not related to the given protein. For example, a target motif or a signal peptide heterologous to a given protein has an amino acid sequence that is different from the amino acid sequence of the target motif or the signal peptide of the given protein by more than 50%, 60%, 70%, 80%, 90%, or by more than 95%. Although heterologous sequences may be derived from the same organism, they naturally (in nature) do not occur in the same nucleic acid molecule, such as in the same mRNA. The target motif or the signal peptide heterologous to a protein and the protein to which the target motif or the signal peptide is heterologous can be of the same or different origin. In some embodiments, they are of eukaryotic origin. In some embodiments, they are of the same eukaryotic organism. In some embodiments, they are of mammalian origin. In some embodiments, they are of the same mammalian organism. In some embodiments, they are human origin. For example, an RNA construct may comprise a nucleic acid sequence encoding the human IL-2 gene and a signal peptide of another human cytokine. In some embodiments, an RNA construct may comprise a signal peptide heterologous to a protein wherein the signal peptide and the protein are of the same origin, namely of human origin.

In some instance, a target motif homologous to a protein encoded by the gene of interest or a signal peptide homologous to a protein encoded by the gene of interest as used herein can refer to a naturally occurring target motif or signal peptide of a protein. A target motif or a signal peptide homologous to a protein is the target motif or the signal peptide encoded by the gene of the protein as it occurs in nature. A target motif or a signal peptide homologous to a protein is usually of eukaryotic origin. In some embodiments, a target motif or a signal peptide homologous to a protein is of mammalian origin. In some embodiments, a target motif or a signal peptide homologous to a protein is of human origin.

In some instances, a naturally occurring amino acid sequence which does not have the function of a target motif in nature or a naturally occurring amino acid sequence which does not have the function of a signal peptide in nature as used herein can refer to an amino acid sequence which occurs in nature and is not identical to the amino acid sequence of any target motif or signal peptide occurring in nature. A naturally occurring amino acid sequence which does not have the function of a target motif or a signal peptide in nature can be between 10-50 amino acids long. In some embodiments, a naturally occurring amino acid sequence which does not have the function of a target motif or a signal peptide in nature is of eukaryotic origin and not identical to any target motif or signal peptide of eukaryotic origin. In some embodiments, a naturally occurring amino acid sequence which does not have the function of a target motif or a signal peptide in nature is of mammalian origin and not identical to any target motif or signal peptide of mammalian origin. In some embodiments, a naturally occurring amino acid sequence which does not have the function of a target motif or a signal peptide in nature is of human origin and not identical to any target motif or signal peptide of human origin occurring in nature. A naturally occurring amino acid sequence which does not have the function of a target motif or a signal peptide in nature is usually an amino acid sequence of the coding sequence of a protein. The terms “naturally occurring,” “natural,” and “in nature” as used herein have the equivalent meaning.

In some instances, amino acids 1-9 of the N-terminal end of the signal peptide as used herein can refer to the first nine amino acids of the N-terminal end of the amino acid sequence of a signal peptide. Analogously, amino acids 1-7 of the N-terminal end of the signal peptide as used herein can refer to the first seven amino acids of the N-terminal end of the amino acid sequence of a signal peptide and amino acids 1-5 of the N-terminal end of the signal peptide can refer to the first five amino acids of the N-terminal end of the amino acid sequence of a signal peptide.

In some instances, amino acid sequence modified by insertion, deletion, and/or substitution of at least one amino acid can refer to an amino acid sequence which includes an amino acid substitution, insertion, and/or deletion of at least one amino acid within the amino acid sequence. For example, target motif heterologous to a protein encoded by the gene of interest is modified by insertion, deletion, and/or substitution of at least one amino acid or signal peptide heterologous to a protein encoded by the gene of interest is modified by insertion, deletion, and/or substitution of at least one amino acid as used herein can refer to an amino acid sequence of a naturally occurring target motif or signal peptide heterologous to a protein which includes an amino acid substitution, insertion, and/or deletion of at least one amino acid within its naturally occurring amino acid sequence. For example, target motif homologous to a protein encoded by the gene of interest is modified by insertion, deletion, and/or substitution of at least one amino acid or signal peptide homologous to a protein encoded by the gene of interest is modified by insertion, deletion, and/or substitution of at least one amino acid as used herein can refer to a naturally occurring target motif or signal peptide homologous to a protein which includes an amino acid substitution, insertion, and/or deletion of at least one amino acid within its naturally occurring amino acid sequence. In some embodiments, naturally occurring amino acid sequence may be modified by insertion, deletion, and/or substitution of at least one amino acid and a naturally occurring amino acid sequence can include an amino acid substitution, insertion, and/or deletion of at least one amino acid within its naturally occurring amino acid sequence. An amino acid substitution or a substitution may refer to replacement of an amino acid at a particular position in an amino acid or polypeptide sequence with another amino acid. For example, the substitution R34K refers to a polypeptide, in which the arginine (Arg or R) at position 34 is replaced with a lysine (Lys or K). For the preceding example, 34K indicates the substitution of an amino acid at position 34 with a lysine (Lys or K). In some embodiments, multiple substitutions are typically separated by a slash. For example, R34K/L38V refers to a variant comprising the substitutions R34K and L38V. An amino acid insertion or an insertion may refer to addition of an amino acid at a particular position in an amino acid or polypeptide sequence. For example, insert −34 designates an insertion at position 34. An amino acid deletion or a deletion may refer to removal of an amino acid at a particular position in an amino acid or polypeptide sequence. For example, R34-designates the deletion of arginine (Arg or R) at position 34.

In some instances, deleted amino acid is an amino acid with a hydrophobic score of below −0.8, −0.7, −0.6, −0.5, −0.4, −0.3, −0.2, −0.1, 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, or below 1.9. In some instances, the substitute amino acid is an amino acid with a hydrophobic score which is higher than the hydrophobic score of the substituted amino acid. For example, the substitute amino acid is an amino acid with a hydrophobic score of 2.8 and higher, or 3.8 and higher. In some instances, the inserted amino acid is an amino acid with a hydrophobic score of 2.8 and higher or 3.8 and higher.

In some instances, an amino acid sequence described herein may comprise 1 to 15 amino acid insertions, deletions, and/or substitutions. In some embodiments, an amino acid sequence described herein may comprise 1 to 7 amino acid insertions, deletions, and/or substitutions. In some instances, an amino acid sequence described herein may not comprise amino acid insertions, deletions, and/or substitutions. In some instances, an amino acid sequence described herein may comprise 1 to 15 amino acid insertions, deletions, and/or substitutions within the amino acids 1-30 of the N-terminal end of the amino acid sequence of the target motif or the signal peptide. In some embodiments, an amino acid sequence described herein may comprise 1 to 9 amino acid insertions, deletions, and/or substitutions within the amino acids 1-30 of the N-terminal end of the amino acid sequence of the target motif or the signal peptide. In some instances, an amino acid sequence described herein may comprise 1 to amino acid insertions, deletions, and/or substitutions within the amino acids 1-20 of the N-terminal end of the amino acid sequence of the target motif or the signal peptide. In some embodiments, an amino acid sequence described herein may comprise 1 to 9 amino acid insertions, deletions, and/or substitutions within the amino acids 1-20 of the N-terminal end of the amino acid sequence of the target motif or the signal peptide. In some instances, at least one amino acid of an amino acid sequence described herein may be optionally modified by deletion, and/or substitution.

In some instances, the average hydrophobic score of the first nine amino acids of the N-terminal end of the amino acid sequence of the modified signal peptide is increased 1.0 unit or above compared to the signal peptide without modification. In some instances, hydrophobic score or hydrophobicity score can be used synonymously to hydropathy score herein and can refer to the degree of hydrophobicity of an amino acid as calculated according to the Kyte-Doolittle scale (Kyte J., Doolittle R. F.; J. Mol. Biol. 157:105-132(1982)). The amino acid hydrophobic scores according to the Kyte-Doolittle scale are as follows:

TABLE B
Amino Acid Hydrophobic Scores

| Amino Acid | One Letter Code | Hydrophobic Score |
|---|---|---|
| Isoleucine | I | 4.5 |
| Valine | V | 4.2 |
| Leucine | L | 3.8 |
| Phenylalanine | F | 2.8 |
| Cysteine | C | 2.5 |
| Methionine | M | 1.9 |
| Alanine | A | 1.8 |
| Glycine | G | −0.4 |
| Threonine | T | −0.7 |
| Serine | S | −0.8 |
| Tryptophan | W | −0.9 |
| Tyrosine | Y | −1.3 |
| Proline | P | −1.6 |
| Histidine | H | −3.2 |
| Glutamic acid | E | −3.5 |
| Glutamine | Q | −3.5 |
| Aspartic acid | D | −3.5 |
| Asparagine | N | −3.5 |
| Lysine | K | −3.9 |
| Arginine | R | −4.5 |

In some instances, average hydrophobic score of an amino acid sequence can be calculated by adding the hydrophobic score according to the Kyte-Doolittle scale of each of the amino acid of the amino acid sequence divided by the number of the amino acids. For example, the average hydrophobic score of the amino acids 1-9 of the N-terminal end of the amino acid sequence of a signal peptide can be calculated by adding the hydrophobic score or each of the nine amino acids divided by nine.

The polarity is calculated according to Zimmerman Polarity index (Zimmerman J. M., Eliezer N., Simha R.; J. Theor. Biol. 21:170-201(1968)). In some embodiments, average polarity of an amino acid sequence can be calculated by adding the polarity value calculated according to Zimmerman Polarity index of each of the amino acid of the amino acid sequence divided by the number of the amino acids. For example, the average polarity of the amino acids 1-9 of the N-terminal end of the amino acid sequence of a signal peptide can be calculated by adding the average polarity of each of the nine amino acids of the amino acids 1-9 of the N-terminal end, divided by nine. The polarity of amino acids according to Zimmerman Polarity index is as follows:

TABLE C
Amino Acid Polarity

| Amino Acid | One Letter Code | Polarity |
|---|---|---|
| Isoleucine | I | 0.13 |
| Valine | V | 0.13 |
| Leucine | L | 0.13 |
| Phenylalanine | F | 0.35 |
| Cysteine | C | 1.48 |
| Methionine | M | 1.43 |
| Alanine | A | 0 |
| Glycine | G | 0 |
| Threonine | T | 1.66 |
| Serine | S | 1.67 |
| Tryptophan | W | 2.1 |
| Tyrosine | Y | 1.61 |
| Proline | P | 1.58 |
| Histidine | H | 51.6 |
| Glutamic acid | E | 49.9 |
| Glutamine | Q | 3.53 |
| Aspartic acid | D | 49.7 |
| Asparagine | N | 3.38 |
| Lysine | K | 49.5 |
| Arginine | R | 52 |

In some instances, a naturally occurring signal peptide of interleukin 2 (IL-2) may be modified by one or more substitutions, deletions, and/or insertions, wherein the naturally occurring signal peptide of IL-2 is referred to the amino acids 1-20 of the IL-2 amino acid sequence in the Uniprot database as P60568 or Q0GK43 and in the Genbank database as NM_000586.3. In some instances, the amino acid sequence of IL-2 signal peptide may be modified by the one or more substitutions, deletions, and/or insertions selected from the group consisting of Y2L, R3K, R3−, M4L, Q5L, S8L, S8A, −13A, L14T, L16A, V17−, and V17A. In some instances, the wild type (WT) IL-2 signal peptide amino acid sequence comprises a sequence comprising SEQ ID NO: 26. In some instances, a modified IL-2 signal peptide has an amino acid sequence comprising a sequence selected from the group consisting of SEQ ID NOs: 27-29. In some instances, a modified IL-2 signal peptide is encoded by a DNA sequence selected from the group consisting of SEQ ID NOs: 31-33.

### Expression Vector and Production of RNA Constructs

Provided herein are compositions comprising recombinant polynucleic acid constructs encoding recombinant RNA constructs comprising: (i) an mRNA encoding a gene of interest; and (ii) at least one siRNA capable of binding to a target mRNA. For example, an mRNA encoding a gene of interest can be IL-2, IL-12, IL-15, IL-7, a fragment thereof, or a functional variant thereof. For example, a target mRNA can be VEGF, VEGFA, an isoform of VEGFA, PIGF, MICA, MICB, ERp5, ADAM, MMP, IDH1, CDK4, CDK6, EGFR, mTOR, KRAS, CD155, PD-L1, or c-Myc. In some embodiments, the ADAM is ADAM17. Further provided herein are compositions comprising recombinant polynucleic acid constructs encoding RNA constructs described herein, e.g., an RNA construct comprising a first RNA encoding for a cytokine linked to a second RNA encoding for a genetic element that can reduce expression of a gene associated with tumor proliferation, angiogenesis, or recognition by the immune system. For example, a cytokine can be IL-2, IL-12, IL-15, IL-7, a fragment thereof, or a functional variant thereof. For example, a gene associated with tumor proliferation or angiogenesis can be VEGF, VEGFA, an isoform of VEGFA, PIGF, IDH1, CDK4, CDK6, EGFR, mTOR, KRAS, CD155, PD-L1, c-Myc, a fragment thereof, or a functional variant thereof. Non-limiting examples of an isoform of VEGFA include VEGF111, VEGF121, VEGF145, VEGF148, VEGF165, VEGF165B, VEGF183, VEGF189, VEGF206, L-VEGF121, L-VEGF165, L-VEGF189, L-VEGF206, Isoform 15, Isoform16, Isoform 17, and Isoform 18. For example, a gene associated with recognition by the immune system can be MICA, MICB, ERp5, ADAM, MMP, a fragment thereof, or a functional variant thereof. In some embodiments, the ADAM is ADAM17. In related aspects, recombinant polynucleic acid constructs encoding recombinant RNA constructs may encode 1, 2, 3, 4, 5, or more siRNA species. In related aspects, recombinant polynucleic acid constructs encoding recombinant RNA constructs may encode 1 siRNA species directed to a target mRNA. In related aspects, recombinant polynucleic acid constructs encoding recombinant RNA constructs may encode 3 siRNAs, each directed to a target mRNA. In related aspects, each of the siRNA species may comprise the same sequence, different sequence, or a combination thereof. For example, recombinant polynucleic acid constructs encoding recombinant RNA constructs may encode 3 siRNAs, each directed to the same region or sequence of the target mRNA. For example, recombinant polynucleic acid constructs encoding recombinant RNA constructs may encode 3 siRNAs, each directed to a different region or sequence of the target mRNA. In some aspects, recombinant polynucleic acid constructs encoding recombinant RNA constructs may encode 3 siRNA species, wherein each of the 3 siRNA species is directed to a different target mRNA. In some embodiments, a target mRNA may be an mRNA of VEGF, VEGFA, an isoform of VEGFA, PIGF, MICA, MICB, ERp5, ADAM17, MMP, IDH1, CDK4, CDK6, EGFR, mTOR, KRAS, CD155, PD-L1, or c-Myc. In related aspects, recombinant polynucleic acid constructs may comprise a sequence selected from the group consisting of SEQ ID NOs: 82-98.

The polynucleic acid constructs, described herein, can be obtained by any method known in the art, such as by chemically synthesizing the DNA chain, by PCR, or by the Gibson Assembly method. The advantage of constructing polynucleic acid constructs by chemical synthesis or a combination of PCR method or Gibson Assembly method is that the codons may be optimized to ensure that the fusion protein is expressed at a high level in a host cell. Codon optimization can refer to a process of modifying a nucleic acid sequence for expression in a host cell of interest by replacing at least one codon (e.g., more than 1, 2, 3, 4, 5, 10, 15, 20, 25, 50, or more codons) of a native sequence with codons that are more frequently or most frequently used in the genes of that host cell while maintaining the native amino acid sequence. Codon usage tables are readily available, for example, at the “Codon Usage Database,” and these tables can be adapted in a number of ways. Computer algorithms for codon optimizing a particular sequence for expression in a particular host cell are also available, such as Gene Forge® (Aptagen, PA) and GeneOptimizer® (ThermoFischer, MA). Once obtained polynucleotides can be incorporated into suitable vectors. Vectors as used herein can refer to naturally occurring or synthetically generated constructs for uptake, proliferation, expression or transmission of nucleic acids in vivo or in vitro, e.g., plasmids, minicircles, phagemids, cosmids, artificial chromosomes/mini-chromosomes, bacteriophages, viruses such as baculovirus, retrovirus, adenovirus, adeno-associated virus, herpes simplex virus, bacteriophages. Methods used to construct vectors are well known to a person skilled in the art and described in various publications. In particular techniques for constructing suitable vectors, including a description of the functional and regulatory components such as promoters, enhancers, termination and polyadenylation signals, selection markers, origins of replication, and splicing signals, are known to the person skilled in the art. A variety of vectors are well known in the art and some are commercially available from companies such as Agilent Technologies, Santa Clara, Calif; Invitrogen, Carlsbad, Calif; Promega, Madison, Wis.; Thermo Fisher Scientific; or Invivogen, San Diego, Calif A non-limiting examples of vectors for in vitro transcription includes pT7CFE1-CHis, pMX (such as pMA-T, pMA-RQ, pMC, pMK, pMS, pMZ), pEVL, pSP73, pSP72, pSP64, and pGEM (such as pGEM®-4Z, pGEM®-5Zf(+), pGEM®-11Zf(+), pGEM®-9Zf(−), pGEM®-3Zf(+/−), pGEM®-7Zf(+/−)). In some instances, recombinant polynucleic acid constructs may be DNA.

The polynucleic acid constructs, as described herein, can be circular or linear. For example, circular polynucleic acid constructs may include vector system such as pMX, pMA-T, pMA-RQ, or pT7CFE1-CHis. For example, linear polynucleic acid constructs may include linear vector such as pEVL or linearized vectors. In some instances, recombinant polynucleic acid constructs may further comprise a promoter. In some instances, the promoter may be present upstream of the sequence encoding for the first RNA or the sequence encoding for the second RNA. Non-limiting examples of a promoter can include T3, T7, SP6, P60, Syn5, and KP34. In some instances, recombinant polynucleic acid constructs provided herein may comprise a T7 promoter comprising a sequence comprising TAATACGACTCACTATA (SEQ ID NO: 18). In some instances, recombinant polynucleic acid constructs further comprises a sequence encoding a Kozak sequence. A Kozak sequence may refer to a nucleic acid sequence motif that functions as the protein translation initiation site. Kozak sequences are described at length in the literature, e.g., by Kozak, M., Gene 299(1-2):1-34, incorporated herein by reference herein in its entirety. In some embodiments, recombinant polynucleic acid constructs comprises a sequence encoding a Kozak sequence comprising a sequence comprising GCCACC (SEQ ID NO: 19). In some instances, recombinant polynucleic acid constructs described herein may be codon-optimized.

Provided herein are compositions comprising recombinant polynucleic acid constructs encoding RNA constructs described herein comprising one or more nucleic acid sequence encoding an siRNA capable of binding to a target RNA and one or more nucleic acid sequence encoding a gene of interest, wherein the siRNA capable of binding to a target RNA is not a part of an intron sequence encoded by the gene of interest. In some instances, the gene of interest is expressed without RNA splicing. In some instances, the siRNA capable of binding to a target RNA binds to an exon of a target mRNA. In some instances, the siRNA capable of binding to a target RNA specifically binds to one target RNA. In some instances, recombinant polynucleic acid constructs may comprise a nucleic acid sequence comprising a sequence selected from the group consisting of SEQ ID NOs: 82-98.

Provided herein are methods of producing RNA construct compositions described herein. For example, recombinant RNA constructs may be produced by in vitro transcription from a polynucleic acid construct comprising a promoter for an RNA polymerase, at least one nucleic acid sequence encoding a gene of interest, at least one nucleic acid sequence encoding an siRNA capable of binding to a target mRNA, and a nucleic acid sequence encoding poly(A) tail. In vitro transcription reaction may further comprise an RNA polymerase, a mixture of nucleotide triphosphates (NTPs), and/or a capping enzyme. Details of producing RNAs using in vitro transcription as well as isolating and purifying transcribed RNAs is well known in the art and can be found, for example, in Beckert & Masquida ((2011) Synthesis of RNA by In vitro Transcription. RNA. Methods in Molecular Biology (Methods and Protocols), vol 703. Humana Press). A non-limiting list of in vitro transcript kits includes MEGAscript™ T3 Transcription Kit, MEGAscript T7 kit, MEGAscript™ SP6 Transcription Kit, MAXIscript™ T3 Transcription Kit, MAXIscript™ T7 Transcription Kit, MAXIscript™ SP6 Transcription Kit, MAXIscript™ T7/T3 Transcription Kit, MAXIscript™ SP6/T7 Transcription Kit, mMESSAGE mMACHINE™ T3 Transcription Kit, mMESSAGE mMACHINE™ T7 Transcription Kit, mMESSAGE mMACHINE™ SP6 Transcription Kit, MEGAshortscript™ T7 Transcription Kit, HiScribe™ T7 High Yield RNA Synthesis Kit, HiScribe™ T7 In Vitro Transcription Kit, AmpliScribe™ T7-Flash™ Transcription Kit, AmpliScribe™ T7 High Yield Transcription Kit, AmpliScribe™ T7-Flash™ Biotin-RNA Transcription Kit, T7 Transcription Kit, HighYield T7 RNA Synthesis Kit, DuraScribe® T7 Transcription Kit, etc.

The in vitro transcription reaction can further comprise a transcription buffer system, nucleotide triphosphates (NTPs), and an RNase inhibitor. In some embodiments, the transcription buffer system may comprise dithiothreitol (DTT) and magnesium ions. The NTPs can be naturally occurring or non-naturally occurring (modified) NTPs. Non-limiting examples of non-naturally occurring (modified) NTPs include N¹-Methylpseudouridine, Pseudouridine, N¹-Ethylpseudouridine, N¹-Methoxymethylpseudouridine, N¹-Propylpseudouridine, 2-thiouridine, 4-thiouridine, 5-methoxyuridine, 5-methylurdine, 5-carboxymethylesteruridine, 5-formyluridine, 5-carboxyuridine, 5-hydroxyuridine, 5-Bromouridine, 5-Iodouridine, 5,6-dihydrouridine, 6-Azauridine, Thienouridine, 3-methyluridine, 1-carboxymethyl-pseudouridine, 4-thio-1-methyl-pseudouridine, 2-thio-1-methyl-pseudouridine, dihydrouridine, dihydropseudouridine, 2-methoxyuridine, 2-methoxy-4-thio-uridine, 4-methoxy-pseudouridine, 4-methoxy-2-thio-pseudouridine, 5-methylcytidine, 5-methoxycytidine, 5-hydroxymethylcytidine, 5-formylcytidine, 5-carboxycytidine, 5-hydroxycytidine, 5-Iodocytidine, 5-Bromocytidine, 2-thiocytidine, 5-azacytidine, pseudoisocytidine, 3-methyl-cytidine, N⁴-acetylcytidine, 5-formylcytidine, N⁴-methylcytidine, 5-hydroxymethylcytidine, 1-methyl-pseudoisocytidine, 4-methoxy-pseudoisocytidine, and 4-methoxy-1-methyl-pseudoisocytidine, N¹-methyladenosine, N⁶-methyladenosine, N⁶-methyl-2-Aminoadenosine, N⁶-isopentenyladenosine, N⁶,N⁶-dimethyladenosine, 7-methyladenine, 2-methylthio-adenine, and 2-methoxy-adenine. Non-limiting examples of DNA-dependent RNA polymerase include T3, T7, SP6, P60, Syn5, and KP34 RNA polymerases. In some embodiments, the RNA polymerase is selected from the group consisting of T3 RNA polymerase, T7 RNA polymerase, SP6 RNA polymerase, P60 RNA polymerase, Syn5 RNA polymerase, and KP34 RNA polymerase.

Transcribed RNAs, as described herein, may be isolated and purified from the in vitro transcription reaction mixture. For example, transcribed RNAs may be isolated and purified using column purification. Details of isolating and purifying transcribed RNAs from in vitro transcription reaction mixture is well known in the art and any commercially available kits may be used. A non-limiting list of RNA purification kits includes MEGAclear kit, Monarch® RNA Cleanup Kit, EasyPure® RNA Purification Kit, NucleoSpin® RNA Clean-up, etc.

### Therapeutic Applications

Provided herein are compositions useful in the treatment of a cancer. In some aspects, compositions are present or administered in an amount sufficient to treat or prevent a disease or condition. Provided herein are compositions comprising a first RNA encoding a cytokine linked to a second RNA encoding a genetic element that can reduce expression of a gene associated with tumor proliferation, angiogenesis, or recognition by the immune system. In some embodiments, a cytokine may comprise IL-2, IL-7, IL-12, IL-15, a fragment thereof, or a functional variant thereof. In some embodiments, a genetic element that can reduce expression of a gene associated with tumor proliferation or angiogenesis may comprise siRNA targeting VEGF, VEGFA, an isoform of VEGFA, PIGF, IDH1, CDK4, CDK6, EGFR, mTOR, KRAS, CD155, PD-L1, c-Myc, a fragment thereof, or a functional variant thereof. In some embodiments, a genetic element that can reduce expression of a gene associated with recognition by the immune system may comprise siRNA targeting MICA, MICB, ERp5, ADAM, MMP, a fragment thereof, or a functional variant thereof. In some embodiments, the ADAM is ADAM17.

Also provided herein are pharmaceutical compositions comprising any RNA composition described herein and a pharmaceutically acceptable excipient. A pharmaceutical composition can denote a mixture or solution comprising a therapeutically effective amount of an active pharmaceutical ingredient together with one or more pharmaceutically acceptable excipients to be administered to a subject in need thereof. The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. The term “pharmaceutically acceptable” can refer to a material, such as a carrier or diluent, which does not abrogate the biological activity or properties of the compound, and is relatively nontoxic, i.e. the material may be administered to an individual without causing undesirable biological effects or interacting in a deleterious manner with any of the components of the composition in which it is contained. A pharmaceutically acceptable excipient can denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products. Pharmaceutical compositions can facilitate administration of the compound to an organism and can be formulated in a conventional manner using one or more pharmaceutically acceptable inactive ingredients that facilitate processing of the active compounds into preparations that can be used pharmaceutically. A proper formulation is dependent upon the route of administration chosen and a summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference. In some embodiments, pharmaceutical compositions can be formulated by dissolving active substances (e.g., recombinant polynucleic acid or RNA constructs described herein) in aqueous solution for injection into diseased tissues or diseased cells. In some embodiments, pharmaceutical compositions can be formulated by dissolving active substances (e.g., recombinant polynucleic acid or RNA constructs described herein) in aqueous solution for direct injection into diseased tissues or diseased cells. In some embodiments, diseased tissues or diseased cells comprise tumors or tumor cells.

Also provided herein are methods of treating a cancer in a subject in need thereof, comprising administering to the subject with the cancer a therapeutically effective amount of compositions or pharmaceutical compositions described herein. The terms “effective amount” or “therapeutically effective amount,” as used herein, refer to a sufficient amount of an agent or a compound being administered which will relieve to some extent one or more of the symptoms of the disease or the condition being treated; for example a reduction and/or alleviation of one or more signs, symptoms, or causes of a disease, or any other desired alteration of a biological system. For example, an “effective amount” for therapeutic uses can be an amount of an agent that provides a clinically significant decrease in one or more disease symptoms. An appropriate “effective” amount may be determined using techniques, such as a dose escalation study, in individual cases.

The terms “treat,” “treating” or “treatment,” as used herein, include alleviating, abating or ameliorating at least one symptom of a disease or a condition, preventing additional symptoms, inhibiting the disease or the condition, e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically. In some embodiments, treating a disease or condition comprises reducing the size of diseased tissues or diseased cells. In some embodiments, treating a disease or a condition in a subject comprises increasing the survival of a subject. In some embodiments, treating a disease or condition comprises reducing or ameliorating the severity of a disease, delaying onset of a disease, inhibiting the progression of a disease, reducing hospitalization of or hospitalization length for a subject, improving the quality of life of a subject, reducing the number of symptoms associated with a disease, reducing or ameliorating the severity of a symptom associated with a disease, reducing the duration of a symptom associated with a disease, preventing the recurrence of a symptom associated with a disease, inhibiting the development or onset of a symptom of a disease, or inhibiting of the progression of a symptom associated with a disease. In some embodiments, treating a cancer comprises reducing the size of tumor or increasing survival of a patient with a cancer.

In some cases, a subject can encompass mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In some cases, the mammal is a human. In some cases, the subject may be an animal. In some cases, an animal may comprise human beings and non-human animals. In one embodiment, a non-human animal may be a mammal, for example a rodent such as rat or a mouse. In another embodiment, a non-human animal may be a mouse. In some instances, the subject is a mammal. In some instances, the subject is a human. In some instances, the subject is an adult, a child, or an infant. In some instances, the subject is a companion animal. In some instances, the subject is a feline, a canine, or a rodent. In some instances, the subject is a dog or a cat.

Further provided herein are methods of treating a cancer comprising administering compositions or pharmaceutical compositions described herein to a subject with a cancer. In some instances, the cancer is a solid tumor. In some instances, a solid tumor may include, but is not limited to, breast cancer, lung cancer, liver cancer, glioblastoma, melanoma, head and neck squamous cell carcinoma, renal cell carcinoma, neuroblastoma, Wilms tumor, retinoblastoma, rhabdomyosarcoma, osteosarcoma, Ewing sarcoma, bladder cancer, cervical cancer, colon cancer, rectal cancer, endometrial cancer, kidney cancer, mesothelioma, non-small cell lung cancer, nonmelanoma skin cancer, ovarian cancer, pancreatic cancer, prostate cancer, small cell lung cancer, colorectal cancer, and thyroid cancer. In some embodiments, a solid tumor may include sarcomas, carcinomas, or lymphomas. In some embodiments, a solid tumor can be benign or malignant.

In some instances, the cancer is a head and neck cancer. Without wishing to be bound to any theory, the head and neck cancer is the sixth most common cancer worldwide and represent 6% of solid tumors. Approximately 650,000 new patients are diagnosed with head and neck cancers annually, and there are 350,000 deaths yearly worldwide with 12,000 deaths in the US despite the availability of advanced treatment options. Risk factors that increase the chance of developing head and neck cancers include use of tobacco and/or alcohol, prolonged sun exposure (e.g., in the lip area or skin of the head and neck), human papillomavirus (HPV), Epstein-Barr virus (EBV), gender (e.g., men versus women), age (e.g., people over the age of are at higher risk), poor oral and dental hygiene, and environmental or occupational inhalants (e.g., asbestos, wood dust, paint fumes, and other certain chemicals), marijuana use, poor nutrition, gastroesophageal reflux disease (GERD) and laryngopharyngeal reflux disease (LPRD), weakened immune system, radiation exposure, or previous history of head and neck cancer. Tobacco use is the single largest risk factor for head and neck cancer, and includes smoking cigarettes, cigars, or pipes; chewing tobacco; using snuff; and secondhand smoke. About 85% of head and neck cancers are linked to tobacco use, and the amount of tobacco use may affect prognosis. In addition, nearly 25% of head and neck cancers are HPV-positive.

Head and neck cancers can include epithelial malignancies of the upper aerodigestive tract, including the paranasal sinuses, nasal cavity, oral cavity, pharynx, and larynx. Non-limiting examples of the head and neck cancer includes laryngeal cancer, hypopharyngeal cancer, tonsil cancer, nasal cavity cancer, paranasal sinus cancer, nasopharyngeal cancer, metastatic squamous neck cancer with occult primary, lip cancer, oral cancer, oropharyngeal cancer, salivary gland cancer, brain tumors, esophageal cancer, eye cancer, parathyroid cancer, sarcoma of the head and neck, and thyroid cancer. The head and neck cancers described herein may be located at an upper aerodigestive tract. Non-limiting examples of the upper aerodigestive tract include a paranasal sinus, a nasal cavity, an oral cavity, a salivary gland, a tongue, a nasopharynx, an oropharynx, a hypopharynx, and a larynx.

In some embodiments, the cancer is selected from the group consisting of a head and neck cancer, melanoma, and renal cell carcinoma. In some embodiments, the cancer is a head and neck cancer. In some embodiments, the head and neck cancer is head and neck squamous cell carcinoma. In some embodiments, the head and neck cancer is laryngeal cancer, hypopharyngeal cancer, tonsil cancer, nasal cavity cancer, paranasal sinus cancer, nasopharyngeal cancer, metastatic squamous neck cancer with occult primary, lip cancer, oral cancer, oropharyngeal cancer, salivary gland cancer, brain tumors, esophageal cancer, eye cancer, parathyroid cancer, sarcoma of the head and neck, or thyroid cancer. In some embodiments, the cancer is melanoma. In some embodiments, the cancer is renal cell carcinoma.

Early treatment for cancers described herein may include surgical removal of tumors, radiation therapy, therapies using medications such as chemotherapy, targeted therapy, immunotherapy, or combinations thereof. Targeted therapy is a treatment that target specific genes, proteins, or the tissue environment that can contribute to cancer growth and survival, and the treatment is designed to block the growth and spread of cancer cells while limiting damage to healthy cells. For head and neck cancers, targeted therapies using antibodies may be used to inhibit cell proliferation, tumor proliferation or growth, or suppress tumor angiogenesis. Immunotherapy is a treatment that can improve, target, or restore immune system function to fight cancer. Non-limiting examples of antibodies include anti-epidermal growth factor receptor (EGFR) antibodies and anti-vascular endothelial growth factor (VEGF) antibodies. Non-limiting examples of cancer immunotherapy include immune system modulators, T-cell transfer therapy, immune checkpoint inhibitors, and monoclonal antibodies. Immune system modulators can enhance immune response against cancer and include cytokines such as interleukins and interferon alpha (IFNα). T-cell transfer therapy can refer to a treatment where immune cells are taken from a cancer patient for ex vivo manipulation and injected back to the same patient. For example, immune cells are taken from a cancer patient for specific expansion of tumor-recognizing lymphocytes (e.g., tumor-infiltrating lymphocytes therapy) or for modification of cells to express chimeric antigen receptors specifically recognizing tumor antigens (e.g., CAR T-cell therapy). Immune checkpoint inhibitors can block immune checkpoints, restoring or allowing immune responses to cancer cells. Non-limiting examples of immune checkpoint inhibitors include programmed death-ligand 1 (PD-L1) inhibitors, programmed death protein 1 (PD1) inhibitors, and cytotoxic T-lymphocyte-associated protein-4 (CTLA-4) inhibitors. Monoclonal antibodies can be designed to bind to specific target proteins to block the activity of target proteins in cancer cells (e.g., anti-EGFR, anti-VEGF, etc.).

In cancers, decreasing expression of genes involved in tumor proliferation, angiogenesis, or recognition by the immune system (e.g., VEGF, VEGFA, an isoform of VEGFA, PIGF, MICA, MICB, ERp5, ADAM, MMP, IDH1, CDK4, CDK6, EGFR, mTOR, KRAS, CD155, PD-L1, or c-Myc, etc.) while increasing expression of cytokines (e.g., IL-2, IL-12, IL-15, or IL-7, etc.) to enhance immune response could have a therapeutic effect. In one example, expression of IL-2, that can decrease proliferation rate of cancer cells such as head and neck squamous cell carcinoma (HNSCC) cells, can be increased. IL-2 is a cytokine that regulates lymphocyte activities and is a potent T-cell growth factor. IL-2 is produced by antigen-stimulated CD4+ T-cells, natural killer cells, or activated dendritic cells and is important for maintenance and differentiation of CD4+ regulatory T-cells. Without wishing to be bound by any theory, local IL-2 therapy can cause stagnation of the blood flow inside or near tumors and of the lymph drainage, leading to tumor necrosis and thrombosis. In another example, expression of VEGF, which can promote angiogenesis around tumor, can be decreased to block the supply of blood required for tumor growth. VEGF described herein may be any VEGF family members including VEGFA, an isoform of VEGFA, or PIGF. Non-limiting examples of VEGFA isoforms include, VEGF111, VEGF121, VEGF145, VEGF148, VEGF165, VEGF165B, VEGF183, VEGF189, VEGF206, L-VEGF121, L-VEGF165, L-VEGF189, L-VEGF206, Isoform 15, Isoform16, Isoform 17, and Isoform 18. In yet another example, expression of MICA and/or MICB (MICA/B), cell surface glycoproteins expressed by tumor cells, can be decreased to restore immune response of natural killer (NK) cells and T-cells to enhance tumor regression. MICA/B is recognized by natural killer group 2 member D (NKG2D) receptor expressed on NK cells and lymphocytes to promote recognition and elimination of tumor cells. Cancer cells may evade immune surveillance by shedding MICA/B from cell surface to impair NKG2D recognition. Cancer cells may also release soluble forms of MICA/B that can bind to NKGD2 receptor during tumor growth and hypoxia, which may induce NKG2D internalization, to escape immune responses and compromise immune surveillance by NK cells. Shedding or releasing of MICA/B from cell surface may be blocked by inhibiting or reducing the expression of proteins involved in shedding of a membrane protein. Examples of proteins involved in shedding include, but are not limited to, matrix metalloproteinases (MMPs) and a disintegrin and metalloproteinases (ADAMs). Non-limiting examples of MMPs include MMP1, MMP2, MMP3, MMP1, MMP8, MMP9, MMP10, MMP11, MMP12, MMP13, MMP14, MMP15, MMP16, MMP17, and MMP19. Shedding or releasing of MICA/B from cell surface may also be blocked by inhibiting or reducing the expression of factors regulating the proteins involved in shedding such as disulfide isomerase ERp5.

In some aspects, provided herein, is a method of treating a cancer in a subject, the method comprising administering to the subject RNA compositions or pharmaceutical compositions, described herein, comprising an mRNA encoding a gene of interest and siRNA capable of binding to a target mRNA. In some aspects, provided herein, are any RNA compositions or pharmaceutical compositions, described herein, comprising an mRNA encoding a gene of interest and siRNA capable of binding to a target mRNA for use in a method for the treatment of cancer. In some aspects, provided herein, is the use of RNA compositions or pharmaceutical compositions, described herein, comprising an mRNA encoding a gene of interest and siRNA capable of binding to a target mRNA for the manufacture of a medicament for treating cancer. In some aspects, provided herein, is the use of RNA compositions or pharmaceutical compositions, described herein, comprising an mRNA encoding a gene of interest and siRNA capable of binding to a target mRNA for treating cancer in a subject. In some embodiments, the siRNA is capable of binding to VEGF, VEGFA, an isoform of VEGFA, PIGF, IDH1, CDK4, CDK6, EGFR, mTOR, KRAS, CD155, PD-L1, c-Myc, a fragment thereof, or a functional variant thereof. In some embodiments, the siRNA is capable of binding to MICA, MICB, both MICA and MICB (MICA/B), ERp5, ADAM, MMP, a fragment thereof, or a functional variant thereof. In some embodiments, the ADAM is ADAM17. In some embodiments, the mRNA encoding the gene of interest encodes a cytokine. In some embodiments, the cytokine is an IL-2, IL-12, IL-15, IL-7, a fragment thereof, or a functional variant thereof.

In some aspects, provided herein, is a method of treating a cancer in a subject, the method comprising administering to the subject recombinant RNA compositions or pharmaceutical compositions, described herein, comprising siRNA capable of binding to VEGFA, IDH1, CDK4, CDK6, EGFR, mTOR, KRAS, CD155, PD-L1, or c-Myc and an mRNA encoding IL-2, IL-12, IL-15, or IL-7. In some aspects, provided herein, are recombinant RNA compositions or pharmaceutical compositions, described herein, comprising siRNA capable of binding to VEGFA, IDH1, CDK4, CDK6, EGFR, mTOR, KRAS, CD155, PD-L1, or c-Myc and an mRNA encoding IL-2, IL-12, IL-15, or IL-7 for use in a method for the treatment of cancer. In some aspects, provided herein, is the use of recombinant RNA compositions or pharmaceutical compositions, described herein, comprising siRNA capable of binding to VEGFA, IDH1, CDK4, CDK6, EGFR, mTOR, KRAS, CD155, PD-L1, or c-Myc and an mRNA encoding IL-2, IL-12, IL-15, or IL-7 for the manufacture of a medicament for treating cancer. In some aspects, provided herein, is the use of recombinant RNA compositions or pharmaceutical compositions, described herein, comprising siRNA capable of binding to VEGFA, IDH1, CDK4, CDK6, EGFR, mTOR, KRAS, CD155, PD-L1, or c-Myc and an mRNA encoding IL-2, IL-12, IL-15, or IL-7 for treating cancer in a subject. In some aspects, provided herein, is a method of treating a cancer in a subject, the method comprising administering to the subject recombinant RNA compositions or pharmaceutical compositions, described herein, comprising siRNA capable of binding to an mRNA of a VEGFA isoform and an mRNA encoding IL-2. In some aspects, provided herein, is a method of treating a cancer in a subject, the method comprising administering to the subject recombinant RNA compositions or pharmaceutical compositions, described herein, comprising siRNA capable of binding to a PIGF mRNA and an mRNA encoding IL-2. In some aspects, provided herein, is a method of treating a cancer in a subject, the method comprising administering to the subject recombinant RNA compositions or pharmaceutical compositions, described herein, comprising siRNA capable of binding to an mRNA of MICA or MICB and an mRNA encoding IL-2. In some aspects, provided herein, is a method of treating a cancer in a subject, the method comprising administering to the subject recombinant RNA compositions or pharmaceutical compositions, described herein, comprising siRNA capable of binding to an mRNA of ERp5, ADAM17, or MMP and an mRNA encoding IL-2. In some aspects, provided herein, is a method of treating a cancer in a subject, the method comprising administering to the subject recombinant RNA compositions or pharmaceutical compositions, described herein, comprising siRNA capable of binding to an mRNA of VEGFA, MICA, MICB, IDH1, CDK4, CDK6, EGFR, mTOR, KRAS, CD155, PD-L1, or c-Myc and an mRNA encoding IL-2, IL-7, IL-12, or IL-15.

In some aspects, compositions or pharmaceutical compositions administered to a subject in need thereof comprise recombinant RNA constructs comprising: (i) an IL-2 mRNA; and (ii) at least one siRNA capable of binding to a VEGFA mRNA. In related aspects, the polynucleic acid construct encodes or comprises at least 1, 2, 3, 4, or 5 siRNAs. In related aspects, recombinant RNA constructs may comprise 1 siRNA directed to a VEGFA mRNA. In related aspects, recombinant RNA constructs may comprise at least 3 or at least 5 siRNAs, each directed to a VEGFA mRNA. In related aspects, each of the at least 3 or at least 5 siRNAs is the same, different, or a combination thereof. In related aspects, recombinant RNA constructs may comprise a sequence as set forth in SEQ ID NO: 1-4 or 125-128 (Cpd.1-Cpd.4). In related aspects, recombinant RNA constructs may comprise a sequence as set forth in SEQ ID NO: 5 (Cpd.5), SEQ ID NO: 7 (Cpd.7), SEQ ID NO: 8 (Cpd.8), SEQ ID NO: 9 (Cpd.9), SEQ ID NO: 10 (Cpd.10), SEQ ID NO: 129 (Cpd.5), SEQ ID NO: 131 (Cpd.7), SEQ ID NO: 132 (Cpd.8), SEQ ID NO: 133 (Cpd.9), or SEQ ID NO: 134 (Cpd.10).

In some aspects, compositions or pharmaceutical compositions administered to a subject in need thereof comprise recombinant RNA constructs comprising: (i) an IL-2 mRNA; and (ii) at least one siRNA capable of binding to a PIGF mRNA. In related aspects, the polynucleic acid construct encodes or comprises at least 1, 2, or 3 siRNAs. In related aspects, recombinant RNA constructs may comprise 1 siRNA directed to a PIGF mRNA. In related aspects, recombinant RNA constructs may comprise at least 3 siRNAs, each directed to a PIGF mRNA. In related aspects, each of the at least 3 siRNAs is the same, different, or a combination thereof.

In some aspects, compositions or pharmaceutical compositions administered to a subject in need thereof comprise recombinant RNA constructs comprising: (i) an IL-2 mRNA; and (ii) at least one siRNA capable of binding to an mRNA of a VEGFA isoform. In related aspects, the polynucleic acid construct encodes or comprises at least 1, 2, or 3 siRNAs. In related aspects, recombinant RNA constructs may comprise 1 siRNA directed to an mRNA of a VEGFA isoform. In related aspects, recombinant RNA constructs may comprise at least 3 siRNAs, each directed to an mRNA of a VEGFA isoform. In related aspects, each of the at least 3 siRNAs is the same, different, or a combination thereof.

In some aspects, compositions or pharmaceutical compositions administered to a subject in need thereof comprise recombinant RNA constructs comprising: (i) an IL-2 mRNA; and (ii) at least one siRNA capable of binding to a MICA or MICB mRNA. In related aspects, recombinant RNA constructs may comprise at least 1, 2, or 3 siRNAs. In related aspects recombinant RNA constructs may comprise 1 siRNA directed to a MICA or MICB mRNA. In related aspects, recombinant RNA constructs may comprise at least 3 siRNAs, each directed to a MICA or MICB mRNA. In related aspects, each of the at least 3 siRNAs is the same, different, or a combination thereof. In related aspects, recombinant RNA constructs may comprise a sequence as set forth in SEQ ID NO: 1-4 or 125-128 (Cpd.1-Cpd.4). In related aspects, recombinant RNA constructs may comprise a sequence as set forth in SEQ ID NO: 6 or SEQ ID NO: 130 (Cpd.6).

In some aspects, compositions or pharmaceutical compositions administered to a subject in need thereof comprise recombinant RNA constructs comprising: (i) an IL-2 mRNA; and (ii) at least one siRNA capable of binding to an mRNA of ERp5, ADAM17, or MMP. In related aspects, recombinant RNA constructs may comprise at least 1, 2, or 3 siRNAs. In related aspects recombinant RNA constructs may comprise 1 siRNA directed to an mRNA of ERp5, ADAM17, or MMP. In related aspects, recombinant RNA constructs may comprise at least 3 siRNAs, each directed to an mRNA of ERp5, ADAM17, or MMP. In related aspects, each of the at least 3 siRNAs is the same, different, or a combination thereof.

In some aspects, compositions or pharmaceutical compositions administered to a subject in need thereof comprise recombinant RNA constructs comprising: (i) an IL-12 mRNA; and (ii) at least one siRNA capable of binding to an mRNA of IDH1, CDK4, and/or CDK6. In related aspects, the polynucleic acid construct encodes or comprises at least 1, 2, or 3 siRNAs. In related aspects, recombinant RNA constructs may comprise 1 siRNA directed to an IDH1 mRNA. In related aspects, recombinant RNA constructs may comprise 1 siRNA directed to a CDK4 mRNA. In related aspects, recombinant RNA constructs may comprise 1 siRNA directed to a CDK6 mRNA. In related aspects, recombinant RNA constructs may comprise 1 siRNA directed to an IDH1 mRNA, 1 siRNA directed to a CDK4 mRNA, and 1 siRNA directed to a CDK6 mRNA. In related aspects, recombinant RNA constructs may comprise at least 3 siRNAs, each directed to an IDH1 mRNA. In related aspects, recombinant RNA constructs may comprise at least 3 siRNAs, each directed to a CDK4 mRNA. In related aspects, recombinant RNA constructs may comprise at least 3 siRNAs, each directed to a CDK6 mRNA. In related aspects, each of the at least 3 siRNAs is the same, different, or a combination thereof. In related aspects, recombinant RNA constructs may comprise a sequence as set forth in SEQ ID NO: 11 or SEQ ID NO: 135 (Cpd.11).

In some aspects, compositions or pharmaceutical compositions administered to a subject in need thereof comprise recombinant RNA constructs comprising: (i) an IL-12 mRNA; and (ii) at least one siRNA capable of binding to an mRNA of EGFR, mTOR, and/or KRAS. In related aspects, the polynucleic acid construct encodes or comprises at least 1, 2, or 3 siRNAs. In related aspects, recombinant RNA constructs may comprise 1 siRNA directed to an EGFR mRNA. In related aspects, recombinant RNA constructs may comprise 1 siRNA directed to an mTOR mRNA. In related aspects, recombinant RNA constructs may comprise 1 siRNA directed to a KRAS mRNA. In related aspects, recombinant RNA constructs may comprise 1 siRNA directed to an EGFR mRNA, 1 siRNA directed to an mTOR mRNA, and 1 siRNA directed to a KRAS mRNA. In related aspects, recombinant RNA constructs may comprise at least 3 siRNAs, each directed to an EGFR mRNA. In related aspects, recombinant RNA constructs may comprise at least 3 siRNAs, each directed to an mTOR mRNA. In related aspects, recombinant RNA constructs may comprise at least 3 siRNAs, each directed to a KRAS mRNA. In related aspects, each of the at least 3 siRNAs is the same, different, or a combination thereof. In related aspects, recombinant RNA constructs may comprise a sequence as set forth in SEQ ID NO: 12 (Cpd.12), SEQ ID NO: 13 (Cpd.13), SEQ ID NO: 14 (Cpd.14), SEQ ID NO: 136 (Cpd.12), SEQ ID NO: 137 (Cpd.13), or SEQ ID NO: 138 (Cpd.14).

In some aspects, compositions or pharmaceutical compositions administered to a subject in need thereof comprise recombinant RNA constructs comprising: (i) an IL-15 mRNA; and (ii) at least one siRNA capable of binding to an mRNA of VEGFA and/or CD155. In related aspects, the polynucleic acid construct encodes or comprises at least 1, 2, or 3 siRNAs. In related aspects, recombinant RNA constructs may comprise 1 siRNA directed to a VEGFA mRNA. In related aspects, recombinant RNA constructs may comprise 1 siRNA directed to a CD155 mRNA. In related aspects, recombinant RNA constructs may comprise 1 siRNA directed to a VEGFA mRNA and 2 siRNAs directed to a CD155 mRNA. In related aspects, recombinant RNA constructs may comprise at least 3 siRNAs, each directed to a VEGFA mRNA. In related aspects, recombinant RNA constructs may comprise at least 3 siRNAs, each directed to a CD155 mRNA. In related aspects, each of the at least 3 siRNAs is the same, different, or a combination thereof. In related aspects, recombinant RNA constructs may comprise a sequence as set forth in SEQ ID NO: 15 or 139 (Cpd.15).

In some aspects, compositions or pharmaceutical compositions administered to a subject in need thereof comprise recombinant RNA constructs comprising: (i) an IL-15 mRNA; and (ii) at least one siRNA capable of binding to an mRNA of VEGFA, PD-L1, and/or c-Myc. In related aspects, the polynucleic acid construct encodes or comprises at least 1, 2, or 3 siRNAs. In related aspects, recombinant RNA constructs may comprise 1 siRNA directed to a VEGFA mRNA. In related aspects, recombinant RNA constructs may comprise 1 siRNA directed to a PD-L1 mRNA. In related aspects, recombinant RNA constructs may comprise 1 siRNA directed to a c-Myc mRNA. In related aspects, recombinant RNA constructs may comprise 1 siRNA directed to a VEGFA mRNA, 1 siRNA directed to a PD-L1 mRNA, and 1 siRNA directed to a c-Myc mRNA. In related aspects, recombinant RNA constructs may comprise at least 3 siRNAs, each directed to a VEGFA mRNA. In related aspects, recombinant RNA constructs may comprise at least 3 siRNAs, each directed to a PD-L1 mRNA. In related aspects, recombinant RNA constructs may comprise at least 3 siRNAs, each directed to a c-Myc mRNA. In related aspects, each of the at least 3 siRNAs is the same, different, or a combination thereof. In related aspects, recombinant RNA constructs may comprise a sequence as set forth in SEQ ID NO: 16 or 140 (Cpd.16).

In some aspects, compositions or pharmaceutical compositions administered to a subject in need thereof comprise recombinant RNA constructs comprising: (i) an IL-7 mRNA; and (ii) at least one siRNA capable of binding to an mRNA of PD-L1. In related aspects, the polynucleic acid construct encodes or comprises at least 1, 2, or 3 siRNAs. In related aspects, recombinant RNA constructs may comprise 1 siRNA directed to a PD-L1 mRNA. In related aspects, recombinant RNA constructs may comprise at least 3 siRNAs, each directed to a PD-L1 mRNA. In related aspects, each of the at least 3 siRNAs is the same, different, or a combination thereof. In related aspects, recombinant RNA constructs may comprise a sequence as set forth in SEQ ID NO: 17 or 141 (Cpd.17)

Recombinant RNA construct compositions described herein may be administered as a combination therapy. Combination therapies with two or more therapeutic agents or therapies may use agents and therapies that work by different mechanisms of action. Combination therapies using agents or therapies with different mechanisms of action can result in additive or synergetic effects. Combination therapies may allow for a lower dose of each agent than is used in monotherapy, thereby reducing toxic side effects and/or increasing the therapeutic index of the agent(s). Combination therapies can decrease the likelihood that resistant cancer cells will develop. In some instances, combination therapies comprise a therapeutic agent or therapy that affects the immune response (e.g., enhances or activates the response) and a therapeutic agent that affects (e.g., inhibits or kills) the tumor/cancer cells. In some instances, combination therapies may comprise (i) recombinant RNA compositions or pharmaceutical compositions described herein; and (ii) one or more additional therapy selected from surgical removal of tumors, radiation therapy, chemotherapy, targeted therapy, and immunotherapy. In some embodiments, recombinant RNA compositions or pharmaceutical compositions described herein may be administered to a subject with a cancer prior to, concurrently with, and/or subsequently to, administration of one or more additional therapy for combination therapies. In some embodiments, the one or more additional therapy comprises 1, 2, 3, or more additional therapeutic agents or therapies.

Compositions and pharmaceutical compositions described herein can be administered to a subject using any suitable methods known in the art. Suitable formulations for use in the present invention and methods of delivery are generally well known in the art. For example, compositions described herein can be administered to the subject in a variety of ways, including parenterally, intravenously, intradermally, intramuscularly, colonically, rectally, or intraperitoneally. In some embodiments, compositions described herein is administered by an injection to a subject. For example, compositions described herein can be administered by intraperitoneal injection, intramuscular injection, subcutaneous injection, intra-tumoral injection, or intravenous injection of the subject. In some embodiments, compositions described herein can be administered by an injection to a diseased organ or a diseased tissue of a subject. In some embodiments, compositions described herein can be administered by an injection to a tumor or cancer cells in a subject. In some embodiments, compositions described herein can be administered parenterally, intravenously, intramuscularly or orally.

Any of compositions and pharmaceutical compositions described herein may be provided together with an instruction manual. The instruction manual may comprise guidance for the skilled person or attending physician how to treat (or prevent) a disease or a disorder as described herein (e.g., a cancer such as a head and neck cancer) in accordance with the present invention. In some embodiments, the instruction manual may comprise guidance as to the herein described mode of delivery/administration and delivery/administration regimen, respectively (e.g., route of delivery/administration, dosage regimen, time of delivery/administration, frequency of delivery/administration, etc.). In some embodiments, the instruction manual may comprise the instruction that how compositions of the present invention is to be administrated or injected and/or is prepared for administration or injection.

In principle, what has been described herein elsewhere with respect to the mode of delivery/administration and delivery/administration regimen, respectively, may be comprised as respective instructions in the instruction manual.

Compositions and pharmaceutical compositions described herein can be used in a gene therapy. In certain embodiments, compositions comprising recombinant polynucleic acids or RNA constructs described herein can be delivered to a cell in gene therapy vectors. Gene therapy vectors and methods of gene delivery are well known in the art. Non-limiting examples of these methods include viral vector delivery systems including DNA and RNA viruses, which have either episomal or integrated genomes after delivery to the cell, non-viral vector delivery systems including DNA plasmids, naked nucleic acid, and nucleic acid complexed with a delivery vehicle, transposon system (for delivery and integration into the host genomes; Moriarity, et al. (2013) Nucleic Acids Res 41(8), e92, Aronovich, et al., (2011) Hum. Mol. Genet. 20(R1), R14-R20), retrovirus-mediated DNA transfer (e.g., Moloney Mouse Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus; see e.g., Kay et al. (1993) Science 262, 117-119, Anderson (1992) Science 256, 808-813), and DNA virus-mediated DNA transfer including adenovirus, herpes virus, parvovirus and adeno-associated virus (e.g., Ali et al. (1994) Gene Therapy 1, 367-384). Viral vectors also include but are not limited to adeno-associated virus, adenoviral virus, lentivirus, retroviral, and herpes simplex virus vectors. Vectors capable of integration in the host genome include but are not limited to retrovirus or lentivirus.

In some embodiments, compositions comprising recombinant polynucleic acid or RNA constructs described herein can be delivered to a cell via direct DNA transfer (Wolff et al. (1990) Science 247, 1465-1468). Recombinant polynucleic acid or RNA constructs can be delivered to cells following mild mechanical disruption of the cell membrane, temporarily permeabilizing the cells. Such a mild mechanical disruption of the membrane can be accomplished by gently forcing cells through a small aperture (Sharei et al. PLOS ONE (2015) 10(4), e0118803). In another embodiment, compositions comprising recombinant polynucleic acid or RNA constructs described herein can be delivered to a cell via liposome-mediated DNA transfer (e.g., Gao & Huang (1991) Biochem. Ciophys. Res. Comm. 179, 280-285, Crystal (1995) Nature Med. 1, 15-17, Caplen et al. (1995) Nature Med. 3, 39-46). A liposome can encompass a variety of single and multilamellar lipid vehicles formed by the generation of enclosed lipid bilayers or aggregates. Recombinant polynucleic acid or RNA constructs can be encapsulated in the aqueous interior of a liposome, interspersed within the lipid bilayer of a liposome, attached to a liposome via a linking molecule that is associated with both the liposome and the oligonucleotide, entrapped in a liposome, or complexed with a liposome.

### Modulation of Gene Expression

Provided herein are methods of simultaneously expressing an siRNA and an mRNA from a single RNA transcript in a cell, comprising introducing into the cell compositions comprising any recombinant polynucleic acid or RNA constructs described herein. Further provided herein are methods of simultaneously modulating expression of two or more genes in a cell, comprising introducing into the cell compositions comprising recombinant polynucleic acid or RNA constructs encoding or comprising a first RNA linked to a second RNA, wherein the first RNA encodes a gene of interest, and wherein the second RNA encodes a small interfering RNA (siRNA) capable of binding to a target messenger RNA (mRNA); wherein the target mRNA is different from an mRNA encoded by the gene of interest, and wherein the expression of the target mRNA and the gene of interest is modulated simultaneously. In some instances, expression of a polynucleic acid, gene, DNA, or RNA, as used herein, can refer to transcription and/or translation of the polynucleic acid, gene, DNA, or RNA. In some instances, modulating, increasing upregulating decreasing or downregulating expression of a polynucleic acid, gene such as a gene of interest, DNA, or RNA such as a target mRNA, as used herein, can refer to modulating, increasing, upregulating, decreasing, downregulating the level of protein encoded by a polynucleic acid, gene such as a gene of interest, DNA, or RNA such as a target mRNA by affecting transcription and/or translation of the polynucleic acid, gene such as a gene of interest, DNA, or RNA such as a target mRNA. In some instances, inhibiting expression of a polynucleic acid, gene such as a gene of interest, DNA, or RNA such as a target mRNA can refer to affect transcription and/or translation of the polynucleic acid, gene such as a gene of interest, DNA, or RNA such as a target mRNA such that the level of protein encoded by the polynucleic acid, gene such as a gene of interest, DNA, or RNA such as a target mRNA is reduced or abolished.

For example, provided herein, are methods of simultaneously modulating expression of two or more genes in a cell, comprising introducing into the cell compositions comprising recombinant polynucleic acid or RNA constructs encoding or comprising a first RNA linked to a second RNA, wherein the first RNA encodes a cytokine, and wherein the second RNA encodes a small interfering RNA (siRNA) capable of binding to an mRNA associated with tumor proliferation, angiogenesis, or recognition by the immune system; wherein the expression of the mRNA of which the protein product is associated with tumor proliferation, angiogenesis, or recognition by the immune system and the cytokine is modulated simultaneously.

Provided herein are methods of simultaneously modulating expression of two or more genes in a cell, comprising introducing into the cell compositions comprising recombinant polynucleic acid or RNA constructs comprising a first RNA linked to a second RNA wherein the first RNA encodes IL-2, and wherein the second RNA encodes a small interfering RNA (siRNA) capable of binding to a VEGFA mRNA; wherein the expression of IL-2 and VEGFA is modulated simultaneously, i.e. the expression of IL-2 is upregulated and the expression of VEGFA is downregulated simultaneously. In related aspects, recombinant polynucleic acid or RNA constructs may encode or comprise at least 1, 2, 3, 4, 5, or more siRNAs. In related aspects, recombinant polynucleic acid or RNA constructs may encode or comprise 3 siRNAs, each directed to the same region of a VEGFA mRNA. In related aspects, recombinant polynucleic acid or RNA constructs may encode or comprise 3 siRNAs, each directed to a different region of a VEGFA mRNA. In related aspects, each of the at least 3 siRNAs is directed to the same, different, or a combination thereof. In related aspects, recombinant polynucleic acid constructs may comprise a sequence comprising in SEQ ID NO: 86 (Cpd.5), SEQ ID NO: 88 (Cpd.7), SEQ ID NO: 89 (Cpd.8), SEQ ID NO: 90 (Cpd.7), or SEQ ID NO: 91 (Cpd.10). In related aspects, recombinant RNA constructs may comprise a sequence comprising in SEQ ID NO: 5 (Cpd.5), SEQ ID NO: 7 (Cpd.7), SEQ ID NO: 8 (Cpd.8), SEQ ID NO: 9 (Cpd.9), SEQ ID NO: 10 (Cpd.10), SEQ ID NO: 129 (Cpd.5), SEQ ID NO: 131 (Cpd.7), SEQ ID NO: 132 (Cpd.8), SEQ ID NO: 133 (Cpd.9), or SEQ ID NO: 134 (Cpd.10).

Also provided herein are methods of simultaneously modulating expression of two or more genes in a cell, comprising introducing into the cell compositions comprising recombinant polynucleic acid or RNA constructs encoding or comprising a first RNA linked to a second RNA wherein the first RNA encodes IL-2, and wherein the second RNA encodes a small interfering RNA (siRNA) capable of binding to an mRNA of a VEGFA isoform; wherein the expression of IL-2 and an isoform of VEGFA is modulated simultaneously, i.e. the expression of IL-2 is upregulated and the expression of an isoform of VEGFA is downregulated simultaneously. In related aspects, recombinant polynucleic acid or RNA constructs may encode or comprise at least 1, 2, 3, 4, 5, or more siRNAs. In related aspects, recombinant polynucleic acid or RNAconstructs may encode or comprise 3 siRNAs, each directed to the same region of an mRNA of a VEGFA isoform. In related aspects, recombinant polynucleic acid or RNA constructs may encode or comprise 3 siRNAs, each directed to a different region of an mRNA of a VEGFA isoform. In related aspects, each of the at least 3 siRNAs is directed to the same, different, or a combination thereof.

Further provided herein are methods of simultaneously modulating expression of two or more genes in a cell, comprising introducing into the cell compositions comprising recombinant polynucleic acid or RNA constructs encoding or comprising a first RNA linked to a second RNA wherein the first RNA encodes IL-2, and wherein the second RNA encodes a small interfering RNA (siRNA) capable of binding to a PIGF mRNA; wherein the expression of IL-2 and PIGF is modulated simultaneously, i.e. the expression of IL-2 is upregulated and the expression of PIGF is downregulated simultaneously. In related aspects, recombinant polynucleic acid or RNA constructs may encode or comprise at least 1, 2, 3, 4, 5, or more siRNAs. In related aspects, recombinant polynucleic acid or RNA constructs may encode or comprise 3 siRNAs, each directed to the same region of a PIGF mRNA. In related aspects, recombinant polynucleic acid or RNA constructs may encode or comprise 3 siRNAs, each directed to a different region of a PIGF mRNA. In related aspects, each of the at least 3 siRNAs is directed to the same, different, or a combination thereof.

Provided herein are methods of simultaneously modulating expression of two or more genes in a cell, comprising introducing into the cell compositions comprising recombinant polynucleic acid or RNA constructs encoding or comprising a first RNA linked to a second RNA wherein the first RNA encodes IL-2, and wherein the second RNA encodes a small interfering RNA (siRNA) capable of binding to a MICA and/or MICB (MICA/B) mRNA; wherein the expression of IL-2 and MICA/B is modulated simultaneously, i.e. the expression of IL-2 is upregulated and the expression of MICA/B is downregulated simultaneously. In related aspects, recombinant polynucleic acid or RNA constructs may encode or comprise at least 1, 2, 3, 4, 5, or more siRNAs. In related aspects, recombinant polynucleic acid or RNA constructs may encode or comprise 3 siRNAs, each directed to the same region of a MICA/B mRNA. In related aspects, recombinant polynucleic acid or RNA constructs may encode or comprise 3 siRNAs, each directed to a different region of a MICA/B mRNA. In related aspects, each of the at least 3 siRNAs is directed to the same, different, or a combination thereof. In related aspects, recombinant polynucleic acid constructs may comprise a sequence comprising in SEQ ID NO: 87 (Cpd.6). In related aspects, recombinant RNA constructs may comprise a sequence comprising in SEQ ID NO: 6 or 130 (Cpd.6).

Also provided herein are methods of simultaneously modulating expression of two or more genes in a cell, comprising introducing into the cell compositions comprising recombinant polynucleic acid or RNA constructs encoding or comprising a first RNA linked to a second RNA wherein the first RNA encodes IL-2, and wherein the second RNA encodes a small interfering RNA (siRNA) capable of binding to an mRNA of ERp5, ADAM, or MMP; wherein the expression of IL-2 and ERp5, ADAM, or MMP is modulated simultaneously, i.e. the expression of IL-2 is upregulated and the expression of ERp5, ADAM, or MMP is downregulated simultaneously. In some embodiments, the ADAM is ADAM17. In related aspects, recombinant polynucleic acid or RNA constructs may encode or comprise at least 1, 2, 3, 4, 5, or more siRNAs. In related aspects, recombinant polynucleic acid or RNA constructs may encode or comprise 3 siRNAs, each directed to the same region of an mRNA of ERp5, ADAM17, or MMP. In related aspects, recombinant polynucleic acid or RNA constructs may encode or comprise 3 siRNAs, each directed to a different region of an mRNA of ERp5, ADAM17, or MMP. In related aspects, each of the at least 3 siRNAs is directed to the same, different, or a combination thereof.

Provided herein are methods of simultaneously modulating expression of two or more genes in a cell, comprising introducing into the cell compositions comprising recombinant polynucleic acid or RNA constructs comprising a first RNA linked to a second RNA wherein the first RNA encodes IL-12, and wherein the second RNA encodes a small interfering RNA (siRNA) capable of binding to an mRNA of IDH1, CDK4, and/or CDK6; wherein the expression of IL-12, IDH1, CDK4, and/or CDK6 is modulated simultaneously, i.e. the expression of IL-12 is upregulated and the expression of IDH1, CDK4, and/or CDK6 is downregulated simultaneously. In related aspects, recombinant polynucleic acid or RNA constructs may encode or comprise at least 1, 2, 3, 4, 5, or more siRNAs. In related aspects, recombinant polynucleic acid or RNA constructs may encode or comprise 3 siRNAs, each directed to the same region of an mRNA of IDH1, CDK4, and/or CDK6. In related aspects, recombinant polynucleic acid or RNA constructs may encode or comprise 3 siRNAs, each directed to a different region of an mRNA of IDH1, CDK4, and/or CDK6. In related aspects, each of the at least 3 siRNAs is directed to the same, different, or a combination thereof. In related aspects, recombinant polynucleic acid or RNA constructs may encode or comprise 1 siRNA directed to an mRNA of IDH1, 1 siRNA directed to an mRNA of CDK4, and 1 siRNA directed to an mRNA of CDK6. In related aspects, recombinant polynucleic acid constructs may comprise a sequence comprising in SEQ ID NO: 92 (Cpd.11). In related aspects, recombinant RNA constructs may comprise a sequence comprising in SEQ ID NO: 11 or 135 (Cpd.11).

Provided herein are methods of simultaneously modulating expression of two or more genes in a cell, comprising introducing into the cell compositions comprising recombinant polynucleic acid or RNA constructs comprising a first RNA linked to a second RNA wherein the first RNA encodes IL-12, and wherein the second RNA encodes a small interfering RNA (siRNA) capable of binding to an mRNA of EGFR, mTOR, and/or KRAS; wherein the expression of IL-12, EGFR, mTOR, and/or KRAS is modulated simultaneously, i.e. the expression of IL-12 is upregulated and the expression of EGFR, mTOR, and/or KRAS is downregulated simultaneously. In related aspects, recombinant polynucleic acid or RNA constructs may encode or comprise at least 1, 2, 3, 4, 5, or more siRNAs. In related aspects, recombinant polynucleic acid or RNA constructs may encode or comprise 3 siRNAs, each directed to the same region of an mRNA of EGFR, mTOR, and/or KRAS. In related aspects, recombinant polynucleic acid or RNA constructs may encode or comprise 3 siRNAs, each directed to a different region of an mRNA of EGFR, mTOR, and/or KRAS. In related aspects, each of the at least 3 siRNAs is directed to the same, different, or a combination thereof. In related aspects, recombinant polynucleic acid or RNA constructs may encode or comprise 1 siRNA directed to an mRNA of EGFR, 1 siRNA directed to an mRNA of mTOR, and 1 siRNA directed to an mRNA of KRAS. In related aspects, recombinant polynucleic acid constructs may comprise a sequence comprising in SEQ ID NO: 93 (Cpd.12), SEQ ID NO: 94 (Cpd.13), or SEQ ID NO: 95 (Cpd.14). In related aspects, recombinant RNA constructs may comprise a sequence comprising in SEQ ID NO: 12 (Cpd.12), SEQ ID NO: 13 (Cpd.13), SEQ ID NO: 14 (Cpd.14), SEQ ID NO: 136 (Cpd.12), SEQ ID NO: 137 (Cpd.13), or SEQ ID NO: 138 (Cpd.14).

Provided herein are methods of simultaneously modulating expression of two or more genes in a cell, comprising introducing into the cell compositions comprising recombinant polynucleic acid or RNA constructs comprising a first RNA linked to a second RNA wherein the first RNA encodes IL-15, and wherein the second RNA encodes a small interfering RNA (siRNA) capable of binding to an mRNA of VEGFA and/or CD155; wherein the expression of IL-15, VEGFA, and/or CD155 is modulated simultaneously, i.e. the expression of IL-15 is upregulated and the expression of VEGFA and/or CD155 is downregulated simultaneously. In related aspects, recombinant polynucleic acid or RNA constructs may encode or comprise at least 1, 2, 3, 4, 5, or more siRNAs. In related aspects, recombinant polynucleic acid or RNA constructs may encode or comprise 3 siRNAs, each directed to the same region of an mRNA of VEGFA and/or CD155. In related aspects, recombinant polynucleic acid or RNA constructs may encode or comprise 3 siRNAs, each directed to a different region of an mRNA of VEGFA and/or CD155. In related aspects, each of the at least 3 siRNAs is directed to the same, different, or a combination thereof. In related aspects, recombinant polynucleic acid or RNA constructs may encode or comprise 1 siRNA directed to an mRNA of VEGFA and 2 siRNAs directed to an mRNA of CD155. In related aspects, recombinant polynucleic acid constructs may comprise a sequence comprising in SEQ ID NO: 96 (Cpd.15). In related aspects, recombinant RNA constructs may comprise a sequence comprising in SEQ ID NO: or 139 (Cpd.15).

Provided herein are methods of simultaneously modulating expression of two or more genes in a cell, comprising introducing into the cell compositions comprising recombinant polynucleic acid or RNA constructs comprising a first RNA linked to a second RNA wherein the first RNA encodes IL-15, and wherein the second RNA encodes a small interfering RNA (siRNA) capable of binding to an mRNA of VEGFA, PD-L1, and/or c-Myc; wherein the expression of IL-15, VEGFA, PD-L1, and/or c-Myc is modulated simultaneously, i.e. the expression of IL-15 is upregulated and the expression of VEGFA, PD-L1, and/or c-Myc is downregulated simultaneously. In related aspects, recombinant polynucleic acid or RNA constructs may encode or comprise at least 1, 2, 3, 4, 5, or more siRNAs. In related aspects, recombinant polynucleic acid or RNA constructs may encode or comprise 3 siRNAs, each directed to the same region of an mRNA of VEGFA, PD-L1, and/or c-Myc. In related aspects, recombinant polynucleic acid or RNA constructs may encode or comprise 3 siRNAs, each directed to a different region of an mRNA of VEGFA, PD-L1, and/or c-Myc. In related aspects, each of the at least 3 siRNAs is directed to the same, different, or a combination thereof. In related aspects, recombinant polynucleic acid or RNA constructs may encode or comprise 1 siRNA directed to an mRNA of VEGFA, 1 siRNA directed to an mRNA of PD-L1, and 1 siRNA directed to an mRNA of c-Myc. In related aspects, recombinant polynucleic acid constructs may comprise a sequence comprising in SEQ ID NO: 97 (Cpd.16). In related aspects, recombinant RNA constructs may comprise a sequence comprising in SEQ ID NO: 16 or 140 (Cpd.16).

Provided herein are methods of simultaneously modulating expression of two or more genes in a cell, comprising introducing into the cell compositions comprising recombinant polynucleic acid or RNA constructs comprising a first RNA linked to a second RNA wherein the first RNA encodes IL-7, and wherein the second RNA encodes a small interfering RNA (siRNA) capable of binding to a PD-L1 mRNA; wherein the expression of IL-7 and PD-L1 is modulated simultaneously, i.e. the expression of IL-7 is upregulated and the expression of PD-L1 is downregulated simultaneously. In related aspects, recombinant polynucleic acid or RNA constructs may encode or comprise at least 1, 2, 3, 4, 5, or more siRNAs. In related aspects, recombinant polynucleic acid or RNA constructs may encode or comprise 3 siRNAs, each directed to the same region of a PD-L1 mRNA. In related aspects, recombinant polynucleic acid or RNA constructs may encode or comprise 3 siRNAs, each directed to a different region of a PD-L1 mRNA. In related aspects, each of the at least 3 siRNAs is directed to the same, different, or a combination thereof. In related aspects, recombinant polynucleic acid constructs may comprise a sequence comprising in SEQ ID NO: 98 (Cpd.17). In related aspects, recombinant RNA constructs may comprise a sequence comprising in SEQ ID NO: 17 or 141 (Cpd.17).

Provided herein are methods of simultaneously upregulating and downregulating expression of two or more genes in a cell, comprising introducing into the cell compositions comprising recombinant polynucleic acid or RNA constructs encoding or comprising a first RNA linked to a second RNA wherein the first RNA encodes a gene of interest (e.g., IL-2, IL-12, IL-15, or IL-7), and wherein the second RNA encodes a small interfering RNA (siRNA) capable of binding to a target mRNA (e.g., VEGFA, a VEGFA isoform, PIGF, MICA, MICB, ERp5, ADAM, MMP, IDH1, CDK4, CDK6, EGFR, mTOR, KRAS, CD155, PD-L1, or c-Myc); wherein the target mRNA is different from an mRNA encoded by the gene of interest, and wherein the expression of the target mRNA is downregulated and the expression of the gene of interest is upregulated simultaneously. In some embodiments, the ADAM is ADAM17. In some embodiments, the expression of the target mRNA is downregulated by the siRNA capable of binding to the target mRNA. In some embodiments, the expression of the gene of interest is upregulated by expressing an mRNA or a protein encoded by the gene of interest.

### ILLUSTRATIVE EMBODIMENTS

In some aspects, provided herein, is a composition comprising a first RNA linked to a second RNA, wherein the first RNA encodes for a cytokine, and wherein the second RNA encodes for a genetic element that modulates expression of a gene associated with tumor proliferation. In some embodiments, the cytokine is interleukin-2 (IL-2), IL-12, IL-15, IL-7, a fragment thereof, or a functional variant thereof. In some embodiments, the cytokine comprises a sequence selected from the group consisting of SEQ ID NOs: 24, 44, 47, 68, and 80. In some embodiments, the cytokine comprises a signal peptide. In some embodiments, the signal peptide comprises an unmodified signal peptide sequence or a modified signal peptide sequence. In some embodiments, the unmodified signal peptide sequence comprises a sequence selected from the group consisting of SEQ ID NOs: 26 and 125-128. In some embodiments, the IL-2 comprises a signal peptide. In some embodiments, the signal peptide comprises an unmodified IL-2 signal peptide sequence. In some embodiments, the unmodified IL-2 signal peptide sequence comprises a sequence listed in SEQ ID NO: 26. In some embodiments, the signal peptide comprises an IL-2 signal peptide sequence modified by insertion, deletion, or substitution of at least one amino acid. In some embodiments, the IL-2 signal peptide sequence modified by insertion, deletion, or substitution of at least one amino acid comprises a sequence selected from the group consisting of SEQ ID NOs: 27-29.

In some embodiments, the first RNA is a messenger RNA (mRNA). In some embodiments, the second RNA is a small interfering RNA (siRNA). In some embodiments, the siRNA is capable of binding to an mRNA of the gene associated with tumor proliferation. In some embodiments, the second RNA comprises 1, 2, 3, 4, 5, or more species of siRNA, wherein each species of siRNA comprises a different sequence targeting a different region of the same mRNA. In some embodiments, the second RNA comprises 1, 2, 3, 4, 5, or more redundant species of siRNA. In some embodiments, each species of the 1, 2, 3, 4, 5, or more species of siRNA is connected by a linker comprising a sequence listed in SEQ ID NO: 22.

In some embodiments, the gene associated with tumor proliferation comprises a gene associated with angiogenesis. In some embodiments, the gene associated with angiogenesis encodes vascular endothelial growth factor (VEGF), a fragment thereof, or a functional variant thereof. In some embodiments, the VEGF is VEGFA, a fragment thereof, or a functional variant thereof. In some embodiments, the VEGFA comprises a sequence listed in SEQ ID NO: 35. In some embodiments, the VEGF is an isoform of VEGFA, a fragment thereof, or a functional variant thereof. In some embodiments, the VEGF is placental growth factor (PIGF), a fragment thereof, or a functional variant thereof. In some embodiments, the gene associated with tumor proliferation comprises isocitrate dehydrogenase (IDH1), cyclin-dependent kinase 4 (CDK4), CDK6, epidermal growth factor receptor (EGFR), mechanistic target of rapamycin (mTOR), Kirsten rat sarcoma viral oncogene (KRAS), cluster of differentiation (CD155), programmed cell death-ligand 1 (PD-L1), or myc proto-oncogene (c-Myc). In some embodiments, the gene associated with tumor proliferation comprises a sequence selected from the group consisting of SEQ ID NOs: 50, 53, 56, 59, 62, 65, 71, 74, and 77.

In some embodiments, the first RNA is linked to the second RNA by a linker. In some embodiments, the linker comprises a tRNA linker or a linker comprising a sequence listed in SEQ ID NO: 21. In some embodiments, the compositions described herein further comprises a poly(A) tail, a 5′ cap, or a Kozak sequence. In some embodiments, the first RNA and the second RNA are both recombinant.

In some aspects, provided herein, is a composition comprising a first RNA linked to a second RNA, wherein the first RNA encodes for a cytokine, and wherein the second RNA encodes for a genetic element that modulates expression of a gene associated with recognition by the immune system. In some embodiments, the cytokine is interleukin-2 (IL-2), a fragment thereof, or a functional variant thereof. In some embodiments, the IL-2 comprises a sequence listed in SEQ ID NO: 24. In some embodiments, the IL-2 comprises a signal peptide. In some embodiments, the signal peptide comprises an unmodified IL-2 signal peptide sequence. In some embodiments, the unmodified IL-2 signal peptide sequence comprises a sequence listed in SEQ ID NO: 26. In some embodiments, the signal peptide comprises an IL-2 signal peptide sequence modified by insertion, deletion, or substitution of at least one amino acid. In some embodiments, the IL-2 signal peptide sequence modified by insertion, deletion, or substitution of at least one amino acid comprises a sequence selected from the group consisting of SEQ ID NOs: 27-29.

In some embodiments, the first RNA is a messenger RNA (mRNA). In some embodiments, the second RNA is a small interfering RNA (siRNA). In some embodiments, the siRNA is capable of binding to an mRNA of the gene associated with recognition by the immune system encoding for cell surface localizing protein. In some embodiments, the gene associated with recognition by the immune system encodes MHC class I chain-related sequence A (MICA), a fragment thereof, or a functional variant thereof. In some embodiments, the MICA comprises a sequence listed in SEQ ID NO: 38. In some embodiments, the gene associated with immune system surveillance encodes MHC class I chain-related sequence B (MICB), a fragment thereof, or a functional variant thereof. In some embodiments, the MICB comprises a sequence listed in SEQ ID NO: 41. In some embodiments, the gene associated with recognition by the immune system encodes endoplasmic reticulum protein (ERp5), a disintegrin and metalloproteinase (ADAM), matrix metalloproteinase (MMP), a fragment thereof, or a functional variant thereof. In some embodiments, the ADAM is ADAM17. In some embodiments, the second RNA comprises 1, 2, 3, 4, 5, or more species of siRNA, wherein each species of siRNA comprises a different sequence targeting a different region of the same mRNA. In some embodiments, the second RNA comprises 1, 2, 3, 4, 5, or more redundant species of siRNA. In some embodiments, each species of the 1, 2, 3, 4, 5, or more species of siRNA is connected by a linker comprising a sequence listed in SEQ ID NO: 22.

In some embodiments, the first RNA is linked to the second RNA by a linker. In some embodiments, the linker comprises a tRNA linker or a linker comprising a sequence listed in SEQ ID NO: 21. In some embodiments, the compositions described herein further comprises a poly(A) tail, a 5′ cap, or a Kozak sequence. In some embodiments, the first RNA and the second RNA are both recombinant.

In some aspects, provided herein, is a composition comprising a first RNA encoding for interleukin-2 (IL-2), IL-15, a fragment thereof, or a functional variant thereof linked to a second RNA encoding for a genetic element that modulates expression of vascular endothelial growth factor A (VEGFA), an isoform of VEGFA, placental growth factor (PIGF), cluster of differentiation 155 (CD155), programmed cell death-ligand 1 (PD-L1), myc proto-oncogene (c-Myc), a fragment thereof, or a functional variant thereof. In some embodiments, the first RNA is a messenger RNA (mRNA). In some embodiments, the IL-2 comprises a sequence listed in SEQ ID NO: 24. In some embodiments, the signal peptide comprises an unmodified IL-2 signal peptide sequence. In some embodiments, the unmodified IL-2 signal peptide sequence comprises a sequence listed in SEQ ID NO: 26. In some embodiments, the signal peptide comprises an IL-2 signal peptide sequence modified by insertion, deletion, or substitution of at least one amino acid. In some embodiments, the IL-2 signal peptide sequence modified by insertion, deletion, or substitution of at least one amino acid comprises a sequence selected from the group consisting of SEQ ID NOs: 27-29. In some embodiments, the IL-15 comprises a sequence comprising SEQ ID NO: 68. In some embodiments, the IL-15 comprises a signal peptide. In some embodiments, the signal peptide comprises an unmodified IL-15 signal peptide sequence. In some embodiments, the unmodified IL-15 signal peptide sequence comprises a sequence listed in SEQ ID NO: 144.

In some embodiments, the second RNA is a small interfering RNA (siRNA). In some embodiments, the siRNA is capable of binding to an mRNA of VEGFA, an isoform of VEGFA, PIGF, CD155, PD-L1, or c-Myc. In some embodiments, the VEGFA comprises a sequence listed in SEQ ID NO: 35. In some embodiments, the CD155 comprises a sequence comprising SEQ ID NO: 71. In some embodiments, the PD-L1 comprises a sequence comprising SEQ ID NO: 74. In some embodiments, the c-Myc comprises a sequence comprising SEQ ID NO: 77. In some embodiments, the second RNA comprises 1, 2, 3, 4, 5, or more species of siRNA, wherein each species of siRNA comprises a different sequence targeting a different region of the same mRNA. In some embodiments, the second RNA comprises 1, 2, 3, 4, 5, or more redundant species of siRNA. In some embodiments, each species of the 1, 2, 3, 4, 5, or more species of siRNA is connected by a linker comprising a sequence listed in SEQ ID NO: 22.

In some embodiments, the first RNA is linked to the second RNA by a linker. In some embodiments, the linker comprises a tRNA linker or a linker comprising a sequence listed in SEQ ID NO: 21. In some embodiments, the compositions described herein further comprises a poly(A) tail, a 5′ cap, or a Kozak sequence. In some embodiments, the first RNA and the second RNA are both recombinant.

In some aspects, provided herein, is a composition comprising a first RNA encoding for interleukin-2 (IL-2), a fragment thereof, or a functional variant thereof linked to a second RNA encoding for a genetic element that modulates expression of MHC class I chain-related sequence A (MICA), MHC class I chain-related sequence B (MICB), endoplasmic reticulum protein (ERp5), a disintegrin and metalloproteinase (ADAM), matrix metalloproteinase (MMP), a fragment thereof, or a functional variant thereof. In some embodiments, the ADAM is ADAM17. In some embodiments, the first RNA is a messenger RNA (mRNA). In some embodiments, the IL-2 comprises a sequence listed in SEQ ID NO: 24. In some embodiments, the signal peptide comprises an unmodified IL-2 signal peptide sequence. In some embodiments, the unmodified IL-2 signal peptide sequence comprises a sequence listed in SEQ ID NO: 26. In some embodiments, the signal peptide comprises an IL-2 signal peptide sequence modified by insertion, deletion, or substitution of at least one amino acid. In some embodiments, the IL-2 signal peptide sequence modified by insertion, deletion, or substitution of at least one amino acid comprises a sequence selected from the group consisting of SEQ ID NOs: 27-29. In some embodiments, the second RNA is a small interfering RNA (siRNA). In some embodiments, the siRNA is capable of binding to an mRNA of MICA, MICB, ERp5, ADAM, or MMP. In some embodiments, the MICA comprises a sequence listed in SEQ ID NO: 38. In some embodiments, the MICB comprises a sequence listed in SEQ ID NO: 41. In some embodiments, the ADAM is ADAM17. In some embodiments, the second RNA comprises 1, 2, 3, 4, 5, or more species of siRNA, wherein each species of siRNA comprises a different sequence targeting a different region of the same mRNA. In some embodiments, the second RNA comprises 1, 2, 3, 4, 5, or more redundant species of siRNA. In some embodiments, each species of the 1, 2, 3, 4, 5, or more species of siRNA is connected by a linker comprising a sequence listed in SEQ ID NO: 22. In some embodiments, the first RNA is linked to the second RNA by a linker. In some embodiments, the linker comprises a tRNA linker or a linker comprising a sequence listed in SEQ ID NO: 21. In some embodiments, the compositions described herein further comprises a poly(A) tail, a 5′ cap, or a Kozak sequence. In some embodiments, the first RNA and the second RNA are both recombinant.

In some aspects, provided herein, is a composition comprising a first RNA encoding for interleukin-12 (IL-12), IL-7, a fragment thereof, or a functional variant thereof linked to a second RNA encoding for a genetic element that modulates expression of isocitrate dehydrogenase (IDH1), cyclin-dependent kinase 4 (CDK4), CDK6, epidermal growth factor receptor (EGFR), mechanistic target of rapamycin (mTOR), Kirsten rat sarcoma viral oncogene (KRAS), programmed cell death-ligand 1 (PD-L1), a fragment thereof, or a functional variant thereof.

In some embodiments, the first RNA is a messenger RNA (mRNA). In some embodiments, the IL-12 comprises a sequence comprising SEQ ID NO: 44 or SEQ ID NO: 47. In some embodiments, the IL-12 comprises a signal peptide. In some embodiments, the signal peptide comprises an unmodified IL-12 signal peptide. In some embodiments, the unmodified IL-12 signal peptide comprises a sequence listed in SEQ ID NO: 142 or SEQ ID NO: 143. In some embodiments, the IL-7 comprises a sequence comprising SEQ ID NO: 80. In some embodiments, the IL-7 comprises a signal peptide. In some embodiments, the signal peptide comprises an unmodified IL-7 signal peptide. In some embodiments, the unmodified IL-7 signal peptide comprises a sequence listed in SEQ ID NO: 128.

In some embodiments, the second RNA is a small interfering RNA (siRNA). In some embodiments, the siRNA is capable of binding to an mRNA of IDH1, CDK4, CDK6, EGFR, mTOR, KRAS, or PD-L1. In some embodiments, IDH1 comprises a sequence comprising SEQ ID NO: 50. In some embodiments, CDK4 comprises a sequence comprising SEQ ID NO: 53. In some embodiments, CDK6 comprises a sequence comprising SEQ ID NO: 56. In some embodiments, mTOR comprises a sequence comprising SEQ ID NO: 62. In some embodiments, EGFR comprises a sequence comprising SEQ ID NO: 59. In some embodiments, KRAS comprises a sequence comprising SEQ ID NO: 65. In some embodiments, PD-L1 comprises a sequence comprising SEQ ID NO: 74.

In some embodiments, the second RNA comprises 1, 2, 3, 4, 5, or more species of siRNA, wherein each species of siRNA comprises a different sequence targeting a different region of the same mRNA. In some embodiments, the second RNA comprises 1, 2, 3, 4, 5, or more redundant species of siRNA. The composition of claim 119 or 120, wherein each species of the 1, 2, 3, 4, 5, or more species of siRNA is connected by a linker comprising a sequence listed in SEQ ID NO: 22.

In some embodiments, the first RNA is linked to the second RNA by a linker. In some embodiments, the linker comprises a tRNA linker or a linker comprising a sequence comprising SEQ ID NO: 21. In some embodiments, the composition further comprises a poly(A) tail, a 5′ cap, or a Kozak sequence. In some embodiments, the first RNA and the second RNA are both recombinant.

In some aspects, provided herein, is a pharmaceutical composition comprising any of the compositions described herein and a pharmaceutically acceptable excipient. In some aspects, provided herein, is a method of treating cancer, comprising administering any of compositions or pharmaceutical compositions described herein to a subject having a cancer. In some aspects, provided herein, are any of compositions or pharmaceutical compositions described herein for use in a method for the treatment of cancer. In some aspects, provided herein, is the use of any of compositions or pharmaceutical compositions described herein for the manufacture of a medicament for treating cancer. In some aspects, provided herein, is the use of any of compositions or pharmaceutical compositions described herein for treating cancer in a subject. In some embodiments, the cancer is a solid tumor. In some embodiments, the cancer is melanoma. In some embodiments, the cancer is renal cell carcinoma. In some embodiments, the cancer is a head and neck cancer. In some embodiments, the head and neck cancer is head and neck squamous cell carcinoma. In some embodiments, the head and neck cancer is laryngeal cancer, hypopharyngeal cancer, tonsil cancer, nasal cavity cancer, paranasal sinus cancer, nasopharyngeal cancer, metastatic squamous neck cancer with occult primary, lip cancer, oral cancer, oral cancer, oropharyngeal cancer, salivary gland cancer, brain tumors, esophageal cancer, eye cancer, parathyroid cancer, sarcoma of the head and neck, or thyroid cancer. In some embodiments, the cancer is located at an upper aerodigestive tract. In some embodiments, the upper aerodigestive tract comprises a paranasal sinus, a nasal cavity, an oral cavity, a salivary gland, a tongue, a nasopharynx, an oropharynx, a hypopharynx, or a larynx. In some embodiments, the subject has a head and neck cancer. In some embodiments, the subject having the head and neck cancer has a history of tobacco usage. In some embodiments, the subject having the head and neck cancer has a human papillomavirus (HPV) DNA. In some embodiments, the subject is a human.

In some aspects, provided herein, is a composition comprising a recombinant polynucleic acid construct comprising a nucleic acid sequence selected from the group consisting of SEQ ID NOs: 1-17 and 125-141.

In some aspects, provided herein, is a composition for use in modulating the expression of two or more genes in a cell. In some aspects, provided herein is a cell comprising any one of the compositions described herein. In some aspects, provided herein is a vector comprising a recombinant polynucleic acid construct encoding any one of the compositions described herein.

In some aspects, provided herein is a method of producing an siRNA and an mRNA from a single RNA transcript in a cell, comprising introducing into the cell any one of the compositions described herein or the vectors described herein. In some aspects, provided herein is a method of modulating protein expression comprising introducing any one of the compositions described herein or the vectors described herein into a cell, wherein the expression of a protein encoded by the second RNA is decreased compared to a cell without the composition or vector. In some aspects, provided herein is a method of modulating protein expression comprising introducing any one of the compositions described herein or the vectors described herein into a cell, wherein the expression of a protein encoded by the first RNA is increased compared to a cell without the composition or vector. In some aspects, provided herein is a method of modulating protein expression comprising introducing any one of the compositions described herein or the vectors described herein into a cell, wherein the expression of a protein encoded by the second RNA is decreased compared to a cell without the composition or vector, and wherein the expression of a protein encoded by the first RNA is increased compared to a cell without the composition or vector.

### EXAMPLES

These examples are provided for illustrative purposes only and not to limit the scope of the claims provided herein.

### Example 1: Construct Design, Sequence, and Synthesis

Construct Design

Both siRNAs and genes of interest are simultaneously expressed from a single transcript generated by in vitro transcription (SEQ ID NOs: 1-17 and 125-141). Polynucleotide or RNA constructs are engineered to include siRNA designs described in Cheng, et al. (2018) J. Mater. Chem. B., 6, 4638-4644, and further comprising one or more gene of interest downstream or upstream of the siRNA sequence (an example of one orientation is shown in FIG. 1). Recombinant constructs may encode or comprise more than one siRNA sequence targeting the same or different target mRNA. Likewise, constructs may comprise nucleic acid sequences of two or more genes of interest. A linker sequence may be present between any two elements of the constructs (e.g., tRNA linker or adapted sequence described by Cheng, et al. 2018).

A polynucleic acid construct may comprise a T7 promoter sequence (5′ TAATACGACTCACTATA 3′; SEQ ID NO: 18) upstream of the gene of interest sequence, for RNA polymerase binding and successful in vitro transcription of both the gene of interest and siRNA in a single transcript. An alternative promoter e.g., SP6, T3, P60, Syn5, and KP34 may be used. A transcription template is generated by PCR to produce mRNA, using primers designed to flank the T7 promoter, gene of interest, and siRNA sequences. The reverse primer includes a stretch of thymidine (T) base (120) (SEQ ID NO: 154) to add the 120 bp length of poly(A) tail (SEQ ID NO: 153) to the mRNA.

Construct Synthesis

The constructs as shown in Table 1 (Compound ID numbers Cpd.1-Cpd.17) were synthesized by GeneArt, Germany (Thermo Fisher Scientific) as vectors containing a T7 RNA polymerase promoter (pMX, e.g., pMA-T, pMK-RQ or pMA-RQ), with codon optimization (GeneOptimizer algorithm). Table 1 shows, for each compound (Cpd.), protein encoding, signal peptide nature, the number of siRNAs of the construct and the protein to be downregulated through siRNA binding to the corresponding mRNA. The sequences of each construct are shown in Table 2 and annotated as indicated below the table (SEQ ID 1-17).

TABLE 1
Summary of Compounds 1-17

| Compound | gene of | Signal | # of | | |
|---|---|---|---|---|---|
| ID | interest | peptide | SIRNAS | siRNA Target | Mechanism |
| Cpd. 1 | IL-2 | Endogenous | NA | NA | Anti-tumor activity |
| Cpd. 2 | IL-2 | Modified | NA | NA | Anti-tumor activity |
| Cpd. 3 | IL-2 | Modified | NA | NA | Anti-tumor activity |
| Cpd. 4 | IL-2 | Modified | NA | NA | Anti-tumor activity |
| Cpd. 5 | IL-2 | Endogenous | 3 | VEGFA | Anti-tumor activity, |
| | | | | | anti-angiogenesis |
| Cpd. 6 | IL-2 | Endogenous | 3 | MICA/B | Anti-tumor activity, |
| | | | | | immune surveillance |
| Cpd. 7 | IL-2 | Modified | 3 | VEGFA | Anti-tumor, anti- |
| | | | | | angiogenesis |
| Cpd. 8 | IL-2 | Modified′ | 5 | VEGFA | Anti-tumor, anti- |
| | | | | | angiogenesis |
| Cpd. 9 | IL-2 | Modified′ | 3 | VEGFA | Anti-tumor, anti- |
| | | | | | angiogenesis |
| Cpd. 10 | IL-2 | Modified′ | 3 | VEGFA | Anti-tumor, anti- |
| | | | | | angiogenesis |
| Cpd. 11 | IL-12 | Endogenous | 3 | IDH1/CDK4/ | Immune-stimulating |
| | | | | CDK6 | cytokine, tumor |
| | | | | | metabolism |
| | | | | | normalizer, cell cycle |
| | | | | | inhibitor |
| Cpd. 12 | IL-12 | Endogenous | 3 | EGFR/mTOR/ | Immune-stimulating |
| | | | | KRAS | cytokine, tumor |
| | | | | | growth inhibitor |
| Cpd. 13 | IL-12 | Endogenous | 3 | EGFR | immune-stimulating |
| | | | | | cytokine, tumor |
| | | | | | growth inhibitor |
| Cpd. 14 | IL-12 | Endogenous | 3 | mTOR | Immune-stimulating |
| | | | | | cytokine, tumor |
| | | | | | growth inhibitor |
| Cpd. 15 | IL-15 | Endogenous | 3 | VEGFA/CD155/ | Immune-stimulating |
| | | | | CD155 | cytokine, anti- |
| | | | | | angiogenesis, |
| | | | | | inhibition of tumor |
| | | | | | immune escape |
| Cpd. 16 | IL-15 | Endogenous | 3 | VEGFA/PD-L1/ | Immune-stimulating |
| | | | | c-Myc | cytokine, anti- |
| | | | | | angiogenesis, |
| | | | | | inhibition of tumor |
| | | | | | immune escape, |
| | | | | | inhibition of tumor |
| | | | | | specific protein |
| | | | | | transcription |
| Cpd. 17 | IL-7 | Endogenous | 3 | PD-L1 | Immune-stimulating |
| | | | | | cytokine, inhibition |
| | | | | | of tumor immune |
| | | | | | escape |

IL-2: Interleukin-2, VEGFA: vascular endothelial growth factor, MICA: MHC class I chain-related sequence A, MICB: MHC class I chain-related sequence B, IL-12: Interleukin-12, IDH1: Isocitrate dehydrogenase; CDK4: Cyclin-dependent kinase 4, CDK6: Cyclin-dependent kinase 6, EGFR: Epidermal growth factor receptor, mTOR: mechanistic target of rapamycin, KRAS: Kirsten rat sarcoma viral oncogene, IL-15: Interleukin-15, CD155: cluster of differentiation 155 (poliovirus receptor), PD-L1: Programmed cell death - ligand 1, c-Myc: Myc proto-oncogene.

TABLE 2
Sequences of Compounds 1-17

| SEQ | | |
|---|---|---|
| ID | | |
| NO | Compound | Sequence (5′ to 3′) |
| 1 | Compound 1 | GCCACCATGTACAGAATGCAGCTGCTGAGCTGTATCGCCCTGTCTCTGGCC |
| | | CTGGTCACAAATAGCGCCCCTACCAGCAGCAGCACCAAGAAAACACAGCTG |
| | | CAACTGGAACACCTCCTGCTGGACCTGCAGATGATCCTGAACGGCATCAAC |
| | | AACTACAAGAACCCCAAGCTGACCCGGATGCTGACCTTCAAGTTCTACATG |
| | | CCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTG |
| | | AGGCCTAGGGACCTGATCAGCAACATCAACGTGATCGTGCTGGAACTGAAA |
| | | GGCAGCGAGACAACCTTCATGTGCGAGTACGCCGACGAGACAGCTACCATC |
| | | GTGGAATTTCTGAACCGGTGGATCACCTTCTGCCAGAGCATCATCAGCACC |
| | | CTGACCTGA |
| 125 | Compound 1 | GCCACCAUGUACAGAAUGCAGCUGCUGAGCUGUAUCGCCCUGUCUCUGGCC |
| | RNA sequence | CUGGUCACAAAUAGCGCCCCUACCAGCAGCAGCACCAAGAAAACACAGCUG |
| | | CAACUGGAACACCUCCUGCUGGACCUGCAGAUGAUCCUGAACGGCAUCAAC |
| | | AACUACAAGAACCCCAAGCUGACCCGGAUGCUGACCUUCAAGUUCUACAUG |
| | | CCCAAGAAGGCCACCGAGCUGAAGCACCUCCAGUGCCUGGAAGAGGAACUG |
| | | AAGCCCCUGGAAGAAGUGCUGAAUCUGGCCCAGAGCAAGAACUUCCACCUG |
| | | AGGCCUAGGGACCUGAUCAGCAACAUCAACGUGAUCGUGCUGGAACUGAAA |
| | | GGCAGCGAGACAACCUUCAUGUGCGAGUACGCCGACGAGACAGCUACCAUC |
| | | GUGGAAUUUCUGAACCGGUGGAUCACCUUCUGCCAGAGCAUCAUCAGCACC |
| | | CUGACCUGA |
| | | (all Us are modified; N¹-methylpseudouridine) |
| 2 | Compound 2* | GCCACCATGCTGAAACTGCTGCTGCTCCTGTGTATCGCCCTGTCTCTGGCC |
| | | GCCACAAATAGCGCCCCTACCAGCAGCTCCACCAAGAAAACACAGCTGCAA |
| | | CTGGAACATCTGCTGCTGGACCTGCAGATGATCCTGAACGGCATCAACAAC |
| | | TACAAGAACCCCAAGCTGACCCGGATGCTGACCTTCAAGTTCTACATGCCC |
| | | AAGAAGGCCACCGAGCTGAAGCACCTCCAGTGCCTGGAAGAGGAACTGAAG |
| | | CCCCTGGAAGAAGTGCTGAATCTGGCCCAGAGCAAGAACTTCCACCTGAGG |
| | | CCTAGGGACCTGATCAGCAACATCAACGTGATCGTGCTGGAACTGAAAGGC |
| | | AGCGAGACAACCTTCATGTGCGAGTACGCCGACGAGACAGCTACCATCGTG |
| | | GAATTTCTGAACCGGTGGATCACCTTCTGCCAGAGCATCATCAGCACCCTG |
| | | ACCTGA |
| 126 | Compound 2 | GCCACCAUGCUGAAACUGCUGCUGCUCCUGUGUAUCGCCCUGUCUCUGGCC |
| | RNA sequence | GCCACAAAUAGCGCCCCUACCAGCAGCUCCACCAAGAAAACACAGCUGCAA |
| | | CUGGAACAUCUGCUGCUGGACCUGCAGAUGAUCCUGAACGGCAUCAACAAC |
| | | UACAAGAACCCCAAGCUGACCCGGAUGCUGACCUUCAAGUUCUACAUGCCC |
| | | AAGAAGGCCACCGAGCUGAAGCACCUCCAGUGCCUGGAAGAGGAACUGAAG |
| | | CCCCUGGAAGAAGUGCUGAAUCUGGCCCAGAGCAAGAACUUCCACCUGAGG |
| | | CCUAGGGACCUGAUCAGCAACAUCAACGUGAUCGUGCUGGAACUGAAAGGC |
| | | AGCGAGACAACCUUCAUGUGCGAGUACGCCGACGAGACAGCUACCAUCGUG |
| | | GAAUUUCUGAACCGGUGGAUCACCUUCUGCCAGAGCAUCAUCAGCACCCUG |
| | | ACCUGA |
| | | (all Us are modified; N¹-methylpseudouridine) |
| 3 | Compound 3* | GCCACCATGTTGTTGCTGCTGCTCGCCTGTATTGCCCTGGCCTCTACAGCC |
| | | GCCGCTACAAATTCTGCCCCTACCAGCAGCTCCACCAAGAAAACCCAGCTG |
| | | CAACTGGAACATCTGCTGCTGGACCTGCAGATGATCCTGAACGGCATCAAC |
| | | AACTACAAGAACCCCAAGCTGACCCGGATGCTGACCTTCAAGTTCTACATG |
| | | CCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTG |
| | | AGGCCTAGGGACCTGATCAGCAACATCAACGTGATCGTGCTGGAACTGAAA |
| | | GGCAGCGAGACAACCTTCATGTGCGAGTACGCCGACGAGACAGCTACCATC |
| | | GTGGAATTTCTGAACCGGTGGATCACCTTCTGCCAGAGCATCATCAGCACC |
| | | CTGACCTGA |
| 127 | Compound 3 | GCCACCAUGUUGUUGCUGCUGCUCGCCUGUAUUGCCCUGGCCUCUACAGCC |
| | RNA sequence | GCCGCUACAAAUUCUGCCCCUACCAGCAGCUCCACCAAGAAAACCCAGCUG |
| | | CAACUGGAACAUCUGCUGCUGGACCUGCAGAUGAUCCUGAACGGCAUCAAC |
| | | AACUACAAGAACCCCAAGCUGACCCGGAUGCUGACCUUCAAGUUCUACAUG |
| | | CCCAAGAAGGCCACCGAGCUGAAGCACCUCCAGUGCCUGGAAGAGGAACUG |
| | | AAGCCCCUGGAAGAAGUGCUGAAUCUGGCCCAGAGCAAGAACUUCCACCUG |
| | | AGGCCUAGGGACCUGAUCAGCAACAUCAACGUGAUCGUGCUGGAACUGAAA |
| | | GGCAGCGAGACAACCUUCAUGUGCGAGUACGCCGACGAGACAGCUACCAUC |
| | | GUGGAAUUUCUGAACCGGUGGAUCACCUUCUGCCAGAGCAUCAUCAGCACC |
| | | CUGACCUGA |
| | | (all Us are modified; N¹-methylpseudouridine) |
| 4 | Compound 4* | GCCACCATGTTGTTGCTGCTGCTCGCCTGTATTGCCCTGGCCTCTACAGCC |
| | | CTGGTCACCAATTCTGCCCCTACCAGCAGCTCCACCAAGAAAACCCAGCTG |
| | | CAACTGGAACATCTGCTGCTGGACCTGCAGATGATCCTGAACGGCATCAAC |
| | | AACTACAAGAACCCCAAGCTGACCCGGATGCTGACCTTCAAGTTCTACATG |
| | | CCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTG |
| | | AGGCCTAGGGACCTGATCAGCAACATCAACGTGATCGTGCTGGAACTGAAA |
| | | GGCAGCGAGACAACCTTCATGTGCGAGTACGCCGACGAGACAGCTACCATC |
| | | GTGGAATTTCTGAACCGGTGGATCACCTTCTGCCAGAGCATCATCAGCACC |
| | | CTGACCTGA |
| 128 | Compound 4 | GCCACCAUGUUGUUGCUGCUGCUCGCCUGUAUUGCCCUGGCCUCUACAGCC |
| | RNA sequence | CUGGUCACCAAUUCUGCCCCUACCAGCAGCUCCACCAAGAAAACCCAGCUG |
| | | CAACUGGAACAUCUGCUGCUGGACCUGCAGAUGAUCCUGAACGGCAUCAAC |
| | | AACUACAAGAACCCCAAGCUGACCCGGAUGCUGACCUUCAAGUUCUACAUG |
| | | CCCAAGAAGGCCACCGAGCUGAAGCACCUCCAGUGCCUGGAAGAGGAACUG |
| | | AAGCCCCUGGAAGAAGUGCUGAAUCUGGCCCAGAGCAAGAACUUCCACCUG |
| | | AGGCCUAGGGACCUGAUCAGCAACAUCAACGUGAUCGUGCUGGAACUGAAA |
| | | GGCAGCGAGACAACCUUCAUGUGCGAGUACGCCGACGAGACAGCUACCAUC |
| | | GUGGAAUUUCUGAACCGGUGGAUCACCUUCUGCCAGAGCAUCAUCAGCACC |
| | | CUGACCUGA |
| | | (all Us are modified; N¹-methylpseudouridine) |
| 5 | Compound 5 | GCCACCATGTACAGAATGCAGCTGCTGAGCTGTATCGCCCTGTCTCTGGCC |
| | | CTGGTCACAAATAGCGCCCCTACCAGCAGCAGCACCAAGAAAACACAGCTG |
| | | CAACTGGAACACCTCCTGCTGGACCTGCAGATGATCCTGAACGGCATCAAC |
| | | AACTACAAGAACCCCAAGCTGACCCGGATGCTGACCTTCAAGTTCTACATG |
| | | CCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTG |
| | | AGGCCTAGGGACCTGATCAGCAACATCAACGTGATCGTGCTGGAACTGAAA |
| | | GGCAGCGAGACAACCTTCATGTGCGAGTACGCCGACGAGACAGCTACCATC |
| | | GTGGAATTTCTGAACCGGTGGATCACCTTCTGCCAGAGCATCATCAGCACC |
| | | CTGACCTGAATAGTGAGTCGTATTAACGTACCAACAAGCAGAATCATCACG |
| | | AAGTGGTACTTG [symbol] [symbol] TTTATCTTAGAGGCATAT |
| | | CCCTACGTACCAACAAGAGCTTCCTACAGCACAACAAACTTG [symbol] |
| | | [symbol] TTTATCTTAGAGGCATATCCCTACGTACCAACAAGATCC |
| | | GCAGACGTGTAAATGTACTTG [symbol] [symbol] TTTATCTTA |
| | | GAGGCATATCCCTTTTATCTTAGAGGCATATCCCT |
| 129 | Compound 5 | GCCACCAUGUACAGAAUGCAGCUGCUGAGCUGUAUCGCCCUGUCUCUGGCC |
| | RNA sequence | CUGGUCACAAAUAGCGCCCCUACCAGCAGCAGCACCAAGAAAACACAGCUG |
| | | CAACUGGAACACCUCCUGCUGGACCUGCAGAUGAUCCUGAACGGCAUCAAC |
| | | AACUACAAGAACCCCAAGCUGACCCGGAUGCUGACCUUCAAGUUCUACAUG |
| | | CCCAAGAAGGCCACCGAGCUGAAGCACCUCCAGUGCCUGGAAGAGGAACUG |
| | | AAGCCCCUGGAAGAAGUGCUGAAUCUGGCCCAGAGCAAGAACUUCCACCUG |
| | | AGGCCUAGGGACCUGAUCAGCAACAUCAACGUGAUCGUGCUGGAACUGAAA |
| | | GGCAGCGAGACAACCUUCAUGUGCGAGUACGCCGACGAGACAGCUACCAUC |
| | | GUGGAAUUUCUGAACCGGUGGAUCACCUUCUGCCAGAGCAUCAUCAGCACC |
| | | CUGACCUGAAUAGUGAGUCGUAUUAACGUACCAACAAGCAGAAUCAUCACG |
| | | AAGUGGUACUUG [symbol] [symbol] UUUAUCUUAGAGGCAUAU |
| | | CCCUACGUACCAACAAGAGCUUCCUACAGCACAACAAACUUG [symbol] |
| | | [symbol] UUUAUCUUAGAGGCAUAUCCCUACGUACCAACAAGAUCC |
| | | GCAGACGUGUAAAUGUACUUG [symbol] [symbol] UUUAUCUUA |
| | | GAGGCAUAUCCCUUUUAUCUUAGAGGCAUAUCCCU |
| | | (all Us are modified; N¹-methylpseudouridine) |
| 6 | Compound 6 | GCCACCATGTACAGAATGCAGCTGCTGAGCTGTATCGCCCTGTCTCTGGCC |
| | | CTGGTCACAAATAGCGCCCCTACCAGCAGCAGCACCAAGAAAACACAGCTG |
| | | CAACTGGAACACCTCCTGCTGGACCTGCAGATGATCCTGAACGGCATCAAC |
| | | AACTACAAGAACCCCAAGCTGACCCGGATGCTGACCTTCAAGTTCTACATG |
| | | CCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTG |
| | | AGGCCTAGGGACCTGATCAGCAACATCAACGTGATCGTGCTGGAACTGAAA |
| | | GGCAGCGAGACAACCTTCATGTGCGAGTACGCCGACGAGACAGCTACCATC |
| | | GTGGAATTTCTGAACCGGTGGATCACCTTCTGCCAGAGCATCATCAGCACC |
| | | CTGACCTGAATAGTGAGTCGTATTAACGTACCAACAAGGAGATTAGGGTCT |
| | | GTGAGATACTTG [symbol] [symbol] TTTATCTTAGAGGCATAT |
| | | CCCTACGTACCAACAAGATGCCATGAAGACCAAGACAACTTG [symbol] |
| | | [symbol] TTTATCTTAGAGGCATATCCCTACGTACCAACAAGCCTG |
| | | ATGGGAATGGAACCTAACTTG [symbol] [symbol] TTTATCTTA |
| | | GAGGCATATCCCTTTTATCTTAGAGGCATATCCCT |
| 130 | Compound 6 | GCCACCAUGUACAGAAUGCAGCUGCUGAGCUGUAUCGCCCUGUCUCUGGCC |
| | RNA sequence | CUGGUCACAAAUAGCGCCCCUACCAGCAGCAGCACCAAGAAAACACAGCUG |
| | | CAACUGGAACACCUCCUGCUGGACCUGCAGAUGAUCCUGAACGGCAUCAAC |
| | | AACUACAAGAACCCCAAGCUGACCCGGAUGCUGACCUUCAAGUUCUACAUG |
| | | CCCAAGAAGGCCACCGAGCUGAAGCACCUCCAGUGCCUGGAAGAGGAACUG |
| | | AAGCCCCUGGAAGAAGUGCUGAAUCUGGCCCAGAGCAAGAACUUCCACCUG |
| | | AGGCCUAGGGACCUGAUCAGCAACAUCAACGUGAUCGUGCUGGAACUGAAA |
| | | GGCAGCGAGACAACCUUCAUGUGCGAGUACGCCGACGAGACAGCUACCAUC |
| | | GUGGAAUUUCUGAACCGGUGGAUCACCUUCUGCCAGAGCAUCAUCAGCACC |
| | | CUGACCUGAAUAGUGAGUCGUAUUAACGUACCAACAAGGAGAUUAGGGUCU |
| | | GUGAGAUACUUG [symbol] [symbol] UUUAUCUUAGAGGCAUAU |
| | | CCCUACGUACCAACAAGAUGCCAUGAAGACCAAGACAACUUG [symbol] |
| | | [symbol] UUUAUCUUAGAGGCAUAUCCCUACGUACCAACAAGCCUG |
| | | AUGGGAAUGGAACCUAACUUG [symbol] [symbol] UUUAUCUUA |
| | | GAGGCAUAUCCCUUUUAUCUUAGAGGCAUAUCCCU |
| | | (all Us are modified; N¹-methylpseudouridine) |
| 7 | Compound 7 | GCCACCATGTTGTTGCTGCTGCTCGCCTGTATTGCCCTGGCCTCTACAGCC |
| | | GCCGCTACAAATTCTGCCCCTACCAGCAGCTCCACCAAGAAAACCCAGCTG |
| | | CAACTGGAACATCTGCTGCTGGACCTGCAGATGATCCTGAACGGCATCAAC |
| | | AACTACAAGAACCCCAAGCTGACCCGGATGCTGACCTTCAAGTTCTACATG |
| | | CCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTG |
| | | AGGCCTAGGGACCTGATCAGCAACATCAACGTGATCGTGCTGGAACTGAAA |
| | | GGCAGCGAGACAACCTTCATGTGCGAGTACGCCGACGAGACAGCTACCATC |
| | | GTGGAATTTCTGAACCGGTGGATCACCTTCTGCCAGAGCATCATCAGCACC |
| | | CTGACCTGAATAGTGAGTCGTATTAACGTACCAACAAGCAGAATCATCACG |
| | | AAGTGGTACTTG [symbol] TTTATCTTAGAGGCATAT |
| | | CCCTACGTACCAACAAGAGCTTCCTACAGCACAACAAACTTG [symbol] |
| | | [symbol] TTTATCTTAGAGGCATATCCCTACGTACCAACAAGATCC |
| | | GCAGACGTGTAAATGTACTTG [symbol] [symbol] TTTATCTTA |
| | | GAGGCATATCCCTTTTATCTTAGAGGCATATCCCT |
| 131 | Compound 7 | GCCACCAUGUUGUUGCUGCUGCUCGCCUGUAUUGCCCUGGCCUCUACAGCC |
| | RNA sequence | GCCGCUACAAAUUCUGCCCCUACCAGCAGCUCCACCAAGAAAACCCAGCUG |
| | | CAACUGGAACAUCUGCUGCUGGACCUGCAGAUGAUCCUGAACGGCAUCAAC |
| | | AACUACAAGAACCCCAAGCUGACCCGGAUGCUGACCUUCAAGUUCUACAUG |
| | | CCCAAGAAGGCCACCGAGCUGAAGCACCUCCAGUGCCUGGAAGAGGAACUG |
| | | AAGCCCCUGGAAGAAGUGCUGAAUCUGGCCCAGAGCAAGAACUUCCACCUG |
| | | AGGCCUAGGGACCUGAUCAGCAACAUCAACGUGAUCGUGCUGGAACUGAAA |
| | | GGCAGCGAGACAACCUUCAUGUGCGAGUACGCCGACGAGACAGCUACCAUC |
| | | GUGGAAUUUCUGAACCGGUGGAUCACCUUCUGCCAGAGCAUCAUCAGCACC |
| | | CUGACCUGAAUAGUGAGUCGUAUUAACGUACCAACAAGCAGAAUCAUCACG |
| | | AAGUGGUACUUG [symbol] [symbol] UUUAUCUUAGAGGCAUAU |
| | | CCCUACGUACCAACAAGAGCUUCCUACAGCACAACAAACUUG [symbol] |
| | | [symbol] UUUAUCUUAGAGGCAUAUCCCUACGUACCAACAAGAUCC |
| | | GCAGACGUGUAAAUGUACUUG [symbol] [symbol] UUUAUCUUA |
| | | GAGGCAUAUCCCUUUUAUCUUAGAGGCAUAUCCCU |
| | | (all Us are modified; N¹-methylpseudouridine) |
| 8 | Compound 8 | GCCACCATGTTGTTGCTGCTGCTCGCCTGTATTGCCCTGGCCTCTACAGCC |
| | | GCCGCTACAAATTCTGCCCCTACCAGCAGCTCCACCAAGAAAACCCAGCTG |
| | | CAACTGGAACATCTGCTGCTGGACCTGCAGATGATCCTGAACGGCATCAAC |
| | | AACTACAAGAACCCCAAGCTGACCCGGATGCTGACCTTCAAGTTCTACATG |
| | | CCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTG |
| | | AGGCCTAGGGACCTGATCAGCAACATCAACGTGATCGTGCTGGAACTGAAA |
| | | GGCAGCGAGACAACCTTCATGTGCGAGTACGCCGACGAGACAGCTACCATC |
| | | GTGGAATTTCTGAACCGGTGGATCACCTTCTGCCAGAGCATCATCAGCACC |
| | | CTGACCTGAATAGTGAGTCGTATTAACGTACCAACAAGCAGAATCATCACG |
| | | AAGTGGTACTTG [symbol] TTTATCTTAGAGGCATAT |
| | | CCCTACGTACCAACAAGAGCTTCCTACAGCACAACAAACTTG [symbol] |
| | | [symbol] TTTATCTTAGAGGCATATCCCTACGTACCAACAAGATCC |
| | | GCAGACGTGTAAATGTACTTG [symbol] [symbol] TTTATCTTA |
| | | GAGGCATATCCCTACGTACCAACAA [symbol] [symbol] ACTTG |
| | | [symbol] [symbol] TTTATCTTAGAGGCATATCCCTACGTACCA |
| | | ACAAGGCGAGGCAGCTTGAGTTAAAACTTG [symbol] [symbol] |
| | | TTTATCTTAGAGGCATATCCCTTTTATCTTAGAGGCATATCCCT |
| 132 | Compound 8 | GCCACCAUGUUGUUGCUGCUGCUCGCCUGUAUUGCCCUGGCCUCUACAGCC |
| | RNA sequence | GCCGCUACAAAUUCUGCCCCUACCAGCAGCUCCACCAAGAAAACCCAGCUG |
| | | CAACUGGAACAUCUGCUGCUGGACCUGCAGAUGAUCCUGAACGGCAUCAAC |
| | | AACUACAAGAACCCCAAGCUGACCCGGAUGCUGACCUUCAAGUUCUACAUG |
| | | CCCAAGAAGGCCACCGAGCUGAAGCACCUCCAGUGCCUGGAAGAGGAACUG |
| | | AAGCCCCUGGAAGAAGUGCUGAAUCUGGCCCAGAGCAAGAACUUCCACCUG |
| | | AGGCCUAGGGACCUGAUCAGCAACAUCAACGUGAUCGUGCUGGAACUGAAA |
| | | GGCAGCGAGACAACCUUCAUGUGCGAGUACGCCGACGAGACAGCUACCAUC |
| | | GUGGAAUUUCUGAACCGGUGGAUCACCUUCUGCCAGAGCAUCAUCAGCACC |
| | | CUGACCUGAAUAGUGAGUCGUAUUAACGUACCAACAAGCAGAAUCAUCACG |
| | | AAGUGGUACUUG [symbol] [symbol] UUAUCUUAGAGGCAUAU |
| | | CCCUACGUACCAACAAGAGCUUCCUACAGCACAACAAACUUG [symbol] |
| | | [symbol] UUUAUCUUAGAGGCAUAUCCCUACGUACCAACAAGAUCC |
| | | GCAGACGUGUAAAUGUACUUG [symbol] [symbol] UUUAUCUUA |
| | | GAGGCAUAUCCCUACGUACCAACAA [symbol] [symbol] ACUUG |
| | | [symbol] [symbol] UUUAUCUUAGAGGCAUAUCCCUACGUACCA |
| | | ACAAGGCGAGGCAGCUUGAGUUAAAACUUG [symbol] [symbol] |
| | | UUUAUCUUAGAGGCAUAUCCCUUUUAUCUUAGAGGCAUAUCCCU |
| | | (all Us are modified; N¹-methylpseudouridine) |
| 9 | Compound 9 | GCCACCATGTTGTTGCTGCTGCTCGCCTGTATTGCCCTGGCCTCTACAGCC |
| | | GCCGCTACAAATTCTGCCCCTACCAGCAGCTCCACCAAGAAAACCCAGCTG |
| | | CAACTGGAACATCTGCTGCTGGACCTGCAGATGATCCTGAACGGCATCAAC |
| | | AACTACAAGAACCCCAAGCTGACCCGGATGCTGACCTTCAAGTTCTACATG |
| | | CCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTG |
| | | AGGCCTAGGGACCTGATCAGCAACATCAACGTGATCGTGCTGGAACTGAAA |
| | | GGCAGCGAGACAACCTTCATGTGCGAGTACGCCGACGAGACAGCTACCATC |
| | | GTGGAATTTCTGAACCGGTGGATCACCTTCTGCCAGAGCATCATCAGCACC |
| | | CTGACCTGAATAGTGAGTCGTATTAACGTACCAACAAGGAGTACCCTGATG |
| | | AGATCACTTG [symbol] [symbol] TTTATCTTAGAGGCATATCCCT |
| | | ACGTACCAACAAGGAGTACCCTGATGAGATCACTTG [symbol] |
| | | CTCCTTTATCTTAGAGGCATATCCCTACGTACCAACAAGGAGTACCCTGAT |
| | | GAGATCACTTG [symbol] [symbol] TTTATCTTAGAGGCATATCCC |
| | | TTTTATCTTAGAGGCATATCCCT |
| 133 | Compound 9 | GCCACCAUGUUGUUGCUGCUGCUCGCCUGUAUUGCCCUGGCCUCUACAGCC |
| | RNA sequence | GCCGCUACAAAUUCUGCCCCUACCAGCAGCUCCACCAAGAAAACCCAGCUG |
| | | CAACUGGAACAUCUGCUGCUGGACCUGCAGAUGAUCCUGAACGGCAUCAAC |
| | | AACUACAAGAACCCCAAGCUGACCCGGAUGCUGACCUUCAAGUUCUACAUG |
| | | CCCAAGAAGGCCACCGAGCUGAAGCACCUCCAGUGCCUGGAAGAGGAACUG |
| | | AAGCCCCUGGAAGAAGUGCUGAAUCUGGCCCAGAGCAAGAACUUCCACCUG |
| | | AGGCCUAGGGACCUGAUCAGCAACAUCAACGUGAUCGUGCUGGAACUGAAA |
| | | GGCAGCGAGACAACCUUCAUGUGCGAGUACGCCGACGAGACAGCUACCAUC |
| | | GUGGAAUUUCUGAACCGGUGGAUCACCUUCUGCCAGAGCAUCAUCAGCACC |
| | | CUGACCUGAAUAGUGAGUCGUAUUAACGUACCAACAAGGAGUACCCUGAUG |
| | | AGAUCACUUG [symbol] [symbol] UUUAUCUUAGAGGCAUAUCCCU |
| | | ACGUACCAACAAGGAGUACCCUGAUGAGAUCACUUG [symbol] |
| | | [symbol] UUUAUCUUAGAGGCAUAUCCCUACGUACCAACAAGGAGUACCCUGAU |
| | | GAGAUCACUUG [symbol] [symbol] UUUAUCUUAGAGGCAUAUCCC |
| | | UUUUAUCUUAGAGGCAUAUCCCU |
| | | (all Us are modified; N¹-methylpseudouridine) |
| 10 | Compound 10 | GCCACCATGTTGTTGCTGCTGCTCGCCTGTATTGCCCTGGCCTCTACAGCC |
| | | GCCGCTACAAATTCTGCCCCTACCAGCAGCTCCACCAAGAAAACCCAGCTG |
| | | CAACTGGAACATCTGCTGCTGGACCTGCAGATGATCCTGAACGGCATCAAC |
| | | AACTACAAGAACCCCAAGCTGACCCGGATGCTGACCTTCAAGTTCTACATG |
| | | CCCAAGAAGGCCACCGAGCTGAAGCACCTCCAGTGCCTGGAAGAGGAACTG |
| | | AAGCCCCTGGAAGAAGTGCTGAATCTGGCCCAGAGCAAGAACTTCCACCTG |
| | | AGGCCTAGGGACCTGATCAGCAACATCAACGTGATCGTGCTGGAACTGAAA |
| | | GGCAGCGAGACAACCTTCATGTGCGAGTACGCCGACGAGACAGCTACCATC |
| | | GTGGAATTTCTGAACCGGTGGATCACCTTCTGCCAGAGCATCATCAGCACC |
| | | CTGACCTGAATAGTGAGTCGTATTAACGTACCAACAAGGAGGGCAGAATCA |
| | | TCACGAAGTGGTGAAGTACTTG [symbol] [symbol] |
| | | [symbol] TTTATCTTAGAGGCATATCCCTACGTACCAACAAGAGATGAGCTTCCTA |
| | | CAGCACAACAAATGTGACTTG [symbol] [symbol] |
| | | [symbol] TTTATCTTAGAGGCATATCCCTACGTACCAACAAGTACAAGATCCGCAGA |
| | | CGTGTAAATGTTCCACTTG [symbol] [symbol] [symbol] TT |
| | | TATCTTAGAGGCATATCCCTTTTATCTTAGAGGCATATCCCT |
| 134 | Compound 10 | GCCACCAUGUUGUUGCUGCUGCUCGCCUGUAUUGCCCUGGCCUCUACAGCC |
| | RNA sequence | GCCGCUACAAAUUCUGCCCCUACCAGCAGCUCCACCAAGAAAACCCAGCUG |
| | | CAACUGGAACAUCUGCUGCUGGACCUGCAGAUGAUCCUGAACGGCAUCAAC |
| | | AACUACAAGAACCCCAAGCUGACCCGGAUGCUGACCUUCAAGUUCUACAUG |
| | | CCCAAGAAGGCCACCGAGCUGAAGCACCUCCAGUGCCUGGAAGAGGAACUG |
| | | AAGCCCCUGGAAGAAGUGCUGAAUCUGGCCCAGAGCAAGAACUUCCACCUG |
| | | AGGCCUAGGGACCUGAUCAGCAACAUCAACGUGAUCGUGCUGGAACUGAAA |
| | | GGCAGCGAGACAACCUUCAUGUGCGAGUACGCCGACGAGACAGCUACCAUC |
| | | GUGGAAUUUCUGAACCGGUGGAUCACCUUCUGCCAGAGCAUCAUCAGCACC |
| | | CUGACCUGAAUAGUGAGUCGUAUUAACGUACCAACAAGGAGGGCAGAAUCA |
| | | UCACGAAGUGGUGAAGUACUUG [symbol] [symbol] [symbol] |
| | | [symbol] UUUAUCUUAGAGGCAUAUCCCUACGUACCAACAAGAGAUGAGCUUCCUA |
| | | CAGCACAACAAAUGUGACUUG [symbol] [symbol] [symbol] |
| | | [symbol] UUUAUCUUAGAGGCAUAUCCCUACGUACCAACAAGUACAAGAUCCGCAGA |
| | | CGUGUAAAUGUUCCACUUG [symbol] [symbol] [symbol] UU |
| | | UAUCUUAGAGGCAUAUCCCUUUUAUCUUAGAGGCAUAUCCCU |
| | | (all Us are modified; N¹-methylpseudouridine) |
| 11 | Compound 11 | GCCACCATGTGTCACCAGCAGCTGGTCATCAGCTGGTTCAGCCTGGTGTTC |
| | | CTGGCCTCTCCTCTGGTGGCCATCTGGGAGCTGAAGAAAGACGTGTACGTG |
| | | GTGGAACTGGACTGGTATCCCGATGCTCCTGGCGAGATGGTGGTGCTGACC |
| | | TGCGATACCCCTGAAGAGGACGGCATCACCTGGACACTGGATCAGTCTAGC |
| | | GAGGTGCTCGGCAGCGGCAAGACCCTGACCATCCAAGTGAAAGAGTTTGGC |
| | | GACGCCGGCCAGTACACCTGTCACAAAGGCGGAGAAGTGCTGAGCCACAGC |
| | | CTGCTGCTGCTCCACAAGAAAGAGGATGGCATTTGGAGCACCGACATCCTG |
| | | AAGGACCAGAAAGAGCCCAAGAACAAGACCTTCCTGAGATGCGAGGCCAAG |
| | | AACTACAGCGGCCGGTTCACATGTTGGTGGCTGACCACCATCAGCACCGAC |
| | | CTGACCTTCAGCGTGAAGTCCAGCAGAGGCAGCAGTGATCCTCAGGGCGTT |
| | | ACATGTGGCGCCGCTACACTGTCTGCCGAAAGAGTGCGGGGCGACAACAAA |
| | | GAATACGAGTACAGCGTGGAATGCCAAGAGGACAGCGCCTGTCCAGCCGCC |
| | | GAAGAGTCTCTGCCTATCGAAGTGATGGTGGACGCCGTGCACAAGCTGAAG |
| | | TACGAGAACTACACCTCCAGCTTTTTCATCCGGGACATCATCAAGCCCGAT |
| | | CCTCCAAAGAACCTGCAGCTGAAGCCTCTGAAGAACAGCAGACAGGTGGAA |
| | | GTGTCCTGGGAGTACCCCGACACCTGGTCTACACCCCACAGCTACTTCAGC |
| | | CTGACCTTTTGCGTGCAAGTGCAGGGCAAGTCCAAGCGCGAGAAAAAGGAC |
| | | CGGGTGTTCACCGACAAGACCAGCGCCACCGTGATCTGCAGAAAGAACGCC |
| | | AGCATCAGCGTCAGAGCCCAGGACCGGTACTACAGCAGCTCTTGGAGCGAA |
| | | TGGGCCAGCGTGCCATGTTCTGGTGGCGGAGGATCTGGCGGAGGTGGAAGC |
| | | GGCGGAGGCGGATCTAGAAATCTGCCTGTGGCCACTCCTGATCCTGGCATG |
| | | TTCCCTTGTCTGCACCACAGCCAGAACCTGCTGAGAGCCGTGTCCAACATG |
| | | CTGCAGAAGGCCAGACAGACCCTGGAATTCTACCCCTGCACCAGCGAGGAA |
| | | ATCGACCACGAGGACATCACCAAGGATAAGACCAGCACCGTGGAAGCCTGC |
| | | CTGCCTCTGGAACTGACCAAGAACGAGAGCTGCCTGAACAGCCGGGAAACC |
| | | AGCTTCATCACCAACGGCTCTTGCCTGGCCAGCAGAAAGACCTCCTTCATG |
| | | ATGGCCCTGTGCCTGAGCAGCATCTACGAGGACCTGAAGATGTACCAGGTG |
| | | GAATTCAAGACCATGAACGCCAAGCTGCTGATGGACCCCAAGCGGCAGATC |
| | | TTCCTGGACCAGAATATGCTGGCCGTGATCGACGAGCTGATGCAGGCCCTG |
| | | AACTTCAACAGCGAGACAGTGCCCCAGAAGTCTAGCCTGGAAGAACCCGAC |
| | | TTCTACAAGACCAAGATCAAGCTGTGCATCCTGCTGCACGCCTTCCGGATC |
| | | AGAGCCGTGACCATCGACAGAGTGATGAGCTACCTGAACGCCTCCTGAATA |
| | | GTGAGTCGTATTAACGTACCAACAAGTTCCTTCCAAATGGCTCTGTACTTG |
| | | [symbol] [symbol] TTTATCTTAGAGGCATATCCCTACGTACCA |
| | | ACAAGCATCGTTCACCGAGATCTGAACTTG [symbol] [symbol] |
| | | TTTATCTTAGAGGCATATCCCTACGTACCAACAAGACCAGCAGCGGACAAA |
| | | TAAAACTTG [symbol] [symbol] TTTATCTTAGAGGCATATCCC |
| | | TTTTATCTTAGAGGCATATCCCT |
| 135 | Compound 11 | GCCACCAUGUGUCACCAGCAGCUGGUCAUCAGCUGGUUCAGCCUGGUGUUC |
| | RNA sequence | CUGGCCUCUCCUCUGGUGGCCAUCUGGGAGCUGAAGAAAGACGUGUACGUG |
| | | GUGGAACUGGACUGGUAUCCCGAUGCUCCUGGCGAGAUGGUGGUGCUGACC |
| | | UGCGAUACCCCUGAAGAGGACGGCAUCACCUGGACACUGGAUCAGUCUAGC |
| | | GAGGUGCUCGGCAGCGGCAAGACCCUGACCAUCCAAGUGAAAGAGUUUGGC |
| | | GACGCCGGCCAGUACACCUGUCACAAAGGCGGAGAAGUGCUGAGCCACAGC |
| | | CUGCUGCUGCUCCACAAGAAAGAGGAUGGCAUUUGGAGCACCGACAUCCUG |
| | | AAGGACCAGAAAGAGCCCAAGAACAAGACCUUCCUGAGAUGCGAGGCCAAG |
| | | AACUACAGCGGCCGGUUCACAUGUUGGUGGCUGACCACCAUCAGCACCGAC |
| | | CUGACCUUCAGCGUGAAGUCCAGCAGAGGCAGCAGUGAUCCUCAGGGCGUU |
| | | ACAUGUGGCGCCGCUACACUGUCUGCCGAAAGAGUGCGGGGCGACAACAAA |
| | | GAAUACGAGUACAGCGUGGAAUGCCAAGAGGACAGCGCCUGUCCAGCCGCC |
| | | GAAGAGUCUCUGCCUAUCGAAGUGAUGGUGGACGCCGUGCACAAGCUGAAG |
| | | UACGAGAACUACACCUCCAGCUUUUUCAUCCGGGACAUCAUCAAGCCCGAU |
| | | CCUCCAAAGAACCUGCAGCUGAAGCCUCUGAAGAACAGCAGACAGGUGGAA |
| | | GUGUCCUGGGAGUACCCCGACACCUGGUCUACACCCCACAGCUACUUCAGC |
| | | CUGACCUUUUGCGUGCAAGUGCAGGGCAAGUCCAAGCGCGAGAAAAAGGAC |
| | | CGGGUGUUCACCGACAAGACCAGCGCCACCGUGAUCUGCAGAAAGAACGCC |
| | | AGCAUCAGCGUCAGAGCCCAGGACCGGUACUACAGCAGCUCUUGGAGCGAA |
| | | UGGGCCAGCGUGCCAUGUUCUGGUGGCGGAGGAUCUGGCGGAGGUGGAAGC |
| | | GGCGGAGGCGGAUCUAGAAAUCUGCCUGUGGCCACUCCUGAUCCUGGCAUG |
| | | UUCCCUUGUCUGCACCACAGCCAGAACCUGCUGAGAGCCGUGUCCAACAUG |
| | | CUGCAGAAGGCCAGACAGACCCUGGAAUUCUACCCCUGCACCAGCGAGGAA |
| | | AUCGACCACGAGGACAUCACCAAGGAUAAGACCAGCACCGUGGAAGCCUGC |
| | | CUGCCUCUGGAACUGACCAAGAACGAGAGCUGCCUGAACAGCCGGGAAACC |
| | | AGCUUCAUCACCAACGGCUCUUGCCUGGCCAGCAGAAAGACCUCCUUCAUG |
| | | AUGGCCCUGUGCCUGAGCAGCAUCUACGAGGACCUGAAGAUGUACCAGGUG |
| | | GAAUUCAAGACCAUGAACGCCAAGCUGCUGAUGGACCCCAAGCGGCAGAUC |
| | | UUCCUGGACCAGAAUAUGCUGGCCGUGAUCGACGAGCUGAUGCAGGCCCUG |
| | | AACUUCAACAGCGAGACAGUGCCCCAGAAGUCUAGCCUGGAAGAACCCGAC |
| | | UUCUACAAGACCAAGAUCAAGCUGUGCAUCCUGCUGCACGCCUUCCGGAUC |
| | | AGAGCCGUGACCAUCGACAGAGUGAUGAGCUACCUGAACGCCUCCUGAAUA |
| | | GUGAGUCGUAUUAACGUACCAACAAGUUCCUUCCAAAUGGCUCUGUACUUG |
| | | [symbol] [symbol] UUUAUCUUAGAGGCAUAUCCCUACGUACCA |
| | | ACAAGCAUCGUUCACCGAGAUCUGAACUUG [symbol] [symbol] |
| | | UUUAUCUUAGAGGCAUAUCCCUACGUACCAACAAGACCAGCAGCGGACAAA |
| | | UAAAACUUG [symbol] [symbol] UUUAUCUUAGAGGCAUAUCCC |
| | | UUUUAUCUUAGAGGCAUAUCCCU |
| | | (all Us are modified; N¹-methylpseudouridine) |
| 12 | Compound 12 | GCCACCATGTGTCACCAGCAGCTGGTCATCAGCTGGTTCAGCCTGGTGTTC |
| | | CTGGCCTCTCCTCTGGTGGCCATCTGGGAGCTGAAGAAAGACGTGTACGTG |
| | | GTGGAACTGGACTGGTATCCCGATGCTCCTGGCGAGATGGTGGTGCTGACC |
| | | TGCGATACCCCTGAAGAGGACGGCATCACCTGGACACTGGATCAGTCTAGC |
| | | GAGGTGCTCGGCAGCGGCAAGACCCTGACCATCCAAGTGAAAGAGTTTGGC |
| | | GACGCCGGCCAGTACACCTGTCACAAAGGCGGAGAAGTGCTGAGCCACAGC |
| | | CTGCTGCTGCTCCACAAGAAAGAGGATGGCATTTGGAGCACCGACATCCTG |
| | | AAGGACCAGAAAGAGCCCAAGAACAAGACCTTCCTGAGATGCGAGGCCAAG |
| | | AACTACAGCGGCCGGTTCACATGTTGGTGGCTGACCACCATCAGCACCGAC |
| | | CTGACCTTCAGCGTGAAGTCCAGCAGAGGCAGCAGTGATCCTCAGGGCGTT |
| | | ACATGTGGCGCCGCTACACTGTCTGCCGAAAGAGTGCGGGGCGACAACAAA |
| | | GAATACGAGTACAGCGTGGAATGCCAAGAGGACAGCGCCTGTCCAGCCGCC |
| | | GAAGAGTCTCTGCCTATCGAAGTGATGGTGGACGCCGTGCACAAGCTGAAG |
| | | TACGAGAACTACACCTCCAGCTTTTTCATCCGGGACATCATCAAGCCCGAT |
| | | CCTCCAAAGAACCTGCAGCTGAAGCCTCTGAAGAACAGCAGACAGGTGGAA |
| | | GTGTCCTGGGAGTACCCCGACACCTGGTCTACACCCCACAGCTACTTCAGC |
| | | CTGACCTTTTGCGTGCAAGTGCAGGGCAAGTCCAAGCGCGAGAAAAAGGAC |
| | | CGGGTGTTCACCGACAAGACCAGCGCCACCGTGATCTGCAGAAAGAACGCC |
| | | AGCATCAGCGTCAGAGCCCAGGACCGGTACTACAGCAGCTCTTGGAGCGAA |
| | | TGGGCCAGCGTGCCATGTTCTGGTGGCGGAGGATCTGGCGGAGGTGGAAGC |
| | | GGCGGAGGCGGATCTAGAAATCTGCCTGTGGCCACTCCTGATCCTGGCATG |
| | | TTCCCTTGTCTGCACCACAGCCAGAACCTGCTGAGAGCCGTGTCCAACATG |
| | | CTGCAGAAGGCCAGACAGACCCTGGAATTCTACCCCTGCACCAGCGAGGAA |
| | | ATCGACCACGAGGACATCACCAAGGATAAGACCAGCACCGTGGAAGCCTGC |
| | | CTGCCTCTGGAACTGACCAAGAACGAGAGCTGCCTGAACAGCCGGGAAACC |
| | | AGCTTCATCACCAACGGCTCTTGCCTGGCCAGCAGAAAGACCTCCTTCATG |
| | | ATGGCCCTGTGCCTGAGCAGCATCTACGAGGACCTGAAGATGTACCAGGTG |
| | | GAATTCAAGACCATGAACGCCAAGCTGCTGATGGACCCCAAGCGGCAGATC |
| | | TTCCTGGACCAGAATATGCTGGCCGTGATCGACGAGCTGATGCAGGCCCTG |
| | | AACTTCAACAGCGAGACAGTGCCCCAGAAGTCTAGCCTGGAAGAACCCGAC |
| | | TTCTACAAGACCAAGATCAAGCTGTGCATCCTGCTGCACGCCTTCCGGATC |
| | | AGAGCCGTGACCATCGACAGAGTGATGAGCTACCTGAACGCCTCCTGAATA |
| | | GTGAGTCGTATTAACGTACCAACAAATAGTGAGTCGTATTAACGTACCAAC |
| | | AAGAAGGAGCTGCCCATGAGAAAACTTG [symbol] [symbol] TT |
| | | TATCTTAGAGGCATATCCCTACGTACCAACAAGTGCAATGAGGGACCAGTA |
| | | CAACTTG [symbol] [symbol] TTTATCTTAGAGGCATATCCCTA |
| | | CGTACCAACAAGAGCTGCTGAAGGACTCATCAACTTG [symbol] |
| | | [symbol] TTTATCTTAGAGGCATATCCCTTTTATCTTAGAGGCATATCCCT |
| 136 | Compound 12 | GCCACCAUGUGUCACCAGCAGCUGGUCAUCAGCUGGUUCAGCCUGGUGUUC |
| | RNA sequence | CUGGCCUCUCCUCUGGUGGCCAUCUGGGAGCUGAAGAAAGACGUGUACGUG |
| | | GUGGAACUGGACUGGUAUCCCGAUGCUCCUGGCGAGAUGGUGGUGCUGACC |
| | | UGCGAUACCCCUGAAGAGGACGGCAUCACCUGGACACUGGAUCAGUCUAGC |
| | | GAGGUGCUCGGCAGCGGCAAGACCCUGACCAUCCAAGUGAAAGAGUUUGGC |
| | | GACGCCGGCCAGUACACCUGUCACAAAGGCGGAGAAGUGCUGAGCCACAGC |
| | | CUGCUGCUGCUCCACAAGAAAGAGGAUGGCAUUUGGAGCACCGACAUCCUG |
| | | AAGGACCAGAAAGAGCCCAAGAACAAGACCUUCCUGAGAUGCGAGGCCAAG |
| | | AACUACAGCGGCCGGUUCACAUGUUGGUGGCUGACCACCAUCAGCACCGAC |
| | | CUGACCUUCAGCGUGAAGUCCAGCAGAGGCAGCAGUGAUCCUCAGGGCGUU |
| | | ACAUGUGGCGCCGCUACACUGUCUGCCGAAAGAGUGCGGGGCGACAACAAA |
| | | GAAUACGAGUACAGCGUGGAAUGCCAAGAGGACAGCGCCUGUCCAGCCGCC |
| | | GAAGAGUCUCUGCCUAUCGAAGUGAUGGUGGACGCCGUGCACAAGCUGAAG |
| | | UACGAGAACUACACCUCCAGCUUUUUCAUCCGGGACAUCAUCAAGCCCGAU |
| | | CCUCCAAAGAACCUGCAGCUGAAGCCUCUGAAGAACAGCAGACAGGUGGAA |
| | | GUGUCCUGGGAGUACCCCGACACCUGGUCUACACCCCACAGCUACUUCAGC |
| | | CUGACCUUUUGCGUGCAAGUGCAGGGCAAGUCCAAGCGCGAGAAAAAGGAC |
| | | CGGGUGUUCACCGACAAGACCAGCGCCACCGUGAUCUGCAGAAAGAACGCC |
| | | AGCAUCAGCGUCAGAGCCCAGGACCGGUACUACAGCAGCUCUUGGAGCGAA |
| | | UGGGCCAGCGUGCCAUGUUCUGGUGGCGGAGGAUCUGGCGGAGGUGGAAGC |
| | | GGCGGAGGCGGAUCUAGAAAUCUGCCUGUGGCCACUCCUGAUCCUGGCAUG |
| | | UUCCCUUGUCUGCACCACAGCCAGAACCUGCUGAGAGCCGUGUCCAACAUG |
| | | CUGCAGAAGGCCAGACAGACCCUGGAAUUCUACCCCUGCACCAGCGAGGAA |
| | | AUCGACCACGAGGACAUCACCAAGGAUAAGACCAGCACCGUGGAAGCCUGC |
| | | CUGCCUCUGGAACUGACCAAGAACGAGAGCUGCCUGAACAGCCGGGAAACC |
| | | AGCUUCAUCACCAACGGCUCUUGCCUGGCCAGCAGAAAGACCUCCUUCAUG |
| | | AUGGCCCUGUGCCUGAGCAGCAUCUACGAGGACCUGAAGAUGUACCAGGUG |
| | | GAAUUCAAGACCAUGAACGCCAAGCUGCUGAUGGACCCCAAGCGGCAGAUC |
| | | UUCCUGGACCAGAAUAUGCUGGCCGUGAUCGACGAGCUGAUGCAGGCCCUG |
| | | AACUUCAACAGCGAGACAGUGCCCCAGAAGUCUAGCCUGGAAGAACCCGAC |
| | | UUCUACAAGACCAAGAUCAAGCUGUGCAUCCUGCUGCACGCCUUCCGGAUC |
| | | AGAGCCGUGACCAUCGACAGAGUGAUGAGCUACCUGAACGCCUCCUGAAUA |
| | | GUGAGUCGUAUUAACGUACCAACAAAUAGUGAGUCGUAUUAACGUACCAAC |
| | | AAGAAGGAGCUGCCCAUGAGAAAACUUG [symbol] [symbol] UU |
| | | UAUCUUAGAGGCAUAUCCCUACGUACCAACAAGUGCAAUGAGGGACCAGUA |
| | | CAACUUG [symbol] [symbol] UUUAUCUUAGAGGCAUAUCCCUA |
| | | CGUACCAACAAGAGCUGCUGAAGGACUCAUCAACUUG [symbol] |
| | | [symbol] UUUAUCUUAGAGGCAUAUCCCUUUUAUCUUAGAGGCAUAUCCCU |
| | | (all Us are modified; N¹-methylpseudouridine) |
| 13 | Compound 13 | GCCACCATGTGTCACCAGCAGCTGGTCATCAGCTGGTTCAGCCTGGTGTTC |
| | | CTGGCCTCTCCTCTGGTGGCCATCTGGGAGCTGAAGAAAGACGTGTACGTG |
| | | GTGGAACTGGACTGGTATCCCGATGCTCCTGGCGAGATGGTGGTGCTGACC |
| | | TGCGATACCCCTGAAGAGGACGGCATCACCTGGACACTGGATCAGTCTAGC |
| | | GAGGTGCTCGGCAGCGGCAAGACCCTGACCATCCAAGTGAAAGAGTTTGGC |
| | | GACGCCGGCCAGTACACCTGTCACAAAGGCGGAGAAGTGCTGAGCCACAGC |
| | | CTGCTGCTGCTCCACAAGAAAGAGGATGGCATTTGGAGCACCGACATCCTG |
| | | AAGGACCAGAAAGAGCCCAAGAACAAGACCTTCCTGAGATGCGAGGCCAAG |
| | | AACTACAGCGGCCGGTTCACATGTTGGTGGCTGACCACCATCAGCACCGAC |
| | | CTGACCTTCAGCGTGAAGTCCAGCAGAGGCAGCAGTGATCCTCAGGGCGTT |
| | | ACATGTGGCGCCGCTACACTGTCTGCCGAAAGAGTGCGGGGCGACAACAAA |
| | | GAATACGAGTACAGCGTGGAATGCCAAGAGGACAGCGCCTGTCCAGCCGCC |
| | | GAAGAGTCTCTGCCTATCGAAGTGATGGTGGACGCCGTGCACAAGCTGAAG |
| | | TACGAGAACTACACCTCCAGCTTTTTCATCCGGGACATCATCAAGCCCGAT |
| | | CCTCCAAAGAACCTGCAGCTGAAGCCTCTGAAGAACAGCAGACAGGTGGAA |
| | | GTGTCCTGGGAGTACCCCGACACCTGGTCTACACCCCACAGCTACTTCAGC |
| | | CTGACCTTTTGCGTGCAAGTGCAGGGCAAGTCCAAGCGCGAGAAAAAGGAC |
| | | CGGGTGTTCACCGACAAGACCAGCGCCACCGTGATCTGCAGAAAGAACGCC |
| | | AGCATCAGCGTCAGAGCCCAGGACCGGTACTACAGCAGCTCTTGGAGCGAA |
| | | TGGGCCAGCGTGCCATGTTCTGGTGGCGGAGGATCTGGCGGAGGTGGAAGC |
| | | GGCGGAGGCGGATCTAGAAATCTGCCTGTGGCCACTCCTGATCCTGGCATG |
| | | TTCCCTTGTCTGCACCACAGCCAGAACCTGCTGAGAGCCGTGTCCAACATG |
| | | CTGCAGAAGGCCAGACAGACCCTGGAATTCTACCCCTGCACCAGCGAGGAA |
| | | ATCGACCACGAGGACATCACCAAGGATAAGACCAGCACCGTGGAAGCCTGC |
| | | CTGCCTCTGGAACTGACCAAGAACGAGAGCTGCCTGAACAGCCGGGAAACC |
| | | AGCTTCATCACCAACGGCTCTTGCCTGGCCAGCAGAAAGACCTCCTTCATG |
| | | ATGGCCCTGTGCCTGAGCAGCATCTACGAGGACCTGAAGATGTACCAGGTG |
| | | GAATTCAAGACCATGAACGCCAAGCTGCTGATGGACCCCAAGCGGCAGATC |
| | | TTCCTGGACCAGAATATGCTGGCCGTGATCGACGAGCTGATGCAGGCCCTG |
| | | AACTTCAACAGCGAGACAGTGCCCCAGAAGTCTAGCCTGGAAGAACCCGAC |
| | | TTCTACAAGACCAAGATCAAGCTGTGCATCCTGCTGCACGCCTTCCGGATC |
| | | AGAGCCGTGACCATCGACAGAGTGATGAGCTACCTGAACGCCTCCTGAATA |
| | | GTGAGTCGTATTAACGTACCAACAAGAAGGAGCTGCCCATGAGAAAACTTG |
| | | [symbol] [symbol] TTATCTTAGAGGCATATCCCTACGTACCA |
| | | ACAAGTCCAACGAATGGGCCTAAGAACTTG [symbol] [symbol] |
| | | TTTATCTTAGAGGCATATCCCTACGTACCAACAAGGACAGCATAGACGACA |
| | | CCTTACTTG [symbol] [symbol] TTTATCTTAGAGGCATATCCC |
| | | TTTTATCTTAGAGGCATATCCCT |
| 137 | Compound 13 | GCCACCAUGUGUCACCAGCAGCUGGUCAUCAGCUGGUUCAGCCUGGUGUUC |
| | RNA sequence | CUGGCCUCUCCUCUGGUGGCCAUCUGGGAGCUGAAGAAAGACGUGUACGUG |
| | | GUGGAACUGGACUGGUAUCCCGAUGCUCCUGGCGAGAUGGUGGUGCUGACC |
| | | UGCGAUACCCCUGAAGAGGACGGCAUCACCUGGACACUGGAUCAGUCUAGC |
| | | GAGGUGCUCGGCAGCGGCAAGACCCUGACCAUCCAAGUGAAAGAGUUUGGC |
| | | GACGCCGGCCAGUACACCUGUCACAAAGGCGGAGAAGUGCUGAGCCACAGC |
| | | CUGCUGCUGCUCCACAAGAAAGAGGAUGGCAUUUGGAGCACCGACAUCCUG |
| | | AAGGACCAGAAAGAGCCCAAGAACAAGACCUUCCUGAGAUGCGAGGCCAAG |
| | | AACUACAGCGGCCGGUUCACAUGUUGGUGGCUGACCACCAUCAGCACCGAC |
| | | CUGACCUUCAGCGUGAAGUCCAGCAGAGGCAGCAGUGAUCCUCAGGGCGUU |
| | | ACAUGUGGCGCCGCUACACUGUCUGCCGAAAGAGUGCGGGGCGACAACAAA |
| | | GAAUACGAGUACAGCGUGGAAUGCCAAGAGGACAGCGCCUGUCCAGCCGCC |
| | | GAAGAGUCUCUGCCUAUCGAAGUGAUGGUGGACGCCGUGCACAAGCUGAAG |
| | | UACGAGAACUACACCUCCAGCUUUUUCAUCCGGGACAUCAUCAAGCCCGAU |
| | | CCUCCAAAGAACCUGCAGCUGAAGCCUCUGAAGAACAGCAGACAGGUGGAA |
| | | GUGUCCUGGGAGUACCCCGACACCUGGUCUACACCCCACAGCUACUUCAGC |
| | | CUGACCUUUUGCGUGCAAGUGCAGGGCAAGUCCAAGCGCGAGAAAAAGGAC |
| | | CGGGUGUUCACCGACAAGACCAGCGCCACCGUGAUCUGCAGAAAGAACGCC |
| | | AGCAUCAGCGUCAGAGCCCAGGACCGGUACUACAGCAGCUCUUGGAGCGAA |
| | | UGGGCCAGCGUGCCAUGUUCUGGUGGCGGAGGAUCUGGCGGAGGUGGAAGC |
| | | GGCGGAGGCGGAUCUAGAAAUCUGCCUGUGGCCACUCCUGAUCCUGGCAUG |
| | | UUCCCUUGUCUGCACCACAGCCAGAACCUGCUGAGAGCCGUGUCCAACAUG |
| | | CUGCAGAAGGCCAGACAGACCCUGGAAUUCUACCCCUGCACCAGCGAGGAA |
| | | AUCGACCACGAGGACAUCACCAAGGAUAAGACCAGCACCGUGGAAGCCUGC |
| | | CUGCCUCUGGAACUGACCAAGAACGAGAGCUGCCUGAACAGCCGGGAAACC |
| | | AGCUUCAUCACCAACGGCUCUUGCCUGGCCAGCAGAAAGACCUCCUUCAUG |
| | | AUGGCCCUGUGCCUGAGCAGCAUCUACGAGGACCUGAAGAUGUACCAGGUG |
| | | GAAUUCAAGACCAUGAACGCCAAGCUGCUGAUGGACCCCAAGCGGCAGAUC |
| | | UUCCUGGACCAGAAUAUGCUGGCCGUGAUCGACGAGCUGAUGCAGGCCCUG |
| | | AACUUCAACAGCGAGACAGUGCCCCAGAAGUCUAGCCUGGAAGAACCCGAC |
| | | UUCUACAAGACCAAGAUCAAGCUGUGCAUCCUGCUGCACGCCUUCCGGAUC |
| | | AGAGCCGUGACCAUCGACAGAGUGAUGAGCUACCUGAACGCCUCCUGAAUA |
| | | GUGAGUCGUAUUAACGUACCAACAAGAAGGAGCUGCCCAUGAGAAAACUUG |
| | | [symbol] [symbol] UUUAUCUUAGAGGCAUAUCCCUACGUACCA |
| | | ACAAGUCCAACGAAUGGGCCUAAGAACUUG [symbol] [symbol] |
| | | UUUAUCUUAGAGGCAUAUCCCUACGUACCAACAAGGACAGCAUAGACGACA |
| | | CCUUACUUG [symbol] [symbol] UUUAUCUUAGAGGCAUAUCCC |
| | | UUUUAUCUUAGAGGCAUAUCCCU |
| | | (all Us are modified; N¹-methylpseudouridine) |
| 14 | Compound 14 | GCCACCATGTGTCACCAGCAGCTGGTCATCAGCTGGTTCAGCCTGGTGTTC |
| | | CTGGCCTCTCCTCTGGTGGCCATCTGGGAGCTGAAGAAAGACGTGTACGTG |
| | | GTGGAACTGGACTGGTATCCCGATGCTCCTGGCGAGATGGTGGTGCTGACC |
| | | TGCGATACCCCTGAAGAGGACGGCATCACCTGGACACTGGATCAGTCTAGC |
| | | GAGGTGCTCGGCAGCGGCAAGACCCTGACCATCCAAGTGAAAGAGTTTGGC |
| | | GACGCCGGCCAGTACACCTGTCACAAAGGCGGAGAAGTGCTGAGCCACAGC |
| | | CTGCTGCTGCTCCACAAGAAAGAGGATGGCATTTGGAGCACCGACATCCTG |
| | | AAGGACCAGAAAGAGCCCAAGAACAAGACCTTCCTGAGATGCGAGGCCAAG |
| | | AACTACAGCGGCCGGTTCACATGTTGGTGGCTGACCACCATCAGCACCGAC |
| | | CTGACCTTCAGCGTGAAGTCCAGCAGAGGCAGCAGTGATCCTCAGGGCGTT |
| | | ACATGTGGCGCCGCTACACTGTCTGCCGAAAGAGTGCGGGGCGACAACAAA |
| | | GAATACGAGTACAGCGTGGAATGCCAAGAGGACAGCGCCTGTCCAGCCGCC |
| | | GAAGAGTCTCTGCCTATCGAAGTGATGGTGGACGCCGTGCACAAGCTGAAG |
| | | TACGAGAACTACACCTCCAGCTTTTTCATCCGGGACATCATCAAGCCCGAT |
| | | CCTCCAAAGAACCTGCAGCTGAAGCCTCTGAAGAACAGCAGACAGGTGGAA |
| | | GTGTCCTGGGAGTACCCCGACACCTGGTCTACACCCCACAGCTACTTCAGC |
| | | CTGACCTTTTGCGTGCAAGTGCAGGGCAAGTCCAAGCGCGAGAAAAAGGAC |
| | | CGGGTGTTCACCGACAAGACCAGCGCCACCGTGATCTGCAGAAAGAACGCC |
| | | AGCATCAGCGTCAGAGCCCAGGACCGGTACTACAGCAGCTCTTGGAGCGAA |
| | | TGGGCCAGCGTGCCATGTTCTGGTGGCGGAGGATCTGGCGGAGGTGGAAGC |
| | | GGCGGAGGCGGATCTAGAAATCTGCCTGTGGCCACTCCTGATCCTGGCATG |
| | | TTCCCTTGTCTGCACCACAGCCAGAACCTGCTGAGAGCCGTGTCCAACATG |
| | | CTGCAGAAGGCCAGACAGACCCTGGAATTCTACCCCTGCACCAGCGAGGAA |
| | | ATCGACCACGAGGACATCACCAAGGATAAGACCAGCACCGTGGAAGCCTGC |
| | | CTGCCTCTGGAACTGACCAAGAACGAGAGCTGCCTGAACAGCCGGGAAACC |
| | | AGCTTCATCACCAACGGCTCTTGCCTGGCCAGCAGAAAGACCTCCTTCATG |
| | | ATGGCCCTGTGCCTGAGCAGCATCTACGAGGACCTGAAGATGTACCAGGTG |
| | | GAATTCAAGACCATGAACGCCAAGCTGCTGATGGACCCCAAGCGGCAGATC |
| | | TTCCTGGACCAGAATATGCTGGCCGTGATCGACGAGCTGATGCAGGCCCTG |
| | | AACTTCAACAGCGAGACAGTGCCCCAGAAGTCTAGCCTGGAAGAACCCGAC |
| | | TTCTACAAGACCAAGATCAAGCTGTGCATCCTGCTGCACGCCTTCCGGATC |
| | | AGAGCCGTGACCATCGACAGAGTGATGAGCTACCTGAACGCCTCCTGAATA |
| | | GTGAGTCGTATTAACGTACCAACAAGACCCTGACATTCGCTACTGTACTTG |
| | | [symbol] [symbol] TTTATCTTAGAGGCATATCCCTACGTACCA |
| | | ACAAGAGCTGCTGAAGGACTCATCAACTTG [symbol] [symbol] |
| | | TTTATCTTAGAGGCATATCCCTACGTACCAACAAGGCCAATGACCCAACAT |
| | | CTCTACTTG [symbol] [symbol] TTTATCTTAGAGGCATATCCC |
| | | TTTTATCTTAGAGGCATATCCCT |
| 138 | Compound 14 | GCCACCAUGUGUCACCAGCAGCUGGUCAUCAGCUGGUUCAGCCUGGUGUUC |
| | RNA sequence | CUGGCCUCUCCUCUGGUGGCCAUCUGGGAGCUGAAGAAAGACGUGUACGUG |
| | | GUGGAACUGGACUGGUAUCCCGAUGCUCCUGGCGAGAUGGUGGUGCUGACC |
| | | UGCGAUACCCCUGAAGAGGACGGCAUCACCUGGACACUGGAUCAGUCUAGC |
| | | GAGGUGCUCGGCAGCGGCAAGACCCUGACCAUCCAAGUGAAAGAGUUUGGC |
| | | GACGCCGGCCAGUACACCUGUCACAAAGGCGGAGAAGUGCUGAGCCACAGC |
| | | CUGCUGCUGCUCCACAAGAAAGAGGAUGGCAUUUGGAGCACCGACAUCCUG |
| | | AAGGACCAGAAAGAGCCCAAGAACAAGACCUUCCUGAGAUGCGAGGCCAAG |
| | | AACUACAGCGGCCGGUUCACAUGUUGGUGGCUGACCACCAUCAGCACCGAC |
| | | CUGACCUUCAGCGUGAAGUCCAGCAGAGGCAGCAGUGAUCCUCAGGGCGUU |
| | | ACAUGUGGCGCCGCUACACUGUCUGCCGAAAGAGUGCGGGGCGACAACAAA |
| | | GAAUACGAGUACAGCGUGGAAUGCCAAGAGGACAGCGCCUGUCCAGCCGCC |
| | | GAAGAGUCUCUGCCUAUCGAAGUGAUGGUGGACGCCGUGCACAAGCUGAAG |
| | | UACGAGAACUACACCUCCAGCUUUUUCAUCCGGGACAUCAUCAAGCCCGAU |
| | | CCUCCAAAGAACCUGCAGCUGAAGCCUCUGAAGAACAGCAGACAGGUGGAA |
| | | GUGUCCUGGGAGUACCCCGACACCUGGUCUACACCCCACAGCUACUUCAGC |
| | | CUGACCUUUUGCGUGCAAGUGCAGGGCAAGUCCAAGCGCGAGAAAAAGGAC |
| | | CGGGUGUUCACCGACAAGACCAGCGCCACCGUGAUCUGCAGAAAGAACGCC |
| | | AGCAUCAGCGUCAGAGCCCAGGACCGGUACUACAGCAGCUCUUGGAGCGAA |
| | | UGGGCCAGCGUGCCAUGUUCUGGUGGCGGAGGAUCUGGCGGAGGUGGAAGC |
| | | GGCGGAGGCGGAUCUAGAAAUCUGCCUGUGGCCACUCCUGAUCCUGGCAUG |
| | | UUCCCUUGUCUGCACCACAGCCAGAACCUGCUGAGAGCCGUGUCCAACAUG |
| | | CUGCAGAAGGCCAGACAGACCCUGGAAUUCUACCCCUGCACCAGCGAGGAA |
| | | AUCGACCACGAGGACAUCACCAAGGAUAAGACCAGCACCGUGGAAGCCUGC |
| | | CUGCCUCUGGAACUGACCAAGAACGAGAGCUGCCUGAACAGCCGGGAAACC |
| | | AGCUUCAUCACCAACGGCUCUUGCCUGGCCAGCAGAAAGACCUCCUUCAUG |
| | | AUGGCCCUGUGCCUGAGCAGCAUCUACGAGGACCUGAAGAUGUACCAGGUG |
| | | GAAUUCAAGACCAUGAACGCCAAGCUGCUGAUGGACCCCAAGCGGCAGAUC |
| | | UUCCUGGACCAGAAUAUGCUGGCCGUGAUCGACGAGCUGAUGCAGGCCCUG |
| | | AACUUCAACAGCGAGACAGUGCCCCAGAAGUCUAGCCUGGAAGAACCCGAC |
| | | UUCUACAAGACCAAGAUCAAGCUGUGCAUCCUGCUGCACGCCUUCCGGAUC |
| | | AGAGCCGUGACCAUCGACAGAGUGAUGAGCUACCUGAACGCCUCCUGAAUA |
| | | GUGAGUCGUAUUAACGUACCAACAAGACCCUGACAUUCGCUACUGUACUUG |
| | | [symbol] [symbol] UUUAUCUUAGAGGCAUAUCCCUACGUACCA |
| | | ACAAGAGCUGCUGAAGGACUCAUCAACUUG [symbol] [symbol] |
| | | UUUAUCUUAGAGGCAUAUCCCUACGUACCAACAAGGCCAAUGACCCAACAU |
| | | CUCUACUUG [symbol] [symbol] UUUAUCUUAGAGGCAUAUCCC |
| | | UUUUAUCUUAGAGGCAUAUCCCU |
| | | (all Us are modified; N¹-methylpseudouridine) |
| 15 | Compound 15 | GCCACCATGAGAATCAGCAAGCCCCACCTGAGATCCATCAGCATCCAGTGC |
| | | TACCTGTGCCTGCTGCTGAACAGCCACTTTCTGACAGAGGCCGGCATCCAC |
| | | GTGTTCATCCTGGGCTGTTTTTCTGCCGGCCTGCCTAAGACCGAGGCCAAC |
| | | TGGGTTAACGTGATCAGCGACCTGAAGAAGATCGAGGACCTGATCCAGAGC |
| | | ATGCACATCGACGCCACACTGTACACCGAGAGCGACGTGCACCCTAGCTGT |
| | | AAAGTGACCGCCATGAAGTGCTTTCTGCTGGAACTGCAAGTGATCAGCCTG |
| | | GAAAGCGGCGACGCCAGCATCCACGACACCGTGGAAAACCTGATCATCCTG |
| | | GCCAACAACAGCCTGAGCAGCAACGGCAATGTGACCGAGTCCGGCTGCAAA |
| | | GAGTGCGAGGAACTGGAAGAGAAGAATATCAAAGAGTTCCTGCAGAGCTTC |
| | | GTGCACATCGTGCAGATGTTCATCAACACCAGCTGAATAGTGAGTCGTATT |
| | | AACGTACCAACAAGGAGTACCCTGATGAGATCACTTG [symbol] |
| | | [symbol] TTTATCTTAGAGGCATATCCCTACGTACCAACAAGGTATCCATCTC |
| | | TGGCTATGAACTTG [symbol] [symbol] TTTATCTTAGAGGCAT |
| | | ATCCCTACGTACCAACAAGTCCCGTAACGCCATCATCTTACTTG [symbol] |
| | | [symbol] TTTATCTTAGAGGCATATCCCTTTTATCTTAGAGGCA |
| | | TATCCCT |
| 139 | Compound 15 | GCCACCAUGAGAAUCAGCAAGCCCCACCUGAGAUCCAUCAGCAUCCAGUGC |
| | RNA sequence | UACCUGUGCCUGCUGCUGAACAGCCACUUUCUGACAGAGGCCGGCAUCCAC |
| | | GUGUUCAUCCUGGGCUGUUUUUCUGCCGGCCUGCCUAAGACCGAGGCCAAC |
| | | UGGGUUAACGUGAUCAGCGACCUGAAGAAGAUCGAGGACCUGAUCCAGAGC |
| | | AUGCACAUCGACGCCACACUGUACACCGAGAGCGACGUGCACCCUAGCUGU |
| | | AAAGUGACCGCCAUGAAGUGCUUUCUGCUGGAACUGCAAGUGAUCAGCCUG |
| | | GAAAGCGGCGACGCCAGCAUCCACGACACCGUGGAAAACCUGAUCAUCCUG |
| | | GCCAACAACAGCCUGAGCAGCAACGGCAAUGUGACCGAGUCCGGCUGCAAA |
| | | GAGUGCGAGGAACUGGAAGAGAAGAAUAUCAAAGAGUUCCUGCAGAGCUUC |
| | | GUGCACAUCGUGCAGAUGUUCAUCAACACCAGCUGAAUAGUGAGUCGUAUU |
| | | AACGUACCAACAAGGAGUACCCUGAUGAGAUCACUUG [symbol] |
| | | ACUCCUUUAUCUUAGAGGCAUAUCCCUACGUACCAACAAGGUAUCCAUCUC |
| | | UGGCUAUGAACUUG [symbol] [symbol] UUUAUCUUAGAGGCAU |
| | | AUCCCUACGUACCAACAAGUCCCGUAACGCCAUCAUCUUACUUG [symbol] |
| | | [symbol] UUUAUCUUAGAGGCAUAUCCCUUUUAUCUUAGAGGCA |
| | | UAUCCCU |
| | | (all Us are modified; N¹-methylpseudouridine) |
| 16 | Compound 16 | GCCACCATGAGAATCAGCAAGCCCCACCTGAGATCCATCAGCATCCAGTGC |
| | | TACCTGTGCCTGCTGCTGAACAGCCACTTTCTGACAGAGGCCGGCATCCAC |
| | | GTGTTCATCCTGGGCTGTTTTTCTGCCGGCCTGCCTAAGACCGAGGCCAAC |
| | | TGGGTTAACGTGATCAGCGACCTGAAGAAGATCGAGGACCTGATCCAGAGC |
| | | ATGCACATCGACGCCACACTGTACACCGAGAGCGACGTGCACCCTAGCTGT |
| | | AAAGTGACCGCCATGAAGTGCTTTCTGCTGGAACTGCAAGTGATCAGCCTG |
| | | GAAAGCGGCGACGCCAGCATCCACGACACCGTGGAAAACCTGATCATCCTG |
| | | GCCAACAACAGCCTGAGCAGCAACGGCAATGTGACCGAGTCCGGCTGCAAA |
| | | GAGTGCGAGGAACTGGAAGAGAAGAATATCAAAGAGTTCCTGCAGAGCTTC |
| | | GTGCACATCGTGCAGATGTTCATCAACACCAGCTGAATAGTGAGTCGTATT |
| | | AACGTACCAACAAGGAGTACCCTGATGAGATCACTTG [symbol] |
| | | [symbol] TTTATCTTAGAGGCATATCCCTACGTACCAACAAGAAGGTTCAGCA |
| | | TAGTAGCTAACTTG [symbol] [symbol] TTTATCTTAGAGGCAT |
| | | ATCCCTACGTACCAACAAGGACGACGAGACCTTCATCAAACTTG [symbol] |
| | | [symbol] TTTATCTTAGAGGCATATCCCTTTTATCTTAGAGGCA |
| | | TATCCCT |
| 140 | Compound 16 | GCCACCAUGAGAAUCAGCAAGCCCCACCUGAGAUCCAUCAGCAUCCAGUGC |
| | RNA sequence | UACCUGUGCCUGCUGCUGAACAGCCACUUUCUGACAGAGGCCGGCAUCCAC |
| | | GUGUUCAUCCUGGGCUGUUUUUCUGCCGGCCUGCCUAAGACCGAGGCCAAC |
| | | UGGGUUAACGUGAUCAGCGACCUGAAGAAGAUCGAGGACCUGAUCCAGAGC |
| | | AUGCACAUCGACGCCACACUGUACACCGAGAGCGACGUGCACCCUAGCUGU |
| | | AAAGUGACCGCCAUGAAGUGCUUUCUGCUGGAACUGCAAGUGAUCAGCCUG |
| | | GAAAGCGGCGACGCCAGCAUCCACGACACCGUGGAAAACCUGAUCAUCCUG |
| | | GCCAACAACAGCCUGAGCAGCAACGGCAAUGUGACCGAGUCCGGCUGCAAA |
| | | GAGUGCGAGGAACUGGAAGAGAAGAAUAUCAAAGAGUUCCUGCAGAGCUUC |
| | | GUGCACAUCGUGCAGAUGUUCAUCAACACCAGCUGAAUAGUGAGUCGUAUU |
| | | AACGUACCAACAAGGAGUACCCUGAUGAGAUCACUUG [symbol] |
| | | ACUCCUUUAUCUUAGAGGCAUAUCCCUACGUACCAACAAGAAGGUUCAGCA |
| | | UAGUAGCUAACUUG [symbol] [symbol] UUUAUCUUAGAGGCAU |
| | | AUCCCUACGUACCAACAAGGACGACGAGACCUUCAUCAAACUUG [symbol] |
| | | [symbol] UUUAUCUUAGAGGCAUAUCCCUUUUAUCUUAGAGGCA |
| | | UAUCCCU |
| | | (all Us are modified; N¹-methylpseudouridine) |
| 17 | Compound 17 | GCCACCATGTTCCACGTGTCCTTCCGGTACATCTTCGGCCTGCCTCCACTG |
| | | ATCCTGGTGCTGCTGCCTGTGGCCAGCAGCGACTGTGATATCGAGGGCAAA |
| | | GACGGCAAGCAGTACGAGAGCGTGCTGATGGTGTCCATCGACCAGCTGCTG |
| | | GACAGCATGAAGGAAATCGGCAGCAACTGCCTGAACAACGAGTTCAACTTC |
| | | TTCAAGCGGCACATCTGCGACGCCAACAAAGAAGGCATGTTCCTGTTCAGA |
| | | GCCGCCAGAAAGCTGCGGCAGTTCCTGAAGATGAACAGCACCGGCGACTTC |
| | | GACCTGCATCTGCTGAAAGTGTCTGAGGGCACCACCATCCTGCTGAATTGC |
| | | ACCGGCCAAGTGAAGGGCAGAAAGCCTGCTGCTCTGGGAGAAGCCCAGCCT |
| | | ACCAAGAGCCTGGAAGAGAACAAGTCCCTGAAAGAGCAGAAGAAGCTGAAC |
| | | GACCTCTGCTTCCTGAAGCGGCTGCTGCAAGAGATCAAGACCTGCTGGAAC |
| | | AAGATCCTGATGGGCACCAAAGAACACTGAATAGTGAGTCGTATTAACGTA |
| | | CCAACAAGAAGGTTCAGCATAGTAGCTAACTTG [symbol] |
| | | TTCTTTATCTTAGAGGCATATCCCTACGTACCAACAAGCGAATTACTGTGA |
| | | AAGTCAAACTTG [symbol] [symbol] TTTATCTTAGAGGCATAT |
| | | CCCTACGTACCAACAAGACCAGCACACTGAGAATCAAACTTG [symbol] |
| | | [symbol] TTTATCTTAGAGGCATATCCCTTTTATCTTAGAGGCATA |
| | | TCCCT |
| 141 | Compound 17 | GCCACCAUGUUCCACGUGUCCUUCCGGUACAUCUUCGGCCUGCCUCCACUG |
| | RNA sequence | AUCCUGGUGCUGCUGCCUGUGGCCAGCAGCGACUGUGAUAUCGAGGGCAAA |
| | | GACGGCAAGCAGUACGAGAGCGUGCUGAUGGUGUCCAUCGACCAGCUGCUG |
| | | GACAGCAUGAAGGAAAUCGGCAGCAACUGCCUGAACAACGAGUUCAACUUC |
| | | UUCAAGCGGCACAUCUGCGACGCCAACAAAGAAGGCAUGUUCCUGUUCAGA |
| | | GCCGCCAGAAAGCUGCGGCAGUUCCUGAAGAUGAACAGCACCGGCGACUUC |
| | | GACCUGCAUCUGCUGAAAGUGUCUGAGGGCACCACCAUCCUGCUGAAUUGC |
| | | ACCGGCCAAGUGAAGGGCAGAAAGCCUGCUGCUCUGGGAGAAGCCCAGCCU |
| | | ACCAAGAGCCUGGAAGAGAACAAGUCCCUGAAAGAGCAGAAGAAGCUGAAC |
| | | GACCUCUGCUUCCUGAAGCGGCUGCUGCAAGAGAUCAAGACCUGCUGGAAC |
| | | AAGAUCCUGAUGGGCACCAAAGAACACUGAAUAGUGAGUCGUAUUAACGUA |
| | | CCAACAAGAAGGUUCAGCAUAGUAGCUAACUUG [symbol] [symbol] |
| | | UUCUUUAUCUUAGAGGCAUAUCCCUACGUACCAACAAGCGAAUUACUGUGA |
| | | AAGUCAAACUUG [symbol] [symbol] UUUAUCUUAGAGGCAUAU |
| | | CCCUACGUACCAACAAGACCAGCACACUGAGAAUCAAACUUG [symbol] |
| | | AGUGUGCUGGUCUUUAUCUUAGAGGCAUAUCCCUUUUAUCUUAGAGGCAUA |
| | | UCCCU |
| | | (all Us are modified; N¹-methylpseudouridine) |

Bold = Sense siRNA strand
Bold and Italics = Anti-Sense siRNA strand
Underline = Signal peptide
Italics = Kozak sequence
*Bolding within the underlined sequence indicates modified signal peptide.

TABLE 3
Table of Sequences Listed

| | | SEQ |
|---|---|---|
| Protein or | | ID |
| Nucleic Acid | Sequence | NO: |
| Compound 1-6 | See Table 2 | 1- |
| nucleic acid | | 17 |
| sequences | | |
| T7 promoter | TAATACGACTCACTATA | 18 |
| Kozak sequence | GCCACC | 19 |
| tRNA linker | AACAAAGCACCAGTGGTCTAGTGGTAGAATAGTACCCTGCCACGGTACA | 20 |
| | GACCCGGGTTCGATTCCCGGCTGGTGCA | |
| mRNA to | ATAGTGAGTCGTATTAACGTACCAACAA | 21 |
| siRNA linker | | |
| siRNA to | TTTATCTTAGAGGCATATCCCTACGTACCAACAA | 22 |
| siRNA linker | | |
| Human IL-2 | MYRMQLLSCIALSLALVTNSAPTSSSTKKTQLQLEHLLLDLQMILNGINNYKNP | 23 |
| amino acid | KLTRMLTFKFYMPKKATELKHLQCLEEELKPLEEVLNLAQSKNFHLRPRDLISN | |
| (Genbank | INVIVLELKGSETTFMCEYADETATIVEFLNRWITFCQSIISTLT | |
| NM_000586.3) | | |
| Underlined: | | |
| signal sequence | | |
| Mature Human | APTSSSTKKTQLQLEHLLLDLQMILNGINNYKNPKLTRMLTFKFYMPKKATELK | 24 |
| IL-2 amino acid | HLQCLEEELKPLEEVLNLAQSKNFHLRPRDLISNINVIVLELKGSETTFMCEYA | |
| (Genbank | DETATIVEFLNRWITFCQSIISTLT | |
| NM_000586.3) | | |
| Underlined: | | |
| signal sequence | | |
| Human IL-2 | AGTTCCCTATCACTCTCTTTAATCACTACTCACAGTAACCTCAACTCCT | 25 |
| nucleic acid | GCCACAATGTACAGGATGCAACTCCTGTCTTGCATTGCACTAAGTCTTG | |
| (Genbank | CACTTGTCACAAACAGTGCACCTACTTCAAGTTCTACAAAGAAAACACA | |
| NM_000586.3) | GCTACAACTGGAGCATTTACTGCTGGATTTACAGATGATTTTGAATGGA | |
| Underlined: | ATTAATAATTACAAGAATCCCAAACTCACCAGGATGCTCACATTTAAGT | |
| coding sequence | TTTACATGCCCAAGAAGGCCACAGAACTGAAACATCTTCAGTGTCTAGA | |
| Bold: signal | AGAAGAACTCAAACCTCTGGAGGAAGTGCTAAATTTAGCTCAAAGCAAA | |
| sequence | AACTTTCACTTAAGACCCAGGGACTTAATCAGCAATATCAACGTAATAG | |
| | TTCTGGAACTAAAGGGATCTGAAACAACATTCATGTGTGAATATGCTGA | |
| | TGAGACAGCAACCATTGTAGAATTTCTGAACAGATGGATTACCTTTTGT | |
| | CAAAGCATCATCTCAACACTGACTTGATAATTAAGTGCTTCCCACTTAA | |
| | AACATATCAGGCCTTCTATTTATTTAAATATTTAAATTTTATATTTATT | |
| | GTTGAATGTATGGTTTGCTACCTATTGTAACTATTATTCTTAATCTTAA | |
| | AACTATAAATATGGATCTTTTATGATTCTTTTTGTAAGCCCTAGGGGCT | |
| | CTAAAATGGTTTCACTTATTTATCCCAAAATATTTATTATTATGTTGAA | |
| | TGTTAAATATAGTATCTATGTAGATTGGTTAGTAAAACTATTTAATAAA | |
| | TTTGATAAATATAAAAAAAAAAAAAAAAAAAAAAAAAA | |
| IL-2 signal | MYRMQLLSCIALSLALVTNS | 26 |
| peptide | | |
| (Genbank | | |
| NM_000586.3) | | |
| Modified IL-2 | MLKLLLLLCIALSLAATNS | 27 |
| signal peptide | | |
| (Cpd.2) amino | | |
| acid | | |
| (Y2L/R3K/M4L/ | | |
| Q5L/S8L/L16A/ | | |
| and V17-) | | |
| Modified IL-2 | MLLLLLACIALASTAAATNS | 28 |
| signal peptide | | |
| (Cpd.3) amino | | |
| acid (Y2L/R3-/ | | |
| M4L/Q5L/S8A/- | | |
| A13/L14T/ | | |
| L16A and | | |
| V17A) | | |
| Modified IL-2 | MLLLLLACIALASTALVTNS | 29 |
| signal peptide | | |
| (Cpd.4) amino | | |
| acid (Y2L/R3-/ | | |
| M4L/Q5L/S8A/- | | |
| A13 and L14T) | | |
| Endogenous IL- | ATGTACAGAATGCAGCTGCTGAGCTGTATCGCCCTGTCTCTGGCC | 30 |
| 2 signal peptide | CTGGTCACAAATAGC | |
| (Cpd.1) nucleic | | |
| acid | | |
| Modified IL-2 | ATGCTGAAACTGCTGCTGCTCCTGTGTATCGCCCTGTCTCTGGCC | 31 |
| signal peptide | GCCACAAATAGC | |
| (Cpd.2) nucleic | | |
| acid | | |
| Modified IL-2 | ATGTTGTTGCTGCTGCTCGCCTGTATTGCCCTGGCCTCTACAGCC | 32 |
| signal peptide | GCCGCTACAAATTCT | |
| (Cpd.3) nucleic | | |
| acid | | |
| Modified IL-2 | ATGTTGTTGCTGCTGCTCGCCTGTATTGCCCTGGCCTCTACAGCC | 33 |
| signal peptide | CTGGTCACCAATTCT | |
| (Cpd.4) nucleic | | |
| acid | | |
| VEGFA amino | MNFLLSWVHWSLALLLYLHHAKWSQAAPMAEGGGQNHHEVVKFMD | 34 |
| acid (Genbank | VYQRSYCHPIETLVDIFQEYPDEIEYIFKPSCVPLMRCGGCCNDE | |
| NM_001171623.1) | GLECVPTEESNITMQIMRIKPHQGQHIGEMSFLQHNKCECRPKKD | |
| (Transcript | RARQEKKSVRGKGKGQKRKRKKSRYKSWSVYVGARCCLMPWSLPG | |
| variant-1; | PHPCGPCSERRKHLFVQDPQTCKCSCKNTDSRCKARQLELNERTC | |
| Canonical | RCDKPRR | |
| sequence; | | |
| Isoform-206) | | |
| VEGFA (SEQ | ATGAACTTTCTGCTGTCTTGGGTGCATTGGAGCCTTGCCTTGCTG | 35 |
| ID NO: 34) | CTCTACCTCCACCATGCCAAGTGGTCCCAGGCTGCACCCATGGCA | |
| encoding DNA | GAAGGAGGAGG [symbol] [symbol] GAAGTTCATGGAT | |
| sequence | GTCTATCAGCGCAGCTACTGCCATCCAATCGAGACCCTGGTGGAC | |
| (from Genbank | ATCTTCCAGGAGTACCCTGATGAGATCGAGTACATCTTCAAGCCA | |
| NM_001171623.1) | TCCTGTGTGCCCCTGATGCGATGCGGGGGCTGCTGCAATGACGAG | |
| Bold: signal | GGCCTGGAGTGTGTGCCCACTGAGGAGTCCAACATCACCATGCAG | |
| peptide | ATTATGCGGATCAAACCTCACCAAGGCCAGCACATAGGAGAGAT [symbol] | |
| sequence | [symbol] [symbol] ATGTGAATGCAGACCAAAGAAAGAT | |
| Bold and | AGAGCAAGACAAGAAAAAAAATCAGTTCGAGGAAAGGGAAAGGGG | |
| italicized: | CAAAAACGAAAGCGCAAGAAATCCCGGTATAAGTCCTGGAGCGTG | |
| siRNA binding | TACGTTGGTGCCCGCTGCTGTCTAATGCCCTGGAGCCTCCCTGGC | |
| regions | CCCCATCCCTGTGGGCCTTGCTCAGAGCGGAGAAAGCATTTGTTT | |
| | GTACAA [symbol] [symbol] TCCTGCAAAAACACAGAC | |
| | TCGCGTTGCAAGGCGAGGCAGCTTGAGTTAAACGAACGTACTTGC | |
| | AGATGTGACAAGCCGAGGCGGTGA | |
| VEGFA (SEQ | AUGAACUUUCUGCUGUCUUGGGUGCAUUGGAGCCUUGCCUUGCUG | 36 |
| ID NO: 34) | CUCUACCUCCACCAUGCCAAGUGGUCCCAGGCUGCACCCAUGGCA | |
| encoding RNA | GAAGGAGGAGG [symbol] [symbol] GAAGUUCAUGGAU | |
| sequence | GUCUAUCAGCGCAGCUACUGCCAUCCAAUCGAGACCCUGGUGGAC | |
| (from Genbank | AUCUUCCAGGAGUACCCUGAUGAGAUCGAGUACAUCUUCAAGCCA | |
| NM_001171623.1) | UCCUGUGUGCCCCUGAUGCGAUGCGGGGGCUGCUGCAAUGACGAG | |
| Bold: signal | GGCCUGGAGUGUGUGCCCACUGAGGAGUCCAACAUCACCAUGCAG | |
| peptide | AUUAUGCGGAUCAAACCUCACCAAGGCCAGCACAUAGGAGAGAU [symbol] | |
| sequence | [symbol] [symbol] AUGUGAAUGCAGACCAAAGAAAGAU | |
| Bold and | AGAGCAAGACAAGAAAAAAAAUCAGUUCGAGGAAAGGGAAAGGGG | |
| italicized: | CAAAAACGAAAGCGCAAGAAAUCCCGGUAUAAGUCCUGGAGCGUG | |
| siRNA binding | UACGUUGGUGCCCGCUGCUGUCUAAUGCCCUGGAGCCUCCCUGGC | |
| regions | CCCCAUCCCUGUGGGCCUUGCUCAGAGCGGAGAAAGCAUUUGUUU | |
| | GUACAA [symbol] [symbol] UCCUGCAAAAACACAGAC | |
| | UCGCGUUGCAAGGCGAGGCAGCUUGAGUUAAACGAACGUACUUGC | |
| | AGAUGUGACAAGCCGAGGCGGUGA | |
| MICA amino | MGLGPVFLLLAGIFPFAPPGAAAEPHSLRYNLTVLSWDGSVQSGF | 37 |
| acid (Genbank | LTEVHLDGQPFLRCDRQKCRAKPQGQWAEDVLGNKTWDRETRDLT | |
| NM_000247.2) | GNGKDLRMTLAHIKDQKEGLHSLQEIRVCETHEDNSTRSSQHFYY | |
| (Transcript | DGELFLSQNLETKEWTMPQSSRAQTLAMNVRNFLKEDAMKTKTHY | |
| variant 1*001) | HAMHADCLQELRRYLKSGVVLRRTVPPMVNVTRSEASEGNITVTC | |
| | RASGFYPWNITLSWRQDGVSLSHDTQQWGDVLPDGNGTYQTWVAT | |
| | RICQGEEQRFTCYMEHSGNHSTHPVPSGKVLVLQSHWQTFHVSAV | |
| | AAAAIFVIIIFYVRCCKKKTSAAEGPELVSLQVLDQHPVGTSDHR | |
| | DATQLGFQPLMSDLGSTGSTEGA | |
| MICA (SEQ ID | ATGGGGCTGGGCCCGGTCTTCCTGCTTCTGGCTGGCATCTTCCCT | 38 |
| NO: 37) | TTTGCACCTCCGGGAGCTGCTGCTGAGCCCCACAGTCTTCGTTAT | |
| encoding DNA | AACCTCACGGTGCTGTCCTGGGATGGATCTGTGCAGTCAGGGTTT | |
| sequence | CTCACTGAGGTACATCTGGATGGTCAGCCCTTCCTGCGCTGTGAC | |
| (from Genbank | AGGCAGAAATGCAGGGCAAAGCCCCAGGGACAGTGGGCAGAAGAT | |
| NM_000247.2) | GTCCTGGGAAATAAGACATGGGACAGAGAGACCAGAGACTTGACA | |
| Bold and | GGGAACGGAAAGGACCTCAGGATGACCCTGGCTCATATCAAGGAC | |
| italicized: | CAGAAAGAAGGCTTGCATTCCCTCCA [symbol] [symbol] | |
| siRNA binding | ATCCATGAAGACAACAGCACCAGGAGCTCCCAGCATTTCTACTAC | |
| regions | GATGGGGAGCTCTTCCTCTCCCAAAACCTGGAGACTAAGGAATGG | |
| | ACAATGCCCCAGTCCTCCAGAGCTCAGACCTTGGCCATGAACGTC | |
| | AGGAATTTCTTGAAGGAA [symbol] [symbol] CACTAT | |
| | CACGCTATGCATGCAGACTGCCTGCAGGAACTACGGCGATATCTA | |
| | AAATCCGGCGTAGTCCTGAGGAGAACAGTGCCCCCCATGGTGAAT | |
| | GTCACCCGCAGCGAGGCCTCAGAGGGCAACATTACCGTGACATGC | |
| | AGGGCTTCTGGCTTCTATCCCTGGAATATCACACTGAGCTGGCGT | |
| | CAGGATGGGGTATCTTTGAGCCACGACACCCAGCAGTGGGGGGAT | |
| | GTCCT [symbol] [symbol] CCAGACCTGGGTGGCCACC | |
| | AGGATTTGCCAAGGAGAGGAGCAGAGGTTCACCTGCTACATGGAA | |
| | CACAGCGGGAATCACAGCACTCACCCTGTGCCCTCTGGGAAAGTG | |
| | CTGGTGCTTCAGAGTCATTGGCAGACATTCCATGTTTCTGCTGTT | |
| | GCTGCTGCTGCTATTTTTGTTATTATTATTTTCTATGTCCGTTGT | |
| | TGTAAGAAGAAAACATCAGCTGCAGAGGGTCCAGAGCTCGTGAGC | |
| | CTGCAGGTCCTGGATCAACACCCAGTTGGGACGAGTGACCACAGG | |
| | GATGCCACACAGCTCGGATTTCAGCCTCTGATGTCAGATCTTGGG | |
| | TCCACTGGCTCCACTGAGGGCGCCTAG | |
| MICA (SEQ ID | AUGGGGCUGGGCCCGGUCUUCCUGCUUCUGGCUGGCAUCUUCCCU | 39 |
| NO: 37) | UUUGCACCUCCGGGAGCUGCUGCUGAGCCCCACAGUCUUCGUUAU | |
| encoding RNA | AACCUCACGGUGCUGUCCUGGGAUGGAUCUGUGCAGUCAGGGUUU | |
| sequence | CUCACUGAGGUACAUCUGGAUGGUCAGCCCUUCCUGCGCUGUGAC | |
| (from Genbank | AGGCAGAAAUGCAGGGCAAAGCCCCAGGGACAGUGGGCAGAAGAU | |
| NM_000247.2) | GUCCUGGGAAAUAAGACAUGGGACAGAGAGACCAGAGACUUGACA | |
| Bold and | GGGAACGGAAAGGACCUCAGGAUGACCCUGGCUCAUAUCAAGGAC | |
| italicized: | CAGAAAGAAGGCUUGCAUUCCCUCCA [symbol] [symbol] | |
| siRNA binding | [symbol] CCAUGAAGACAACAGCACCAGGAGCUCCCAGCAUUUCUACUAC | |
| regions | GAUGGGGAGCUCUUCCUCUCCCAAAACCUGGAGACUAAGGAAUGG | |
| | ACAAUGCCCCAGUCCUCCAGAGCUCAGACCUUGGCCAUGAACGUC | |
| | AGGAAUUUCUUGAAGGAA [symbol] [symbol] CACUAU | |
| | CACGCUAUGCAUGCAGACUGCCUGCAGGAACUACGGCGAUAUCUA | |
| | AAAUCCGGCGUAGUCCUGAGGAGAACAGUGCCCCCCAUGGUGAAU | |
| | GUCACCCGCAGCGAGGCCUCAGAGGGCAACAUUACCGUGACAUGC | |
| | AGGGCUUCUGGCUUCUAUCCCUGGAAUAUCACACUGAGCUGGCGU | |
| | CAGGAUGGGGUAUCUUUGAGCCACGACACCCAGCAGUGGGGGGAU | |
| | GUCCU [symbol] [symbol] CCAGACCUGGGUGGCCACC | |
| | AGGAUUUGCCAAGGAGAGGAGCAGAGGUUCACCUGCUACAUGGAA | |
| | CACAGCGGGAAUCACAGCACUCACCCUGUGCCCUCUGGGAAAGUG | |
| | CUGGUGCUUCAGAGUCAUUGGCAGACAUUCCAUGUUUCUGCUGUU | |
| | GCUGCUGCUGCUAUUUUUGUUAUUAUUAUUUUCUAUGUCCGUUGU | |
| | UGUAAGAAGAAAACAUCAGCUGCAGAGGGUCCAGAGCUCGUGAGC | |
| | CUGCAGGUCCUGGAUCAACACCCAGUUGGGACGAGUGACCACAGG | |
| | GAUGCCACACAGCUCGGAUUUCAGCCUCUGAUGUCAGAUCUUGGG | |
| | UCCACUGGCUCCACUGAGGGCGCCUAG | |
| MICB amino | MGLGRVLLFLAVAFPFAPPAAAAEPHSLRYNLMVLSQDGSVQSGF | 40 |
| acid (Genbank | LAEGHLDGQPFLRYDRQKRRAKPQGQWAENVLGAKTWDTETEDLT | |
| NM_005931.4) | ENGQDLRRTLTHIKDQKGGLHSLQEIRVCEIHEDSSTRGSRHFYY | |
| (Transcript | DGELFLSQNLETQESTVPQSSRAQTLAMNVTNFWKEDAMKTKTHY | |
| variant 1) | RAMQADCLQKLQRYLKSGVAIRRTVPPMVNVTCSEVSEGNITVTC | |
| | RASSFYPRNITLTWRQDGVSLSHNTQQWGDVLPDGNGTYQTWVAT | |
| | RIRQGEEQRFTCYMEHSGNHGTHPVPSGKALVLQSQRTDFPYVSA | |
| | AMPCFVIIIILCVPCCKKKTSAAEGPELVSLQVLDQHPVGTGDHR | |
| | DAAQLGFQPLMSATGSTGSTEGT | |
| MICB (SEQ ID | ATGGGGCTGGGCCGGGTCCTGCTGTTTCTGGCCGTCGCCTTCCCT | 41 |
| NO: 40) | TTTGCACCCCCGGCAGCCGCCGCTGAGCCCCACAGTCTTCGTTAC | |
| encoding DNA | AACCTCATGGTGCTGTCCCAGGATGGATCTGTGCAGTCAGGGTTT | |
| sequence | CTCGCTGAGGGACATCTGGATGGTCAGCCCTTCCTGCGCTATGAC | |
| (from Genbank | AGGCAGAAACGCAGGGCAAAGCCCCAGGGACAGTGGGCAGAAAAT | |
| NM_005931.4) | GTCCTGGGAGCTAAGACCTGGGACACAGAGACCGAGGACTTGACA | |
| Bold and | GAGAATGGGCAAGACCTCAGGAGGACCCTGACTCATATCAAGGAC | |
| italicized: | CAGAAAGGAGGCTTGCATTCCCTCCA [symbol] [symbol] | |
| siRNA binding | ATCCATGAAGACAGCAGCACCAGGGGCTCCCGGCATTTCTACTAC | |
| regions | GATGGGGAGCTCTTCCTCTCCCAAAACCTGGAGACTCAAGAATCG | |
| | ACAGTGCCCCAGTCCTCCAGAGCTCAGACCTTGGCTATGAACGTC | |
| | ACAAATTTCTGGAAGGAA [symbol] [symbol] CACTAT | |
| | CGCGCTATGCAGGCAGACTGCCTGCAGAAACTACAGCGATATCTG | |
| | AAATCCGGGGTGGCCATCAGGAGAACAGTGCCCCCCATGGTGAAT | |
| | GTCACCTGCAGCGAGGTCTCAGAGGGCAACATCACCGTGACATGC | |
| | AGGGCTTCCAGCTTCTATCCCCGGAATATCACACTGACCTGGCGT | |
| | CAGGATGGGGTATCTTTGAGCCACAACACCCAGCAGTGGGGGGAT | |
| | GTCCT [symbol] [symbol] CCAGACCTGGGTGGCCACC | |
| | AGGATTCGCCAAGGAGAGGAGCAGAGGTTCACCTGCTACATGGAA | |
| | CACAGCGGGAATCACGGCACTCACCCTGTGCCCTCTGGGAAGGCG | |
| | CTGGTGCTTCAGAGTCAACGGACAGACTTTCCATATGTTTCTGCT | |
| | GCTATGCCATGTTTTGTTATTATTATTATTCTCTGTGTCCCTTGT | |
| | TGCAAGAAGAAAACATCAGCGGCAGAGGGTCCAGAGCTTGTGAGC | |
| | CTGCAGGTCCTGGATCAACACCCAGTTGGGACAGGAGACCACAGG | |
| | GATGCAGCACAGCTGGGATTTCAGCCTCTGATGTCAGCTACTGGG | |
| | TCCACTGGTTCCACTGAGGGCACCTAG | |
| MICB (SEQ ID | AUGGGGCUGGGCCGGGUCCUGCUGUUUCUGGCCGUCGCCUUCCCU | 42 |
| NO: 40) | UUUGCACCCCCGGCAGCCGCCGCUGAGCCCCACAGUCUUCGUUAC | |
| encoding RNA | AACCUCAUGGUGCUGUCCCAGGAUGGAUCUGUGCAGUCAGGGUUU | |
| sequence | CUCGCUGAGGGACAUCUGGAUGGUCAGCCCUUCCUGCGCUAUGAC | |
| (from Genbank | AGGCAGAAACGCAGGGCAAAGCCCCAGGGACAGUGGGCAGAAAAU | |
| NM_005931.4) | GUCCUGGGAGCUAAGACCUGGGACACAGAGACCGAGGACUUGACA | |
| Bold and | GAGAAUGGGCAAGACCUCAGGAGGACCCUGACUCAUAUCAAGGAC | |
| italicized: | CAGAAAGGAGGCUUGCAUUCCCUCCA [symbol] [symbol] | |
| siRNA binding | [symbol] CCAUGAAGACAGCAGCACCAGGGGCUCCCGGCAUUUCUACUAC | |
| regions | GAUGGGGAGCUCUUCCUCUCCCAAAACCUGGAGACUCAAGAAUCG | |
| | ACAGUGCCCCAGUCCUCCAGAGCUCAGACCUUGGCUAUGAACGUC | |
| | ACAAAUUUCUGGAAGGAA [symbol] [symbol] CACUAU | |
| | CGCGCUAUGCAGGCAGACUGCCUGCAGAAACUACAGCGAUAUCUG | |
| | AAAUCCGGGGUGGCCAUCAGGAGAACAGUGCCCCCCAUGGUGAAU | |
| | GUCACCUGCAGCGAGGUCUCAGAGGGCAACAUCACCGUGACAUGC | |
| | AGGGCUUCCAGCUUCUAUCCCCGGAAUAUCACACUGACCUGGCGU | |
| | CAGGAUGGGGUAUCUUUGAGCCACAACACCCAGCAGUGGGGGGAU | |
| | GUCCU [symbol] [symbol] CCAGACCUGGGUGGCCACC | |
| | AGGAUUCGCCAAGGAGAGGAGCAGAGGUUCACCUGCUACAUGGAA | |
| | CACAGCGGGAAUCACGGCACUCACCCUGUGCCCUCUGGGAAGGCG | |
| | CUGGUGCUUCAGAGUCAACGGACAGACUUUCCAUAUGUUUCUGCU | |
| | GCUAUGCCAUGUUUUGUUAUUAUUAUUAUUCUCUGUGUCCCUUGU | |
| | UGCAAGAAGAAAACAUCAGCGGCAGAGGGUCCAGAGCUUGUGAGC | |
| | CUGCAGGUCCUGGAUCAACACCCAGUUGGGACAGGAGACCACAGG | |
| | GAUGCAGCACAGCUGGGAUUUCAGCCUCUGAUGUCAGCUACUGGG | |
| | UCCACUGGUUCCACUGAGGGCACCUAG | |
| Human IL-12 | MCPARSLLLVATLVLLDHLSLARNLPVATPDPGMFPCLHHSQNLL | 43 |
| alpha amino | RAVSNMLQKARQTLEFYPCTSEEIDHEDITKDKTSTVEACLPLEL | |
| acid (Genbank | TKNESCLNSRETSFITNGSCLASRKTSFMMALCLSSIYEDLKMYQ | |
| NM_000882.4) | VEFKTMNAKLLMDPKRQIFLDQNMLAVIDELMQALNFNSETVPQK | |
| Underlined: | SSLEEPDFYKTKIKLCILLHAFRIRAVTIDRVMSYLNAS | |
| signal sequence | | |
| Mature Human | RNLPVATPDPGMFPCLHHSQNLLRAVSNMLQKARQTLEFYPCTSE | 44 |
| IL-12 alpha | EIDHEDITKDKTSTVEACLPLELTKNESCLNSRETSFITNGSCLA | |
| amino acid | SRKTSFMMALCLSSIYEDLKMYQVEFKTMNAKLLMDPKRQIFLDQ | |
| (Genbank | NMLAVIDELMQALNFNSETVPQKSSLEEPDFYKTKIKLCILLHAF | |
| NM_000882.4) | RIRAVTIDRVMSYLNAS | |
| Human IL-12 | ATTTCGCTTTCATTTTGGGCCGAGCTGGAGGCGGCGGGGCCGTCC | 45 |
| alpha | CGGAACGGCTGCGGCCGGGCACCCCGGGAGTTAATCCGAAAGCGC | |
| nucleic acid | CGCAAGCCCCGCGGGCCGGCCGCACCGCACGTGTCACCGAGAAGC | |
| (Genbank | TGATGTAGAGAGAGACACAGAAGGAGACAGAAAGCAAGAGACCAG | |
| NM_000882.4) | AGTCCCGGGAAAGTCCTGCCGCGCCTCGGGACAATTATAAAAATG | |
| Underlined: | TGGCCCCCTGGGTCAGCCTCCCAGCCACCGCCCTCACCTGCCGCG | |
| coding sequence | GCCACAGGTCTGCATCCAGCGGCTCGCCCTGTGTCCCTGCAGTGC | |
| Bold: signal | CGGCTCAGCATGTGTCCAGCGCGCAGCCTCCTCCTTGTGGCTACC | |
| sequence | CTGGTCCTCCTGGACCACCTCAGTTTGGCCAGAAACCTCCCCGTG | |
| | GCCACTCCAGACCCAGGAATGTTCCCATGCCTTCACCACTCCCAA | |
| | AACCTGCTGAGGGCCGTCAGCAACATGCTCCAGAAGGCCAGACAA | |
| | ACTCTAGAATTTTACCCTTGCACTTCTGAAGAGATTGATCATGAA | |
| | GATATCACAAAAGATAAAACCAGCACAGTGGAGGCCTGTTTACCA | |
| | TTGGAATTAACCAAGAATGAGAGTTGCCTAAATTCCAGAGAGACC | |
| | TCTTTCATAACTAATGGGAGTTGCCTGGCCTCCAGAAAGACCTCT | |
| | TTTATGATGGCCCTGTGCCTTAGTAGTATTTATGAAGACTTGAAG | |
| | ATGTACCAGGTGGAGTTCAAGACCATGAATGCAAAGCTTCTGATG | |
| | GATCCTAAGAGGCAGATCTTTCTAGATCAAAACATGCTGGCAGTT | |
| | ATTGATGAGCTGATGCAGGCCCTGAATTTCAACAGTGAGACTGTG | |
| | CCACAAAAATCCTCCCTTGAAGAACCGGATTTTTATAAAACTAAA | |
| | ATCAAGCTCTGCATACTTCTTCATGCTTTCAGAATTCGGGCAGTG | |
| | ACTATTGATAGAGTGATGAGCTATCTGAATGCTTCCTAAAAAGCG | |
| | AGGTCCCTCCAAACCGTTGTCATTTTTATAAAACTTTGAAATGAG | |
| | GAAACTTTGATAGGATGTGGATTAAGAACTAGGGAGGGGGAAAGA | |
| | AGGATGGGACTATTACATCCACATGATACCTCTGATCAAGTATTT | |
| | TTGACATTTACTGTGGATAAATTGTTTTTAAGTTTTCATGAATGA | |
| | ATTGCTAAGAAGGGAAAATATCCATCCTGAAGGTGTTTTTCATTC | |
| | ACTTTAATAGAAGGG | |
| Human IL-12 | MCHQQLVISWFSLVFLASPLVAIWELKKDVYVVELDWYPDAPGEM | 46 |
| beta amino acid | VVLTCDTPEEDGITWTLDQSSEVLGSGKTLTIQVKEFGDAGQYTC | |
| (Genbank | HKGGEVLSHSLLLLHKKEDGIWSTDILKDQKEPKNKTFLRCEAKN | |
| NM_002187.2) | YSGRFTCWWLTTISTDLTFSVKSSRGSSDPQGVTCGAATLSAERV | |
| Underlined; | RGDNKEYEYSVECQEDSACPAAEESLPIEVMVDAVHKLKYENYTS | |
| signal sequence | SFFIRDIIKPDPPKNLQLKPLKNSRQVEVSWEYPDTWSTPHSYFS | |
| | LTFCVQVQGKSKREKKDRVFTDKTSATVICRKNASISVRAQDRYY | |
| | SSSWSEWASVPCS | |
| Mature Human | IWELKKDVYVVELDWYPDAPGEMVVLTCDTPEEDGITWTLDQSSE | 47 |
| IL-12 beta | VLGSGKTLTIQVKEFGDAGQYTCHKGGEVLSHSLLLLHKKEDGIW | |
| amino acid | STDILKDQKEPKNKTFLRCEAKNYSGRFTCWWLTTISTDLTFSVK | |
| (Genbank | SSRGSSDPQGVTCGAATLSAERVRGDNKEYEYSVECQEDSACPAA | |
| NM_002187.2) | EESLPIEVMVDAVHKLKYENYTSSFFIRDIIKPDPPKNLQLKPLK | |
| | NSRQVEVSWEYPDTWSTPHSYFSLTFCVQVQGKSKREKKDRVFTD | |
| | KTSATVICRKNASISVRAQDRYYSSSWSEWASVPCS | |
| Human IL-12 | CTGTTTCAGGGCCATTGGACTCTCCGTCCTGCCCAGAGCAAGATG | 48 |
| beta | TGTCACCAGCAGTTGGTCATCTCTTGGTTTTCCCTGGTTTTTCTG | |
| nucleic acid | GCATCTCCCCTCGTGGCCATATGGGAACTGAAGAAAGATGTTTAT | |
| (Genbank | GTCGTAGAATTGGATTGGTATCCGGATGCCCCTGGAGAAATGGTG | |
| NM_002187.2) | GTCCTCACCTGTGACACCCCTGAAGAAGATGGTATCACCTGGACC | |
| Underlined: | TTGGACCAGAGCAGTGAGGTCTTAGGCTCTGGCAAAACCCTGACC | |
| coding sequence | ATCCAAGTCAAAGAGTTTGGAGATGCTGGCCAGTACACCTGTCAC | |
| Bold: signal | AAAGGAGGCGAGGTTCTAAGCCATTCGCTCCTGCTGCTTCACAAA | |
| sequence | AAGGAAGATGGAATTTGGTCCACTGATATTTTAAAGGACCAGAAA | |
| | GAACCCAAAAATAAGACCTTTCTAAGATGCGAGGCCAAGAATTAT | |
| | TCTGGACGTTTCACCTGCTGGTGGCTGACGACAATCAGTACTGAT | |
| | TTGACATTCAGTGTCAAAAGCAGCAGAGGCTCTTCTGACCCCCAA | |
| | GGGGTGACGTGCGGAGCTGCTACACTCTCTGCAGAGAGAGTCAGA | |
| | GGGGACAACAAGGAGTATGAGTACTCAGTGGAGTGCCAGGAGGAC | |
| | AGTGCCTGCCCAGCTGCTGAGGAGAGTCTGCCCATTGAGGTCATG | |
| | GTGGATGCCGTTCACAAGCTCAAGTATGAAAACTACACCAGCAGC | |
| | TTCTTCATCAGGGACATCATCAAACCTGACCCACCCAAGAACTTG | |
| | CAGCTGAAGCCATTAAAGAATTCTCGGCAGGTGGAGGTCAGCTGG | |
| | GAGTACCCTGACACCTGGAGTACTCCACATTCCTACTTCTCCCTG | |
| | ACATTCTGCGTTCAGGTCCAGGGCAAGAGCAAGAGAGAAAAGAAA | |
| | GATAGAGTCTTCACGGACAAGACCTCAGCCACGGTCATCTGCCGC | |
| | AAAAATGCCAGCATTAGCGTGCGGGCCCAGGACCGCTACTATAGC | |
| | TCATCTTGGAGCGAATGGGCATCTGTGCCCTGCAGTTAGGTTCTG | |
| | ATCCAGGATGAAAATTTGGAGGAAAAGTGGAAGATATTAAGCAAA | |
| | ATGTTTAAAGACACAACGGAATAGACCCAAAAAGATAATTTCTAT | |
| | CTGATTTGCTTTAAAACGTTTTTTTAGGATCACAATGATATCTTT | |
| | GCTGTATTTGTATAGTTAGATGCTAAATGCTCATTGAAACAATCA | |
| | GCTAATTTATGTATAGATTTTCCAGCTCTCAAGTTGCCATGGGCC | |
| | TTCATGCTATTTAAATATTTAAGTAATTTATGTATTTATTAGTAT | |
| | ATTACTGTTATTTAACGTTTGTCTGCCAGGATGTATGGAATGTTT | |
| | CATACTCTTATGACCTGATCCATCAGGATCAGTCCCTATTATGCA | |
| | AAATGTGAATTTAAT | |
| IDH1 amino | MSKKISGGSVVEMQGDEMTRIIWELIKEKLIFPYVELDLHSYDLG | 49 |
| acid (Genbank | IENRDATNDQVTKDAAEAIKKHNVGVKCATITPDEKRVEEFKLKQ | |
| NM_005896.3) | MWKSPNGTIRNILGGTVFREAIICKNIPRLVSGWVKPIIIGRHAY | |
| (Transcript | GDQYRATDFVVPGPGKVEITYTPSDGTQKVTYLVHNFEEGGGVAM | |
| variant 1) | GMYNQDKSIEDFAHSSFQMALSKGWPLYLSTKNTILKKYDGRFKD | |
| | IFQEIYDKQYKSQFEAQKIWYEHRLIDDMVAQAMKSEGGFIWACK | |
| | NYDGDVQSDSVAQGYGSLGMMTSVLVCPDGKTVEAEAAHGTVTRH | |
| | YRMYQKGQETSTNPIASIFAWTRGLAHRAKLDNNKELAFFANALE | |
| | EVSIETIEAGFMTKDLAACIKGLPNVQRSDYLNTFEFMDKLGENL | |
| | KIKLAçAKL | |
| IDH1 amino | ATGTCCAAAAAAATCAGTGGCGGTTCTGTGGTAGAGATGCAAGGA | 50 |
| acid encoding | GATGAAATGACACGAATCATTTGGGAATTGATTAAAGAGAAACTC | |
| DNA sequence | ATTTTTCCCTACGTGGAATTGGATCTACATAGCTATGATTTAGGC | |
| (from Genbank | ATAGAGAATCGTGATGCCACCAACGACCAAGTCACCAAGGATGCT | |
| NM_005896.3) | GCAGAAGCTATAAAGAAGCATAATGTTGGCGTCAAATGTGCCACT | |
| Bold and | ATCACTCCTGATGAGAAGAGGGTTGAGGAGTTCAAGTTGAAACAA | |
| italicized: | ATGTGGAAATCACCAAATGGCACCATACGAAATATTCTGGGTGGC | |
| siRNA binding | ACGGTCTTCAGAGAAGCCATTATCTGCAAAAATATCCCCCGGCTT | |
| region | GTGAGTGGATGGGTAAAACCTATCATCATAGGTCGTCATGCTTAT | |
| | GGGGATCAATACAGAGCAACTGATTTTGTTGTTCCTGGGCCTGGA | |
| | AAAGTAGAGATAACCTACACACCAAGTGACGGAACCCAAAAGGTG | |
| | ACATACCTGGTACATAACTTTGAAGAAGGTGGTGGTGTTGCCATG | |
| | GGGATGTATAATCAAGATAAGTCAATTGAAGATTTTGCACACA [symbol] | |
| | [symbol] [symbol] CTAAGGGTTGGCCTTTGTATCTGAGC | |
| | ACCAAAAACACTATTCTGAAGAAATATGATGGGCGTTTTAAAGAC | |
| | ATCTTTCAGGAGATATATGACAAGCAGTACAAGTCCCAGTTTGAA | |
| | GCTCAAAAGATCTGGTATGAGCATAGGCTCATCGACGACATGGTG | |
| | GCCCAAGCTATGAAATCAGAGGGAGGCTTCATCTGGGCCTGTAAA | |
| | AACTATGATGGTGACGTGCAGTCGGACTCTGTGGCCCAAGGGTAT | |
| | GGCTCTCTCGGCATGATGACCAGCGTGCTGGTTTGTCCAGATGGC | |
| | AAGACAGTAGAAGCAGAGGCTGCCCACGGGACTGTAACCCGTCAC | |
| | TACCGCATGTACCAGAAAGGACAGGAGACGTCCACCAATCCCATT | |
| | GCTTCCATTTTTGCCTGGACCAGAGGGTTAGCCCACAGAGCAAAG | |
| | CTTGATAACAATAAAGAGCTTGCCTTCTTTGCAAATGCTTTGGAA | |
| | GAAGTCTCTATTGAGACAATTGAGGCTGGCTTCATGACCAAGGAC | |
| | TTGGCTGCTTGCATTAAAGGTTTACCCAATGTGCAACGTTCTGAC | |
| | TACTTGAATACATTTGAGTTCATGGATAAACTTGGAGAAAACTTG | |
| | AAGATCAAACTAGCTCAGGCCAAACTTTAA | |
| IDH1 amino | AUGUCCAAAAAAAUCAGUGGCGGUUCUGUGGUAGAGAUGCAAGGA | 51 |
| acid encoding | GAUGAAAUGACACGAAUCAUUUGGGAAUUGAUUAAAGAGAAACUC | |
| RNA sequence | AUUUUUCCCUACGUGGAAUUGGAUCUACAUAGCUAUGAUUUAGGC | |
| (from Genbank | AUAGAGAAUCGUGAUGCCACCAACGACCAAGUCACCAAGGAUGCU | |
| NM_005896.3) | GCAGAAGCUAUAAAGAAGCAUAAUGUUGGCGUCAAAUGUGCCACU | |
| Bold and | AUCACUCCUGAUGAGAAGAGGGUUGAGGAGUUCAAGUUGAAACAA | |
| italicized: | AUGUGGAAAUCACCAAAUGGCACCAUACGAAAUAUUCUGGGUGGC | |
| siRNA binding | ACGGUCUUCAGAGAAGCCAUUAUCUGCAAAAAUAUCCCCCGGCUU | |
| region | GUGAGUGGAUGGGUAAAACCUAUCAUCAUAGGUCGUCAUGCUUAU | |
| | GGGGAUCAAUACAGAGCAACUGAUUUUGUUGUUCCUGGGCCUGGA | |
| | AAAGUAGAGAUAACCUACACACCAAGUGACGGAACCCAAAAGGUG | |
| | ACAUACCUGGUACAUAACUUUGAAGAAGGUGGUGGUGUUGCCAUG | |
| | GGGAUGUAUAAUCAAGAUAAGUCAAUUGAAGAUUUUGCACACA [symbol] | |
| | [symbol] [symbol] CUAAGGGUUGGCCUUUGUAUCUGAGC | |
| | ACCAAAAACACUAUUCUGAAGAAAUAUGAUGGGCGUUUUAAAGAC | |
| | AUCUUUCAGGAGAUAUAUGACAAGCAGUACAAGUCCCAGUUUGAA | |
| | GCUCAAAAGAUCUGGUAUGAGCAUAGGCUCAUCGACGACAUGGUG | |
| | GCCCAAGCUAUGAAAUCAGAGGGAGGCUUCAUCUGGGCCUGUAAA | |
| | AACUAUGAUGGUGACGUGCAGUCGGACUCUGUGGCCCAAGGGUAU | |
| | GGCUCUCUCGGCAUGAUGACCAGCGUGCUGGUUUGUCCAGAUGGC | |
| | AAGACAGUAGAAGCAGAGGCUGCCCACGGGACUGUAACCCGUCAC | |
| | UACCGCAUGUACCAGAAAGGACAGGAGACGUCCACCAAUCCCAUU | |
| | GCUUCCAUUUUUGCCUGGACCAGAGGGUUAGCCCACAGAGCAAAG | |
| | CUUGAUAACAAUAAAGAGCUUGCCUUCUUUGCAAAUGCUUUGGAA | |
| | GAAGUCUCUAUUGAGACAAUUGAGGCUGGCUUCAUGACCAAGGAC | |
| | UUGGCUGCUUGCAUUAAAGGUUUACCCAAUGUGCAACGUUCUGAC | |
| | UACUUGAAUACAUUUGAGUUCAUGGAUAAACUUGGAGAAAACUUG | |
| | AAGAUCAAACUAGCUCAGGCCAAACUUUAA | |
| CDK4 amino | MATSRYEPVAEIGVGAYGTVYKARDPHSGHFVALKSVRVPNGGGG | 52 |
| acid (Genbank | GGGLPISTVREVALLRRLEAFEHPNVVRLMDVCATSRTDREIKVT | |
| NM_000075.3) | LVFEHVDQDLRTYLDKAPPPGLPAETIKDLMRQFLRGLDFLHANC | |
| | IVHRDLKPENILVTSGGTVKLADFGLARIYSYQMALTPVVVTLWY | |
| | RAPEVLLQSTYATPVDMWSVGCIFAEMFRRKPLFCGNSEADQLGK | |
| | IFDLIGLPPEDDWPRDVSLPRGAFPPRGPRPVQSVVPEMEESGAQ | |
| | LLLEMLTFNPHKRISAFRALQHSYLHKDEGNPE | |
| CDK4 | ATGGCTACCTCTCGATATGAGCCAGTGGCTGAAATTGGTGTCGGT | 53 |
| encoding DNA | GCCTATGGGACAGTGTACAAGGCCCGTGATCCCCACAGTGGCCAC | |
| sequence | TTTGTGGCCCTCAAGAGTGTGAGAGTCCCCAATGGAGGAGGAGGT | |
| (from Genbank | GGAGGAGGCCTTCCCATCAGCACAGTTCGTGAGGTGGCTTTACTG | |
| NM_000075.3) | AGGCGACTGGAGGCTTTTGAGCATCCCAATGTTGTCCGGCTGATG | |
| Bold and | GACGTCTGTGCCACATCCCGAACTGACCGGGAGATCAAGGTAACC | |
| italicized: | CTGGTGTTTGAGCATGTAGACCAGGACCTAAGGACATATCTGGAC | |
| siRNA binding | AAGGCACCCCCACCAGGCTTGCCAGCCGAAACGATCAAGGATCTG | |
| regions | ATGCGCCAGTTTCTAAGAGGCCTAGATTTCCTTCATGCCAATT [symbol] | |
| | [symbol] [symbol] AGCCAGAGAACATTCTGGTGACAAGT | |
| | GGTGGAACAGTCAAGCTGGCTGACTTTGGCCTGGCCAGAATCTAC | |
| | AGCTACCAGATGGCACTTACACCCGTGGTTGTTACACTCTGGTAC | |
| | CGAGCTCCCGAAGTTCTTCTGCAGTCCACATATGCAACACCTGTG | |
| | GACATGTGGAGTGTTGGCTGTATCTTTGCAGAGATGTTTCGTCGA | |
| | AAGCCTCTCTTCTGTGGAAACTCTGAAGCCGACCAGTTGGGCAAA | |
| | ATCTTTGACCTGATTGGGCTGCCTCCAGAGGATGACTGGCCTCGA | |
| | GATGTATCCCTGCCCCGTGGAGCCTTTCCCCCCAGAGGGCCCCGC | |
| | CCAGTGCAGTCGGTGGTACCTGAGATGGAGGAGTCGGGAGCACAG | |
| | CTGCTGCTGGAAATGCTGACTTTTAACCCACACAAGCGAATCTCT | |
| | GCCTTTCGAGCTCTGCAGCACTCTTATCTACATAAGGATGAAGGT | |
| | AATCCGGAGTGA | |
| CDK4 encoding | AUGGCUACCUCUCGAUAUGAGCCAGUGGCUGAAAUUGGUGUCGGU | 54 |
| RNA sequence | GCCUAUGGGACAGUGUACAAGGCCCGUGAUCCCCACAGUGGCCAC | |
| (from Genbank | UUUGUGGCCCUCAAGAGUGUGAGAGUCCCCAAUGGAGGAGGAGGU | |
| NM_000075.3) | GGAGGAGGCCUUCCCAUCAGCACAGUUCGUGAGGUGGCUUUACUG | |
| Bold and | AGGCGACUGGAGGCUUUUGAGCAUCCCAAUGUUGUCCGGCUGAUG | |
| italicized: | GACGUCUGUGCCACAUCCCGAACUGACCGGGAGAUCAAGGUAACC | |
| siRNA binding | CUGGUGUUUGAGCAUGUAGACCAGGACCUAAGGACAUAUCUGGAC | |
| regions | AAGGCACCCCCACCAGGCUUGCCAGCCGAAACGAUCAAGGAUCUG | |
| | AUGCGCCAGUUUCUAAGAGGCCUAGAUUUCCUUCAUGCCAAUU [symbol] | |
| | [symbol] [symbol] AGCCAGAGAACAUUCUGGUGACAAGU | |
| | GGUGGAACAGUCAAGCUGGCUGACUUUGGCCUGGCCAGAAUCUAC | |
| | AGCUACCAGAUGGCACUUACACCCGUGGUUGUUACACUCUGGUAC | |
| | CGAGCUCCCGAAGUUCUUCUGCAGUCCACAUAUGCAACACCUGUG | |
| | GACAUGUGGAGUGUUGGCUGUAUCUUUGCAGAGAUGUUUCGUCGA | |
| | AAGCCUCUCUUCUGUGGAAACUCUGAAGCCGACCAGUUGGGCAAA | |
| | AUCUUUGACCUGAUUGGGCUGCCUCCAGAGGAUGACUGGCCUCGA | |
| | GAUGUAUCCCUGCCCCGUGGAGCCUUUCCCCCCAGAGGGCCCCGC | |
| | CCAGUGCAGUCGGUGGUACCUGAGAUGGAGGAGUCGGGAGCACAG | |
| | CUGCUGCUGGAAAUGCUGACUUUUAACCCACACAAGCGAAUCUCU | |
| | GCCUUUCGAGCUCUGCAGCACUCUUAUCUACAUAAGGAUGAAGGU | |
| | AAUCCGGAGUGA | |
| CDK6 amino | MEKDGLCRADQQYECVAEIGEGAYGKVFKARDLKNGGRFVALKRV | 55 |
| acid (Genbank | RVQTGEEGMPLSTIREVAVLRHLETFEHPNVVRLFDVCTVSRTDR | |
| NM_001259.6) | ETKLTLVFEHVDQDLTTYLDKVPEPGVPTETIKDMMFQLLRGLDF | |
| | LHSHRVVHRDLKPQNILVTSSGQIKLADFGLARIYSFQMALTSVV | |
| | VTLWYRAPEVLLQSSYATPVDLWSVGCIFAEMFRRKPLFRGSSDV | |
| | DQLGKILDVIGLPGEEDWPRDVALPRQAFHSKSAQPIEKFVTDID | |
| | ELGKDLLLKCLTFNPAKRISAYSALSHPYFQDLERCKENLDSHLP | |
| | PSQNTSELNTA | |
| CDK6 | ATGGAGAAGGACGGCCTGTGCCGCGCTGACCAGCAGTACGAATGC | 56 |
| encoding DNA | GTGGCGGAGATCGGGGAGGGCGCCTATGGGAAGGTGTTCAAGGCC | |
| sequence | CGCGACTTGAAGAACGGAGGCCGTTTCGTGGCGTTGAAGCGCGTG | |
| (from Genbank | CGGGTGCAGACCGGCGAGGAGGGCATGCCGCTCTCCACCATCCGC | |
| NM_001259.6) | GAGGTGGCGGTGCTGAGGCACCTGGAGACCTTCGAGCACCCCAAC | |
| Bold and | GTGGTCAGGTTGTTTGATGTGTGCACAGTGTCACGAACAGACAGA | |
| italicized: | GAAACCAAACTAACTTTAGTGTTTGAACATGTCGATCAAGACTTG | |
| siRNA binding | ACCACTTACTTGGATAAAGTTCCAGAGCCTGGAGTGCCCACTGAA | |
| regions | ACCATAAAGGATATGATGTTTCAGCTTCTCCGAGGTCTGGACTTT | |
| | CTTCATTCACACCGAGTAGTGCATCGCGATCTAAAACCACAGAAC | |
| | ATTCTGGT [symbol] [symbol] ACTCGCTGACTTCGGC | |
| | CTTGCCCGCATCTATAGTTTCCAGATGGCTCTAACCTCAGTGGTC | |
| | GTCACGCTGTGGTACAGAGCACCCGAAGTCTTGCTCCAGTCCAGC | |
| | TACGCCACCCCCGTGGATCTCTGGAGTGTTGGCTGCATATTTGCA | |
| | GAAATGTTTCGTAGAAAGCCTCTTTTTCGTGGAAGTTCAGATGTT | |
| | GATCAACTAGGAAAAATCTTGGACGTGATTGGACTCCCAGGAGAA | |
| | GAAGACTGGCCTAGAGATGTTGCCCTTCCCAGGCAGGCTTTTCAT | |
| | TCAAAATCTGCCCAACCAATTGAGAAGTTTGTAACAGATATCGAT | |
| | GAACTAGGCAAAGACCTACTTCTGAAGTGTTTGACATTTAACCCA | |
| | GCCAAAAGAATATCTGCCTACAGTGCCCTGTCTCACCCATACTTC | |
| | CAGGACCTGGAAAGGTGCAAAGAAAACCTGGATTCCCACCTGCCG | |
| | CCCAGCCAGAACACCTCGGAGCTGAATACAGCCTGA | |
| CDK6 encoding | AUGGAGAAGGACGGCCUGUGCCGCGCUGACCAGCAGUACGAAUGC | 57 |
| RNA sequence | GUGGCGGAGAUCGGGGAGGGCGCCUAUGGGAAGGUGUUCAAGGCC | |
| (from Genbank | CGCGACUUGAAGAACGGAGGCCGUUUCGUGGCGUUGAAGCGCGUG | |
| NM_001259.6) | CGGGUGCAGACCGGCGAGGAGGGCAUGCCGCUCUCCACCAUCCGC | |
| Bold and | GAGGUGGCGGUGCUGAGGCACCUGGAGACCUUCGAGCACCCCAAC | |
| italicized: | GUGGUCAGGUUGUUUGAUGUGUGCACAGUGUCACGAACAGACAGA | |
| siRNA binding | GAAACCAAACUAACUUUAGUGUUUGAACAUGUCGAUCAAGACUUG | |
| regions | ACCACUUACUUGGAUAAAGUUCCAGAGCCUGGAGUGCCCACUGAA | |
| | ACCAUAAAGGAUAUGAUGUUUCAGCUUCUCCGAGGUCUGGACUUU | |
| | CUUCAUUCACACCGAGUAGUGCAUCGCGAUCUAAAACCACAGAAC | |
| | AUUCUGGU [symbol] [symbol] ACUCGCUGACUUCGGC | |
| | CUUGCCCGCAUCUAUAGUUUCCAGAUGGCUCUAACCUCAGUGGUC | |
| | GUCACGCUGUGGUACAGAGCACCCGAAGUCUUGCUCCAGUCCAGC | |
| | UACGCCACCCCCGUGGAUCUCUGGAGUGUUGGCUGCAUAUUUGCA | |
| | GAAAUGUUUCGUAGAAAGCCUCUUUUUCGUGGAAGUUCAGAUGUU | |
| | GAUCAACUAGGAAAAAUCUUGGACGUGAUUGGACUCCCAGGAGAA | |
| | GAAGACUGGCCUAGAGAUGUUGCCCUUCCCAGGCAGGCUUUUCAU | |
| | UCAAAAUCUGCCCAACCAAUUGAGAAGUUUGUAACAGAUAUCGAU | |
| | GAACUAGGCAAAGACCUACUUCUGAAGUGUUUGACAUUUAACCCA | |
| | GCCAAAAGAAUAUCUGCCUACAGUGCCCUGUCUCACCCAUACUUC | |
| | CAGGACCUGGAAAGGUGCAAAGAAAACCUGGAUUCCCACCUGCCG | |
| | CCCAGCCAGAACACCUCGGAGCUGAAUACAGCCUGA | |
| EGFR amino | MRPSGTAGAALLALLAALCPASRALEEKKVCQGTSNKLTQLGTFE | 58 |
| acid (Genbank | DHFLSLQRMFNNCEVVLGNLEITYVQRNYDLSFLKTIQEVAGYVL | |
| NM_005228.4) | IALNTVERIPLENLQIIRGNMYYENSYALAVLSNYDANKTGLKEL | |
| (Transcript | PMRNLQEILHGAVRFSNNPALCNVESIQWRDIVSSDFLSNMSMDF | |
| variant 1) | QNHLGSCQKCDPSCPNGSCWGAGEENCQKLTKIICAQQCSGRCRG | |
| | KSPSDCCHNQCAAGCTGPRESDCLVCRKFRDEATCKDTCPPLMLY | |
| | NPTTYQMDVNPEGKYSFGATCVKKCPRNYVVTDHGSCVRACGADS | |
| | YEMEEDGVRKCKKCEGPCRKVCNGIGIGEFKDSLSINATNIKHFK | |
| | NCTSISGDLHILPVAFRGDSFTHTPPLDPQELDILKTVKEITGFL | |
| | LIQAWPENRTDLHAFENLEIIRGRTKQHGQFSLAVVSLNITSLGL | |
| | RSLKEISDGDVIISGNKNLCYANTINWKKLFGTSGQKTKIISNRG | |
| | ENSCKATGQVCHALCSPEGCWGPEPRDCVSCRNVSRGRECVDKCN | |
| | LLEGEPREFVENSECIQCHPECLPQAMNITCTGRGPDNCIQCAHY | |
| | IDGPHCVKTCPAGVMGENNTLVWKYADAGHVCHLCHPNCTYGCTG | |
| | PGLEGCPTNGPKIPSIATGMVGALLLLLVVALGIGLFMRRRHIVR | |
| | KRTLRRLLQERELVEPLTPSGEAPNQALLRILKETEFKKIKVLGS | |
| | GAFGTVYKGLWIPEGEKVKIPVAIKELREATSPKANKEILDEAYV | |
| | MASVDNPHVCRLLGICLTSTVQLITQLMPFGCLLDYVREHKDNIG | |
| | SQYLLNWCVQIAKGMNYLEDRRLVHRDLAARNVLVKTPQHVKITD | |
| | FGLAKLLGAEEKEYHAEGGKVPIKWMALESILHRIYTHQSDVWSY | |
| | GVTVWELMTFGSKPYDGIPASEISSILEKGERLPQPPICTIDVYM | |
| | IMVKCWMIDADSRPKFRELIIEFSKMARDPQRYLVIQGDERMHLP | |
| | SPTDSNFYRALMDEEDMDDVVDADEYLIPQQGFFSSPSTSRTPLL | |
| | SSLSATSNNSTVACIDRNGLQSCPIKEDSFLQRYSSDPTGALTED | |
| | SIDDTFLPVPEYINQSVPKRPAGSVQNPVYHNQPLNPAPSRDPHY | |
| | QDPHSTAVGNPEYLNTVQPTCVNSTFDSPAHWAQKGSHQISLDNP | |
| | DYQQDFFPKEAKPNGIFKGSTAENAEYLRVAPQSSEFIGA | |
| EGFR encoding | ATGCGACCCTCCGGGACGGCCGGGGCAGCGCTCCTGGCGCTGCTG | 59 |
| DNA sequence | GCTGCGCTCTGCCCGGCGAGTCGGGCTCTGGAGGAAAAGAAAGTT | |
| (from Genbank | TGCCAAGGCACGAGTAACAAGCTCACGCAGTTGGGCACTTTTGAA | |
| NM_005228.4) | GATCATTTTCTCAGCCTCCAGAGGATGTTCAATAACTGTGAGGTG | |
| Bold and | GTCCTTGGGAATTTGGAAATTACCTATGTGCAGAGGAATTATGAT | |
| italicized: | CTTTCCTTCTTAAAGACCATCCAGGAGGTGGCTGGTTATGTCCTC | |
| siRNA binding | ATTGCCCTCAACACAGTGGAGCGAATTCCTTTGGAAAACCTGCAG | |
| regions | ATCATCAGAGGAAATATGTACTACGAAAATTCCTATGCCTTAGCA | |
| | GTCTTATCTAACTATGATGCAAATAAAACCGGACT [symbol] | |
| | [symbol] TTTACAGGAAATCCTGCATGGCGCCGTGCGGTTC | |
| | AGCAACAACCCTGCCCTGTGCAACGTGGAGAGCATCCAGTGGCGG | |
| | GACATAGTCAGCAGTGACTTTCTCAGCAACATGTCGATGGACTTC | |
| | CAGAACCACCTGGGCAGCTGCCAAAAGTGTGATCCAAGCTGTCCC | |
| | AATGGGAGCTGCTGGGGTGCAGGAGAGGAGAACTGCCAGAAACTG | |
| | ACCAAAATCATCTGTGCCCAGCAGTGCTCCGGGCGCTGCCGTGGC | |
| | AAGTCCCCCAGTGACTGCTGCCACAACCAGTGTGCTGCAGGCTGC | |
| | ACAGGCCCCCGGGAGAGCGACTGCCTGGTCTGCCGCAAATTCCGA | |
| | GACGAAGCCACGTGCAAGGACACCTGCCCCCCACTCATGCTCTAC | |
| | AACCCCACCACGTACCAGATGGATGTGAACCCCGAGGGCAAATAC | |
| | AGCTTTGGTGCCACCTGCGTGAAGAAGTGTCCCCGTAATTATGTG | |
| | GTGACAGATCACGGCTCGTGCGTCCGAGCCTGTGGGGCCGACAGC | |
| | TATGAGATGGAGGAAGACGGCGTCCGCAAGTGTAAGAAGTGCGAA | |
| | GGGCCTTGCCGCAAAGTGTGTAACGGAATAGGTATTGGTGAATTT | |
| | AAAGACTCACTCTCCATAAATGCTACGAATATTAAACACTTCAAA | |
| | AACTGCACCTCCATCAGTGGCGATCTCCACATCCTGCCGGTGGCA | |
| | TTTAGGGGTGACTCCTTCACACATACTCCTCCTCTGGATCCACAG | |
| | GAACTGGATATTCTGAAAACCGTAAAGGAAATCACAGGGTTTTTG | |
| | CTGATTCAGGCTTGGCCTGAAAACAGGACGGACCTCCATGCCTTT | |
| | GAGAACCTAGAAATCATACGCGGCAGGACCAAGCAACATGGTCAG | |
| | TTTTCTCTTGCAGTCGTCAGCCTGAACATAACATCCTTGGGATTA | |
| | CGCTCCCTCAAGGAGATAAGTGATGGAGATGTGATAATTTCAGGA | |
| | AACAAAAATTTGTGCTATGCAAATACAATAAACTGGAAAAAACTG | |
| | TTTGGGACCTCCGGTCAGAAAACCAAAATTATAAGCAACAGAGGT | |
| | GAAAACAGCTGCAAGGCCACAGGCCAGGTCTGCCATGCCTTGTGC | |
| | TCCCCCGAGGGCTGCTGGGGCCCGGAGCCCAGGGACTGCGTCTCT | |
| | TGCCGGAATGTCAGCCGAGGCAGGGAATGCGTGGACAAGTGCAAC | |
| | CTTCTGGAGGGTGAGCCAAGGGAGTTTGTGGAGAACTCTGAGTGC | |
| | ATACAGTGCCACCCAGAGTGCCTGCCTCAGGCCATGAACATCACC | |
| | TGCACAGGACGGGGACCAGACAACTGTATCCAGTGTGCCCACTAC | |
| | ATTGACGGCCCCCACTGCGTCAAGACCTGCCCGGCAGGAGTCATG | |
| | GGAGAAAACAACACCCTGGTCTGGAAGTACGCAGACGCCGGCCAT | |
| | GTGTGCCACCTGTGCCATCCAAACTGCACCTACGGATGCACTGGG | |
| | CCAGGTCTTGAAGGCT [symbol] [symbol] TCCCGTCC | |
| | ATCGCCACTGGGATGGTGGGGGCCCTCCTCTTGCTGCTGGTGGTG | |
| | GCCCTGGGGATCGGCCTCTTCATGCGAAGGCGCCACATCGTTCGG | |
| | AAGCGCACGCTGCGGAGGCTGCTGCAGGAGAGGGAGCTTGTGGAG | |
| | CCTCTTACACCCAGTGGAGAAGCTCCCAACCAAGCTCTCTTGAGG | |
| | ATCTTGAAGGAAACTGAATTCAAAAAGATCAAAGTGCTGGGCTCC | |
| | GGTGCGTTCGGCACGGTGTATAAGGGACTCTGGATCCCAGAAGGT | |
| | GAGAAAGTTAAAATTCCCGTCGCTATCAAGGAATTAAGAGAAGCA | |
| | ACATCTCCGAAAGCCAACAAGGAAATCCTCGATGAAGCCTACGTG | |
| | ATGGCCAGCGTGGACAACCCCCACGTGTGCCGCCTGCTGGGCATC | |
| | TGCCTCACCTCCACCGTGCAGCTCATCACGCAGCTCATGCCCTTC | |
| | GGCTGCCTCCTGGACTATGTCCGGGAACACAAAGACAATATTGGC | |
| | TCCCAGTACCTGCTCAACTGGTGTGTGCAGATCGCAAAGGGCATG | |
| | AACTACTTGGAGGACCGTCGCTTGGTGCACCGCGACCTGGCAGCC | |
| | AGGAACGTACTGGTGAAAACACCGCAGCATGTCAAGATCACAGAT | |
| | TTTGGGCTGGCCAAACTGCTGGGTGCGGAAGAGAAAGAATACCAT | |
| | GCAGAAGGAGGCAAAGTGCCTATCAAGTGGATGGCATTGGAATCA | |
| | ATTTTACACAGAATCTATACCCACCAGAGTGATGTCTGGAGCTAC | |
| | GGGGTGACCGTTTGGGAGTTGATGACCTTTGGATCCAAGCCATAT | |
| | GACGGAATCCCTGCCAGCGAGATCTCCTCCATCCTGGAGAAAGGA | |
| | GAACGCCTCCCTCAGCCACCCATATGTACCATCGATGTCTACATG | |
| | ATCATGGTCAAGTGCTGGATGATAGACGCAGATAGTCGCCCAAAG | |
| | TTCCGTGAGTTGATCATCGAATTCTCCAAAATGGCCCGAGACCCC | |
| | CAGCGCTACCTTGTCATTCAGGGGGATGAAAGAATGCATTTGCCA | |
| | AGTCCTACAGACTCCAACTTCTACCGTGCCCTGATGGATGAAGAA | |
| | GACATGGACGACGTGGTGGATGCCGACGAGTACCTCATCCCACAG | |
| | CAGGGCTTCTTCAGCAGCCCCTCCACGTCACGGACTCCCCTCCTG | |
| | AGCTCTCTGAGTGCAACCAGCAACAATTCCACCGTGGCTTGCATT | |
| | GATAGAAATGGGCTGCAAAGCTGTCCCATCAAGGAAGACAGCTTC | |
| | TTGCAGCGATACAGCTCAGACCCCACAGGCGCCTTGACTGA [symbol] | |
| | [symbol] CCTCCCAGTGCCTGAATACATAAACCAG | |
| | TCCGTTCCCAAAAGGCCCGCTGGCTCTGTGCAGAATCCTGTCTAT | |
| | CACAATCAGCCTCTGAACCCCGCGCCCAGCAGAGACCCACACTAC | |
| | CAGGACCCCCACAGCACTGCAGTGGGCAACCCCGAGTATCTCAAC | |
| | ACTGTCCAGCCCACCTGTGTCAACAGCACATTCGACAGCCCTGCC | |
| | CACTGGGCCCAGAAAGGCAGCCACCAAATTAGCCTGGACAACCCT | |
| | GACTACCAGCAGGACTTCTTTCCCAAGGAAGCCAAGCCAAATGGC | |
| | ATCTTTAAGGGCTCCACAGCTGAAAATGCAGAATACCTAAGGGTC | |
| | GCGCCACAAAGCAGTGAATTTATTGGAGCATGA | |
| EGFR encoding | AUGCGACCCUCCGGGACGGCCGGGGCAGCGCUCCUGGCGCUGCUG | 60 |
| RNA sequence | GCUGCGCUCUGCCCGGCGAGUCGGGCUCUGGAGGAAAAGAAAGUU | |
| (from Genbank | UGCCAAGGCACGAGUAACAAGCUCACGCAGUUGGGCACUUUUGAA | |
| NM_005228.4) | GAUCAUUUUCUCAGCCUCCAGAGGAUGUUCAAUAACUGUGAGGUG | |
| Bold and | GUCCUUGGGAAUUUGGAAAUUACCUAUGUGCAGAGGAAUUAUGAU | |
| italicized: | CUUUCCUUCUUAAAGACCAUCCAGGAGGUGGCUGGUUAUGUCCUC | |
| siRNA binding | AUUGCCCUCAACACAGUGGAGCGAAUUCCUUUGGAAAACCUGCAG | |
| regions | AUCAUCAGAGGAAAUAUGUACUACGAAAAUUCCUAUGCCUUAGCA | |
| | GUCUUAUCUAACUAUGAUGCAAAUAAAACCGGACU [symbol] | |
| | [symbol] UUUACAGGAAAUCCUGCAUGGCGCCGUGCGGUUC | |
| | AGCAACAACCCUGCCCUGUGCAACGUGGAGAGCAUCCAGUGGCGG | |
| | GACAUAGUCAGCAGUGACUUUCUCAGCAACAUGUCGAUGGACUUC | |
| | CAGAACCACCUGGGCAGCUGCCAAAAGUGUGAUCCAAGCUGUCCC | |
| | AAUGGGAGCUGCUGGGGUGCAGGAGAGGAGAACUGCCAGAAACUG | |
| | ACCAAAAUCAUCUGUGCCCAGCAGUGCUCCGGGCGCUGCCGUGGC | |
| | AAGUCCCCCAGUGACUGCUGCCACAACCAGUGUGCUGCAGGCUGC | |
| | ACAGGCCCCCGGGAGAGCGACUGCCUGGUCUGCCGCAAAUUCCGA | |
| | GACGAAGCCACGUGCAAGGACACCUGCCCCCCACUCAUGCUCUAC | |
| | AACCCCACCACGUACCAGAUGGAUGUGAACCCCGAGGGCAAAUAC | |
| | AGCUUUGGUGCCACCUGCGUGAAGAAGUGUCCCCGUAAUUAUGUG | |
| | GUGACAGAUCACGGCUCGUGCGUCCGAGCCUGUGGGGCCGACAGC | |
| | UAUGAGAUGGAGGAAGACGGCGUCCGCAAGUGUAAGAAGUGCGAA | |
| | GGGCCUUGCCGCAAAGUGUGUAACGGAAUAGGUAUUGGUGAAUUU | |
| | AAAGACUCACUCUCCAUAAAUGCUACGAAUAUUAAACACUUCAAA | |
| | AACUGCACCUCCAUCAGUGGCGAUCUCCACAUCCUGCCGGUGGCA | |
| | UUUAGGGGUGACUCCUUCACACAUACUCCUCCUCUGGAUCCACAG | |
| | GAACUGGAUAUUCUGAAAACCGUAAAGGAAAUCACAGGGUUUUUG | |
| | CUGAUUCAGGCUUGGCCUGAAAACAGGACGGACCUCCAUGCCUUU | |
| | GAGAACCUAGAAAUCAUACGCGGCAGGACCAAGCAACAUGGUCAG | |
| | UUUUCUCUUGCAGUCGUCAGCCUGAACAUAACAUCCUUGGGAUUA | |
| | CGCUCCCUCAAGGAGAUAAGUGAUGGAGAUGUGAUAAUUUCAGGA | |
| | AACAAAAAUUUGUGCUAUGCAAAUACAAUAAACUGGAAAAAACUG | |
| | UUUGGGACCUCCGGUCAGAAAACCAAAAUUAUAAGCAACAGAGGU | |
| | GAAAACAGCUGCAAGGCCACAGGCCAGGUCUGCCAUGCCUUGUGC | |
| | UCCCCCGAGGGCUGCUGGGGCCCGGAGCCCAGGGACUGCGUCUCU | |
| | UGCCGGAAUGUCAGCCGAGGCAGGGAAUGCGUGGACAAGUGCAAC | |
| | CUUCUGGAGGGUGAGCCAAGGGAGUUUGUGGAGAACUCUGAGUGC | |
| | AUACAGUGCCACCCAGAGUGCCUGCCUCAGGCCAUGAACAUCACC | |
| | UGCACAGGACGGGGACCAGACAACUGUAUCCAGUGUGCCCACUAC | |
| | AUUGACGGCCCCCACUGCGUCAAGACCUGCCCGGCAGGAGUCAUG | |
| | GGAGAAAACAACACCCUGGUCUGGAAGUACGCAGACGCCGGCCAU | |
| | GUGUGCCACCUGUGCCAUCCAAACUGCACCUACGGAUGCACUGGG | |
| | CCAGGUCUUGAAGGCU [symbol] [symbol] UCCCGUCC | |
| | AUCGCCACUGGGAUGGUGGGGGCCCUCCUCUUGCUGCUGGUGGUG | |
| | GCCCUGGGGAUCGGCCUCUUCAUGCGAAGGCGCCACAUCGUUCGG | |
| | AAGCGCACGCUGCGGAGGCUGCUGCAGGAGAGGGAGCUUGUGGAG | |
| | CCUCUUACACCCAGUGGAGAAGCUCCCAACCAAGCUCUCUUGAGG | |
| | AUCUUGAAGGAAACUGAAUUCAAAAAGAUCAAAGUGCUGGGCUCC | |
| | GGUGCGUUCGGCACGGUGUAUAAGGGACUCUGGAUCCCAGAAGGU | |
| | GAGAAAGUUAAAAUUCCCGUCGCUAUCAAGGAAUUAAGAGAAGCA | |
| | ACAUCUCCGAAAGCCAACAAGGAAAUCCUCGAUGAAGCCUACGUG | |
| | AUGGCCAGCGUGGACAACCCCCACGUGUGCCGCCUGCUGGGCAUC | |
| | UGCCUCACCUCCACCGUGCAGCUCAUCACGCAGCUCAUGCCCUUC | |
| | GGCUGCCUCCUGGACUAUGUCCGGGAACACAAAGACAAUAUUGGC | |
| | UCCCAGUACCUGCUCAACUGGUGUGUGCAGAUCGCAAAGGGCAUG | |
| | AACUACUUGGAGGACCGUCGCUUGGUGCACCGCGACCUGGCAGCC | |
| | AGGAACGUACUGGUGAAAACACCGCAGCAUGUCAAGAUCACAGAU | |
| | UUUGGGCUGGCCAAACUGCUGGGUGCGGAAGAGAAAGAAUACCAU | |
| | GCAGAAGGAGGCAAAGUGCCUAUCAAGUGGAUGGCAUUGGAAUCA | |
| | AUUUUACACAGAAUCUAUACCCACCAGAGUGAUGUCUGGAGCUAC | |
| | GGGGUGACCGUUUGGGAGUUGAUGACCUUUGGAUCCAAGCCAUAU | |
| | GACGGAAUCCCUGCCAGCGAGAUCUCCUCCAUCCUGGAGAAAGGA | |
| | GAACGCCUCCCUCAGCCACCCAUAUGUACCAUCGAUGUCUACAUG | |
| | AUCAUGGUCAAGUGCUGGAUGAUAGACGCAGAUAGUCGCCCAAAG | |
| | UUCCGUGAGUUGAUCAUCGAAUUCUCCAAAAUGGCCCGAGACCCC | |
| | CAGCGCUACCUUGUCAUUCAGGGGGAUGAAAGAAUGCAUUUGCCA | |
| | AGUCCUACAGACUCCAACUUCUACCGUGCCCUGAUGGAUGAAGAA | |
| | GACAUGGACGACGUGGUGGAUGCCGACGAGUACCUCAUCCCACAG | |
| | CAGGGCUUCUUCAGCAGCCCCUCCACGUCACGGACUCCCCUCCUG | |
| | AGCUCUCUGAGUGCAACCAGCAACAAUUCCACCGUGGCUUGCAUU | |
| | GAUAGAAAUGGGCUGCAAAGCUGUCCCAUCAAGGAAGACAGCUUC | |
| | UUGCAGCGAUACAGCUCAGACCCCACAGGCGCCUUGACUGA [symbol] | |
| | [symbol] [symbol] CCUCCCAGUGCCUGAAUACAUAAACCAG | |
| | UCCGUUCCCAAAAGGCCCGCUGGCUCUGUGCAGAAUCCUGUCUAU | |
| | CACAAUCAGCCUCUGAACCCCGCGCCCAGCAGAGACCCACACUAC | |
| | CAGGACCCCCACAGCACUGCAGUGGGCAACCCCGAGUAUCUCAAC | |
| | ACUGUCCAGCCCACCUGUGUCAACAGCACAUUCGACAGCCCUGCC | |
| | CACUGGGCCCAGAAAGGCAGCCACCAAAUUAGCCUGGACAACCCU | |
| | GACUACCAGCAGGACUUCUUUCCCAAGGAAGCCAAGCCAAAUGGC | |
| | AUCUUUAAGGGCUCCACAGCUGAAAAUGCAGAAUACCUAAGGGUC | |
| | GCGCCACAAAGCAGUGAAUUUAUUGGAGCAUGA | |
| mTOR amino | MLGTGPAAATTAATTSSNVSVLQQFASGLKSRNEETRAKAAKELQ | 61 |
| acid (Genbank | HYVTMELREMSQEESTRFYDQLNHHIFELVSSSDANERKGGILAI | |
| NM_005931.4) | ASLIGVEGGNATRIGRFANYLRNLLPSNDPVVMEMASKAIGRLAM | |
| | AGDTFTAEYVEFEVKRALEWLGADRNEGRRHAAVLVLRELAISVP | |
| | TFFFQQVQPFFDNIFVAVWDPKQAIREGAVAALRACLILTTQREP | |
| | KEMQKPQWYRHTFEEAEKGFDETLAKEKGMNRDDRIHGALLILNE | |
| | LVRISSMEGERLREEMEEITQQQLVHDKYCKDLMGFGTKPRHITP | |
| | FTSFQAVQPQQSNALVGLLGYSSHQGLMGFGTSPSPAKSTLVESR | |
| | CCRDLMEEKFDQVCQWVLKCRNSKNSLIQMTILNLLPRLAAFRPS | |
| | AFTDTQYLQDTMNHVLSCVKKEKERTAAFQALGLLSVAVRSEFKV | |
| | YLPRVLDIIRAALPPKDFAHKRQKAMQVDATVFTCISMLARAMGP | |
| | GIQQDIKELLEPMLAVGLSPALTAVLYDLSRQIPQLKKDIQDGLL | |
| | KMLSLVLMHKPLRHPGMPKGLAHQLASPGLTTLPEASDVGSITLA | |
| | LRTLGSFEFEGHSLTQFVRHCADHFLNSEHKEIRMEAARTCSRLL | |
| | TPSIHLISGHAHVVSQTAVQVVADVLSKLLVVGITDPDPDIRYCV | |
| | LASLDERFDAHLAQAENLQALFVALNDQVFEIRELAICTVGRLSS | |
| | MNPAFVMPFLRKMLIQILTELEHSGIGRIKEQSARMLGHLVSNAP | |
| | RLIRPYMEPILKALILKLKDPDPDPNPGVINNVLATIGELAQVSG | |
| | LEMRKWVDELFIIIMDMLQDSSLLAKRQVALWTLGQLVASTGYVV | |
| | EPYRKYPTLLEVLLNFLKTEQNQGTRREAIRVLGLLGALDPYKHK | |
| | VNIGMIDQSRDASAVSLSESKSSQDSSDYSTSEMLVNMGNLPLDE | |
| | FYPAVSMVALMRIFRDQSLSHHHTMVVQAITFIFKSLGLKCVQFL | |
| | PQVMPTFLNVIRVCDGAIREFLFQQLGMLVSFVKSHIRPYMDEIV | |
| | TLMREFWVMNTSIQSTIILLIEQIVVALGGEFKLYLPQLIPHMLR | |
| | VFMHDNSPGRIVSIKLLAAIQLFGANLDDYLHLLLPPIVKLFDAP | |
| | EAPLPSRKAALETVDRLTESLDFTDYASRIIHPIVRTLDQSPELR | |
| | STAMDTLSSLVFQLGKKYQIFIPMVNKVLVRHRINHQRYDVLICR | |
| | IVKGYTLADEEEDPLIYQHRMLRSGQGDALASGPVETGPMKKLHV | |
| | STINLQKAWGAARRVSKDDWLEWLRRLSLELLKDSSSPSLRSCWA | |
| | LAQAYNPMARDLFNAAFVSCWSELNEDQQDELIRSIELALTSQDI | |
| | AEVTQTLLNLAEFMEHSDKGPLPLRDDNGIVLLGERAAKCRAYAK | |
| | ALHYKELEFQKGPTPAILESLISINNKLQQPEAAAGVLEYAMKHF | |
| | GELEIQATWYEKLHEWEDALVAYDKKMDTNKDDPELMLGRMRCLE | |
| | ALGEWGQLHQQCCEKWTLVNDETQAKMARMAAAAAWGLGQWDSME | |
| | EYTCMIPRDTHDGAFYRAVLALHQDLFSLAQQCIDKARDLLDAEL | |
| | TAMAGESYSRAYGAMVSCHMLSELEEVIQYKLVPERRETIRQIWW | |
| | ERLQGCQRIVEDWQKILMVRSLVVSPHEDMRTWLKYASLCGKSGR | |
| | LALAHKTLVLLLGVDPSRQLDHPLPTVHPQVTYAYMKNMWKSARK | |
| | IDAFQHMQHFVQTMQQQAQHAIATEDQQHKQELHKLMARCFLKLG | |
| | EWQLNLQGINESTIPKVLQYYSAATEHDRSWYKAWHAWAVMNFEA | |
| | VLHYKHQNQARDEKKKLRHASGANITNATTAATTAATATTTASTE | |
| | GSNSESEAESTENSPTPSPLQKKVTEDLSKTLLMYTVPAVQGFFR | |
| | SISLSRGNNLQDTLRVLTLWFDYGHWPDVNEALVEGVKAIQIDTW | |
| | LQVIPQLIARIDTPRPLVGRLIHQLLTDIGRYHPQALIYPLTVAS | |
| | KSTTTARHNAANKILKNMCEHSNTLVQQAMMVSEELIRVAILWHE | |
| | MWHEGLEEASRLYFGERNVKGMFEVLEPLHAMMERGPQTLKETSF | |
| | NQAYGRDLMEAQEWCRKYMKSGNVKDLTQAWDLYYHVFRRISKQL | |
| | PQLTSLELQYVSPKLLMCRDLELAVPGTYDPNQPIIRIQSIAPSL | |
| | QVITSKQRPRKLTLMGSNGHEFVFLLKGHEDLRQDERVMQLFGLV | |
| | NTLLANDPTSLRKNLSIQRYAVIPLSTNSGLIGWVPHCDTLHALI | |
| | RDYREKKKILLNIEHRIMLRMAPDYDHLTLMQKVEVFEHAVNNTA | |
| | GDDLAKLLWLKSPSSEVWFDRRTNYTRSLAVMSMVGYILGLGDRH | |
| | PSNLMLDRLSGKILHIDFGDCFEVAMTREKFPEKIPFRLTRMLTN | |
| | AMEVTGLDGNYRITCHTVMEVLREHKDSVMAVLEAFVYDPLLNWR | |
| | LMDTNTKGNKRSRTRTDSYSAGQSVEILDGVELGEPAHKKTGTTV | |
| | PESIHSFIGDGLVKPEALNKKAIQIINRVRDKLTGRDFSHDDTLD | |
| | VPTQVELLIKQATSHENLCQCYIGWCPFW | |
| mTOR encoding | ATGCTTGGAACCGGACCTGCCGCCGCCACCACCGCTGCCACCACA | 62 |
| DNA sequence | TCTAGCAATGTGAGCGTCCTGCAGCAGTTTGCCAGTGGCCTAAAG | |
| (from Genbank | AGCCGGAATGAGGAAACCAGGGCCAAAGCCGCCAAGGAGCTCCAG | |
| NM_005931.4) | CACTATGTCACCATGGAACTCCGAGAGATGAGTCAAGAGGAGTCT | |
| Bold and | ACTCGCTTCTATGACCAACTGAACCATCACATTTTTGAATTGGTT | |
| italicized: | TCCAGCTCAGATGCCAATGAGAGGAAAGGTGGCATCTTGGCCATA | |
| siRNA binding | GCTAGCCTCATAGGAGTGGAAGGTGGGAATGCCACCCGAATTGGC | |
| regions | AGATTTGCCAACTATCTTCGGAACCTCCTCCCCTCCAATGACCCA | |
| | GTTGTCATGGAAATGGCATCCAAGGCCATTGGCCGTCTTGCCATG | |
| | GCAGGGGACACTTTTACCGCTGAGTACGTGGAATTTGAGGTGAAG | |
| | CGAGCCCTGGAATGGCTGGGTGCTGACCGCAATGAGGGCCGGAGA | |
| | CATGCAGCTGTCCTGGTTCTCCGTGAGCTGGCCATCAGCGTCCCT | |
| | ACCTTCTTCTTCCAGCAAGTGCAACCCTTCTTTGACAACATTTTT | |
| | GTGGCCGTGTGGGACCCCAAACAGGCCATCCGTGAGGGAGCTGTA | |
| | GCCGCCCTTCGTGCCTGTCTGATTCTCACAACCCAGCGTGAGCCG | |
| | AAGGAGATGCAGAAGCCTCAGTGGTACAGGCACACATTTGAAGAA | |
| | GCAGAGAAGGGATTTGATGAGACCTTGGCCAAAGAGAAGGGCATG | |
| | AATCGGGATGATCGGATCCATGGAGCCTTGTTGATCCTTAACGAG | |
| | CTGGTCCGAATCAGCAGCATGGAGGGAGAGCGTCTGAGAGAAGAA | |
| | ATGGAAGAAATCACACAGCAGCAGCTGGTACACGACAAGTACTGC | |
| | AAAGATCTCATGGGCTTCGGAACAAAACCTCGTCACATTACCCCC | |
| | TTCACCAGTTTCCAGGCTGTACAGCCCCAGCAGTCAAATGCCTTG | |
| | GTGGGGCTGCTGGGGTACAGCTCTCACCAAGGCCTCATGGGATTT | |
| | GGGACCTCCCCCAGTCCAGCTAAGTCCACCCTGGTGGAGAGCCGG | |
| | TGTTGCAGAGACTTGATGGAGGAGAAATTTGATCAGGTGTGCCAG | |
| | TGGGTGCTGAAATGCAGGAATAGCAAGAACTCGCTGATCCAAATG | |
| | ACAATCCTTAATTTGTTGCCCCGCTTGGCTGCATTCCGACCTTCT | |
| | GCCTTCACAGATACCCAGTATCTCCAAGATACCATGAACCATGTC | |
| | CTAAGCTGTGTCAAGAAGGAGAAGGAACGTACAGCGGCCTTCCAA | |
| | GCCCTGGGGCTACTTTCTGTGGCTGTGAGGTCTGAGTTTAAGGTC | |
| | TATTTGCCTCGCGTGCTGGACATCATCCGAGCGGCCCTGCCCCCA | |
| | AAGGACTTCGCCCATAAGAGGCAGAAGGCAATGCAGGTGGATGCC | |
| | ACAGTCTTCACTTGCATCAGCATGCTGGCTCGAGCAATGGGGCCA | |
| | GGCATCCAGCAGGATATCAAGGAGCTGCTGGAGCCCATGCTGGCA | |
| | GTGGGACTAAGCCCTGCCCTCACTGCAGTGCTCTACGACCTGAGC | |
| | CGTCAGATTCCACAGCTAAAGAAGGACATTCAAGATGGGCTACTG | |
| | AAAATGCTGTCCCTGGTCCTTATGCACAAACCCCTTCGCCACCCA | |
| | GGCATGCCCAAGGGCCTGGCCCATCAGCTGGCCTCTCCTGGCCTC | |
| | ACGACCCTCCCTGAGGCCAGCGATGTGGGCAGCATCACTCTTGCC | |
| | CTCCGAACGCTTGGCAGCTTTGAATTTGAAGGCCACTCTCTGACC | |
| | CAATTTGTTCGCCACTGTGCGGATCATTTCCTGAACAGTGAGCAC | |
| | AAGGAGATCCGCATGGAGGCTGCCCGCACCTGCTCCCGCCTGCTC | |
| | ACACCCTCCATCCACCTCATCAGTGGCCATGCTCATGTGGTTAGC | |
| | CAGACCGCAGTGCAAGTGGTGGCAGATGTGCTTAGCAAACTGCTC | |
| | GTAGTTGGGATAACAGATCCT [symbol] [symbol] GTC | |
| | TTGGCGTCCCTGGACGAGCGCTTTGATGCACACCTGGCCCAGGCG | |
| | GAGAACTTGCAGGCCTTGTTTGTGGCTCTGAATGACCAGGTGTTT | |
| | GAGATCCGGGAGCTGGCCATCTGCACTGTGGGCCGACTCAGTAGC | |
| | ATGAACCCTGCCTTTGTCATGCCTTTCCTGCGCAAGATGCTCATC | |
| | CAGATTTTGACAGAGTTGGAGCACAGTGGGATTGGAAGAATCAAA | |
| | GAGCAGAGTGCCCGCATGCTGGGGCACCTGGTCTCCAATGCCCCC | |
| | CGACTCATCCGCCCCTACATGGAGCCTATTCTGAAGGCATTAATT | |
| | TTGAAACTGAAAGATCCAGACCCTGATCCAAACCCAGGTGTGATC | |
| | AATAATGTCCTGGCAACAATAGGAGAATTGGCACAGGTTAGTGGC | |
| | CTGGAAATGAGGAAATGGGTTGATGAACTTTTTATTATCATCATG | |
| | GACATGCTCCAGGATTCCTCTTTGTTGGCCAAAAGGCAGGTGGCT | |
| | CTGTGGACCCTGGGACAGTTGGTGGCCAGCACTGGCTATGTAGTA | |
| | GAGCCCTACAGGAAGTACCCTACTTTGCTTGAGGTGCTACTGAAT | |
| | TTTCTGAAGACTGAGCAGAACCAGGGTACACGCAGAGAGGCCATC | |
| | CGTGTGTTAGGGCTTTTAGGGGCTTTGGATCCTTACAAGCACAAA | |
| | GTGAACATTGGCATGATAGACCAGTCCCGGGATGCCTCTGCTGTC | |
| | AGCCTGTCAGAATCCAAGTCAAGTCAGGATTCCTCTGACTATAGC | |
| | ACTAGTGAAATGCTGGTCAACATGGGAAACTTGCCTCTGGATGAG | |
| | TTCTACCCAGCTGTGTCCATGGTGGCCCTGATGCGGATCTTCCGA | |
| | GACCAGTCACTCTCTCATCATCACACCATGGTTGTCCAGGCCATC | |
| | ACCTTCATCTTCAAGTCCCTGGGACTCAAATGTGTGCAGTTCCTG | |
| | CCCCAGGTCATGCCCACGTTCCTTAACGTCATTCGAGTCTGTGAT | |
| | GGGGCCATCCGGGAATTTTTGTTCCAGCAGCTGGGAATGTTGGTG | |
| | TCCTTTGTGAAGAGCCACATCAGACCTTATATGGATGAAATAGTC | |
| | ACCCTCATGAGAGAATTCTGGGTCATGAACACCTCAATTCAGAGC | |
| | ACGATCATTCTTCTCATTGAGCAAATTGTGGTAGCTCTTGGGGGT | |
| | GAATTTAAGCTCTACCTGCCCCAGCTGATCCCACACATGCTGCGT | |
| | GTCTTCATGCATGACAACAGCCCAGGCCGCATTGTCTCTATCAAG | |
| | TTACTGGCTGCAATCCAGCTGTTTGGCGCCAACCTGGATGACTAC | |
| | CTGCATTTACTGCTGCCTCCTATTGTTAAGTTGTTTGATGCCCCT | |
| | GAAGCTCCACTGCCATCTCGAAAGGCAGCGCTAGAGACTGTGGAC | |
| | CGCCTGACGGAGTCCCTGGATTTCACTGACTATGCCTCCCGGATC | |
| | ATTCACCCTATTGTTCGAACACTGGACCAGAGCCCAGAACTGCGC | |
| | TCCACAGCCATGGACACGCTGTCTTCACTTGTTTTTCAGCTGGGG | |
| | AAGAAGTACCAAATTTTCATTCCAATGGTGAATAAAGTTCTGGTG | |
| | CGACACCGAATCAATCATCAGCGCTATGATGTGCTCATCTGCAGA | |
| | ATTGTCAAGGGATACACACTTGCTGATGAAGAGGAGGATCCTTTG | |
| | ATTTACCAGCATCGGATGCTTAGGAGTGGCCAAGGGGATGCATTG | |
| | GCTAGTGGACCAGTGGAAACAGGACCCATGAAGAAACTGCACGTC | |
| | AGCACCATCAACCTCCAAAAGGCCTGGGGCGCTGCCAGGAGGGTC | |
| | TCCAAAGATGACTGGCTGGAATGGCTGAGACGGCTGAGCCTG [symbol] | |
| | [symbol] [symbol] TCGCCCTCCCTGCGCTCCTGCTGGGCC | |
| | CTGGCACAGGCCTACAACCCGATGGCCAGGGATCTCTTCAATGCT | |
| | GCATTTGTGTCCTGCTGGTCTGAACTGAATGAAGATCAACAGGAT | |
| | GAGCTCATCAGAAGCATCGAGTTGGCCCTCACCTCACAAGACATC | |
| | GCTGAAGTCACACAGACCCTCTTAAACTTGGCTGAATTCATGGAA | |
| | CACAGTGACAAGGGCCCCCTGCCACTGAGAGATGACAATGGCATT | |
| | GTTCTGCTGGGTGAGAGAGCTGCCAAGTGCCGAGCATATGCCAAA | |
| | GCACTACACTACAAAGAACTGGAGTTCCAGAAAGGCCCCACCCCT | |
| | GCCATTCTAGAATCTCTCATCAGCATTAATAATAAGCTACAGCAG | |
| | CCGGAGGCAGCGGCCGGAGTGTTAGAATATGCCATGAAACACTTT | |
| | GGAGAGCTGGAGATCCAGGCTACCTGGTATGAGAAACTGCACGAG | |
| | TGGGAGGATGCCCTTGTGGCCTATGACAAGAAAATGGACACCAAC | |
| | AAGGACGACCCAGAGCTGATGCTGGGCCGCATGCGCTGCCTCGAG | |
| | GCCTTGGGGGAATGGGGTCAACTCCACCAGCAGTGCTGTGAAAAG | |
| | TGGACCCTGGTTAATGATGAGACCCAAGCCAAGATGGCCCGGATG | |
| | GCTGCTGCAGCTGCATGGGGTTTAGGTCAGTGGGACAGCATGGAA | |
| | GAATACACCTGTATGATCCCTCGGGACACCCATGATGGGGCATTT | |
| | TATAGAGCTGTGCTGGCACTGCATCAGGACCTCTTCTCCTTGGCA | |
| | CAACAGTGCATTGACAAGGCCAGGGACCTGCTGGATGCTGAATTA | |
| | ACTGCGATGGCAGGAGAGAGTTACAGTCGGGCATATGGGGCCATG | |
| | GTTTCTTGCCACATGCTGTCCGAGCTGGAGGAGGTTATCCAGTAC | |
| | AAACTTGTCCCCGAGCGACGAGAGATCATCCGCCAGATCTGGTGG | |
| | GAGAGACTGCAGGGCTGCCAGCGTATCGTAGAGGACTGGCAGAAA | |
| | ATCCTTATGGTGCGGTCCCTTGTGGTCAGCCCTCATGAAGACATG | |
| | AGAACCTGGCTCAAGTATGCAAGCCTGTGCGGCAAGAGTGGCAGG | |
| | CTGGCTCTTGCTCATAAAACTTTAGTGTTGCTCCTGGGAGTTGAT | |
| | CCGTCTCGGCAACTTGACCATCCTCTGCCAACAGTTCACCCTCAG | |
| | GTGACCTATGCCTACATGAAAAACATGTGGAAGAGTGCCCGCAAG | |
| | ATCGATGCCTTCCAGCACATGCAGCATTTTGTCCAGACCATGCAG | |
| | CAACAGGCCCAGCATGCCATCGCTACTGAGGACCAGCAGCATAAG | |
| | CAGGAACTGCACAAGCTCATGGCCCGATGCTTCCTGAAACTTGGA | |
| | GAGTGGCAGCTGAATCTACAGGGCATCAATGAGAGCACAATCCCC | |
| | AAAGTGCTGCAGTACTACAGCGCCGCCACAGAGCACGACCGCAGC | |
| | TGGTACAAGGCCTGGCATGCGTGGGCAGTGATGAACTTCGAAGCT | |
| | GTGCTACACTACAAACATCAGAACCAAGCCCGCGATGAGAAGAAG | |
| | AAACTGCGTCATGCCAGCGGGGCCAACATCACCAACGCCACCACT | |
| | GCCGCCACCACGGCCGCCACTGCCACCACCACTGCCAGCACCGAG | |
| | GGCAGCAACAGTGAGAGCGAGGCCGAGAGCACCGAGAACAGCCCC | |
| | ACCCCATCGCCGCTGCAGAAGAAGGTCACTGAGGATCTGTCCAAA | |
| | ACCCTCCTGATGTACACGGTGCCTGCCGTCCAGGGCTTCTTCCGT | |
| | TCCATCTCCTTGTCACGAGGCAACAACCTCCAGGATACACTCAGA | |
| | GTTCTCACCTTATGGTTTGATTATGGTCACTGGCCAGATGTCAAT | |
| | GAGGCCTTAGTGGAGGGGGTGAAAGCCATCCAGATTGATACCTGG | |
| | CTACAGGTTATACCTCAGCTCATTGCAAGAATTGATACGCCCAGA | |
| | CCCTTGGTGGGACGTCTCATTCACCAGCTTCTCACAGACATTGGT | |
| | CGGTACCACCCCCAGGCCCTCATCTACCCACTGACAGTGGCTTCT | |
| | AAGTCTACCACGACAGCCCGGCACAATGCAGCCAACAAGATTCTG | |
| | AAGAACATGTGTGAGCACAGCAACACCCTGGTCCAGCAGGCCATG | |
| | ATGGTGAGCGAGGAGCTGATCCGAGTGGCCATCCTCTGGCATGAG | |
| | ATGTGGCATGAAGGCCTGGAAGAGGCATCTCGTTTGTACTTTGGG | |
| | GAAAGGAACGTGAAAGGCATGTTTGAGGTGCTGGAGCCCTTGCAT | |
| | GCTATGATGGAACGGGGCCCCCAGACTCTGAAGGAAACATCCTTT | |
| | AATCAGGCCTATGGTCGAGATTTAATGGAGGCCCAAGAGTGGTGC | |
| | AGGAAGTACATGAAATCAGGGAATGTCAAGGACCTCACCCAAGCC | |
| | TGGGACCTCTATTATCATGTGTTCCGACGAATCTCAAAGCAGCTG | |
| | CCTCAGCTCACATCCTTAGAGCTGCAATATGTTTCCCCAAAACTT | |
| | CTGATGTGCCGGGACCTTGAATTGGCTGTGCCAGGAACATATGAC | |
| | CCCAACCAGCCAATCATTCGCATTCAGTCCATAGCACCGTCTTTG | |
| | CAAGTCATCACATCCAAGCAGAGGCCCCGGAAATTGACACTTATG | |
| | GGCAGCAACGGACATGAGTTTGTTTTCCTTCTAAAAGGCCATGAA | |
| | GATCTGCGCCAGGATGAGCGTGTGATGCAGCTCTTCGGCCTGGTT | |
| | AACACCCTTCT [symbol] [symbol] TCGGAAAAACCTC | |
| | AGCATCCAGAGATACGCTGTCATCCCTTTATCGACCAACTCGGGC | |
| | CTCATTGGCTGGGTTCCCCACTGTGACACACTGCACGCCCTCATC | |
| | CGGGACTACAGGGAGAAGAAGAAGATCCTTCTCAACATCGAGCAT | |
| | CGCATCATGTTGCGGATGGCTCCGGACTATGACCACTTGACTCTG | |
| | ATGCAGAAGGTGGAGGTGTTTGAGCATGCCGTCAATAATACAGCT | |
| | GGGGACGACCTGGCCAAGCTGCTGTGGCTGAAAAGCCCCAGCTCC | |
| | GAGGTGTGGTTTGACCGAAGAACCAATTATACCCGTTCTTTAGCG | |
| | GTCATGTCAATGGTTGGGTATATTTTAGGCCTGGGAGATAGACAC | |
| | CCATCCAACCTGATGCTGGACCGTCTGAGTGGGAAGATCCTGCAC | |
| | ATTGACTTTGGGGACTGCTTTGAGGTTGCTATGACCCGAGAGAAG | |
| | TTTCCAGAGAAGATTCCATTTAGACTAACAAGAATGTTGACCAAT | |
| | GCTATGGAGGTTACAGGCCTGGATGGCAACTACAGAATCACATGC | |
| | CACACAGTGATGGAGGTGCTGCGAGAGCACAAGGACAGTGTCATG | |
| | GCCGTGCTGGAAGCCTTTGTCTATGACCCCTTGCTGAACTGGAGG | |
| | CTGATGGACACAAATACCAAAGGCAACAAGCGATCCCGAACGAGG | |
| | ACGGATTCCTACTCTGCTGGCCAGTCAGTCGAAATTTTGGACGGT | |
| | GTGGAACTTGGAGAGCCAGCCCATAAGAAAACGGGGACCACAGTG | |
| | CCAGAATCTATTCATTCTTTCATTGGAGACGGTTTGGTGAAACCA | |
| | GAGGCCCTAAATAAGAAAGCTATCCAGATTATTAACAGGGTTCGA | |
| | GATAAGCTCACTGGTCGGGACTTCTCTCATGATGACACTTTGGAT | |
| | GTTCCAACGCAAGTTGAGCTGCTCATCAAACAAGCGACATCCCAT | |
| | GAAAACCTCTGCCAGTGCTATATTGGCTGGTGCCCTTTCTGGTAA | |
| mTOR encoding | AUGCUUGGAACCGGACCUGCCGCCGCCACCACCGCUGCCACCACA | 63 |
| RNA sequence | UCUAGCAAUGUGAGCGUCCUGCAGCAGUUUGCCAGUGGCCUAAAG | |
| (from Genbank | AGCCGGAAUGAGGAAACCAGGGCCAAAGCCGCCAAGGAGCUCCAG | |
| NM_005931.4) | CACUAUGUCACCAUGGAACUCCGAGAGAUGAGUCAAGAGGAGUCU | |
| Bold and | ACUCGCUUCUAUGACCAACUGAACCAUCACAUUUUUGAAUUGGUU | |
| italicized: | UCCAGCUCAGAUGCCAAUGAGAGGAAAGGUGGCAUCUUGGCCAUA | |
| siRNA binding | GCUAGCCUCAUAGGAGUGGAAGGUGGGAAUGCCACCCGAAUUGGC | |
| regions | AGAUUUGCCAACUAUCUUCGGAACCUCCUCCCCUCCAAUGACCCA | |
| | GUUGUCAUGGAAAUGGCAUCCAAGGCCAUUGGCCGUCUUGCCAUG | |
| | GCAGGGGACACUUUUACCGCUGAGUACGUGGAAUUUGAGGUGAAG | |
| | CGAGCCCUGGAAUGGCUGGGUGCUGACCGCAAUGAGGGCCGGAGA | |
| | CAUGCAGCUGUCCUGGUUCUCCGUGAGCUGGCCAUCAGCGUCCCU | |
| | ACCUUCUUCUUCCAGCAAGUGCAACCCUUCUUUGACAACAUUUUU | |
| | GUGGCCGUGUGGGACCCCAAACAGGCCAUCCGUGAGGGAGCUGUA | |
| | GCCGCCCUUCGUGCCUGUCUGAUUCUCACAACCCAGCGUGAGCCG | |
| | AAGGAGAUGCAGAAGCCUCAGUGGUACAGGCACACAUUUGAAGAA | |
| | GCAGAGAAGGGAUUUGAUGAGACCUUGGCCAAAGAGAAGGGCAUG | |
| | AAUCGGGAUGAUCGGAUCCAUGGAGCCUUGUUGAUCCUUAACGAG | |
| | CUGGUCCGAAUCAGCAGCAUGGAGGGAGAGCGUCUGAGAGAAGAA | |
| | AUGGAAGAAAUCACACAGCAGCAGCUGGUACACGACAAGUACUGC | |
| | AAAGAUCUCAUGGGCUUCGGAACAAAACCUCGUCACAUUACCCCC | |
| | UUCACCAGUUUCCAGGCUGUACAGCCCCAGCAGUCAAAUGCCUUG | |
| | GUGGGGCUGCUGGGGUACAGCUCUCACCAAGGCCUCAUGGGAUUU | |
| | GGGACCUCCCCCAGUCCAGCUAAGUCCACCCUGGUGGAGAGCCGG | |
| | UGUUGCAGAGACUUGAUGGAGGAGAAAUUUGAUCAGGUGUGCCAG | |
| | UGGGUGCUGAAAUGCAGGAAUAGCAAGAACUCGCUGAUCCAAAUG | |
| | ACAAUCCUUAAUUUGUUGCCCCGCUUGGCUGCAUUCCGACCUUCU | |
| | GCCUUCACAGAUACCCAGUAUCUCCAAGAUACCAUGAACCAUGUC | |
| | CUAAGCUGUGUCAAGAAGGAGAAGGAACGUACAGCGGCCUUCCAA | |
| | GCCCUGGGGCUACUUUCUGUGGCUGUGAGGUCUGAGUUUAAGGUC | |
| | UAUUUGCCUCGCGUGCUGGACAUCAUCCGAGCGGCCCUGCCCCCA | |
| | AAGGACUUCGCCCAUAAGAGGCAGAAGGCAAUGCAGGUGGAUGCC | |
| | ACAGUCUUCACUUGCAUCAGCAUGCUGGCUCGAGCAAUGGGGCCA | |
| | GGCAUCCAGCAGGAUAUCAAGGAGCUGCUGGAGCCCAUGCUGGCA | |
| | GUGGGACUAAGCCCUGCCCUCACUGCAGUGCUCUACGACCUGAGC | |
| | CGUCAGAUUCCACAGCUAAAGAAGGACAUUCAAGAUGGGCUACUG | |
| | AAAAUGCUGUCCCUGGUCCUUAUGCACAAACCCCUUCGCCACCCA | |
| | GGCAUGCCCAAGGGCCUGGCCCAUCAGCUGGCCUCUCCUGGCCUC | |
| | ACGACCCUCCCUGAGGCCAGCGAUGUGGGCAGCAUCACUCUUGCC | |
| | CUCCGAACGCUUGGCAGCUUUGAAUUUGAAGGCCACUCUCUGACC | |
| | CAAUUUGUUCGCCACUGUGCGGAUCAUUUCCUGAACAGUGAGCAC | |
| | AAGGAGAUCCGCAUGGAGGCUGCCCGCACCUGCUCCCGCCUGCUC | |
| | ACACCCUCCAUCCACCUCAUCAGUGGCCAUGCUCAUGUGGUUAGC | |
| | CAGACCGCAGUGCAAGUGGUGGCAGAUGUGCUUAGCAAACUGCUC | |
| | GUAGUUGGGAUAACAGAUCCU [symbol] [symbol] GUC | |
| | UUGGCGUCCCUGGACGAGCGCUUUGAUGCACACCUGGCCCAGGCG | |
| | GAGAACUUGCAGGCCUUGUUUGUGGCUCUGAAUGACCAGGUGUUU | |
| | GAGAUCCGGGAGCUGGCCAUCUGCACUGUGGGCCGACUCAGUAGC | |
| | AUGAACCCUGCCUUUGUCAUGCCUUUCCUGCGCAAGAUGCUCAUC | |
| | CAGAUUUUGACAGAGUUGGAGCACAGUGGGAUUGGAAGAAUCAAA | |
| | GAGCAGAGUGCCCGCAUGCUGGGGCACCUGGUCUCCAAUGCCCCC | |
| | CGACUCAUCCGCCCCUACAUGGAGCCUAUUCUGAAGGCAUUAAUU | |
| | UUGAAACUGAAAGAUCCAGACCCUGAUCCAAACCCAGGUGUGAUC | |
| | AAUAAUGUCCUGGCAACAAUAGGAGAAUUGGCACAGGUUAGUGGC | |
| | CUGGAAAUGAGGAAAUGGGUUGAUGAACUUUUUAUUAUCAUCAUG | |
| | GACAUGCUCCAGGAUUCCUCUUUGUUGGCCAAAAGGCAGGUGGCU | |
| | CUGUGGACCCUGGGACAGUUGGUGGCCAGCACUGGCUAUGUAGUA | |
| | GAGCCCUACAGGAAGUACCCUACUUUGCUUGAGGUGCUACUGAAU | |
| | UUUCUGAAGACUGAGCAGAACCAGGGUACACGCAGAGAGGCCAUC | |
| | CGUGUGUUAGGGCUUUUAGGGGCUUUGGAUCCUUACAAGCACAAA | |
| | GUGAACAUUGGCAUGAUAGACCAGUCCCGGGAUGCCUCUGCUGUC | |
| | AGCCUGUCAGAAUCCAAGUCAAGUCAGGAUUCCUCUGACUAUAGC | |
| | ACUAGUGAAAUGCUGGUCAACAUGGGAAACUUGCCUCUGGAUGAG | |
| | UUCUACCCAGCUGUGUCCAUGGUGGCCCUGAUGCGGAUCUUCCGA | |
| | GACCAGUCACUCUCUCAUCAUCACACCAUGGUUGUCCAGGCCAUC | |
| | ACCUUCAUCUUCAAGUCCCUGGGACUCAAAUGUGUGCAGUUCCUG | |
| | CCCCAGGUCAUGCCCACGUUCCUUAACGUCAUUCGAGUCUGUGAU | |
| | GGGGCCAUCCGGGAAUUUUUGUUCCAGCAGCUGGGAAUGUUGGUG | |
| | UCCUUUGUGAAGAGCCACAUCAGACCUUAUAUGGAUGAAAUAGUC | |
| | ACCCUCAUGAGAGAAUUCUGGGUCAUGAACACCUCAAUUCAGAGC | |
| | ACGAUCAUUCUUCUCAUUGAGCAAAUUGUGGUAGCUCUUGGGGGU | |
| | GAAUUUAAGCUCUACCUGCCCCAGCUGAUCCCACACAUGCUGCGU | |
| | GUCUUCAUGCAUGACAACAGCCCAGGCCGCAUUGUCUCUAUCAAG | |
| | UUACUGGCUGCAAUCCAGCUGUUUGGCGCCAACCUGGAUGACUAC | |
| | CUGCAUUUACUGCUGCCUCCUAUUGUUAAGUUGUUUGAUGCCCCU | |
| | GAAGCUCCACUGCCAUCUCGAAAGGCAGCGCUAGAGACUGUGGAC | |
| | CGCCUGACGGAGUCCCUGGAUUUCACUGACUAUGCCUCCCGGAUC | |
| | AUUCACCCUAUUGUUCGAACACUGGACCAGAGCCCAGAACUGCGC | |
| | UCCACAGCCAUGGACACGCUGUCUUCACUUGUUUUUCAGCUGGGG | |
| | AAGAAGUACCAAAUUUUCAUUCCAAUGGUGAAUAAAGUUCUGGUG | |
| | CGACACCGAAUCAAUCAUCAGCGCUAUGAUGUGCUCAUCUGCAGA | |
| | AUUGUCAAGGGAUACACACUUGCUGAUGAAGAGGAGGAUCCUUUG | |
| | AUUUACCAGCAUCGGAUGCUUAGGAGUGGCCAAGGGGAUGCAUUG | |
| | GCUAGUGGACCAGUGGAAACAGGACCCAUGAAGAAACUGCACGUC | |
| | AGCACCAUCAACCUCCAAAAGGCCUGGGGCGCUGCCAGGAGGGUC | |
| | UCCAAAGAUGACUGGCUGGAAUGGCUGAGACGGCUGAGCCUG [symbol] | |
| | [symbol] [symbol] UCGCCCUCCCUGCGCUCCUGCUGGGCC | |
| | CUGGCACAGGCCUACAACCCGAUGGCCAGGGAUCUCUUCAAUGCU | |
| | GCAUUUGUGUCCUGCUGGUCUGAACUGAAUGAAGAUCAACAGGAU | |
| | GAGCUCAUCAGAAGCAUCGAGUUGGCCCUCACCUCACAAGACAUC | |
| | GCUGAAGUCACACAGACCCUCUUAAACUUGGCUGAAUUCAUGGAA | |
| | CACAGUGACAAGGGCCCCCUGCCACUGAGAGAUGACAAUGGCAUU | |
| | GUUCUGCUGGGUGAGAGAGCUGCCAAGUGCCGAGCAUAUGCCAAA | |
| | GCACUACACUACAAAGAACUGGAGUUCCAGAAAGGCCCCACCCCU | |
| | GCCAUUCUAGAAUCUCUCAUCAGCAUUAAUAAUAAGCUACAGCAG | |
| | CCGGAGGCAGCGGCCGGAGUGUUAGAAUAUGCCAUGAAACACUUU | |
| | GGAGAGCUGGAGAUCCAGGCUACCUGGUAUGAGAAACUGCACGAG | |
| | UGGGAGGAUGCCCUUGUGGCCUAUGACAAGAAAAUGGACACCAAC | |
| | AAGGACGACCCAGAGCUGAUGCUGGGCCGCAUGCGCUGCCUCGAG | |
| | GCCUUGGGGGAAUGGGGUCAACUCCACCAGCAGUGCUGUGAAAAG | |
| | UGGACCCUGGUUAAUGAUGAGACCCAAGCCAAGAUGGCCCGGAUG | |
| | GCUGCUGCAGCUGCAUGGGGUUUAGGUCAGUGGGACAGCAUGGAA | |
| | GAAUACACCUGUAUGAUCCCUCGGGACACCCAUGAUGGGGCAUUU | |
| | UAUAGAGCUGUGCUGGCACUGCAUCAGGACCUCUUCUCCUUGGCA | |
| | CAACAGUGCAUUGACAAGGCCAGGGACCUGCUGGAUGCUGAAUUA | |
| | ACUGCGAUGGCAGGAGAGAGUUACAGUCGGGCAUAUGGGGCCAUG | |
| | GUUUCUUGCCACAUGCUGUCCGAGCUGGAGGAGGUUAUCCAGUAC | |
| | AAACUUGUCCCCGAGCGACGAGAGAUCAUCCGCCAGAUCUGGUGG | |
| | GAGAGACUGCAGGGCUGCCAGCGUAUCGUAGAGGACUGGCAGAAA | |
| | AUCCUUAUGGUGCGGUCCCUUGUGGUCAGCCCUCAUGAAGACAUG | |
| | AGAACCUGGCUCAAGUAUGCAAGCCUGUGCGGCAAGAGUGGCAGG | |
| | CUGGCUCUUGCUCAUAAAACUUUAGUGUUGCUCCUGGGAGUUGAU | |
| | CCGUCUCGGCAACUUGACCAUCCUCUGCCAACAGUUCACCCUCAG | |
| | GUGACCUAUGCCUACAUGAAAAACAUGUGGAAGAGUGCCCGCAAG | |
| | AUCGAUGCCUUCCAGCACAUGCAGCAUUUUGUCCAGACCAUGCAG | |
| | CAACAGGCCCAGCAUGCCAUCGCUACUGAGGACCAGCAGCAUAAG | |
| | CAGGAACUGCACAAGCUCAUGGCCCGAUGCUUCCUGAAACUUGGA | |
| | GAGUGGCAGCUGAAUCUACAGGGCAUCAAUGAGAGCACAAUCCCC | |
| | AAAGUGCUGCAGUACUACAGCGCCGCCACAGAGCACGACCGCAGC | |
| | UGGUACAAGGCCUGGCAUGCGUGGGCAGUGAUGAACUUCGAAGCU | |
| | GUGCUACACUACAAACAUCAGAACCAAGCCCGCGAUGAGAAGAAG | |
| | AAACUGCGUCAUGCCAGCGGGGCCAACAUCACCAACGCCACCACU | |
| | GCCGCCACCACGGCCGCCACUGCCACCACCACUGCCAGCACCGAG | |
| | GGCAGCAACAGUGAGAGCGAGGCCGAGAGCACCGAGAACAGCCCC | |
| | ACCCCAUCGCCGCUGCAGAAGAAGGUCACUGAGGAUCUGUCCAAA | |
| | ACCCUCCUGAUGUACACGGUGCCUGCCGUCCAGGGCUUCUUCCGU | |
| | UCCAUCUCCUUGUCACGAGGCAACAACCUCCAGGAUACACUCAGA | |
| | GUUCUCACCUUAUGGUUUGAUUAUGGUCACUGGCCAGAUGUCAAU | |
| | GAGGCCUUAGUGGAGGGGGUGAAAGCCAUCCAGAUUGAUACCUGG | |
| | CUACAGGUUAUACCUCAGCUCAUUGCAAGAAUUGAUACGCCCAGA | |
| | CCCUUGGUGGGACGUCUCAUUCACCAGCUUCUCACAGACAUUGGU | |
| | CGGUACCACCCCCAGGCCCUCAUCUACCCACUGACAGUGGCUUCU | |
| | AAGUCUACCACGACAGCCCGGCACAAUGCAGCCAACAAGAUUCUG | |
| | AAGAACAUGUGUGAGCACAGCAACACCCUGGUCCAGCAGGCCAUG | |
| | AUGGUGAGCGAGGAGCUGAUCCGAGUGGCCAUCCUCUGGCAUGAG | |
| | AUGUGGCAUGAAGGCCUGGAAGAGGCAUCUCGUUUGUACUUUGGG | |
| | GAAAGGAACGUGAAAGGCAUGUUUGAGGUGCUGGAGCCCUUGCAU | |
| | GCUAUGAUGGAACGGGGCCCCCAGACUCUGAAGGAAACAUCCUUU | |
| | AAUCAGGCCUAUGGUCGAGAUUUAAUGGAGGCCCAAGAGUGGUGC | |
| | AGGAAGUACAUGAAAUCAGGGAAUGUCAAGGACCUCACCCAAGCC | |
| | UGGGACCUCUAUUAUCAUGUGUUCCGACGAAUCUCAAAGCAGCUG | |
| | CCUCAGCUCACAUCCUUAGAGCUGCAAUAUGUUUCCCCAAAACUU | |
| | CUGAUGUGCCGGGACCUUGAAUUGGCUGUGCCAGGAACAUAUGAC | |
| | CCCAACCAGCCAAUCAUUCGCAUUCAGUCCAUAGCACCGUCUUUG | |
| | CAAGUCAUCACAUCCAAGCAGAGGCCCCGGAAAUUGACACUUAUG | |
| | GGCAGCAACGGACAUGAGUUUGUUUUCCUUCUAAAAGGCCAUGAA | |
| | GAUCUGCGCCAGGAUGAGCGUGUGAUGCAGCUCUUCGGCCUGGUU | |
| | AACACCCUUCU [symbol] [symbol] UCGGAAAAACCUC | |
| | AGCAUCCAGAGAUACGCUGUCAUCCCUUUAUCGACCAACUCGGGC | |
| | CUCAUUGGCUGGGUUCCCCACUGUGACACACUGCACGCCCUCAUC | |
| | CGGGACUACAGGGAGAAGAAGAAGAUCCUUCUCAACAUCGAGCAU | |
| | CGCAUCAUGUUGCGGAUGGCUCCGGACUAUGACCACUUGACUCUG | |
| | AUGCAGAAGGUGGAGGUGUUUGAGCAUGCCGUCAAUAAUACAGCU | |
| | GGGGACGACCUGGCCAAGCUGCUGUGGCUGAAAAGCCCCAGCUCC | |
| | GAGGUGUGGUUUGACCGAAGAACCAAUUAUACCCGUUCUUUAGCG | |
| | GUCAUGUCAAUGGUUGGGUAUAUUUUAGGCCUGGGAGAUAGACAC | |
| | CCAUCCAACCUGAUGCUGGACCGUCUGAGUGGGAAGAUCCUGCAC | |
| | AUUGACUUUGGGGACUGCUUUGAGGUUGCUAUGACCCGAGAGAAG | |
| | UUUCCAGAGAAGAUUCCAUUUAGACUAACAAGAAUGUUGACCAAU | |
| | GCUAUGGAGGUUACAGGCCUGGAUGGCAACUACAGAAUCACAUGC | |
| | CACACAGUGAUGGAGGUGCUGCGAGAGCACAAGGACAGUGUCAUG | |
| | GCCGUGCUGGAAGCCUUUGUCUAUGACCCCUUGCUGAACUGGAGG | |
| | CUGAUGGACACAAAUACCAAAGGCAACAAGCGAUCCCGAACGAGG | |
| | ACGGAUUCCUACUCUGCUGGCCAGUCAGUCGAAAUUUUGGACGGU | |
| | GUGGAACUUGGAGAGCCAGCCCAUAAGAAAACGGGGACCACAGUG | |
| | CCAGAAUCUAUUCAUUCUUUCAUUGGAGACGGUUUGGUGAAACCA | |
| | GAGGCCCUAAAUAAGAAAGCUAUCCAGAUUAUUAACAGGGUUCGA | |
| | GAUAAGCUCACUGGUCGGGACUUCUCUCAUGAUGACACUUUGGAU | |
| | GUUCCAACGCAAGUUGAGCUGCUCAUCAAACAAGCGACAUCCCAU | |
| | GAAAACCUCUGCCAGUGCUAUAUUGGCUGGUGCCCUUUCUGGUAA | |
| KRAS amino | MTEYKLVVVGAGGVGKSALTIQLIQNHFVDEYDPTIEDSYRKQVV | 64 |
| acid (Genbank | IDGETCLLDILDTAGQEEYSAMRDQYMRTGEGFLCVFAINNTKSF | |
| NM_004985.4) | EDIHHYREQIKRVKDSEDVPMVLVGNKCDLPSRTVDTKQAQDLAR | |
| (Transcript | SYGIPFIETSAKTRQGVDDAFYTLVREIRKHKEKMSKDGKKKKKK | |
| variant b) | SKTKCVIM | |
| KRAS encoding | ATGACTGAATATAAACTTGTGGTAGTTGGAGCTGGTGGCGTAGGC | 65 |
| DNA sequence | AAGAGTGCCTTGACGATACAGCTAATTCAGAATCATTTTGTGGAC | |
| (from Genbank | GAATATGATCCAACAATAGAGGATTCCTACAGGAAGCAAGTAGTA | |
| NM_004985.4) | ATTGATGGAGAAACCTGTCTCTTGGATATTCTCGACACAGCAGGT | |
| Bold and | CAAGAGGAGTACA [symbol] [symbol] TGAGGACTGGG | |
| italicized: | GAGGGCTTTCTTTGTGTATTTGCCATAAATAATACTAAATCATTT | |
| siRNA binding | GAAGATATTCACCATTATAGAGAACAAATTAAAAGAGTTAAGGAC | |
| regions | TCTGAAGATGTACCTATGGTCCTAGTAGGAAATAAATGTGATTTG | |
| | CCTTCTAGAACAGTAGACACAAAACAGGCTCAGGACTTAGCAAGA | |
| | AGTTATGGAATTCCTTTTATTGAAACATCAGCAAAGACAAGACAG | |
| | GGTGTTGATGATGCCTTCTATACATTAGTTCGAGAAATTCGAAAA | |
| | CATAAAGAAAAGATGAGCAAAGATGGTAAAAAGAAGAAAAAGAAG | |
| | TCAAAGACAAAGTGTGTAATTATGTAA | |
| KRAS encoding | AUGACUGAAUAUAAACUUGUGGUAGUUGGAGCUGGUGGCGUAGGC | 66 |
| RNA sequence | AAGAGUGCCUUGACGAUACAGCUAAUUCAGAAUCAUUUUGUGGAC | |
| (from Genbank | GAAUAUGAUCCAACAAUAGAGGAUUCCUACAGGAAGCAAGUAGUA | |
| NM_004985.4) | AUUGAUGGAGAAACCUGUCUCUUGGAUAUUCUCGACACAGCAGGU | |
| Bold and | CAAGAGGAGUACA [symbol] [symbol] UGAGGACUGGG | |
| italicized: | GAGGGCUUUCUUUGUGUAUUUGCCAUAAAUAAUACUAAAUCAUUU | |
| siRNA binding | GAAGAUAUUCACCAUUAUAGAGAACAAAUUAAAAGAGUUAAGGAC | |
| regions | UCUGAAGAUGUACCUAUGGUCCUAGUAGGAAAUAAAUGUGAUUUG | |
| | CCUUCUAGAACAGUAGACACAAAACAGGCUCAGGACUUAGCAAGA | |
| | AGUUAUGGAAUUCCUUUUAUUGAAACAUCAGCAAAGACAAGACAG | |
| | GGUGUUGAUGAUGCCUUCUAUACAUUAGUUCGAGAAAUUCGAAAA | |
| | CAUAAAGAAAAGAUGAGCAAAGAUGGUAAAAAGAAGAAAAAGAAG | |
| | UCAAAGACAAAGUGUGUAAUUAUGUAA | |
| Human IL-15 | MRISKPHLRSISIQCYLCLLLNSHFLTEAGIHVFILGCFSAGLPK | 67 |
| amino acid | TEANWVNVISDLKKIEDLIQSMHIDATLYTESDVHPSCKVTAMKC | |
| (Genbank | FLLELQVISLESGDASIHDTVENLIILANNSLSSNGNVTESGCKE | |
| NM_000585.4) | CEELEEKNIKEFLQSFVHIVQMFINTS | |
| Underlined: | | |
| signal sequence | | |
| Mature Human | GIHVFILGCFSAGLPKTEANWVNVISDLKKIEDLIQSMHIDATLY | 68 |
| IL-15 amino | TESDVHPSCKVTAMKCFLLELQVISLESGDASIHDTVENLIILAN | |
| acid (Genbank | NSLSSNGNVTESGCKECEELEEKNIKEFLQSFVHIVQMFINTS | |
| NM_000585.4) | | |
| Human IL-15 | ATGTTCCATCATGTTCCATGCTGCTGACGTCACATGGAGCACAGA | 69 |
| nucleic acid | AATCAATGTTAGCAGATAGCCAGCCCATACAAGATCGTATTGTAT | |
| (Genbank | TGTAGGAGGCATTGTGGATGGATGGCTGCTGGAAACCCCTTGCCA | |
| NM_000585.4) | TAGCCAGCTCTTCTTCAATACTTAAGGATTTACCGTGGCTTTGAG | |
| Underlined: | TAATGAGAATTTCGAAACCACATTTGAGAAGTATTTCCATCCAGT | |
| coding sequence | GCTACTTGTGTTTACTTCTAAACAGTCATTTTCTAACTGAAGCTG | |
| Bold: signal | GCATTCATGTCTTCATTTTGGGCTGTTTCAGTGCAGGGCTTCCTA | |
| sequence | AAACAGAAGCCAACTGGGTGAATGTAATAAGTGATTTGAAAAAAA | |
| | TTGAAGATCTTATTCAATCTATGCATATTGATGCTACTTTATATA | |
| | CGGAAAGTGATGTTCACCCCAGTTGCAAAGTAACAGCAATGAAGT | |
| | GCTTTCTCTTGGAGTTACAAGTTATTTCACTTGAGTCCGGAGATG | |
| | CAAGTATTCATGATACAGTAGAAAATCTGATCATCCTAGCAAACA | |
| | ACAGTTTGTCTTCTAATGGGAATGTAACAGAATCTGGATGCAAAG | |
| | AATGTGAGGAACTGGAGGAAAAAAATATTAAAGAATTTTTGCAGA | |
| | GTTTTGTACATATTGTCCAAATGTTCATCAACACTTCTTGATTGC | |
| | AATTGATTCTTTTTAAAGTGTTTCTGTTATTAACAAACATCACTC | |
| | TGCTGCTTAGACATAACAAAACACTCGGCATTTCAAATGTGCTGT | |
| | CAAAACAAGTTTTTCTGTCAAGAAGATGATCAGACCTTGGATCAG | |
| | ATGAACTCTTAGAAATGAAGGCAGAAAAATGTCATTGAGTAATAT | |
| | AGT | |
| CD155 amino | MARAMAAAWPLLLVALLVLSWPPPGTGDVVVQAPTQVPGFLGDSV | 70 |
| acid (Genbank | TLPCYLQVPNMEVTHVSQLTWARHGESGSMAVFHQTQGPSYSESK | |
| NM_006505.4) | RLEFVAARLGAELRNASLRMFGLRVEDEGNYTCLFVTFPQGSRSV | |
| (Transcript | DIWLRVLAKPQNTAEVQKVQLTGEPVPMARCVSTGGRPPAQITWH | |
| variant 1) | SDLGGMPNTSQVPGFLSGTVTVTSLWILVPSSQVDGKNVTCKVEH | |
| | ESFEKPQLLTVNLTVYYPPEVSISGYDNNWYLGQNEATLTCDARS | |
| | NPEPTGYNWSTTMGPLPPFAVAQGAQLLIRPVDKPINTTLICNVT | |
| | NALGARQAELTVQVKEGPPSEHSGMSRNAIIFLVLGILVFLILLG | |
| | IGIYFYWSKCSREVLWHCHLCPSSTEHASASANGHVSYSAVSREN | |
| | SSSQDPQTEGTR | |
| CD155 | ATGGCCCGAGCCATGGCCGCCGCGTGGCCGCTGCTGCTGGTGGCG | 71 |
| encoding DNA | CTACTGGTGCTGTCCTGGCCACCCCCAGGAACCGGGGACGTCGTC | |
| sequence | GTGCAGGCGCCCACCCAGGTGCCCGGCTTCTTGGGCGACTCCGTG | |
| (from Genbank | ACGCTGCCCTGCTACCTACAGGTGCCCAACATGGAGGTGACGCAT | |
| NM_006505.4) | GTGTCACAGCTGACTTGGGCGCGGCATGGTGAATCTGGCAGCATG | |
| Bold and | GCCGTCTTCCACCAAACGCAGGGCCCCAGCTATTCGGAGTCCAAA | |
| italicized: | CGGCTGGAATTCGTGGCAGCCAGACTGGGCGCGGAGCTGCGGAAT | |
| siRNA binding | GCCTCGCTGAGGATGTTCGGGTTGCGCGTAGAGGATGAAGGCAAC | |
| regions | TACACCTGCCTGTTCGTCACGTTCCCGCAGGGCAGCAGGAGCGTG | |
| | GATATCTGGCTCCGAGTGCTTGCCAAGCCCCAGAACACAGCTGAG | |
| | GTTCAGAAGGTCCAGCTCACTGGAGAGCCAGTGCCCATGGCCCGC | |
| | TGCGTCTCCACAGGGGGTCGCCCGCCAGCCCAAATCACCTGGCAC | |
| | TCAGACCTGGGCGGGATGCCCAATACGAGCCAGGTGCCAGGGTTC | |
| | CTGTCTGGCACAGTCACTGTCACCAGCCTCTGGATATTGGTGCCC | |
| | TCAAGCCAGGTGGACGGCAAGAATGTGACCTGCAAGGTGGAGCAC | |
| | GAGAGCTTTGAGAAGCCTCAGCTGCTGACTGTGAACCTCACCGTG | |
| | TACTACCCCCCAGA [symbol] [symbol] TAACAACTGG | |
| | TACCTTGGCCAGAATGAGGCCACCCTGACCTGCGATGCTCGCAGC | |
| | AACCCAGAGCCCACAGGCTATAATTGGAGCACGACCATGGGTCCC | |
| | CTGCCACCCTTTGCTGTGGCCCAGGGCGCCCAGCTCCTGATCCGT | |
| | CCTGTGGACAAACCAATCAACACAACTTTAATCTGCAACGTCACC | |
| | AATGCCCTAGGAGCTCGCCAGGCAGAACTGACCGTCCAGGTCAAA | |
| | GAGGGACCTCCCAGTGAGCACTCAGGCAT [symbol] | |
| | [symbol] CCTGGTTCTGGGAATCCTGGTTTTTCTGATCCTGCTGGGG | |
| | ATCGGGATTTATTTCTATTGGTCCAAATGTTCCCGTGAGGTCCTT | |
| | TGGCACTGTCATCTGTGTCCCTCGAGTACAGAGCATGCCAGCGCC | |
| | TCAGCTAATGGGCATGTCTCCTATTCAGCTGTGAGCAGAGAGAAC | |
| | AGCTCTTCCCAGGATCCACAGACAGAGGGCACAAGGTGA | |
| CD155 | AUGGCCCGAGCCAUGGCCGCCGCGUGGCCGCUGCUGCUGGUGGCG | 72 |
| encoding RNA | CUACUGGUGCUGUCCUGGCCACCCCCAGGAACCGGGGACGUCGUC | |
| sequence | GUGCAGGCGCCCACCCAGGUGCCCGGCUUCUUGGGCGACUCCGUG | |
| (from Genbank | ACGCUGCCCUGCUACCUACAGGUGCCCAACAUGGAGGUGACGCAU | |
| NM_006505.4) | GUGUCACAGCUGACUUGGGCGCGGCAUGGUGAAUCUGGCAGCAUG | |
| Bold and | GCCGUCUUCCACCAAACGCAGGGCCCCAGCUAUUCGGAGUCCAAA | |
| italicized: | CGGCUGGAAUUCGUGGCAGCCAGACUGGGCGCGGAGCUGCGGAAU | |
| siRNA binding | GCCUCGCUGAGGAUGUUCGGGUUGCGCGUAGAGGAUGAAGGCAAC | |
| regions | UACACCUGCCUGUUCGUCACGUUCCCGCAGGGCAGCAGGAGCGUG | |
| | GAUAUCUGGCUCCGAGUGCUUGCCAAGCCCCAGAACACAGCUGAG | |
| | GUUCAGAAGGUCCAGCUCACUGGAGAGCCAGUGCCCAUGGCCCGC | |
| | UGCGUCUCCACAGGGGGUCGCCCGCCAGCCCAAAUCACCUGGCAC | |
| | UCAGACCUGGGCGGGAUGCCCAAUACGAGCCAGGUGCCAGGGUUC | |
| | CUGUCUGGCACAGUCACUGUCACCAGCCUCUGGAUAUUGGUGCCC | |
| | UCAAGCCAGGUGGACGGCAAGAAUGUGACCUGCAAGGUGGAGCAC | |
| | GAGAGCUUUGAGAAGCCUCAGCUGCUGACUGUGAACCUCACCGUG | |
| | UACUACCCCCCAGA [symbol] [symbol] UAACAACUGG | |
| | UACCUUGGCCAGAAUGAGGCCACCCUGACCUGCGAUGCUCGCAGC | |
| | AACCCAGAGCCCACAGGCUAUAAUUGGAGCACGACCAUGGGUCCC | |
| | CUGCCACCCUUUGCUGUGGCCCAGGGCGCCCAGCUCCUGAUCCGU | |
| | CCUGUGGACAAACCAAUCAACACAACUUUAAUCUGCAACGUCACC | |
| | AAUGCCCUAGGAGCUCGCCAGGCAGAACUGACCGUCCAGGUCAAA | |
| | GAGGGACCUCCCAGUGAGCACUCAGGCAU [symbol] | |
| | [symbol] CCUGGUUCUGGGAAUCCUGGUUUUUCUGAUCCUGCUGGGG | |
| | AUCGGGAUUUAUUUCUAUUGGUCCAAAUGUUCCCGUGAGGUCCUU | |
| | UGGCACUGUCAUCUGUGUCCCUCGAGUACAGAGCAUGCCAGCGCC | |
| | UCAGCUAAUGGGCAUGUCUCCUAUUCAGCUGUGAGCAGAGAGAAC | |
| | AGCUCUUCCCAGGAUCCACAGACAGAGGGCACAAGGUGA | |
| PD-L1 amino | MRIFAVFIFMTYWHLLNAFTVTVPKDLYVVEYGSNMTIECKFPVE | 73 |
| acid (Genbank | KQLDLAALIVYWEMEDKNIIQFVHGEEDLKVQHSSYRQRARLLKD | |
| NM_014143.3) | QLSLGNAALQITDVKLQDAGVYRCMISYGGADYKRITVKVNAPYN | |
| (Transcript | KINQRILVVDPVTSEHELTCQAEGYPKAEVIWTSSDHQVLSGKTT | |
| variant 1) | TTNSKREEKLFNVTSTLRINTTTNEIFYCTFRRLDPEENHTAELV | |
| | IPELPLAHPPNERTHLVILGAILLCLGVALTFIFRLRKGRMMDVK | |
| | KCGIQDTNSKKQSDTHLEET | |
| PD-L1 encoding | ATGAGGATATTTGCTGTCTTTATATTCATGACCTACTGGCATTTG | 74 |
| DNA sequence | CTGAACGCATTTACTGTCACGGTTCCCAAGGACCTATATGTGGTA | |
| (from Genbank | GAGTATGGTAGCAATATGACAATTGAATGCAAATTCCCAGTAGAA | |
| NM_014143.3) | AAACAATTAGACCTGGCTGCACTAATTGTCTATTGGGAAATGGAG | |
| Bold and | GATAAGAACATTATTCAATTTGTGCATGGAGAGGAAGACCT [symbol] | |
| italicized: | [symbol] CAGACAGAGGGCCCGGCTGTTGAAGGAC | |
| siRNA binding | CAGCTCTCCCTGGGAAATGCTGCACTTCAGATCACAGATGTGAAA | |
| regions | TTGCAGGATGCAGGGGTGTACCGCTGCATGATCAGCTATGGTGGT | |
| | GCCGACTACAA [symbol] [symbol] TGCCCCATACAAC | |
| | AAAATCAACCAAAGAATTTTGGTTGTGGATCCAGTCACCTCTGAA | |
| | CATGAACTGACATGTCAGGCTGAGGGCTACCCCAAGGCCGAAGTC | |
| | ATCTGGACAAGCAGTGACCATCAAGTCCTGAGTGGTAAGACCACC | |
| | ACCACCAATTCCAAGAGAGAGGAGAAGCTTTTCAATGT [symbol] | |
| | [symbol] CACAACAACTAATGAGATTTTCTACTGCACT | |
| | TTTAGGAGATTAGATCCTGAGGAAAACCATACAGCTGAATTGGTC | |
| | ATCCCAGAACTACCTCTGGCACATCCTCCAAATGAAAGGACTCAC | |
| | TTGGTAATTCTGGGAGCCATCTTATTATGCCTTGGTGTAGCACTG | |
| | ACATTCATCTTCCGTTTAAGAAAAGGGAGAATGATGGATGTGAAA | |
| | AAATGTGGCATCCAAGATACAAACTCAAAGAAGCAAAGTGATACA | |
| | CATTTGGAGGAGACGTAA | |
| PD-L1 encoding | AUGAGGAUAUUUGCUGUCUUUAUAUUCAUGACCUACUGGCAUUUG | 75 |
| RNA sequence | CUGAACGCAUUUACUGUCACGGUUCCCAAGGACCUAUAUGUGGUA | |
| (from Genbank | GAGUAUGGUAGCAAUAUGACAAUUGAAUGCAAAUUCCCAGUAGAA | |
| NM_014143.3) | AAACAAUUAGACCUGGCUGCACUAAUUGUCUAUUGGGAAAUGGAG | |
| Bold and | GAUAAGAACAUUAUUCAAUUUGUGCAUGGAGAGGAAGACCU [symbol] | |
| italicized: | [symbol] CAGACAGAGGGCCCGGCUGUUGAAGGAC | |
| siRNA binding | CAGCUCUCCCUGGGAAAUGCUGCACUUCAGAUCACAGAUGUGAAA | |
| regions | UUGCAGGAUGCAGGGGUGUACCGCUGCAUGAUCAGCUAUGGUGGU | |
| | GCCGACUACAA [symbol] [symbol] UGCCCCAUACAAC | |
| | AAAAUCAACCAAAGAAUUUUGGUUGUGGAUCCAGUCACCUCUGAA | |
| | CAUGAACUGACAUGUCAGGCUGAGGGCUACCCCAAGGCCGAAGUC | |
| | AUCUGGACAAGCAGUGACCAUCAAGUCCUGAGUGGUAAGACCACC | |
| | ACCACCAAUUCCAAGAGAGAGGAGAAGCUUUUCAAUGU [symbol] | |
| | [symbol] CACAACAACUAAUGAGAUUUUCUACUGCACU | |
| | UUUAGGAGAUUAGAUCCUGAGGAAAACCAUACAGCUGAAUUGGUC | |
| | AUCCCAGAACUACCUCUGGCACAUCCUCCAAAUGAAAGGACUCAC | |
| | UUGGUAAUUCUGGGAGCCAUCUUAUUAUGCCUUGGUGUAGCACUG | |
| | ACAUUCAUCUUCCGUUUAAGAAAAGGGAGAAUGAUGGAUGUGAAA | |
| | AAAUGUGGCAUCCAAGAUACAAACUCAAAGAAGCAAAGUGAUACA | |
| | CAUUUGGAGGAGACGUAA | |
| c-Myc amino | MDFFRVVENQQPPATMPLNVSFTNRNYDLDYDSVQPYFYCDEEEN | 76 |
| acid (Genbank | FYQQQQQSELQPPAPSEDIWKKFELLPTPPLSPSRRSGLCSPSYV | |
| NM_002467.4) | AVTPFSLRGDNDGGGGSFSTADQLEMVTELLGGDMVNQSFICDPD | |
| | DETFIKNIIIQDCMWSGFSAAAKLVSEKLASYQAARKDSGSPNPA | |
| | RGHSVCSTSSLYLQDLSAAASECIDPSVVFPYPLNDSSSPKSCAS | |
| | QDSSAFSPSSDSLLSSTESSPQGSPEPLVLHEETPPTTSSDSEEE | |
| | QEDEEEIDVVSVEKRQAPGKRSESGSPSAGGHSKPPHSPLVLKRC | |
| | HVSTHQHNYAAPPSTRKDYPAAKRVKLDSVRVLRQISNNRKCTSP | |
| | RSSDTEENVKRRTHNVLERQRRNELKRSFFALRDQIPELENNEKA | |
| | PKVVILKKATAYILSVQAEEQKLISEEDLLRKRREQLKHKLEQLR | |
| | NSCA | |
| c-Myc encoding | ATGGATTTTTTTCGGGTAGTGGAAAACCAGCAGCCTCCCGCGACG | 77 |
| DNA sequence | ATGCCCCTCAACGTTAGCTTCACCAACAGGAACTATGACCTCGAC | |
| (from Genbank | TACGACTCGGTGCAGCCGTATTTCTACTGCGACGAGGAGGAGAAC | |
| NM_002467.4) | TTCTACCAGCAGCAGCAGCAGAGCGAGCTGCAGCCCCCGGCGCCC | |
| Bold and | AGCGAGGATATCTGGAAGAAATTCGAGCTGCTGCCCACCCCGCCC | |
| italicized: | CTGTCCCCTAGCCGCCGCTCCGGGCTCTGCTCGCCCTCCTACGTT | |
| siRNA binding | GCGGTCACACCCTTCTCCCTTCGGGGAGACAACGACGGCGGTGGC | |
| regions | GGGAGCTTCTCCACGGCCGACCAGCTGGAGATGGTGACCGAGCTG | |
| | CTGGGAGGAGACATGGTGAACCAGAGTTTCATCTGCGACCC [symbol] | |
| | [symbol] AAACATCATCATCCAGGACTGTATGTGG | |
| | AGCGGCTTCTCGGCCGCCGCCAAGCTCGTCTCAGAGAAGCTGGCC | |
| | TCCTACCAGGCTGCGCGCAAAGACAGCGGCAGCCCGAACCCCGCC | |
| | CGCGGCCACAGCGTCTGCTCCACCTCCAGCTTGTACCTGCAGGAT | |
| | CTGAGCGCCGCCGCCTCAGAGTGCATCGACCCCTCGGTGGTCTTC | |
| | CCCTACCCTCTCAACGACAGCAGCTCGCCCAAGTCCTGCGCCTCG | |
| | CAAGACTCCAGCGCCTTCTCTCCGTCCTCGGATTCTCTGCTCTCC | |
| | TCGACGGAGTCCTCCCCGCAGGGCAGCCCCGAGCCCCTGGTGCTC | |
| | CATGAGGAGACACCGCCCACCACCAGCAGCGACTCTGAGGAGGAA | |
| | CAAGAAGATGAGGAAGAAATCGATGTTGTTTCTGTGGAAAAGAGG | |
| | CAGGCTCCTGGCAAAAGGTCAGAGTCTGGATCACCTTCTGCTGGA | |
| | GGCCACAGCAAACCTCCTCACAGCCCACTGGTCCTCAAGAGGTGC | |
| | CACGTCTCCACACATCAGCACAACTACGCAGCGCCTCCCTCCACT | |
| | CGGAAGGACTATCCTGCTGCCAAGAGGGTCAAGTTGGACAGTGTC | |
| | AGAGTCCTGAGACAGATCAGCAACAACCGAAAATGCACCAGCCCC | |
| | AGGTCCTCGGACACCGAGGAGAATGTCAAGAGGCGAACACACAAC | |
| | GTCTTGGAGCGCCAGAGGAGGAACGAGCTAAAACGGAGCTTTTTT | |
| | GCCCTGCGTGACCAGATCCCGGAGTTGGAAAACAATGAAAAGGCC | |
| | CCCAAGGTAGTTATCCTTAAAAAAGCCACAGCATACATCCTGTCC | |
| | GTCCAAGCAGAGGAGCAAAAGCTCATTTCTGAAGAGGACTTGTTG | |
| | CGGAAACGACGAGAACAGTTGAAACACAAACTTGAACAGCTACGG | |
| | AACTCTTGTGCGTAA | |
| c-Myc encoding | AUGGAUUUUUUUCGGGUAGUGGAAAACCAGCAGCCUCCCGCGACG | 78 |
| RNA sequence | AUGCCCCUCAACGUUAGCUUCACCAACAGGAACUAUGACCUCGAC | |
| (from Genbank | UACGACUCGGUGCAGCCGUAUUUCUACUGCGACGAGGAGGAGAAC | |
| NM_002467.4) | UUCUACCAGCAGCAGCAGCAGAGCGAGCUGCAGCCCCCGGCGCCC | |
| Bold and | AGCGAGGAUAUCUGGAAGAAAUUCGAGCUGCUGCCCACCCCGCCC | |
| italicized: | CUGUCCCCUAGCCGCCGCUCCGGGCUCUGCUCGCCCUCCUACGUU | |
| siRNA binding | GCGGUCACACCCUUCUCCCUUCGGGGAGACAACGACGGCGGUGGC | |
| regions | GGGAGCUUCUCCACGGCCGACCAGCUGGAGAUGGUGACCGAGCUG | |
| | CUGGGAGGAGACAUGGUGAACCAGAGUUUCAUCUGCGACCC [symbol] | |
| | GACGAGACCUUCAUCAAAAACAUCAUCAUCCAGGACUGUAUGUGG | |
| | AGCGGCUUCUCGGCCGCCGCCAAGCUCGUCUCAGAGAAGCUGGCC | |
| | UCCUACCAGGCUGCGCGCAAAGACAGCGGCAGCCCGAACCCCGCC | |
| | CGCGGCCACAGCGUCUGCUCCACCUCCAGCUUGUACCUGCAGGAU | |
| | CUGAGCGCCGCCGCCUCAGAGUGCAUCGACCCCUCGGUGGUCUUC | |
| | CCCUACCCUCUCAACGACAGCAGCUCGCCCAAGUCCUGCGCCUCG | |
| | CAAGACUCCAGCGCCUUCUCUCCGUCCUCGGAUUCUCUGCUCUCC | |
| | UCGACGGAGUCCUCCCCGCAGGGCAGCCCCGAGCCCCUGGUGCUC | |
| | CAUGAGGAGACACCGCCCACCACCAGCAGCGACUCUGAGGAGGAA | |
| | CAAGAAGAUGAGGAAGAAAUCGAUGUUGUUUCUGUGGAAAAGAGG | |
| | CAGGCUCCUGGCAAAAGGUCAGAGUCUGGAUCACCUUCUGCUGGA | |
| | GGCCACAGCAAACCUCCUCACAGCCCACUGGUCCUCAAGAGGUGC | |
| | CACGUCUCCACACAUCAGCACAACUACGCAGCGCCUCCCUCCACU | |
| | CGGAAGGACUAUCCUGCUGCCAAGAGGGUCAAGUUGGACAGUGUC | |
| | AGAGUCCUGAGACAGAUCAGCAACAACCGAAAAUGCACCAGCCCC | |
| | AGGUCCUCGGACACCGAGGAGAAUGUCAAGAGGCGAACACACAAC | |
| | GUCUUGGAGCGCCAGAGGAGGAACGAGCUAAAACGGAGCUUUUUU | |
| | GCCCUGCGUGACCAGAUCCCGGAGUUGGAAAACAAUGAAAAGGCC | |
| | CCCAAGGUAGUUAUCCUUAAAAAAGCCACAGCAUACAUCCUGUCC | |
| | GUCCAAGCAGAGGAGCAAAAGCUCAUUUCUGAAGAGGACUUGUUG | |
| | CGGAAACGACGAGAACAGUUGAAACACAAACUUGAACAGCUACGG | |
| | AACUCUUGUGCGUAA | |
| Human IL-7 | MFHVSFRYIFGLPPLILVLLPVASSDCDIEGKDGKQYESVLMVSI | 79 |
| amino acid | DQLLDSMKEIGSNCLNNEFNFFKRHICDANKEGMFLFRAARKLRQ | |
| (Genbank | FLKMNSTGDFDLHLLKVSEGTTILLNCTGQVKGRKPAALGEAQPT | |
| NM_000880.3) | KSLEENKSLKEQKKLNDLCFLKRLLQEIKTCWNKILMGTKEH | |
| Underlined: | | |
| signal sequence | | |
| Mature Human | DCDIEGKDGKQYESVLMVSIDQLLDSMKEIGSNCLNNEFNFFKRH | 80 |
| IL-7 amino acid | ICDANKEGMFLFRAARKLRQFLKMNSTGDFDLHLLKVSEGTTILL | |
| (Genbank | NCTGQVKGRKPAALGEAQPTKSLEENKSLKEQKKLNDLCFLKRLL | |
| NM_000880.3) | QEIKTCWNKILMGTKEH | |
| Human IL-7 | ATGTTCCATGTTTCTTTTAGGTATATCTTTGGACTTCCTCCCCTG | 81 |
| nucleic acid | ATCCTTGTTCTGTTGCCAGTAGCATCATCTGATTGTGATATTGAA | |
| (Genbank | GGTAAAGATGGCAAACAATATGAGAGTGTTCTAATGGTCAGCATC | |
| NM_000880.3) | GATCAATTATTGGACAGCATGAAAGAAATTGGTAGCAATTGCCTG | |
| Underlined: | AATAATGAATTTAACTTTTTTAAAAGACATATCTGTGATGCTAAT | |
| coding sequence | AAGGAAGGTATGTTTTTATTCCGTGCTGCTCGCAAGTTGAGGCAA | |
| Bold: signal | TTTCTTAAAATGAATAGCACTGGTGATTTTGATCTCCACTTATTA | |
| sequence | AAAGTTTCAGAAGGCACAACAATACTGTTGAACTGCACTGGCCAG | |
| | GTTAAAGGAAGAAAACCAGCTGCCCTGGGTGAAGCCCAACCAACA | |
| | AAGAGTTTGGAAGAAAATAAATCTTTAAAGGAACAGAAAAAACTG | |
| | AATGACTTGTGTTTCCTAAAGAGACTATTACAAGAGATAAAAACT | |
| | TGTTGGAATAAAATTTTGATGGGCACTAAAGAACACTGA | |
| Human IL-12 | MCPARSLLLVATLVLLDHLSLA | 142 |
| alpha signal | | |
| peptide | | |
| (Genbank | | |
| NM_000882.4) | | |
| Human IL-12 | MCHQQLVISWFSLVFLASPLVA | 143 |
| beta signal | | |
| peptide | | |
| (Genbank | | |
| NM_002187.2) | | |
| Human IL-15 | MRISKPHLRSISIQCYLCLLLNSHFLTEA | 144 |
| signal peptide | | |
| (Genbank | | |
| NM_000585.4) | | |
| Human IL-7 | MFHVSFRYIFGLPPLILVLLPVASS | 145 |
| signal peptide | | |
| (Genbank | | |
| NM_000880.3) | | |
| Endogenous IL- | ATGTGTCCAGCGCGCAGCCTCCTCCTTGTGGCTACCCTGGTCCTC | 146 |
| 12 alpha signal | CTGGACCACCTCAGTTTGGCC | |
| peptide nucleic | | |
| acid | | |
| Endogenous IL- | ATGTGTCACCAGCAGTTGGTCATCTCTTGGTTTTCCCTGGTTTTT | 147 |
| 12 beta signal | CTGGCATCTCCCCTCGTGGCC | |
| peptide nucleic | | |
| acid | | |
| Endogenous IL- | ATGAGAATTTCGAAACCACATTTGAGAAGTATTTCCATCCAGTGC | 148 |
| 15 signal | TACTTGTGTTTACTTCTAAACAGTCATTTTCTAACTGAAGCT | |
| peptide nucleic | | |
| acid | | |
| Endogenous IL- | ATGTTCCACGTGTCCTTCCGGTACATCTTCGGCCTGCCTCCACTG | 149 |
| 7 signal peptide | ATCCTGGTGCTGCTGCCTGTGGCCAGCAGC | |
| nucleic acid | | |

TABLE 4
Plasmid Vector Sequences for Compounds 1-17

| SEQ ID NO | Compound | Sequence (5′ to 3′) |
|---|---|---|
| 82 | Compound 1 | CTAAATTGTAAGCGTTAATATTTTGTTAAAATTCGCGTTAAAT |
| | (pMA-T) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCACGTGTCTTGTCCAGAGCTC [symbol] |
| | | [symbol] ATGTACAGAATGCAGCTGCTGAGCTGTATCGCCCTGTCT |
| | | CTGGCCCTGGTCACAAATAGCGCCCCTACCAGCAGCAGCACCA |
| | | AGAAAACACAGCTGCAACTGGAACACCTCCTGCTGGACCTGCA |
| | | GATGATCCTGAACGGCATCAACAACTACAAGAACCCCAAGCTG |
| | | ACCCGGATGCTGACCTTCAAGTTCTACATGCCCAAGAAGGCCA |
| | | CCGAGCTGAAGCACCTCCAGTGCCTGGAAGAGGAACTGAAGCC |
| | | CCTGGAAGAAGTGCTGAATCTGGCCCAGAGCAAGAACTTCCAC |
| | | CTGAGGCCTAGGGACCTGATCAGCAACATCAACGTGATCGTGC |
| | | TGGAACTGAAAGGCAGCGAGACAACCTTCATGTGCGAGTACGC |
| | | CGACGAGACAGCTACCATCGTGGAATTTCTGAACCGGTGGATC |
| | | ACCTTCTGCCAGAGCATCATCAGCACCCTGACCTGAGGTACCT |
| | | GGAGCACAAGACTGGCCTCATGGGCCTTCCGCTCACTGCCCGC |
| | | TTTCCAGTCGGGAAACCTGTCGTGCCAGCTGCATTAACATGGT |
| | | CATAGCTGTTTCCTTGCGTATTGGGCGCTCTCCGCTTCCTCGC |
| | | TCACTGACTCGCTGCGCTCGGTCGTTCGGGTAAAGCCTGGGGT |
| | | GCCTAATGAGCAAAAGGCCAGCAAAAGGCCAGGAACCGTAAAA |
| | | AGGCCGCGTTGCTGGCGTTTTTCCATAGGCTCCGCCCCCCTGA |
| | | CGAGCATCACAAAAATCGACGCTCAAGTCAGAGGTGGCGAAAC |
| | | CCGACAGGACTATAAAGATACCAGGCGTTTCCCCCTGGAAGCT |
| | | CCCTCGTGCGCTCTCCTGTTCCGACCCTGCCGCTTACCGGATA |
| | | CCTGTCCGCCTTTCTCCCTTCGGGAAGCGTGGCGCTTTCTCAT |
| | | AGCTCACGCTGTAGGTATCTCAGTTCGGTGTAGGTCGTTCGCT |
| | | CCAAGCTGGGCTGTGTGCACGAACCCCCCGTTCAGCCCGACCG |
| | | CTGCGCCTTATCCGGTAACTATCGTCTTGAGTCCAACCCGGTA |
| | | AGACACGACTTATCGCCACTGGCAGCAGCCACTGGTAACAGGA |
| | | TTAGCAGAGCGAGGTATGTAGGCGGTGCTACAGAGTTCTTGAA |
| | | GTGGTGGCCTAACTACGGCTACACTAGAAGAACAGTATTTGGT |
| | | ATCTGCGCTCTGCTGAAGCCAGTTACCTTCGGAAAAAGAGTTG |
| | | GTAGCTCTTGATCCGGCAAACAAACCACCGCTGGTAGCGGTGG |
| | | TTTTTTTGTTTGCAAGCAGCAGATTACGCGCAGAAAAAAAGGA |
| | | TCTCAAGAAGATCCTTTGATCTTTTCTACGGGGTCTGACGCTC |
| | | AGTGGAACGAAAACTCACGTTAAGGGATTTTGGTCATGAGATT |
| | | ATCAAAAAGGATCTTCACCTAGATCCTTTTAAATTAAAAATGA |
| | | AGTTTTAAATCAATCTAAAGTATATATGAGTAAACTTGGTCTG |
| | | ACAGTTACCAATGCTTAATCAGTGAGGCACCTATCTCAGCGAT |
| | | CTGTCTATTTCGTTCATCCATAGTTGCCTGACTCCCCGTCGTG |
| | | TAGATAACTACGATACGGGAGGGCTTACCATCTGGCCCCAGTG |
| | | CTGCAATGATACCGCGAGAACCACGCTCACCGGCTCCAGATTT |
| | | ATCAGCAATAAACCAGCCAGCCGGAAGGGCCGAGCGCAGAAGT |
| | | GGTCCTGCAACTTTATCCGCCTCCATCCAGTCTATTAATTGTT |
| | | GCCGGGAAGCTAGAGTAAGTAGTTCGCCAGTTAATAGTTTGCG |
| | | CAACGTTGTTGCCATTGCTACAGGCATCGTGGTGTCACGCTCG |
| | | TCGTTTGGTATGGCTTCATTCAGCTCCGGTTCCCAACGATCAA |
| | | GGCGAGTTACATGATCCCCCATGTTGTGCAAAAAAGCGGTTAG |
| | | CTCCTTCGGTCCTCCGATCGTTGTCAGAAGTAAGTTGGCCGCA |
| | | GTGTTATCACTCATGGTTATGGCAGCACTGCATAATTCTCTTA |
| | | CTGTCATGCCATCCGTAAGATGCTTTTCTGTGACTGGTGAGTA |
| | | CTCAACCAAGTCATTCTGAGAATAGTGTATGCGGCGACCGAGT |
| | | TGCTCTTGCCCGGCGTCAATACGGGATAATACCGCGCCACATA |
| | | GCAGAACTTTAAAAGTGCTCATCATTGGAAAACGTTCTTCGGG |
| | | GCGAAAACTCTCAAGGATCTTACCGCTGTTGAGATCCAGTTCG |
| | | ATGTAACCCACTCGTGCACCCAACTGATCTTCAGCATCTTTTA |
| | | CTTTCACCAGCGTTTCTGGGTGAGCAAAAACAGGAAGGCAAAA |
| | | TGCCGCAAAAAAGGGAATAAGGGCGACACGGAAATGTTGAATA |
| | | CTCATACTCTTCCTTTTTCAATATTATTGAAGCATTTATCAGG |
| | | GTTATTGTCTCATGAGCGGATACATATTTGAATGTATTTAGAA |
| | | AAATAAACAAATAGGGGTTCCGCGCACATTTCCCCGAAAAGTG |
| | | CCAC |
| 83 | Compound 2* | CTAAATTGTAAGCGTTAATATTTTGTTAAAATTCGCGTTAAAT |
| | (pMA-T) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCACGTGTCTTGTCCAGAGCTC [symbol] |
| | | [symbol] ATGCTGAAACTGCTGCTGCTCCTGTGTATCGCCCTGTCT |
| | | CTGGCCGCCACAAATAGCGCCCCTACCAGCAGCTCCACCAAGA |
| | | AAACACAGCTGCAACTGGAACATCTGCTGCTGGACCTGCAGAT |
| | | GATCCTGAACGGCATCAACAACTACAAGAACCCCAAGCTGACC |
| | | CGGATGCTGACCTTCAAGTTCTACATGCCCAAGAAGGCCACCG |
| | | AGCTGAAGCACCTCCAGTGCCTGGAAGAGGAACTGAAGCCCCT |
| | | GGAAGAAGTGCTGAATCTGGCCCAGAGCAAGAACTTCCACCTG |
| | | AGGCCTAGGGACCTGATCAGCAACATCAACGTGATCGTGCTGG |
| | | AACTGAAAGGCAGCGAGACAACCTTCATGTGCGAGTACGCCGA |
| | | CGAGACAGCTACCATCGTGGAATTTCTGAACCGGTGGATCACC |
| | | TTCTGCCAGAGCATCATCAGCACCCTGACCTGAGGTACCTGGA |
| | | GCACAAGACTGGCCTCATGGGCCTTCCGCTCACTGCCCGCTTT |
| | | CCAGTCGGGAAACCTGTCGTGCCAGCTGCATTAACATGGTCAT |
| | | AGCTGTTTCCTTGCGTATTGGGCGCTCTCCGCTTCCTCGCTCA |
| | | CTGACTCGCTGCGCTCGGTCGTTCGGGTAAAGCCTGGGGTGCC |
| | | TAATGAGCAAAAGGCCAGCAAAAGGCCAGGAACCGTAAAAAGG |
| | | CCGCGTTGCTGGCGTTTTTCCATAGGCTCCGCCCCCCTGACGA |
| | | GCATCACAAAAATCGACGCTCAAGTCAGAGGTGGCGAAACCCG |
| | | ACAGGACTATAAAGATACCAGGCGTTTCCCCCTGGAAGCTCCC |
| | | TCGTGCGCTCTCCTGTTCCGACCCTGCCGCTTACCGGATACCT |
| | | GTCCGCCTTTCTCCCTTCGGGAAGCGTGGCGCTTTCTCATAGC |
| | | TCACGCTGTAGGTATCTCAGTTCGGTGTAGGTCGTTCGCTCCA |
| | | AGCTGGGCTGTGTGCACGAACCCCCCGTTCAGCCCGACCGCTG |
| | | CGCCTTATCCGGTAACTATCGTCTTGAGTCCAACCCGGTAAGA |
| | | CACGACTTATCGCCACTGGCAGCAGCCACTGGTAACAGGATTA |
| | | GCAGAGCGAGGTATGTAGGCGGTGCTACAGAGTTCTTGAAGTG |
| | | GTGGCCTAACTACGGCTACACTAGAAGAACAGTATTTGGTATC |
| | | TGCGCTCTGCTGAAGCCAGTTACCTTCGGAAAAAGAGTTGGTA |
| | | GCTCTTGATCCGGCAAACAAACCACCGCTGGTAGCGGTGGTTT |
| | | TTTTGTTTGCAAGCAGCAGATTACGCGCAGAAAAAAAGGATCT |
| | | CAAGAAGATCCTTTGATCTTTTCTACGGGGTCTGACGCTCAGT |
| | | GGAACGAAAACTCACGTTAAGGGATTTTGGTCATGAGATTATC |
| | | AAAAAGGATCTTCACCTAGATCCTTTTAAATTAAAAATGAAGT |
| | | TTTAAATCAATCTAAAGTATATATGAGTAAACTTGGTCTGACA |
| | | GTTACCAATGCTTAATCAGTGAGGCACCTATCTCAGCGATCTG |
| | | TCTATTTCGTTCATCCATAGTTGCCTGACTCCCCGTCGTGTAG |
| | | ATAACTACGATACGGGAGGGCTTACCATCTGGCCCCAGTGCTG |
| | | CAATGATACCGCGAGAACCACGCTCACCGGCTCCAGATTTATC |
| | | AGCAATAAACCAGCCAGCCGGAAGGGCCGAGCGCAGAAGTGGT |
| | | CCTGCAACTTTATCCGCCTCCATCCAGTCTATTAATTGTTGCC |
| | | GGGAAGCTAGAGTAAGTAGTTCGCCAGTTAATAGTTTGCGCAA |
| | | CGTTGTTGCCATTGCTACAGGCATCGTGGTGTCACGCTCGTCG |
| | | TTTGGTATGGCTTCATTCAGCTCCGGTTCCCAACGATCAAGGC |
| | | GAGTTACATGATCCCCCATGTTGTGCAAAAAAGCGGTTAGCTC |
| | | CTTCGGTCCTCCGATCGTTGTCAGAAGTAAGTTGGCCGCAGTG |
| | | TTATCACTCATGGTTATGGCAGCACTGCATAATTCTCTTACTG |
| | | TCATGCCATCCGTAAGATGCTTTTCTGTGACTGGTGAGTACTC |
| | | AACCAAGTCATTCTGAGAATAGTGTATGCGGCGACCGAGTTGC |
| | | TCTTGCCCGGCGTCAATACGGGATAATACCGCGCCACATAGCA |
| | | GAACTTTAAAAGTGCTCATCATTGGAAAACGTTCTTCGGGGCG |
| | | AAAACTCTCAAGGATCTTACCGCTGTTGAGATCCAGTTCGATG |
| | | TAACCCACTCGTGCACCCAACTGATCTTCAGCATCTTTTACTT |
| | | TCACCAGCGTTTCTGGGTGAGCAAAAACAGGAAGGCAAAATGC |
| | | CGCAAAAAAGGGAATAAGGGCGACACGGAAATGTTGAATACTC |
| | | ATACTCTTCCTTTTTCAATATTATTGAAGCATTTATCAGGGTT |
| | | ATTGTCTCATGAGCGGATACATATTTGAATGTATTTAGAAAAA |
| | | TAAACAAATAGGGGTTCCGCGCACATTTCCCCGAAAAGTGCCA |
| | | C |
| 84 | Compound 3* | CTAAATTGTAAGCGTTAATATTTTGTTAAAATTCGCGTTAAAT |
| | (pMA-T) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCACGTGTCTTGTCCAGAGCTC [symbol] |
| | | [symbol] ATGTTGTTGCTGCTGCTCGCCTGTATTGCCCTGGCCTCT |
| | | ACAGCCGCCGCTACAAATTCTGCCCCTACCAGCAGCTCCACCA |
| | | AGAAAACCCAGCTGCAACTGGAACATCTGCTGCTGGACCTGCA |
| | | GATGATCCTGAACGGCATCAACAACTACAAGAACCCCAAGCTG |
| | | ACCCGGATGCTGACCTTCAAGTTCTACATGCCCAAGAAGGCCA |
| | | CCGAGCTGAAGCACCTCCAGTGCCTGGAAGAGGAACTGAAGCC |
| | | CCTGGAAGAAGTGCTGAATCTGGCCCAGAGCAAGAACTTCCAC |
| | | CTGAGGCCTAGGGACCTGATCAGCAACATCAACGTGATCGTGC |
| | | TGGAACTGAAAGGCAGCGAGACAACCTTCATGTGCGAGTACGC |
| | | CGACGAGACAGCTACCATCGTGGAATTTCTGAACCGGTGGATC |
| | | ACCTTCTGCCAGAGCATCATCAGCACCCTGACCTGAGGTACCT |
| | | GGAGCACAAGACTGGCCTCATGGGCCTTCCGCTCACTGCCCGC |
| | | TTTCCAGTCGGGAAACCTGTCGTGCCAGCTGCATTAACATGGT |
| | | CATAGCTGTTTCCTTGCGTATTGGGCGCTCTCCGCTTCCTCGC |
| | | TCACTGACTCGCTGCGCTCGGTCGTTCGGGTAAAGCCTGGGGT |
| | | GCCTAATGAGCAAAAGGCCAGCAAAAGGCCAGGAACCGTAAAA |
| | | AGGCCGCGTTGCTGGCGTTTTTCCATAGGCTCCGCCCCCCTGA |
| | | CGAGCATCACAAAAATCGACGCTCAAGTCAGAGGTGGCGAAAC |
| | | CCGACAGGACTATAAAGATACCAGGCGTTTCCCCCTGGAAGCT |
| | | CCCTCGTGCGCTCTCCTGTTCCGACCCTGCCGCTTACCGGATA |
| | | CCTGTCCGCCTTTCTCCCTTCGGGAAGCGTGGCGCTTTCTCAT |
| | | AGCTCACGCTGTAGGTATCTCAGTTCGGTGTAGGTCGTTCGCT |
| | | CCAAGCTGGGCTGTGTGCACGAACCCCCCGTTCAGCCCGACCG |
| | | CTGCGCCTTATCCGGTAACTATCGTCTTGAGTCCAACCCGGTA |
| | | AGACACGACTTATCGCCACTGGCAGCAGCCACTGGTAACAGGA |
| | | TTAGCAGAGCGAGGTATGTAGGCGGTGCTACAGAGTTCTTGAA |
| | | GTGGTGGCCTAACTACGGCTACACTAGAAGAACAGTATTTGGT |
| | | ATCTGCGCTCTGCTGAAGCCAGTTACCTTCGGAAAAAGAGTTG |
| | | GTAGCTCTTGATCCGGCAAACAAACCACCGCTGGTAGCGGTGG |
| | | TTTTTTTGTTTGCAAGCAGCAGATTACGCGCAGAAAAAAAGGA |
| | | TCTCAAGAAGATCCTTTGATCTTTTCTACGGGGTCTGACGCTC |
| | | AGTGGAACGAAAACTCACGTTAAGGGATTTTGGTCATGAGATT |
| | | ATCAAAAAGGATCTTCACCTAGATCCTTTTAAATTAAAAATGA |
| | | AGTTTTAAATCAATCTAAAGTATATATGAGTAAACTTGGTCTG |
| | | ACAGTTACCAATGCTTAATCAGTGAGGCACCTATCTCAGCGAT |
| | | CTGTCTATTTCGTTCATCCATAGTTGCCTGACTCCCCGTCGTG |
| | | TAGATAACTACGATACGGGAGGGCTTACCATCTGGCCCCAGTG |
| | | CTGCAATGATACCGCGAGAACCACGCTCACCGGCTCCAGATTT |
| | | ATCAGCAATAAACCAGCCAGCCGGAAGGGCCGAGCGCAGAAGT |
| | | GGTCCTGCAACTTTATCCGCCTCCATCCAGTCTATTAATTGTT |
| | | GCCGGGAAGCTAGAGTAAGTAGTTCGCCAGTTAATAGTTTGCG |
| | | CAACGTTGTTGCCATTGCTACAGGCATCGTGGTGTCACGCTCG |
| | | TCGTTTGGTATGGCTTCATTCAGCTCCGGTTCCCAACGATCAA |
| | | GGCGAGTTACATGATCCCCCATGTTGTGCAAAAAAGCGGTTAG |
| | | CTCCTTCGGTCCTCCGATCGTTGTCAGAAGTAAGTTGGCCGCA |
| | | GTGTTATCACTCATGGTTATGGCAGCACTGCATAATTCTCTTA |
| | | CTGTCATGCCATCCGTAAGATGCTTTTCTGTGACTGGTGAGTA |
| | | CTCAACCAAGTCATTCTGAGAATAGTGTATGCGGCGACCGAGT |
| | | TGCTCTTGCCCGGCGTCAATACGGGATAATACCGCGCCACATA |
| | | GCAGAACTTTAAAAGTGCTCATCATTGGAAAACGTTCTTCGGG |
| | | GCGAAAACTCTCAAGGATCTTACCGCTGTTGAGATCCAGTTCG |
| | | ATGTAACCCACTCGTGCACCCAACTGATCTTCAGCATCTTTTA |
| | | CTTTCACCAGCGTTTCTGGGTGAGCAAAAACAGGAAGGCAAAA |
| | | TGCCGCAAAAAAGGGAATAAGGGCGACACGGAAATGTTGAATA |
| | | CTCATACTCTTCCTTTTTCAATATTATTGAAGCATTTATCAGG |
| | | GTTATTGTCTCATGAGCGGATACATATTTGAATGTATTTAGAA |
| | | AAATAAACAAATAGGGGTTCCGCGCACATTTCCCCGAAAAGTG |
| | | CCAC |
| 85 | Compound 4* | CTAAATTGTAAGCGTTAATATTTTGTTAAAATTCGCGTTAAAT |
| | (pMA-T) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCACGTGTCTTGTCCAGAGCTC [symbol] |
| | | [symbol] ATGTTGTTGCTGCTGCTCGCCTGTATTGCCCTGGCCTCT |
| | | ACAGCCCTGGTCACCAATTCTGCCCCTACCAGCAGCTCCACCA |
| | | AGAAAACCCAGCTGCAACTGGAACATCTGCTGCTGGACCTGCA |
| | | GATGATCCTGAACGGCATCAACAACTACAAGAACCCCAAGCTG |
| | | ACCCGGATGCTGACCTTCAAGTTCTACATGCCCAAGAAGGCCA |
| | | CCGAGCTGAAGCACCTCCAGTGCCTGGAAGAGGAACTGAAGCC |
| | | CCTGGAAGAAGTGCTGAATCTGGCCCAGAGCAAGAACTTCCAC |
| | | CTGAGGCCTAGGGACCTGATCAGCAACATCAACGTGATCGTGC |
| | | TGGAACTGAAAGGCAGCGAGACAACCTTCATGTGCGAGTACGC |
| | | CGACGAGACAGCTACCATCGTGGAATTTCTGAACCGGTGGATC |
| | | ACCTTCTGCCAGAGCATCATCAGCACCCTGACCTGAGGTACCT |
| | | GGAGCACAAGACTGGCCTCATGGGCCTTCCGCTCACTGCCCGC |
| | | TTTCCAGTCGGGAAACCTGTCGTGCCAGCTGCATTAACATGGT |
| | | CATAGCTGTTTCCTTGCGTATTGGGCGCTCTCCGCTTCCTCGC |
| | | TCACTGACTCGCTGCGCTCGGTCGTTCGGGTAAAGCCTGGGGT |
| | | GCCTAATGAGCAAAAGGCCAGCAAAAGGCCAGGAACCGTAAAA |
| | | AGGCCGCGTTGCTGGCGTTTTTCCATAGGCTCCGCCCCCCTGA |
| | | CGAGCATCACAAAAATCGACGCTCAAGTCAGAGGTGGCGAAAC |
| | | CCGACAGGACTATAAAGATACCAGGCGTTTCCCCCTGGAAGCT |
| | | CCCTCGTGCGCTCTCCTGTTCCGACCCTGCCGCTTACCGGATA |
| | | CCTGTCCGCCTTTCTCCCTTCGGGAAGCGTGGCGCTTTCTCAT |
| | | AGCTCACGCTGTAGGTATCTCAGTTCGGTGTAGGTCGTTCGCT |
| | | CCAAGCTGGGCTGTGTGCACGAACCCCCCGTTCAGCCCGACCG |
| | | CTGCGCCTTATCCGGTAACTATCGTCTTGAGTCCAACCCGGTA |
| | | AGACACGACTTATCGCCACTGGCAGCAGCCACTGGTAACAGGA |
| | | TTAGCAGAGCGAGGTATGTAGGCGGTGCTACAGAGTTCTTGAA |
| | | GTGGTGGCCTAACTACGGCTACACTAGAAGAACAGTATTTGGT |
| | | ATCTGCGCTCTGCTGAAGCCAGTTACCTTCGGAAAAAGAGTTG |
| | | GTAGCTCTTGATCCGGCAAACAAACCACCGCTGGTAGCGGTGG |
| | | TTTTTTTGTTTGCAAGCAGCAGATTACGCGCAGAAAAAAAGGA |
| | | TCTCAAGAAGATCCTTTGATCTTTTCTACGGGGTCTGACGCTC |
| | | AGTGGAACGAAAACTCACGTTAAGGGATTTTGGTCATGAGATT |
| | | ATCAAAAAGGATCTTCACCTAGATCCTTTTAAATTAAAAATGA |
| | | AGTTTTAAATCAATCTAAAGTATATATGAGTAAACTTGGTCTG |
| | | ACAGTTACCAATGCTTAATCAGTGAGGCACCTATCTCAGCGAT |
| | | CTGTCTATTTCGTTCATCCATAGTTGCCTGACTCCCCGTCGTG |
| | | TAGATAACTACGATACGGGAGGGCTTACCATCTGGCCCCAGTG |
| | | CTGCAATGATACCGCGAGAACCACGCTCACCGGCTCCAGATTT |
| | | ATCAGCAATAAACCAGCCAGCCGGAAGGGCCGAGCGCAGAAGT |
| | | GGTCCTGCAACTTTATCCGCCTCCATCCAGTCTATTAATTGTT |
| | | GCCGGGAAGCTAGAGTAAGTAGTTCGCCAGTTAATAGTTTGCG |
| | | CAACGTTGTTGCCATTGCTACAGGCATCGTGGTGTCACGCTCG |
| | | TCGTTTGGTATGGCTTCATTCAGCTCCGGTTCCCAACGATCAA |
| | | GGCGAGTTACATGATCCCCCATGTTGTGCAAAAAAGCGGTTAG |
| | | CTCCTTCGGTCCTCCGATCGTTGTCAGAAGTAAGTTGGCCGCA |
| | | GTGTTATCACTCATGGTTATGGCAGCACTGCATAATTCTCTTA |
| | | CTGTCATGCCATCCGTAAGATGCTTTTCTGTGACTGGTGAGTA |
| | | CTCAACCAAGTCATTCTGAGAATAGTGTATGCGGCGACCGAGT |
| | | TGCTCTTGCCCGGCGTCAATACGGGATAATACCGCGCCACATA |
| | | GCAGAACTTTAAAAGTGCTCATCATTGGAAAACGTTCTTCGGG |
| | | GCGAAAACTCTCAAGGATCTTACCGCTGTTGAGATCCAGTTCG |
| | | ATGTAACCCACTCGTGCACCCAACTGATCTTCAGCATCTTTTA |
| | | CTTTCACCAGCGTTTCTGGGTGAGCAAAAACAGGAAGGCAAAA |
| | | TGCCGCAAAAAAGGGAATAAGGGCGACACGGAAATGTTGAATA |
| | | CTCATACTCTTCCTTTTTCAATATTATTGAAGCATTTATCAGG |
| | | GTTATTGTCTCATGAGCGGATACATATTTGAATGTATTTAGAA |
| | | AAATAAACAAATAGGGGTTCCGCGCACATTTCCCCGAAAAGTG |
| | | CCAC |
| 86 | Compound 5 | CTAAATTGTAAGCGTTAATATTTTGTTAAAATTCGCGTTAAAT |
| | (pMK-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGAAGGAAGGCCGTCAAGGCCGCAT [symbol] ATGTACAGAATG |
| | | CAGCTGCTGAGCTGTATCGCCCTGTCTCTGGCCCTGGTCACAA |
| | | ATAGCGCCCCTACCAGCAGCAGCACCAAGAAAACACAGCTGCA |
| | | ACTGGAACACCTCCTGCTGGACCTGCAGATGATCCTGAACGGC |
| | | ATCAACAACTACAAGAACCCCAAGCTGACCCGGATGCTGACCT |
| | | TCAAGTTCTACATGCCCAAGAAGGCCACCGAGCTGAAGCACCT |
| | | CCAGTGCCTGGAAGAGGAACTGAAGCCCCTGGAAGAAGTGCTG |
| | | AATCTGGCCCAGAGCAAGAACTTCCACCTGAGGCCTAGGGACC |
| | | TGATCAGCAACATCAACGTGATCGTGCTGGAACTGAAAGGCAG |
| | | CGAGACAACCTTCATGTGCGAGTACGCCGACGAGACAGCTACC |
| | | ATCGTGGAATTTCTGAACCGGTGGATCACCTTCTGCCAGAGCA |
| | | TCATCAGCACCCTGACCTGAATAGTGAGTCGTATTAACGTACC |
| | | AACAAGCAGAATCATCACGAAGTGGTACTTGACCACTTCGTGA |
| | | TGATTCTGCTTTATCTTAGAGGCATATCCCTACGTACCAACAA |
| | | GAGCTTCCTACAGCACAACAAACTTGTTGTTGTGCTGTAGGAA |
| | | GCTCTTTATCTTAGAGGCATATCCCTACGTACCAACAAGATCC |
| | | GCAGACGTGTAAATGTACTTGACATTTACACGTCTGCGGATCT |
| | | TTATCTTAGAGGCATATCCCTTTTATCTTAGAGGCATATCCCT |
| | | CTGGGCCTCATGGGCCTTCCTTTCACTGCCCGCTTTCCAGTCG |
| | | GGAAACCTGTCGTGCCAGCTGCATTAACATGGTCATAGCTGTT |
| | | TCCTTGCGTATTGGGCGCTCTCCGCTTCCTCGCTCACTGACTC |
| | | GCTGCGCTCGGTCGTTCGGGTAAAGCCTGGGGTGCCTAATGAG |
| | | CAAAAGGCCAGCAAAAGGCCAGGAACCGTAAAAAGGCCGCGTT |
| | | GCTGGCGTTTTTCCATAGGCTCCGCCCCCCTGACGAGCATCAC |
| | | AAAAATCGACGCTCAAGTCAGAGGTGGCGAAACCCGACAGGAC |
| | | TATAAAGATACCAGGCGTTTCCCCCTGGAAGCTCCCTCGTGCG |
| | | CTCTCCTGTTCCGACCCTGCCGCTTACCGGATACCTGTCCGCC |
| | | TTTCTCCCTTCGGGAAGCGTGGCGCTTTCTCATAGCTCACGCT |
| | | GTAGGTATCTCAGTTCGGTGTAGGTCGTTCGCTCCAAGCTGGG |
| | | CTGTGTGCACGAACCCCCCGTTCAGCCCGACCGCTGCGCCTTA |
| | | TCCGGTAACTATCGTCTTGAGTCCAACCCGGTAAGACACGACT |
| | | TATCGCCACTGGCAGCAGCCACTGGTAACAGGATTAGCAGAGC |
| | | GAGGTATGTAGGCGGTGCTACAGAGTTCTTGAAGTGGTGGCCT |
| | | AACTACGGCTACACTAGAAGAACAGTATTTGGTATCTGCGCTC |
| | | TGCTGAAGCCAGTTACCTTCGGAAAAAGAGTTGGTAGCTCTTG |
| | | ATCCGGCAAACAAACCACCGCTGGTAGCGGTGGTTTTTTTGTT |
| | | TGCAAGCAGCAGATTACGCGCAGAAAAAAAGGATCTCAAGAAG |
| | | ATCCTTTGATCTTTTCTACGGGGTCTGACGCTCAGTGGAACGA |
| | | AAACTCACGTTAAGGGATTTTGGTCATGAGATTATCAAAAAGG |
| | | ATCTTCACCTAGATCCTTTTAAATTAAAAATGAAGTTTTAAAT |
| | | CAATCTAAAGTATATATGAGTAAACTTGGTCTGACAGTTATTA |
| | | GAAAAATTCATCCAGCAGACGATAAAACGCAATACGCTGGCTA |
| | | TCCGGTGCCGCAATGCCATACAGCACCAGAAAACGATCCGCCC |
| | | ATTCGCCGCCCAGTTCTTCCGCAATATCACGGGTGGCCAGCGC |
| | | AATATCCTGATAACGATCCGCCACGCCCAGACGGCCGCAATCA |
| | | ATAAAGCCGCTAAAACGGCCATTTTCCACCATAATGTTCGGCA |
| | | GGCACGCATCACCATGGGTCACCACCAGATCTTCGCCATCCGG |
| | | CATGCTCGCTTTCAGACGCGCAAACAGCTCTGCCGGTGCCAGG |
| | | CCCTGATGTTCTTCATCCAGATCATCCTGATCCACCAGGCCCG |
| | | CTTCCATACGGGTACGCGCACGTTCAATACGATGTTTCGCCTG |
| | | ATGATCAAACGGACAGGTCGCCGGGTCCAGGGTATGCAGACGA |
| | | CGCATGGCATCCGCCATAATGCTCACTTTTTCTGCCGGCGCCA |
| | | GATGGCTAGACAGCAGATCCTGACCCGGCACTTCGCCCAGCAG |
| | | CAGCCAATCACGGCCCGCTTCGGTCACCACATCCAGCACCGCC |
| | | GCACACGGAACACCGGTGGTGGCCAGCCAGCTCAGACGCGCCG |
| | | CTTCATCCTGCAGCTCGTTCAGCGCACCGCTCAGATCGGTTTT |
| | | CACAAACAGCACCGGACGACCCTGCGCGCTCAGACGAAACACC |
| | | GCCGCATCAGAGCAGCCAATGGTCTGCTGCGCCCAATCATAGC |
| | | CAAACAGACGTTCCACCCACGCTGCCGGGCTACCCGCATGCAG |
| | | GCCATCCTGTTCAATCATACTCTTCCTTTTTCAATATTATTGA |
| | | AGCATTTATCAGGGTTATTGTCTCATGAGCGGATACATATTTG |
| | | AATGTATTTAGAAAAATAAACAAATAGGGGTTCCGCGCACATT |
| | | TCCCCGAAAAGTGCCAC |
| 87 | Compound 6 | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCGCATGCCACCATGTACAGAATG |
| | | CAGCTGCTGAGCTGTATCGCCCTGTCTCTGGCCCTGGTCACAA |
| | | ATAGCGCCCCTACCAGCAGCAGCACCAAGAAAACACAGCTGCA |
| | | ACTGGAACACCTCCTGCTGGACCTGCAGATGATCCTGAACGGC |
| | | ATCAACAACTACAAGAACCCCAAGCTGACCCGGATGCTGACCT |
| | | TCAAGTTCTACATGCCCAAGAAGGCCACCGAGCTGAAGCACCT |
| | | CCAGTGCCTGGAAGAGGAACTGAAGCCCCTGGAAGAAGTGCTG |
| | | AATCTGGCCCAGAGCAAGAACTTCCACCTGAGGCCTAGGGACC |
| | | TGATCAGCAACATCAACGTGATCGTGCTGGAACTGAAAGGCAG |
| | | CGAGACAACCTTCATGTGCGAGTACGCCGACGAGACAGCTACC |
| | | ATCGTGGAATTTCTGAACCGGTGGATCACCTTCTGCCAGAGCA |
| | | TCATCAGCACCCTGACCTGAATAGTGAGTCGTATTAACGTACC |
| | | AACAAGGAGATTAGGGTCTGTGAGATACTTGATCTCACAGACC |
| | | CTAATCTCCTTTATCTTAGAGGCATATCCCTACGTACCAACAA |
| | | GATGCCATGAAGACCAAGACAACTTGTGTCTTGGTCTTCATGG |
| | | CATCTTTATCTTAGAGGCATATCCCTACGTACCAACAAGCCTG |
| | | ATGGGAATGGAACCTAACTTGTAGGTTCCATTCCCATCAGGCT |
| | | TTATCTTAGAGGCATATCCCTTTTATCTTAGAGGCATATCCCT |
| | | CTGGGCCTCATGGGCCTTCCGCTCACTGCCCGCTTTCCAGTCG |
| | | GGAAACCTGTCGTGCCAGCTGCATTAACATGGTCATAGCTGTT |
| | | TCCTTGCGTATTGGGCGCTCTCCGCTTCCTCGCTCACTGACTC |
| | | GCTGCGCTCGGTCGTTCGGGTAAAGCCTGGGGTGCCTAATGAG |
| | | CAAAAGGCCAGCAAAAGGCCAGGAACCGTAAAAAGGCCGCGTT |
| | | GCTGGCGTTTTTCCATAGGCTCCGCCCCCCTGACGAGCATCAC |
| | | AAAAATCGACGCTCAAGTCAGAGGTGGCGAAACCCGACAGGAC |
| | | TATAAAGATACCAGGCGTTTCCCCCTGGAAGCTCCCTCGTGCG |
| | | CTCTCCTGTTCCGACCCTGCCGCTTACCGGATACCTGTCCGCC |
| | | TTTCTCCCTTCGGGAAGCGTGGCGCTTTCTCATAGCTCACGCT |
| | | GTAGGTATCTCAGTTCGGTGTAGGTCGTTCGCTCCAAGCTGGG |
| | | CTGTGTGCACGAACCCCCCGTTCAGCCCGACCGCTGCGCCTTA |
| | | TCCGGTAACTATCGTCTTGAGTCCAACCCGGTAAGACACGACT |
| | | TATCGCCACTGGCAGCAGCCACTGGTAACAGGATTAGCAGAGC |
| | | GAGGTATGTAGGCGGTGCTACAGAGTTCTTGAAGTGGTGGCCT |
| | | AACTACGGCTACACTAGAAGAACAGTATTTGGTATCTGCGCTC |
| | | TGCTGAAGCCAGTTACCTTCGGAAAAAGAGTTGGTAGCTCTTG |
| | | ATCCGGCAAACAAACCACCGCTGGTAGCGGTGGTTTTTTTGTT |
| | | TGCAAGCAGCAGATTACGCGCAGAAAAAAAGGATCTCAAGAAG |
| | | ATCCTTTGATCTTTTCTACGGGGTCTGACGCTCAGTGGAACGA |
| | | AAACTCACGTTAAGGGATTTTGGTCATGAGATTATCAAAAAGG |
| | | ATCTTCACCTAGATCCTTTTAAATTAAAAATGAAGTTTTAAAT |
| | | CAATCTAAAGTATATATGAGTAAACTTGGTCTGACAGTTACCA |
| | | ATGCTTAATCAGTGAGGCACCTATCTCAGCGATCTGTCTATTT |
| | | CGTTCATCCATAGTTGCCTGACTCCCCGTCGTGTAGATAACTA |
| | | CGATACGGGAGGGCTTACCATCTGGCCCCAGTGCTGCAATGAT |
| | | ACCGCGAGAACCACGCTCACCGGCTCCAGATTTATCAGCAATA |
| | | AACCAGCCAGCCGGAAGGGCCGAGCGCAGAAGTGGTCCTGCAA |
| | | CTTTATCCGCCTCCATCCAGTCTATTAATTGTTGCCGGGAAGC |
| | | TAGAGTAAGTAGTTCGCCAGTTAATAGTTTGCGCAACGTTGTT |
| | | GCCATTGCTACAGGCATCGTGGTGTCACGCTCGTCGTTTGGTA |
| | | TGGCTTCATTCAGCTCCGGTTCCCAACGATCAAGGCGAGTTAC |
| | | ATGATCCCCCATGTTGTGCAAAAAAGCGGTTAGCTCCTTCGGT |
| | | CCTCCGATCGTTGTCAGAAGTAAGTTGGCCGCAGTGTTATCAC |
| | | TCATGGTTATGGCAGCACTGCATAATTCTCTTACTGTCATGCC |
| | | ATCCGTAAGATGCTTTTCTGTGACTGGTGAGTACTCAACCAAG |
| | | TCATTCTGAGAATAGTGTATGCGGCGACCGAGTTGCTCTTGCC |
| | | CGGCGTCAATACGGGATAATACCGCGCCACATAGCAGAACTTT |
| | | AAAAGTGCTCATCATTGGAAAACGTTCTTCGGGGCGAAAACTC |
| | | TCAAGGATCTTACCGCTGTTGAGATCCAGTTCGATGTAACCCA |
| | | CTCGTGCACCCAACTGATCTTCAGCATCTTTTACTTTCACCAG |
| | | CGTTTCTGGGTGAGCAAAAACAGGAAGGCAAAATGCCGCAAAA |
| | | AAGGGAATAAGGGCGACACGGAAATGTTGAATACTCATACTCT |
| | | TCCTTTTTCAATATTATTGAAGCATTTATCAGGGTTATTGTCT |
| | | CATGAGCGGATACATATTTGAATGTATTTAGAAAAATAAACAA |
| | | ATAGGGGTTCCGCGCACATTTCCCCGAAAAGTGCCAC |
| 88 | Compound 7* | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCGCAT [symbol] ATGTTGTTGCTG |
| | | CTGCTCGCCTGTATTGCCCTGGCCTCTACAGCCGCCGCTACAA |
| | | ATTCTGCCCCTACCAGCAGCTCCACCAAGAAAACCCAGCTGCA |
| | | ACTGGAACATCTGCTGCTGGACCTGCAGATGATCCTGAACGGC |
| | | ATCAACAACTACAAGAACCCCAAGCTGACCCGGATGCTGACCT |
| | | TCAAGTTCTACATGCCCAAGAAGGCCACCGAGCTGAAGCACCT |
| | | CCAGTGCCTGGAAGAGGAACTGAAGCCCCTGGAAGAAGTGCTG |
| | | AATCTGGCCCAGAGCAAGAACTTCCACCTGAGGCCTAGGGACC |
| | | TGATCAGCAACATCAACGTGATCGTGCTGGAACTGAAAGGCAG |
| | | CGAGACAACCTTCATGTGCGAGTACGCCGACGAGACAGCTACC |
| | | ATCGTGGAATTTCTGAACCGGTGGATCACCTTCTGCCAGAGCA |
| | | TCATCAGCACCCTGACCTGAATAGTGAGTCGTATTAACGTACC |
| | | AACAAGCAGAATCATCACGAAGTGGTACTTGACCACTTCGTGA |
| | | TGATTCTGCTTTATCTTAGAGGCATATCCCTACGTACCAACAA |
| | | GAGCTTCCTACAGCACAACAAACTTGTTGTTGTGCTGTAGGAA |
| | | GCTCTTTATCTTAGAGGCATATCCCTACGTACCAACAAGATCC |
| | | GCAGACGTGTAAATGTACTTGACATTTACACGTCTGCGGATCT |
| | | TTATCTTAGAGGCATATCCCTTTTATCTTAGAGGCATATCCCT |
| | | CTGGGCCTCATGGGCCTTCCGCTCACTGCCCGCTTTCCAGTCG |
| | | GGAAACCTGTCGTGCCAGCTGCATTAACATGGTCATAGCTGTT |
| | | TCCTTGCGTATTGGGCGCTCTCCGCTTCCTCGCTCACTGACTC |
| | | GCTGCGCTCGGTCGTTCGGGTAAAGCCTGGGGTGCCTAATGAG |
| | | CAAAAGGCCAGCAAAAGGCCAGGAACCGTAAAAAGGCCGCGTT |
| | | GCTGGCGTTTTTCCATAGGCTCCGCCCCCCTGACGAGCATCAC |
| | | AAAAATCGACGCTCAAGTCAGAGGTGGCGAAACCCGACAGGAC |
| | | TATAAAGATACCAGGCGTTTCCCCCTGGAAGCTCCCTCGTGCG |
| | | CTCTCCTGTTCCGACCCTGCCGCTTACCGGATACCTGTCCGCC |
| | | TTTCTCCCTTCGGGAAGCGTGGCGCTTTCTCATAGCTCACGCT |
| | | GTAGGTATCTCAGTTCGGTGTAGGTCGTTCGCTCCAAGCTGGG |
| | | CTGTGTGCACGAACCCCCCGTTCAGCCCGACCGCTGCGCCTTA |
| | | TCCGGTAACTATCGTCTTGAGTCCAACCCGGTAAGACACGACT |
| | | TATCGCCACTGGCAGCAGCCACTGGTAACAGGATTAGCAGAGC |
| | | GAGGTATGTAGGCGGTGCTACAGAGTTCTTGAAGTGGTGGCCT |
| | | AACTACGGCTACACTAGAAGAACAGTATTTGGTATCTGCGCTC |
| | | TGCTGAAGCCAGTTACCTTCGGAAAAAGAGTTGGTAGCTCTTG |
| | | ATCCGGCAAACAAACCACCGCTGGTAGCGGTGGTTTTTTTGTT |
| | | TGCAAGCAGCAGATTACGCGCAGAAAAAAAGGATCTCAAGAAG |
| | | ATCCTTTGATCTTTTCTACGGGGTCTGACGCTCAGTGGAACGA |
| | | AAACTCACGTTAAGGGATTTTGGTCATGAGATTATCAAAAAGG |
| | | ATCTTCACCTAGATCCTTTTAAATTAAAAATGAAGTTTTAAAT |
| | | CAATCTAAAGTATATATGAGTAAACTTGGTCTGACAGTTACCA |
| | | ATGCTTAATCAGTGAGGCACCTATCTCAGCGATCTGTCTATTT |
| | | CGTTCATCCATAGTTGCCTGACTCCCCGTCGTGTAGATAACTA |
| | | CGATACGGGAGGGCTTACCATCTGGCCCCAGTGCTGCAATGAT |
| | | ACCGCGAGAACCACGCTCACCGGCTCCAGATTTATCAGCAATA |
| | | AACCAGCCAGCCGGAAGGGCCGAGCGCAGAAGTGGTCCTGCAA |
| | | CTTTATCCGCCTCCATCCAGTCTATTAATTGTTGCCGGGAAGC |
| | | TAGAGTAAGTAGTTCGCCAGTTAATAGTTTGCGCAACGTTGTT |
| | | GCCATTGCTACAGGCATCGTGGTGTCACGCTCGTCGTTTGGTA |
| | | TGGCTTCATTCAGCTCCGGTTCCCAACGATCAAGGCGAGTTAC |
| | | ATGATCCCCCATGTTGTGCAAAAAAGCGGTTAGCTCCTTCGGT |
| | | CCTCCGATCGTTGTCAGAAGTAAGTTGGCCGCAGTGTTATCAC |
| | | TCATGGTTATGGCAGCACTGCATAATTCTCTTACTGTCATGCC |
| | | ATCCGTAAGATGCTTTTCTGTGACTGGTGAGTACTCAACCAAG |
| | | TCATTCTGAGAATAGTGTATGCGGCGACCGAGTTGCTCTTGCC |
| | | CGGCGTCAATACGGGATAATACCGCGCCACATAGCAGAACTTT |
| | | AAAAGTGCTCATCATTGGAAAACGTTCTTCGGGGCGAAAACTC |
| | | TCAAGGATCTTACCGCTGTTGAGATCCAGTTCGATGTAACCCA |
| | | CTCGTGCACCCAACTGATCTTCAGCATCTTTTACTTTCACCAG |
| | | CGTTTCTGGGTGAGCAAAAACAGGAAGGCAAAATGCCGCAAAA |
| | | AAGGGAATAAGGGCGACACGGAAATGTTGAATACTCATACTCT |
| | | TCCTTTTTCAATATTATTGAAGCATTTATCAGGGTTATTGTCT |
| | | CATGAGCGGATACATATTTGAATGTATTTAGAAAAATAAACAA |
| | | ATAGGGGTTCCGCGCACATTTCCCCGAAAAGTGCCAC |
| 89 | Compound 8* | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCGCAT [symbol] ATGTTGTTGCTG |
| | | CTGCTCGCCTGTATTGCCCTGGCCTCTACAGCCGCCGCTACAA |
| | | ATTCTGCCCCTACCAGCAGCTCCACCAAGAAAACCCAGCTGCA |
| | | ACTGGAACATCTGCTGCTGGACCTGCAGATGATCCTGAACGGC |
| | | ATCAACAACTACAAGAACCCCAAGCTGACCCGGATGCTGACCT |
| | | TCAAGTTCTACATGCCCAAGAAGGCCACCGAGCTGAAGCACCT |
| | | CCAGTGCCTGGAAGAGGAACTGAAGCCCCTGGAAGAAGTGCTG |
| | | AATCTGGCCCAGAGCAAGAACTTCCACCTGAGGCCTAGGGACC |
| | | TGATCAGCAACATCAACGTGATCGTGCTGGAACTGAAAGGCAG |
| | | CGAGACAACCTTCATGTGCGAGTACGCCGACGAGACAGCTACC |
| | | ATCGTGGAATTTCTGAACCGGTGGATCACCTTCTGCCAGAGCA |
| | | TCATCAGCACCCTGACCTGAATAGTGAGTCGTATTAACGTACC |
| | | AACAAGCAGAATCATCACGAAGTGGTACTTGACCACTTCGTGA |
| | | TGATTCTGCTTTATCTTAGAGGCATATCCCTACGTACCAACAA |
| | | GAGCTTCCTACAGCACAACAAACTTGTTGTTGTGCTGTAGGAA |
| | | GCTCTTTATCTTAGAGGCATATCCCTACGTACCAACAAGATCC |
| | | GCAGACGTGTAAATGTACTTGACATTTACACGTCTGCGGATCT |
| | | TTATCTTAGAGGCATATCCCTACGTACCAACAAGCGCAAGAAA |
| | | TCCCGGTATAAACTTGTTATACCGGGATTTCTTGCGCTTTATC |
| | | TTAGAGGCATATCCCTACGTACCAACAAGGCGAGGCAGCTTGA |
| | | GTTAAAACTTGTTTAACTCAAGCTGCCTCGCCTTTATCTTAGA |
| | | GGCATATCCCTTTTATCTTAGAGGCATATCCCTCTGGGCCTCA |
| | | TGGGCCTTCCGCTCACTGCCCGCTTTCCAGTCGGGAAACCTGT |
| | | CGTGCCAGCTGCATTAACATGGTCATAGCTGTTTCCTTGCGTA |
| | | TTGGGCGCTCTCCGCTTCCTCGCTCACTGACTCGCTGCGCTCG |
| | | GTCGTTCGGGTAAAGCCTGGGGTGCCTAATGAGCAAAAGGCCA |
| | | GCAAAAGGCCAGGAACCGTAAAAAGGCCGCGTTGCTGGCGTTT |
| | | TTCCATAGGCTCCGCCCCCCTGACGAGCATCACAAAAATCGAC |
| | | GCTCAAGTCAGAGGTGGCGAAACCCGACAGGACTATAAAGATA |
| | | CCAGGCGTTTCCCCCTGGAAGCTCCCTCGTGCGCTCTCCTGTT |
| | | CCGACCCTGCCGCTTACCGGATACCTGTCCGCCTTTCTCCCTT |
| | | CGGGAAGCGTGGCGCTTTCTCATAGCTCACGCTGTAGGTATCT |
| | | CAGTTCGGTGTAGGTCGTTCGCTCCAAGCTGGGCTGTGTGCAC |
| | | GAACCCCCCGTTCAGCCCGACCGCTGCGCCTTATCCGGTAACT |
| | | ATCGTCTTGAGTCCAACCCGGTAAGACACGACTTATCGCCACT |
| | | GGCAGCAGCCACTGGTAACAGGATTAGCAGAGCGAGGTATGTA |
| | | GGCGGTGCTACAGAGTTCTTGAAGTGGTGGCCTAACTACGGCT |
| | | ACACTAGAAGAACAGTATTTGGTATCTGCGCTCTGCTGAAGCC |
| | | AGTTACCTTCGGAAAAAGAGTTGGTAGCTCTTGATCCGGCAAA |
| | | CAAACCACCGCTGGTAGCGGTGGTTTTTTTGTTTGCAAGCAGC |
| | | AGATTACGCGCAGAAAAAAAGGATCTCAAGAAGATCCTTTGAT |
| | | CTTTTCTACGGGGTCTGACGCTCAGTGGAACGAAAACTCACGT |
| | | TAAGGGATTTTGGTCATGAGATTATCAAAAAGGATCTTCACCT |
| | | AGATCCTTTTAAATTAAAAATGAAGTTTTAAATCAATCTAAAG |
| | | TATATATGAGTAAACTTGGTCTGACAGTTACCAATGCTTAATC |
| | | AGTGAGGCACCTATCTCAGCGATCTGTCTATTTCGTTCATCCA |
| | | TAGTTGCCTGACTCCCCGTCGTGTAGATAACTACGATACGGGA |
| | | GGGCTTACCATCTGGCCCCAGTGCTGCAATGATACCGCGAGAA |
| | | CCACGCTCACCGGCTCCAGATTTATCAGCAATAAACCAGCCAG |
| | | CCGGAAGGGCCGAGCGCAGAAGTGGTCCTGCAACTTTATCCGC |
| | | CTCCATCCAGTCTATTAATTGTTGCCGGGAAGCTAGAGTAAGT |
| | | AGTTCGCCAGTTAATAGTTTGCGCAACGTTGTTGCCATTGCTA |
| | | CAGGCATCGTGGTGTCACGCTCGTCGTTTGGTATGGCTTCATT |
| | | CAGCTCCGGTTCCCAACGATCAAGGCGAGTTACATGATCCCCC |
| | | ATGTTGTGCAAAAAAGCGGTTAGCTCCTTCGGTCCTCCGATCG |
| | | TTGTCAGAAGTAAGTTGGCCGCAGTGTTATCACTCATGGTTAT |
| | | GGCAGCACTGCATAATTCTCTTACTGTCATGCCATCCGTAAGA |
| | | TGCTTTTCTGTGACTGGTGAGTACTCAACCAAGTCATTCTGAG |
| | | AATAGTGTATGCGGCGACCGAGTTGCTCTTGCCCGGCGTCAAT |
| | | ACGGGATAATACCGCGCCACATAGCAGAACTTTAAAAGTGCTC |
| | | ATCATTGGAAAACGTTCTTCGGGGCGAAAACTCTCAAGGATCT |
| | | TACCGCTGTTGAGATCCAGTTCGATGTAACCCACTCGTGCACC |
| | | CAACTGATCTTCAGCATCTTTTACTTTCACCAGCGTTTCTGGG |
| | | TGAGCAAAAACAGGAAGGCAAAATGCCGCAAAAAAGGGAATAA |
| | | GGGCGACACGGAAATGTTGAATACTCATACTCTTCCTTTTTCA |
| | | ATATTATTGAAGCATTTATCAGGGTTATTGTCTCATGAGCGGA |
| | | TACATATTTGAATGTATTTAGAAAAATAAACAAATAGGGGTTC |
| | | CGCGCACATTTCCCCGAAAAGTGCCAC |
| 90 | Compound 9* | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGAAGGAAGGCCGTCAAGGCCGCAT [symbol] ATGTTGTTGCTG |
| | | CTGCTCGCCTGTATTGCCCTGGCCTCTACAGCCGCCGCTACAA |
| | | ATTCTGCCCCTACCAGCAGCTCCACCAAGAAAACCCAGCTGCA |
| | | ACTGGAACATCTGCTGCTGGACCTGCAGATGATCCTGAACGGC |
| | | ATCAACAACTACAAGAACCCCAAGCTGACCCGGATGCTGACCT |
| | | TCAAGTTCTACATGCCCAAGAAGGCCACCGAGCTGAAGCACCT |
| | | CCAGTGCCTGGAAGAGGAACTGAAGCCCCTGGAAGAAGTGCTG |
| | | AATCTGGCCCAGAGCAAGAACTTCCACCTGAGGCCTAGGGACC |
| | | TGATCAGCAACATCAACGTGATCGTGCTGGAACTGAAAGGCAG |
| | | CGAGACAACCTTCATGTGCGAGTACGCCGACGAGACAGCTACC |
| | | ATCGTGGAATTTCTGAACCGGTGGATCACCTTCTGCCAGAGCA |
| | | TCATCAGCACCCTGACCTGAATAGTGAGTCGTATTAACGTACC |
| | | AACAAGGAGTACCCTGATGAGATCACTTGGATCTCATCAGGGT |
| | | ACTCCTTTATCTTAGAGGCATATCCCTACGTACCAACAAGGAG |
| | | TACCCTGATGAGATCACTTGGATCTCATCAGGGTACTCCTTTA |
| | | TCTTAGAGGCATATCCCTACGTACCAACAAGGAGTACCCTGAT |
| | | GAGATCACTTGGATCTCATCAGGGTACTCCTTTATCTTAGAGG |
| | | CATATCCCTTTTATCTTAGAGGCATATCCCTCTGGGCCTCATG |
| | | GGCCTTCCTTTCACTGCCCGCTTTCCAGTCGGGAAACCTGTCG |
| | | TGCCAGCTGCATTAACATGGTCATAGCTGTTTCCTTGCGTATT |
| | | GGGCGCTCTCCGCTTCCTCGCTCACTGACTCGCTGCGCTCGGT |
| | | CGTTCGGGTAAAGCCTGGGGTGCCTAATGAGCAAAAGGCCAGC |
| | | AAAAGGCCAGGAACCGTAAAAAGGCCGCGTTGCTGGCGTTTTT |
| | | CCATAGGCTCCGCCCCCCTGACGAGCATCACAAAAATCGACGC |
| | | TCAAGTCAGAGGTGGCGAAACCCGACAGGACTATAAAGATACC |
| | | AGGCGTTTCCCCCTGGAAGCTCCCTCGTGCGCTCTCCTGTTCC |
| | | GACCCTGCCGCTTACCGGATACCTGTCCGCCTTTCTCCCTTCG |
| | | GGAAGCGTGGCGCTTTCTCATAGCTCACGCTGTAGGTATCTCA |
| | | GTTCGGTGTAGGTCGTTCGCTCCAAGCTGGGCTGTGTGCACGA |
| | | ACCCCCCGTTCAGCCCGACCGCTGCGCCTTATCCGGTAACTAT |
| | | CGTCTTGAGTCCAACCCGGTAAGACACGACTTATCGCCACTGG |
| | | CAGCAGCCACTGGTAACAGGATTAGCAGAGCGAGGTATGTAGG |
| | | CGGTGCTACAGAGTTCTTGAAGTGGTGGCCTAACTACGGCTAC |
| | | ACTAGAAGAACAGTATTTGGTATCTGCGCTCTGCTGAAGCCAG |
| | | TTACCTTCGGAAAAAGAGTTGGTAGCTCTTGATCCGGCAAACA |
| | | AACCACCGCTGGTAGCGGTGGTTTTTTTGTTTGCAAGCAGCAG |
| | | ATTACGCGCAGAAAAAAAGGATCTCAAGAAGATCCTTTGATCT |
| | | TTTCTACGGGGTCTGACGCTCAGTGGAACGAAAACTCACGTTA |
| | | AGGGATTTTGGTCATGAGATTATCAAAAAGGATCTTCACCTAG |
| | | ATCCTTTTAAATTAAAAATGAAGTTTTAAATCAATCTAAAGTA |
| | | TATATGAGTAAACTTGGTCTGACAGTTATTAGAAAAATTCATC |
| | | CAGCAGACGATAAAACGCAATACGCTGGCTATCCGGTGCCGCA |
| | | ATGCCATACAGCACCAGAAAACGATCCGCCCATTCGCCGCCCA |
| | | GTTCTTCCGCAATATCACGGGTGGCCAGCGCAATATCCTGATA |
| | | ACGATCCGCCACGCCCAGACGGCCGCAATCAATAAAGCCGCTA |
| | | AAACGGCCATTTTCCACCATAATGTTCGGCAGGCACGCATCAC |
| | | CATGGGTCACCACCAGATCTTCGCCATCCGGCATGCTCGCTTT |
| | | CAGACGCGCAAACAGCTCTGCCGGTGCCAGGCCCTGATGTTCT |
| | | TCATCCAGATCATCCTGATCCACCAGGCCCGCTTCCATACGGG |
| | | TACGCGCACGTTCAATACGATGTTTCGCCTGATGATCAAACGG |
| | | ACAGGTCGCCGGGTCCAGGGTATGCAGACGACGCATGGCATCC |
| | | GCCATAATGCTCACTTTTTCTGCCGGCGCCAGATGGCTAGACA |
| | | GCAGATCCTGACCCGGCACTTCGCCCAGCAGCAGCCAATCACG |
| | | GCCCGCTTCGGTCACCACATCCAGCACCGCCGCACACGGAACA |
| | | CCGGTGGTGGCCAGCCAGCTCAGACGCGCCGCTTCATCCTGCA |
| | | GCTCGTTCAGCGCACCGCTCAGATCGGTTTTCACAAACAGCAC |
| | | CGGACGACCCTGCGCGCTCAGACGAAACACCGCCGCATCAGAG |
| | | CAGCCAATGGTCTGCTGCGCCCAATCATAGCCAAACAGACGTT |
| | | CCACCCACGCTGCCGGGCTACCCGCATGCAGGCCATCCTGTTC |
| | | AATCATACTCTTCCTTTTTCAATATTATTGAAGCATTTATCAG |
| | | GGTTATTGTCTCATGAGCGGATACATATTTGAATGTATTTAGA |
| | | AAAATAAACAAATAGGGGTTCCGCGCACATTTCCCCGAAAAGT |
| | | GCCAC |
| 91 | Compound 10* | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGAAGGAAGGCCGTCAAGGCCGCAT [symbol] ATGTTGTTGCTG |
| | | CTGCTCGCCTGTATTGCCCTGGCCTCTACAGCCGCCGCTACAA |
| | | ATTCTGCCCCTACCAGCAGCTCCACCAAGAAAACCCAGCTGCA |
| | | ACTGGAACATCTGCTGCTGGACCTGCAGATGATCCTGAACGGC |
| | | ATCAACAACTACAAGAACCCCAAGCTGACCCGGATGCTGACCT |
| | | TCAAGTTCTACATGCCCAAGAAGGCCACCGAGCTGAAGCACCT |
| | | CCAGTGCCTGGAAGAGGAACTGAAGCCCCTGGAAGAAGTGCTG |
| | | AATCTGGCCCAGAGCAAGAACTTCCACCTGAGGCCTAGGGACC |
| | | TGATCAGCAACATCAACGTGATCGTGCTGGAACTGAAAGGCAG |
| | | CGAGACAACCTTCATGTGCGAGTACGCCGACGAGACAGCTACC |
| | | ATCGTGGAATTTCTGAACCGGTGGATCACCTTCTGCCAGAGCA |
| | | TCATCAGCACCCTGACCTGAATAGTGAGTCGTATTAACGTACC |
| | | AACAAGGAGGGCAGAATCATCACGAAGTGGTGAAGTACTTGAC |
| | | TTCACCACTTCGTGATGATTCTGCCCTCCTTTATCTTAGAGGC |
| | | ATATCCCTACGTACCAACAAGAGATGAGCTTCCTACAGCACAA |
| | | CAAATGTGACTTGCACATTTGTTGTGCTGTAGGAAGCTCATCT |
| | | CTTTATCTTAGAGGCATATCCCTACGTACCAACAAGTACAAGA |
| | | TCCGCAGACGTGTAAATGTTCCACTTGGGAACATTTACACGTC |
| | | TGCGGATCTTGTACTTTATCTTAGAGGCATATCCCTTTTATCT |
| | | TAGAGGCATATCCCTCTGGGCCTCATGGGCCTTCCTTTCACTG |
| | | CCCGCTTTCCAGTCGGGAAACCTGTCGTGCCAGCTGCATTAAC |
| | | ATGGTCATAGCTGTTTCCTTGCGTATTGGGCGCTCTCCGCTTC |
| | | CTCGCTCACTGACTCGCTGCGCTCGGTCGTTCGGGTAAAGCCT |
| | | GGGGTGCCTAATGAGCAAAAGGCCAGCAAAAGGCCAGGAACCG |
| | | TAAAAAGGCCGCGTTGCTGGCGTTTTTCCATAGGCTCCGCCCC |
| | | CCTGACGAGCATCACAAAAATCGACGCTCAAGTCAGAGGTGGC |
| | | GAAACCCGACAGGACTATAAAGATACCAGGCGTTTCCCCCTGG |
| | | AAGCTCCCTCGTGCGCTCTCCTGTTCCGACCCTGCCGCTTACC |
| | | GGATACCTGTCCGCCTTTCTCCCTTCGGGAAGCGTGGCGCTTT |
| | | CTCATAGCTCACGCTGTAGGTATCTCAGTTCGGTGTAGGTCGT |
| | | TCGCTCCAAGCTGGGCTGTGTGCACGAACCCCCCGTTCAGCCC |
| | | GACCGCTGCGCCTTATCCGGTAACTATCGTCTTGAGTCCAACC |
| | | CGGTAAGACACGACTTATCGCCACTGGCAGCAGCCACTGGTAA |
| | | CAGGATTAGCAGAGCGAGGTATGTAGGCGGTGCTACAGAGTTC |
| | | TTGAAGTGGTGGCCTAACTACGGCTACACTAGAAGAACAGTAT |
| | | TTGGTATCTGCGCTCTGCTGAAGCCAGTTACCTTCGGAAAAAG |
| | | AGTTGGTAGCTCTTGATCCGGCAAACAAACCACCGCTGGTAGC |
| | | GGTGGTTTTTTTGTTTGCAAGCAGCAGATTACGCGCAGAAAAA |
| | | AAGGATCTCAAGAAGATCCTTTGATCTTTTCTACGGGGTCTGA |
| | | CGCTCAGTGGAACGAAAACTCACGTTAAGGGATTTTGGTCATG |
| | | AGATTATCAAAAAGGATOTTCACCTAGATCCTTTTAAATTAAA |
| | | AATGAAGTTTTAAATCAATCTAAAGTATATATGAGTAAACTTG |
| | | GTCTGACAGTTATTAGAAAAATTCATCCAGCAGACGATAAAAC |
| | | GCAATACGCTGGCTATCCGGTGCCGCAATGCCATACAGCACCA |
| | | GAAAACGATCCGCCCATTCGCCGCCCAGTTCTTCCGCAATATC |
| | | ACGGGTGGCCAGCGCAATATCCTGATAACGATCCGCCACGCCC |
| | | AGACGGCCGCAATCAATAAAGCCGCTAAAACGGCCATTTTCCA |
| | | CCATAATGTTCGGCAGGCACGCATCACCATGGGTCACCACCAG |
| | | ATCTTCGCCATCCGGCATGCTCGCTTTCAGACGCGCAAACAGC |
| | | TCTGCCGGTGCCAGGCCCTGATGTTCTTCATCCAGATCATCCT |
| | | GATCCACCAGGCCCGCTTCCATACGGGTACGCGCACGTTCAAT |
| | | ACGATGTTTCGCCTGATGATCAAACGGACAGGTCGCCGGGTCC |
| | | AGGGTATGCAGACGACGCATGGCATCCGCCATAATGCTCACTT |
| | | TTTCTGCCGGCGCCAGATGGCTAGACAGCAGATCCTGACCCGG |
| | | CACTTCGCCCAGCAGCAGCCAATCACGGCCCGCTTCGGTCACC |
| | | ACATCCAGCACCGCCGCACACGGAACACCGGTGGTGGCCAGCC |
| | | AGCTCAGACGCGCCGCTTCATCCTGCAGCTCGTTCAGCGCACC |
| | | GCTCAGATCGGTTTTCACAAACAGCACCGGACGACCCTGCGCG |
| | | CTCAGACGAAACACCGCCGCATCAGAGCAGCCAATGGTCTGCT |
| | | GCGCCCAATCATAGCCAAACAGACGTTCCACCCACGCTGCCGG |
| | | GCTACCCGCATGCAGGCCATCCTGTTCAATCATACTCTTCCTT |
| | | TTTCAATATTATTGAAGCATTTATCAGGGTTATTGTCTCATGA |
| | | GCGGATACATATTTGAATGTATTTAGAAAAATAAACAAATAGG |
| | | GGTTCCGCGCACATTTCCCCGAAAAGTGCCAC |
| 92 | Compound 11 | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCGCAT [symbol] ATGTGTCACCAG |
| | | CAGCTGGTCATCAGCTGGTTCAGCCTGGTGTTCCTGGCCTCTC |
| | | CTCTGGTGGCCATCTGGGAGCTGAAGAAAGACGTGTACGTGGT |
| | | GGAACTGGACTGGTATCCCGATGCTCCTGGCGAGATGGTGGTG |
| | | CTGACCTGCGATACCCCTGAAGAGGACGGCATCACCTGGACAC |
| | | TGGATCAGTCTAGCGAGGTGCTCGGCAGCGGCAAGACCCTGAC |
| | | CATCCAAGTGAAAGAGTTTGGCGACGCCGGCCAGTACACCTGT |
| | | CACAAAGGCGGAGAAGTGCTGAGCCACAGCCTGCTGCTGCTCC |
| | | ACAAGAAAGAGGATGGCATTTGGAGCACCGACATCCTGAAGGA |
| | | CCAGAAAGAGCCCAAGAACAAGACCTTCCTGAGATGCGAGGCC |
| | | AAGAACTACAGCGGCCGGTTCACATGTTGGTGGCTGACCACCA |
| | | TCAGCACCGACCTGACCTTCAGCGTGAAGTCCAGCAGAGGCAG |
| | | CAGTGATCCTCAGGGCGTTACATGTGGCGCCGCTACACTGTCT |
| | | GCCGAAAGAGTGCGGGGCGACAACAAAGAATACGAGTACAGCG |
| | | TGGAATGCCAAGAGGACAGCGCCTGTCCAGCCGCCGAAGAGTC |
| | | TCTGCCTATCGAAGTGATGGTGGACGCCGTGCACAAGCTGAAG |
| | | TACGAGAACTACACCTCCAGCTTTTTCATCCGGGACATCATCA |
| | | AGCCCGATCCTCCAAAGAACCTGCAGCTGAAGCCTCTGAAGAA |
| | | CAGCAGACAGGTGGAAGTGTCCTGGGAGTACCCCGACACCTGG |
| | | TCTACACCCCACAGCTACTTCAGCCTGACCTTTTGCGTGCAAG |
| | | TGCAGGGCAAGTCCAAGCGCGAGAAAAAGGACCGGGTGTTCAC |
| | | CGACAAGACCAGCGCCACCGTGATCTGCAGAAAGAACGCCAGC |
| | | ATCAGCGTCAGAGCCCAGGACCGGTACTACAGCAGCTCTTGGA |
| | | GCGAATGGGCCAGCGTGCCATGTTCTGGTGGCGGAGGATCTGG |
| | | CGGAGGTGGAAGCGGCGGAGGCGGATCTAGAAATCTGCCTGTG |
| | | GCCACTCCTGATCCTGGCATGTTCCCTTGTCTGCACCACAGCC |
| | | AGAACCTGCTGAGAGCCGTGTCCAACATGCTGCAGAAGGCCAG |
| | | ACAGACCCTGGAATTCTACCCCTGCACCAGCGAGGAAATCGAC |
| | | CACGAGGACATCACCAAGGATAAGACCAGCACCGTGGAAGCCT |
| | | GCCTGCCTCTGGAACTGACCAAGAACGAGAGCTGCCTGAACAG |
| | | CCGGGAAACCAGCTTCATCACCAACGGCTCTTGCCTGGCCAGC |
| | | AGAAAGACCTCCTTCATGATGGCCCTGTGCCTGAGCAGCATCT |
| | | ACGAGGACCTGAAGATGTACCAGGTGGAATTCAAGACCATGAA |
| | | CGCCAAGCTGCTGATGGACCCCAAGCGGCAGATCTTCCTGGAC |
| | | CAGAATATGCTGGCCGTGATCGACGAGCTGATGCAGGCCCTGA |
| | | ACTTCAACAGCGAGACAGTGCCCCAGAAGTCTAGCCTGGAAGA |
| | | ACCCGACTTCTACAAGACCAAGATCAAGCTGTGCATCCTGCTG |
| | | CACGCCTTCCGGATCAGAGCCGTGACCATCGACAGAGTGATGA |
| | | GCTACCTGAACGCCTCCTGAATAGTGAGTCGTATTAACGTACC |
| | | AACAAGTTCCTTCCAAATGGCTCTGTACTTGACAGAGCCATTT |
| | | GGAAGGAACTTTATCTTAGAGGCATATCCCTACGTACCAACAA |
| | | GCATCGTTCACCGAGATCTGAACTTGTCAGATCTCGGTGAACG |
| | | ATGCTTTATCTTAGAGGCATATCCCTACGTACCAACAAGACCA |
| | | GCAGCGGACAAATAAAACTTGTTTATTTGTCCGCTGCTGGTCT |
| | | TTATCTTAGAGGCATATCCCTTTTATCTTAGAGGCATATCCCT |
| | | CTGGGCCTCATGGGCCTTCCGCTCACTGCCCGCTTTCCAGTCG |
| | | GGAAACCTGTCGTGCCAGCTGCATTAACATGGTCATAGCTGTT |
| | | TCCTTGCGTATTGGGCGCTCTCCGCTTCCTCGCTCACTGACTC |
| | | GCTGCGCTCGGTCGTTCGGGTAAAGCCTGGGGTGCCTAATGAG |
| | | CAAAAGGCCAGCAAAAGGCCAGGAACCGTAAAAAGGCCGCGTT |
| | | GCTGGCGTTTTTCCATAGGCTCCGCCCCCCTGACGAGCATCAC |
| | | AAAAATCGACGCTCAAGTCAGAGGTGGCGAAACCCGACAGGAC |
| | | TATAAAGATACCAGGCGTTTCCCCCTGGAAGCTCCCTCGTGCG |
| | | CTCTCCTGTTCCGACCCTGCCGCTTACCGGATACCTGTCCGCC |
| | | TTTCTCCCTTCGGGAAGCGTGGCGCTTTCTCATAGCTCACGCT |
| | | GTAGGTATCTCAGTTCGGTGTAGGTCGTTCGCTCCAAGCTGGG |
| | | CTGTGTGCACGAACCCCCCGTTCAGCCCGACCGCTGCGCCTTA |
| | | TCCGGTAACTATCGTCTTGAGTCCAACCCGGTAAGACACGACT |
| | | TATCGCCACTGGCAGCAGCCACTGGTAACAGGATTAGCAGAGC |
| | | GAGGTATGTAGGCGGTGCTACAGAGTTCTTGAAGTGGTGGCCT |
| | | AACTACGGCTACACTAGAAGAACAGTATTTGGTATCTGCGCTC |
| | | TGCTGAAGCCAGTTACCTTCGGAAAAAGAGTTGGTAGCTCTTG |
| | | ATCCGGCAAACAAACCACCGCTGGTAGCGGTGGTTTTTTTGTT |
| | | TGCAAGCAGCAGATTACGCGCAGAAAAAAAGGATCTCAAGAAG |
| | | ATCCTTTGATCTTTTCTACGGGGTCTGACGCTCAGTGGAACGA |
| | | AAACTCACGTTAAGGGATTTTGGTCATGAGATTATCAAAAAGG |
| | | ATCTTCACCTAGATCCTTTTAAATTAAAAATGAAGTTTTAAAT |
| | | CAATCTAAAGTATATATGAGTAAACTTGGTCTGACAGTTACCA |
| | | ATGCTTAATCAGTGAGGCACCTATCTCAGCGATCTGTCTATTT |
| | | CGTTCATCCATAGTTGCCTGACTCCCCGTCGTGTAGATAACTA |
| | | CGATACGGGAGGGCTTACCATCTGGCCCCAGTGCTGCAATGAT |
| | | ACCGCGAGAACCACGCTCACCGGCTCCAGATTTATCAGCAATA |
| | | AACCAGCCAGCCGGAAGGGCCGAGCGCAGAAGTGGTCCTGCAA |
| | | CTTTATCCGCCTCCATCCAGTCTATTAATTGTTGCCGGGAAGC |
| | | TAGAGTAAGTAGTTCGCCAGTTAATAGTTTGCGCAACGTTGTT |
| | | GCCATTGCTACAGGCATCGTGGTGTCACGCTCGTCGTTTGGTA |
| | | TGGCTTCATTCAGCTCCGGTTCCCAACGATCAAGGCGAGTTAC |
| | | ATGATCCCCCATGTTGTGCAAAAAAGCGGTTAGCTCCTTCGGT |
| | | CCTCCGATCGTTGTCAGAAGTAAGTTGGCCGCAGTGTTATCAC |
| | | TCATGGTTATGGCAGCACTGCATAATTCTCTTACTGTCATGCC |
| | | ATCCGTAAGATGCTTTTCTGTGACTGGTGAGTACTCAACCAAG |
| | | TCATTCTGAGAATAGTGTATGCGGCGACCGAGTTGCTCTTGCC |
| | | CGGCGTCAATACGGGATAATACCGCGCCACATAGCAGAACTTT |
| | | AAAAGTGCTCATCATTGGAAAACGTTCTTCGGGGCGAAAACTC |
| | | TCAAGGATCTTACCGCTGTTGAGATCCAGTTCGATGTAACCCA |
| | | CTCGTGCACCCAACTGATCTTCAGCATCTTTTACTTTCACCAG |
| | | CGTTTCTGGGTGAGCAAAAACAGGAAGGCAAAATGCCGCAAAA |
| | | AAGGGAATAAGGGCGACACGGAAATGTTGAATACTCATACTCT |
| | | TCCTTTTTCAATATTATTGAAGCATTTATCAGGGTTATTGTCT |
| | | CATGAGCGGATACATATTTGAATGTATTTAGAAAAATAAACAA |
| | | ATAGGGGTTCCGCGCACATTTCCCCGAAAAGTGCCAC |
| 93 | Compound 12 | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCGCATG [symbol] ATGTGTCACCAG |
| | | CAGCTGGTCATCAGCTGGTTCAGCCTGGTGTTCCTGGCCTCTC |
| | | CTCTGGTGGCCATCTGGGAGCTGAAGAAAGACGTGTACGTGGT |
| | | GGAACTGGACTGGTATCCCGATGCTCCTGGCGAGATGGTGGTG |
| | | CTGACCTGCGATACCCCTGAAGAGGACGGCATCACCTGGACAC |
| | | TGGATCAGTCTAGCGAGGTGCTCGGCAGCGGCAAGACCCTGAC |
| | | CATCCAAGTGAAAGAGTTTGGCGACGCCGGCCAGTACACCTGT |
| | | CACAAAGGCGGAGAAGTGCTGAGCCACAGCCTGCTGCTGCTCC |
| | | ACAAGAAAGAGGATGGCATTTGGAGCACCGACATCCTGAAGGA |
| | | CCAGAAAGAGCCCAAGAACAAGACCTTCCTGAGATGCGAGGCC |
| | | AAGAACTACAGCGGCCGGTTCACATGTTGGTGGCTGACCACCA |
| | | TCAGCACCGACCTGACCTTCAGCGTGAAGTCCAGCAGAGGCAG |
| | | CAGTGATCCTCAGGGCGTTACATGTGGCGCCGCTACACTGTCT |
| | | GCCGAAAGAGTGCGGGGCGACAACAAAGAATACGAGTACAGCG |
| | | TGGAATGCCAAGAGGACAGCGCCTGTCCAGCCGCCGAAGAGTC |
| | | TCTGCCTATCGAAGTGATGGTGGACGCCGTGCACAAGCTGAAG |
| | | TACGAGAACTACACCTCCAGCTTTTTCATCCGGGACATCATCA |
| | | AGCCCGATCCTCCAAAGAACCTGCAGCTGAAGCCTCTGAAGAA |
| | | CAGCAGACAGGTGGAAGTGTCCTGGGAGTACCCCGACACCTGG |
| | | TCTACACCCCACAGCTACTTCAGCCTGACCTTTTGCGTGCAAG |
| | | TGCAGGGCAAGTCCAAGCGCGAGAAAAAGGACCGGGTGTTCAC |
| | | CGACAAGACCAGCGCCACCGTGATCTGCAGAAAGAACGCCAGC |
| | | ATCAGCGTCAGAGCCCAGGACCGGTACTACAGCAGCTCTTGGA |
| | | GCGAATGGGCCAGCGTGCCATGTTCTGGTGGCGGAGGATCTGG |
| | | CGGAGGTGGAAGCGGCGGAGGCGGATCTAGAAATCTGCCTGTG |
| | | GCCACTCCTGATCCTGGCATGTTCCCTTGTCTGCACCACAGCC |
| | | AGAACCTGCTGAGAGCCGTGTCCAACATGCTGCAGAAGGCCAG |
| | | ACAGACCCTGGAATTCTACCCCTGCACCAGCGAGGAAATCGAC |
| | | CACGAGGACATCACCAAGGATAAGACCAGCACCGTGGAAGCCT |
| | | GCCTGCCTCTGGAACTGACCAAGAACGAGAGCTGCCTGAACAG |
| | | CCGGGAAACCAGCTTCATCACCAACGGCTCTTGCCTGGCCAGC |
| | | AGAAAGACCTCCTTCATGATGGCCCTGTGCCTGAGCAGCATCT |
| | | ACGAGGACCTGAAGATGTACCAGGTGGAATTCAAGACCATGAA |
| | | CGCCAAGCTGCTGATGGACCCCAAGCGGCAGATCTTCCTGGAC |
| | | CAGAATATGCTGGCCGTGATCGACGAGCTGATGCAGGCCCTGA |
| | | ACTTCAACAGCGAGACAGTGCCCCAGAAGTCTAGCCTGGAAGA |
| | | ACCCGACTTCTACAAGACCAAGATCAAGCTGTGCATCCTGCTG |
| | | CACGCCTTCCGGATCAGAGCCGTGACCATCGACAGAGTGATGA |
| | | GCTACCTGAACGCCTCCTGAATAGTGAGTCGTATTAACGTACC |
| | | AACAAGAAGGAGCTGCCCATGAGAAAACTTGTTTCTCATGGGC |
| | | AGCTCCTTCTTTATCTTAGAGGCATATCCCTACGTACCAACAA |
| | | GTGCAATGAGGGACCAGTACAACTTGTGTACTGGTCCCTCATT |
| | | GCACTTTATCTTAGAGGCATATCCCTACGTACCAACAAGAGCT |
| | | GCTGAAGGACTCATCAACTTGTGATGAGTCCTTCAGCAGCTCT |
| | | TTATCTTAGAGGCATATCCCTTTTATCTTAGAGGCATATCCCT |
| | | CTGGGCCTCATGGGCCTTCCGCTCACTGCCCGCTTTCCAGTCG |
| | | GGAAACCTGTCGTGCCAGCTGCATTAACATGGTCATAGCTGTT |
| | | TCCTTGCGTATTGGGCGCTCTCCGCTTCCTCGCTCACTGACTC |
| | | GCTGCGCTCGGTCGTTCGGGTAAAGCCTGGGGTGCCTAATGAG |
| | | CAAAAGGCCAGCAAAAGGCCAGGAACCGTAAAAAGGCCGCGTT |
| | | GCTGGCGTTTTTCCATAGGCTCCGCCCCCCTGACGAGCATCAC |
| | | AAAAATCGACGCTCAAGTCAGAGGTGGCGAAACCCGACAGGAC |
| | | TATAAAGATACCAGGCGTTTCCCCCTGGAAGCTCCCTCGTGCG |
| | | CTCTCCTGTTCCGACCCTGCCGCTTACCGGATACCTGTCCGCC |
| | | TTTCTCCCTTCGGGAAGCGTGGCGCTTTCTCATAGCTCACGCT |
| | | GTAGGTATCTCAGTTCGGTGTAGGTCGTTCGCTCCAAGCTGGG |
| | | CTGTGTGCACGAACCCCCCGTTCAGCCCGACCGCTGCGCCTTA |
| | | TCCGGTAACTATCGTCTTGAGTCCAACCCGGTAAGACACGACT |
| | | TATCGCCACTGGCAGCAGCCACTGGTAACAGGATTAGCAGAGC |
| | | GAGGTATGTAGGCGGTGCTACAGAGTTCTTGAAGTGGTGGCCT |
| | | AACTACGGCTACACTAGAAGAACAGTATTTGGTATCTGCGCTC |
| | | TGCTGAAGCCAGTTACCTTCGGAAAAAGAGTTGGTAGCTCTTG |
| | | ATCCGGCAAACAAACCACCGCTGGTAGCGGTGGTTTTTTTGTT |
| | | TGCAAGCAGCAGATTACGCGCAGAAAAAAAGGATCTCAAGAAG |
| | | ATCCTTTGATCTTTTCTACGGGGTCTGACGCTCAGTGGAACGA |
| | | AAACTCACGTTAAGGGATTTTGGTCATGAGATTATCAAAAAGG |
| | | ATCTTCACCTAGATCCTTTTAAATTAAAAATGAAGTTTTAAAT |
| | | CAATCTAAAGTATATATGAGTAAACTTGGTCTGACAGTTACCA |
| | | ATGCTTAATCAGTGAGGCACCTATCTCAGCGATCTGTCTATTT |
| | | CGTTCATCCATAGTTGCCTGACTCCCCGTCGTGTAGATAACTA |
| | | CGATACGGGAGGGCTTACCATCTGGCCCCAGTGCTGCAATGAT |
| | | ACCGCGAGAACCACGCTCACCGGCTCCAGATTTATCAGCAATA |
| | | AACCAGCCAGCCGGAAGGGCCGAGCGCAGAAGTGGTCCTGCAA |
| | | CTTTATCCGCCTCCATCCAGTCTATTAATTGTTGCCGGGAAGC |
| | | TAGAGTAAGTAGTTCGCCAGTTAATAGTTTGCGCAACGTTGTT |
| | | GCCATTGCTACAGGCATCGTGGTGTCACGCTCGTCGTTTGGTA |
| | | TGGCTTCATTCAGCTCCGGTTCCCAACGATCAAGGCGAGTTAC |
| | | ATGATCCCCCATGTTGTGCAAAAAAGCGGTTAGCTCCTTCGGT |
| | | CCTCCGATCGTTGTCAGAAGTAAGTTGGCCGCAGTGTTATCAC |
| | | TCATGGTTATGGCAGCACTGCATAATTCTCTTACTGTCATGCC |
| | | ATCCGTAAGATGCTTTTCTGTGACTGGTGAGTACTCAACCAAG |
| | | TCATTCTGAGAATAGTGTATGCGGCGACCGAGTTGCTCTTGCC |
| | | CGGCGTCAATACGGGATAATACCGCGCCACATAGCAGAACTTT |
| | | AAAAGTGCTCATCATTGGAAAACGTTCTTCGGGGCGAAAACTC |
| | | TCAAGGATCTTACCGCTGTTGAGATCCAGTTCGATGTAACCCA |
| | | CTCGTGCACCCAACTGATCTTCAGCATCTTTTACTTTCACCAG |
| | | CGTTTCTGGGTGAGCAAAAACAGGAAGGCAAAATGCCGCAAAA |
| | | AAGGGAATAAGGGCGACACGGAAATGTTGAATACTCATACTCT |
| | | TCCTTTTTCAATATTATTGAAGCATTTATCAGGGTTATTGTCT |
| | | CATGAGCGGATACATATTTGAATGTATTTAGAAAAATAAACAA |
| | | ATAGGGGTTCCGCGCACATTTCCCCGAAAAGTGCCAC |
| 94 | Compound 13 | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCGCAT [symbol] ATGTGTCACCAG |
| | | CAGCTGGTCATCAGCTGGTTCAGCCTGGTGTTCCTGGCCTCTC |
| | | CTCTGGTGGCCATCTGGGAGCTGAAGAAAGACGTGTACGTGGT |
| | | GGAACTGGACTGGTATCCCGATGCTCCTGGCGAGATGGTGGTG |
| | | CTGACCTGCGATACCCCTGAAGAGGACGGCATCACCTGGACAC |
| | | TGGATCAGTCTAGCGAGGTGCTCGGCAGCGGCAAGACCCTGAC |
| | | CATCCAAGTGAAAGAGTTTGGCGACGCCGGCCAGTACACCTGT |
| | | CACAAAGGCGGAGAAGTGCTGAGCCACAGCCTGCTGCTGCTCC |
| | | ACAAGAAAGAGGATGGCATTTGGAGCACCGACATCCTGAAGGA |
| | | CCAGAAAGAGCCCAAGAACAAGACCTTCCTGAGATGCGAGGCC |
| | | AAGAACTACAGCGGCCGGTTCACATGTTGGTGGCTGACCACCA |
| | | TCAGCACCGACCTGACCTTCAGCGTGAAGTCCAGCAGAGGCAG |
| | | CAGTGATCCTCAGGGCGTTACATGTGGCGCCGCTACACTGTCT |
| | | GCCGAAAGAGTGCGGGGCGACAACAAAGAATACGAGTACAGCG |
| | | TGGAATGCCAAGAGGACAGCGCCTGTCCAGCCGCCGAAGAGTC |
| | | TCTGCCTATCGAAGTGATGGTGGACGCCGTGCACAAGCTGAAG |
| | | TACGAGAACTACACCTCCAGCTTTTTCATCCGGGACATCATCA |
| | | AGCCCGATCCTCCAAAGAACCTGCAGCTGAAGCCTCTGAAGAA |
| | | CAGCAGACAGGTGGAAGTGTCCTGGGAGTACCCCGACACCTGG |
| | | TCTACACCCCACAGCTACTTCAGCCTGACCTTTTGCGTGCAAG |
| | | TGCAGGGCAAGTCCAAGCGCGAGAAAAAGGACCGGGTGTTCAC |
| | | CGACAAGACCAGCGCCACCGTGATCTGCAGAAAGAACGCCAGC |
| | | ATCAGCGTCAGAGCCCAGGACCGGTACTACAGCAGCTCTTGGA |
| | | GCGAATGGGCCAGCGTGCCATGTTCTGGTGGCGGAGGATCTGG |
| | | CGGAGGTGGAAGCGGCGGAGGCGGATCTAGAAATCTGCCTGTG |
| | | GCCACTCCTGATCCTGGCATGTTCCCTTGTCTGCACCACAGCC |
| | | AGAACCTGCTGAGAGCCGTGTCCAACATGCTGCAGAAGGCCAG |
| | | ACAGACCCTGGAATTCTACCCCTGCACCAGCGAGGAAATCGAC |
| | | CACGAGGACATCACCAAGGATAAGACCAGCACCGTGGAAGCCT |
| | | GCCTGCCTCTGGAACTGACCAAGAACGAGAGCTGCCTGAACAG |
| | | CCGGGAAACCAGCTTCATCACCAACGGCTCTTGCCTGGCCAGC |
| | | AGAAAGACCTCCTTCATGATGGCCCTGTGCCTGAGCAGCATCT |
| | | ACGAGGACCTGAAGATGTACCAGGTGGAATTCAAGACCATGAA |
| | | CGCCAAGCTGCTGATGGACCCCAAGCGGCAGATCTTCCTGGAC |
| | | CAGAATATGCTGGCCGTGATCGACGAGCTGATGCAGGCCCTGA |
| | | ACTTCAACAGCGAGACAGTGCCCCAGAAGTCTAGCCTGGAAGA |
| | | ACCCGACTTCTACAAGACCAAGATCAAGCTGTGCATCCTGCTG |
| | | CACGCCTTCCGGATCAGAGCCGTGACCATCGACAGAGTGATGA |
| | | GCTACCTGAACGCCTCCTGAATAGTGAGTCGTATTAACGTACC |
| | | AACAAGAAGGAGCTGCCCATGAGAAAACTTGTTTCTCATGGGC |
| | | AGCTCCTTCTTTATCTTAGAGGCATATCCCTACGTACCAACAA |
| | | GTCCAACGAATGGGCCTAAGAACTTGTCTTAGGCCCATTCGTT |
| | | GGACTTTATCTTAGAGGCATATCCCTACGTACCAACAAGGACA |
| | | GCATAGACGACACCTTACTTGAAGGTGTCGTCTATGCTGTCCT |
| | | TTATCTTAGAGGCATATCCCTTTTATCTTAGAGGCATATCCCT |
| | | CTGGGCCTCATGGGCCTTCCGCTCACTGCCCGCTTTCCAGTCG |
| | | GGAAACCTGTCGTGCCAGCTGCATTAACATGGTCATAGCTGTT |
| | | TCCTTGCGTATTGGGCGCTCTCCGCTTCCTCGCTCACTGACTC |
| | | GCTGCGCTCGGTCGTTCGGGTAAAGCCTGGGGTGCCTAATGAG |
| | | CAAAAGGCCAGCAAAAGGCCAGGAACCGTAAAAAGGCCGCGTT |
| | | GCTGGCGTTTTTCCATAGGCTCCGCCCCCCTGACGAGCATCAC |
| | | AAAAATCGACGCTCAAGTCAGAGGTGGCGAAACCCGACAGGAC |
| | | TATAAAGATACCAGGCGTTTCCCCCTGGAAGCTCCCTCGTGCG |
| | | CTCTCCTGTTCCGACCCTGCCGCTTACCGGATACCTGTCCGCC |
| | | TTTCTCCCTTCGGGAAGCGTGGCGCTTTCTCATAGCTCACGCT |
| | | GTAGGTATCTCAGTTCGGTGTAGGTCGTTCGCTCCAAGCTGGG |
| | | CTGTGTGCACGAACCCCCCGTTCAGCCCGACCGCTGCGCCTTA |
| | | TCCGGTAACTATCGTCTTGAGTCCAACCCGGTAAGACACGACT |
| | | TATCGCCACTGGCAGCAGCCACTGGTAACAGGATTAGCAGAGC |
| | | GAGGTATGTAGGCGGTGCTACAGAGTTCTTGAAGTGGTGGCCT |
| | | AACTACGGCTACACTAGAAGAACAGTATTTGGTATCTGCGCTC |
| | | TGCTGAAGCCAGTTACCTTCGGAAAAAGAGTTGGTAGCTCTTG |
| | | ATCCGGCAAACAAACCACCGCTGGTAGCGGTGGTTTTTTTGTT |
| | | TGCAAGCAGCAGATTACGCGCAGAAAAAAAGGATCTCAAGAAG |
| | | ATCCTTTGATCTTTTCTACGGGGTCTGACGCTCAGTGGAACGA |
| | | AAACTCACGTTAAGGGATTTTGGTCATGAGATTATCAAAAAGG |
| | | ATCTTCACCTAGATCCTTTTAAATTAAAAATGAAGTTTTAAAT |
| | | CAATCTAAAGTATATATGAGTAAACTTGGTCTGACAGTTACCA |
| | | ATGCTTAATCAGTGAGGCACCTATCTCAGCGATCTGTCTATTT |
| | | CGTTCATCCATAGTTGCCTGACTCCCCGTCGTGTAGATAACTA |
| | | CGATACGGGAGGGCTTACCATCTGGCCCCAGTGCTGCAATGAT |
| | | ACCGCGAGAACCACGCTCACCGGCTCCAGATTTATCAGCAATA |
| | | AACCAGCCAGCCGGAAGGGCCGAGCGCAGAAGTGGTCCTGCAA |
| | | CTTTATCCGCCTCCATCCAGTCTATTAATTGTTGCCGGGAAGC |
| | | TAGAGTAAGTAGTTCGCCAGTTAATAGTTTGCGCAACGTTGTT |
| | | GCCATTGCTACAGGCATCGTGGTGTCACGCTCGTCGTTTGGTA |
| | | TGGCTTCATTCAGCTCCGGTTCCCAACGATCAAGGCGAGTTAC |
| | | ATGATCCCCCATGTTGTGCAAAAAAGCGGTTAGCTCCTTCGGT |
| | | CCTCCGATCGTTGTCAGAAGTAAGTTGGCCGCAGTGTTATCAC |
| | | TCATGGTTATGGCAGCACTGCATAATTCTCTTACTGTCATGCC |
| | | ATCCGTAAGATGCTTTTCTGTGACTGGTGAGTACTCAACCAAG |
| | | TCATTCTGAGAATAGTGTATGCGGCGACCGAGTTGCTCTTGCC |
| | | CGGCGTCAATACGGGATAATACCGCGCCACATAGCAGAACTTT |
| | | AAAAGTGCTCATCATTGGAAAACGTTCTTCGGGGCGAAAACTC |
| | | TCAAGGATCTTACCGCTGTTGAGATCCAGTTCGATGTAACCCA |
| | | CTCGTGCACCCAACTGATCTTCAGCATCTTTTACTTTCACCAG |
| | | CGTTTCTGGGTGAGCAAAAACAGGAAGGCAAAATGCCGCAAAA |
| | | AAGGGAATAAGGGCGACACGGAAATGTTGAATACTCATACTCT |
| | | TCCTTTTTCAATATTATTGAAGCATTTATCAGGGTTATTGTCT |
| | | CATGAGCGGATACATATTTGAATGTATTTAGAAAAATAAACAA |
| | | ATAGGGGTTCCGCGCACATTTCCCCGAAAAGTGCCAC |
| 95 | Compound 14 | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCGCAT [symbol] ATGTGTCACCAG |
| | | CAGCTGGTCATCAGCTGGTTCAGCCTGGTGTTCCTGGCCTCTC |
| | | CTCTGGTGGCCATCTGGGAGCTGAAGAAAGACGTGTACGTGGT |
| | | GGAACTGGACTGGTATCCCGATGCTCCTGGCGAGATGGTGGTG |
| | | CTGACCTGCGATACCCCTGAAGAGGACGGCATCACCTGGACAC |
| | | TGGATCAGTCTAGCGAGGTGCTCGGCAGCGGCAAGACCCTGAC |
| | | CATCCAAGTGAAAGAGTTTGGCGACGCCGGCCAGTACACCTGT |
| | | CACAAAGGCGGAGAAGTGCTGAGCCACAGCCTGCTGCTGCTCC |
| | | ACAAGAAAGAGGATGGCATTTGGAGCACCGACATCCTGAAGGA |
| | | CCAGAAAGAGCCCAAGAACAAGACCTTCCTGAGATGCGAGGCC |
| | | AAGAACTACAGCGGCCGGTTCACATGTTGGTGGCTGACCACCA |
| | | TCAGCACCGACCTGACCTTCAGCGTGAAGTCCAGCAGAGGCAG |
| | | CAGTGATCCTCAGGGCGTTACATGTGGCGCCGCTACACTGTCT |
| | | GCCGAAAGAGTGCGGGGCGACAACAAAGAATACGAGTACAGCG |
| | | TGGAATGCCAAGAGGACAGCGCCTGTCCAGCCGCCGAAGAGTC |
| | | TCTGCCTATCGAAGTGATGGTGGACGCCGTGCACAAGCTGAAG |
| | | TACGAGAACTACACCTCCAGCTTTTTCATCCGGGACATCATCA |
| | | AGCCCGATCCTCCAAAGAACCTGCAGCTGAAGCCTCTGAAGAA |
| | | CAGCAGACAGGTGGAAGTGTCCTGGGAGTACCCCGACACCTGG |
| | | TCTACACCCCACAGCTACTTCAGCCTGACCTTTTGCGTGCAAG |
| | | TGCAGGGCAAGTCCAAGCGCGAGAAAAAGGACCGGGTGTTCAC |
| | | CGACAAGACCAGCGCCACCGTGATCTGCAGAAAGAACGCCAGC |
| | | ATCAGCGTCAGAGCCCAGGACCGGTACTACAGCAGCTCTTGGA |
| | | GCGAATGGGCCAGCGTGCCATGTTCTGGTGGCGGAGGATCTGG |
| | | CGGAGGTGGAAGCGGCGGAGGCGGATCTAGAAATCTGCCTGTG |
| | | GCCACTCCTGATCCTGGCATGTTCCCTTGTCTGCACCACAGCC |
| | | AGAACCTGCTGAGAGCCGTGTCCAACATGCTGCAGAAGGCCAG |
| | | ACAGACCCTGGAATTCTACCCCTGCACCAGCGAGGAAATCGAC |
| | | CACGAGGACATCACCAAGGATAAGACCAGCACCGTGGAAGCCT |
| | | GCCTGCCTCTGGAACTGACCAAGAACGAGAGCTGCCTGAACAG |
| | | CCGGGAAACCAGCTTCATCACCAACGGCTCTTGCCTGGCCAGC |
| | | AGAAAGACCTCCTTCATGATGGCCCTGTGCCTGAGCAGCATCT |
| | | ACGAGGACCTGAAGATGTACCAGGTGGAATTCAAGACCATGAA |
| | | CGCCAAGCTGCTGATGGACCCCAAGCGGCAGATCTTCCTGGAC |
| | | CAGAATATGCTGGCCGTGATCGACGAGCTGATGCAGGCCCTGA |
| | | ACTTCAACAGCGAGACAGTGCCCCAGAAGTCTAGCCTGGAAGA |
| | | ACCCGACTTCTACAAGACCAAGATCAAGCTGTGCATCCTGCTG |
| | | CACGCCTTCCGGATCAGAGCCGTGACCATCGACAGAGTGATGA |
| | | GCTACCTGAACGCCTCCTGAATAGTGAGTCGTATTAACGTACC |
| | | AACAAGACCCTGACATTCGCTACTGTACTTGACAGTAGCGAAT |
| | | GTCAGGGTCTTTATCTTAGAGGCATATCCCTACGTACCAACAA |
| | | GAGCTGCTGAAGGACTCATCAACTTGTGATGAGTCCTTCAGCA |
| | | GCTCTTTATCTTAGAGGCATATCCCTACGTACCAACAAGGCCA |
| | | ATGACCCAACATCTCTACTTGAGAGATGTTGGGTCATTGGCCT |
| | | TTATCTTAGAGGCATATCCCTTTTATCTTAGAGGCATATCCCT |
| | | CTGGGCCTCATGGGCCTTCCGCTCACTGCCCGCTTTCCAGTCG |
| | | GGAAACCTGTCGTGCCAGCTGCATTAACATGGTCATAGCTGTT |
| | | TCCTTGCGTATTGGGCGCTCTCCGCTTCCTCGCTCACTGACTC |
| | | GCTGCGCTCGGTCGTTCGGGTAAAGCCTGGGGTGCCTAATGAG |
| | | CAAAAGGCCAGCAAAAGGCCAGGAACCGTAAAAAGGCCGCGTT |
| | | GCTGGCGTTTTTCCATAGGCTCCGCCCCCCTGACGAGCATCAC |
| | | AAAAATCGACGCTCAAGTCAGAGGTGGCGAAACCCGACAGGAC |
| | | TATAAAGATACCAGGCGTTTCCCCCTGGAAGCTCCCTCGTGCG |
| | | CTCTCCTGTTCCGACCCTGCCGCTTACCGGATACCTGTCCGCC |
| | | TTTCTCCCTTCGGGAAGCGTGGCGCTTTCTCATAGCTCACGCT |
| | | GTAGGTATCTCAGTTCGGTGTAGGTCGTTCGCTCCAAGCTGGG |
| | | CTGTGTGCACGAACCCCCCGTTCAGCCCGACCGCTGCGCCTTA |
| | | TCCGGTAACTATCGTCTTGAGTCCAACCCGGTAAGACACGACT |
| | | TATCGCCACTGGCAGCAGCCACTGGTAACAGGATTAGCAGAGC |
| | | GAGGTATGTAGGCGGTGCTACAGAGTTCTTGAAGTGGTGGCCT |
| | | AACTACGGCTACACTAGAAGAACAGTATTTGGTATCTGCGCTC |
| | | TGCTGAAGCCAGTTACCTTCGGAAAAAGAGTTGGTAGCTCTTG |
| | | ATCCGGCAAACAAACCACCGCTGGTAGCGGTGGTTTTTTTGTT |
| | | TGCAAGCAGCAGATTACGCGCAGAAAAAAAGGATCTCAAGAAG |
| | | ATCCTTTGATCTTTTCTACGGGGTCTGACGCTCAGTGGAACGA |
| | | AAACTCACGTTAAGGGATTTTGGTCATGAGATTATCAAAAAGG |
| | | ATCTTCACCTAGATCCTTTTAAATTAAAAATGAAGTTTTAAAT |
| | | CAATCTAAAGTATATATGAGTAAACTTGGTCTGACAGTTACCA |
| | | ATGCTTAATCAGTGAGGCACCTATCTCAGCGATCTGTCTATTT |
| | | CGTTCATCCATAGTTGCCTGACTCCCCGTCGTGTAGATAACTA |
| | | CGATACGGGAGGGCTTACCATCTGGCCCCAGTGCTGCAATGAT |
| | | ACCGCGAGAACCACGCTCACCGGCTCCAGATTTATCAGCAATA |
| | | AACCAGCCAGCCGGAAGGGCCGAGCGCAGAAGTGGTCCTGCAA |
| | | CTTTATCCGCCTCCATCCAGTCTATTAATTGTTGCCGGGAAGC |
| | | TAGAGTAAGTAGTTCGCCAGTTAATAGTTTGCGCAACGTTGTT |
| | | GCCATTGCTACAGGCATCGTGGTGTCACGCTCGTCGTTTGGTA |
| | | TGGCTTCATTCAGCTCCGGTTCCCAACGATCAAGGCGAGTTAC |
| | | ATGATCCCCCATGTTGTGCAAAAAAGCGGTTAGCTCCTTCGGT |
| | | CCTCCGATCGTTGTCAGAAGTAAGTTGGCCGCAGTGTTATCAC |
| | | TCATGGTTATGGCAGCACTGCATAATTCTCTTACTGTCATGCC |
| | | ATCCGTAAGATGCTTTTCTGTGACTGGTGAGTACTCAACCAAG |
| | | TCATTCTGAGAATAGTGTATGCGGCGACCGAGTTGCTCTTGCC |
| | | CGGCGTCAATACGGGATAATACCGCGCCACATAGCAGAACTTT |
| | | AAAAGTGCTCATCATTGGAAAACGTTCTTCGGGGCGAAAACTC |
| | | TCAAGGATCTTACCGCTGTTGAGATCCAGTTCGATGTAACCCA |
| | | CTCGTGCACCCAACTGATCTTCAGCATCTTTTACTTTCACCAG |
| | | CGTTTCTGGGTGAGCAAAAACAGGAAGGCAAAATGCCGCAAAA |
| | | AAGGGAATAAGGGCGACACGGAAATGTTGAATACTCATACTCT |
| | | TCCTTTTTCAATATTATTGAAGCATTTATCAGGGTTATTGTCT |
| | | CATGAGCGGATACATATTTGAATGTATTTAGAAAAATAAACAA |
| | | ATAGGGGTTCCGCGCACATTTCCCCGAAAAGTGCCAC |
| 96 | Compound 15 | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCGCAT [symbol] ATGAGAATCAGC |
| | | AAGCCCCACCTGAGATCCATCAGCATCCAGTGCTACCTGTGCC |
| | | TGCTGCTGAACAGCCACTTTCTGACAGAGGCCGGCATCCACGT |
| | | GTTCATCCTGGGCTGTTTTTCTGCCGGCCTGCCTAAGACCGAG |
| | | GCCAACTGGGTTAACGTGATCAGCGACCTGAAGAAGATCGAGG |
| | | ACCTGATCCAGAGCATGCACATCGACGCCACACTGTACACCGA |
| | | GAGCGACGTGCACCCTAGCTGTAAAGTGACCGCCATGAAGTGC |
| | | TTTCTGCTGGAACTGCAAGTGATCAGCCTGGAAAGCGGCGACG |
| | | CCAGCATCCACGACACCGTGGAAAACCTGATCATCCTGGCCAA |
| | | CAACAGCCTGAGCAGCAACGGCAATGTGACCGAGTCCGGCTGC |
| | | AAAGAGTGCGAGGAACTGGAAGAGAAGAATATCAAAGAGTTCC |
| | | TGCAGAGCTTCGTGCACATCGTGCAGATGTTCATCAACACCAG |
| | | CTGAATAGTGAGTCGTATTAACGTACCAACAAGGAGTACCCTG |
| | | ATGAGATCACTTGGATCTCATCAGGGTACTCCTTTATCTTAGA |
| | | GGCATATCCCTACGTACCAACAAGGTATCCATCTCTGGCTATG |
| | | AACTTGTCATAGCCAGAGATGGATACCTTTATCTTAGAGGCAT |
| | | ATCCCTACGTACCAACAAGTCCCGTAACGCCATCATCTTACTT |
| | | GAAGATGATGGCGTTACGGGACTTTATCTTAGAGGCATATCCC |
| | | TTTTATCTTAGAGGCATATCCCTCTGGGCCTCATGGGCCTTCC |
| | | GCTCACTGCCCGCTTTCCAGTCGGGAAACCTGTCGTGCCAGCT |
| | | GCATTAACATGGTCATAGCTGTTTCCTTGCGTATTGGGCGCTC |
| | | TCCGCTTCCTCGCTCACTGACTCGCTGCGCTCGGTCGTTCGGG |
| | | TAAAGCCTGGGGTGCCTAATGAGCAAAAGGCCAGCAAAAGGCC |
| | | AGGAACCGTAAAAAGGCCGCGTTGCTGGCGTTTTTCCATAGGC |
| | | TCCGCCCCCCTGACGAGCATCACAAAAATCGACGCTCAAGTCA |
| | | GAGGTGGCGAAACCCGACAGGACTATAAAGATACCAGGCGTTT |
| | | CCCCCTGGAAGCTCCCTCGTGCGCTCTCCTGTTCCGACCCTGC |
| | | CGCTTACCGGATACCTGTCCGCCTTTCTCCCTTCGGGAAGCGT |
| | | GGCGCTTTCTCATAGCTCACGCTGTAGGTATCTCAGTTCGGTG |
| | | TAGGTCGTTCGCTCCAAGCTGGGCTGTGTGCACGAACCCCCCG |
| | | TTCAGCCCGACCGCTGCGCCTTATCCGGTAACTATCGTCTTGA |
| | | GTCCAACCCGGTAAGACACGACTTATCGCCACTGGCAGCAGCC |
| | | ACTGGTAACAGGATTAGCAGAGCGAGGTATGTAGGCGGTGCTA |
| | | CAGAGTTCTTGAAGTGGTGGCCTAACTACGGCTACACTAGAAG |
| | | AACAGTATTTGGTATCTGCGCTCTGCTGAAGCCAGTTACCTTC |
| | | GGAAAAAGAGTTGGTAGCTCTTGATCCGGCAAACAAACCACCG |
| | | CTGGTAGCGGTGGTTTTTTTGTTTGCAAGCAGCAGATTACGCG |
| | | CAGAAAAAAAGGATCTCAAGAAGATCCTTTGATCTTTTCTACG |
| | | GGGTCTGACGCTCAGTGGAACGAAAACTCACGTTAAGGGATTT |
| | | TGGTCATGAGATTATCAAAAAGGATCTTCACCTAGATCCTTTT |
| | | AAATTAAAAATGAAGTTTTAAATCAATCTAAAGTATATATGAG |
| | | TAAACTTGGTCTGACAGTTACCAATGCTTAATCAGTGAGGCAC |
| | | CTATCTCAGCGATCTGTCTATTTCGTTCATCCATAGTTGCCTG |
| | | ACTCCCCGTCGTGTAGATAACTACGATACGGGAGGGCTTACCA |
| | | TCTGGCCCCAGTGCTGCAATGATACCGCGAGAACCACGCTCAC |
| | | CGGCTCCAGATTTATCAGCAATAAACCAGCCAGCCGGAAGGGC |
| | | CGAGCGCAGAAGTGGTCCTGCAACTTTATCCGCCTCCATCCAG |
| | | TCTATTAATTGTTGCCGGGAAGCTAGAGTAAGTAGTTCGCCAG |
| | | TTAATAGTTTGCGCAACGTTGTTGCCATTGCTACAGGCATCGT |
| | | GGTGTCACGCTCGTCGTTTGGTATGGCTTCATTCAGCTCCGGT |
| | | TCCCAACGATCAAGGCGAGTTACATGATCCCCCATGTTGTGCA |
| | | AAAAAGCGGTTAGCTCCTTCGGTCCTCCGATCGTTGTCAGAAG |
| | | TAAGTTGGCCGCAGTGTTATCACTCATGGTTATGGCAGCACTG |
| | | CATAATTCTCTTACTGTCATGCCATCCGTAAGATGCTTTTCTG |
| | | TGACTGGTGAGTACTCAACCAAGTCATTCTGAGAATAGTGTAT |
| | | GCGGCGACCGAGTTGCTCTTGCCCGGCGTCAATACGGGATAAT |
| | | ACCGCGCCACATAGCAGAACTTTAAAAGTGCTCATCATTGGAA |
| | | AACGTTCTTCGGGGCGAAAACTCTCAAGGATCTTACCGCTGTT |
| | | GAGATCCAGTTCGATGTAACCCACTCGTGCACCCAACTGATCT |
| | | TCAGCATCTTTTACTTTCACCAGCGTTTCTGGGTGAGCAAAAA |
| | | CAGGAAGGCAAAATGCCGCAAAAAAGGGAATAAGGGCGACACG |
| | | GAAATGTTGAATACTCATACTCTTCCTTTTTCAATATTATTGA |
| | | AGCATTTATCAGGGTTATTGTCTCATGAGCGGATACATATTTG |
| | | AATGTATTTAGAAAAATAAACAAATAGGGGTTCCGCGCACATT |
| | | TCCCCGAAAAGTGCCAC |
| 97 | Compound 16 | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCGCAT [symbol] ATGAGAATCAGC |
| | | AAGCCCCACCTGAGATCCATCAGCATCCAGTGCTACCTGTGCC |
| | | TGCTGCTGAACAGCCACTTTCTGACAGAGGCCGGCATCCACGT |
| | | GTTCATCCTGGGCTGTTTTTCTGCCGGCCTGCCTAAGACCGAG |
| | | GCCAACTGGGTTAACGTGATCAGCGACCTGAAGAAGATCGAGG |
| | | ACCTGATCCAGAGCATGCACATCGACGCCACACTGTACACCGA |
| | | GAGCGACGTGCACCCTAGCTGTAAAGTGACCGCCATGAAGTGC |
| | | TTTCTGCTGGAACTGCAAGTGATCAGCCTGGAAAGCGGCGACG |
| | | CCAGCATCCACGACACCGTGGAAAACCTGATCATCCTGGCCAA |
| | | CAACAGCCTGAGCAGCAACGGCAATGTGACCGAGTCCGGCTGC |
| | | AAAGAGTGCGAGGAACTGGAAGAGAAGAATATCAAAGAGTTCC |
| | | TGCAGAGCTTCGTGCACATCGTGCAGATGTTCATCAACACCAG |
| | | CTGAATAGTGAGTCGTATTAACGTACCAACAAGGAGTACCCTG |
| | | ATGAGATCACTTGGATCTCATCAGGGTACTCCTTTATCTTAGA |
| | | GGCATATCCCTACGTACCAACAAGAAGGTTCAGCATAGTAGCT |
| | | AACTTGTAGCTACTATGCTGAACCTTCTTTATCTTAGAGGCAT |
| | | ATCCCTACGTACCAACAAGGACGACGAGACCTTCATCAAACTT |
| | | GTTGATGAAGGTCTCGTCGTCCTTTATCTTAGAGGCATATCCC |
| | | TTTTATCTTAGAGGCATATCCCTCTGGGCCTCATGGGCCTTCC |
| | | GCTCACTGCCCGCTTTCCAGTCGGGAAACCTGTCGTGCCAGCT |
| | | GCATTAACATGGTCATAGCTGTTTCCTTGCGTATTGGGCGCTC |
| | | TCCGCTTCCTCGCTCACTGACTCGCTGCGCTCGGTCGTTCGGG |
| | | TAAAGCCTGGGGTGCCTAATGAGCAAAAGGCCAGCAAAAGGCC |
| | | AGGAACCGTAAAAAGGCCGCGTTGCTGGCGTTTTTCCATAGGC |
| | | TCCGCCCCCCTGACGAGCATCACAAAAATCGACGCTCAAGTCA |
| | | GAGGTGGCGAAACCCGACAGGACTATAAAGATACCAGGCGTTT |
| | | CCCCCTGGAAGCTCCCTCGTGCGCTCTCCTGTTCCGACCCTGC |
| | | CGCTTACCGGATACCTGTCCGCCTTTCTCCCTTCGGGAAGCGT |
| | | GGCGCTTTCTCATAGCTCACGCTGTAGGTATCTCAGTTCGGTG |
| | | TAGGTCGTTCGCTCCAAGCTGGGCTGTGTGCACGAACCCCCCG |
| | | TTCAGCCCGACCGCTGCGCCTTATCCGGTAACTATCGTCTTGA |
| | | GTCCAACCCGGTAAGACACGACTTATCGCCACTGGCAGCAGCC |
| | | ACTGGTAACAGGATTAGCAGAGCGAGGTATGTAGGCGGTGCTA |
| | | CAGAGTTCTTGAAGTGGTGGCCTAACTACGGCTACACTAGAAG |
| | | AACAGTATTTGGTATCTGCGCTCTGCTGAAGCCAGTTACCTTC |
| | | GGAAAAAGAGTTGGTAGCTCTTGATCCGGCAAACAAACCACCG |
| | | CTGGTAGCGGTGGTTTTTTTGTTTGCAAGCAGCAGATTACGCG |
| | | CAGAAAAAAAGGATCTCAAGAAGATCCTTTGATCTTTTCTACG |
| | | GGGTCTGACGCTCAGTGGAACGAAAACTCACGTTAAGGGATTT |
| | | TGGTCATGAGATTATCAAAAAGGATCTTCACCTAGATCCTTTT |
| | | AAATTAAAAATGAAGTTTTAAATCAATCTAAAGTATATATGAG |
| | | TAAACTTGGTCTGACAGTTACCAATGCTTAATCAGTGAGGCAC |
| | | CTATCTCAGCGATCTGTCTATTTCGTTCATCCATAGTTGCCTG |
| | | ACTCCCCGTCGTGTAGATAACTACGATACGGGAGGGCTTACCA |
| | | TCTGGCCCCAGTGCTGCAATGATACCGCGAGAACCACGCTCAC |
| | | CGGCTCCAGATTTATCAGCAATAAACCAGCCAGCCGGAAGGGC |
| | | CGAGCGCAGAAGTGGTCCTGCAACTTTATCCGCCTCCATCCAG |
| | | TCTATTAATTGTTGCCGGGAAGCTAGAGTAAGTAGTTCGCCAG |
| | | TTAATAGTTTGCGCAACGTTGTTGCCATTGCTACAGGCATCGT |
| | | GGTGTCACGCTCGTCGTTTGGTATGGCTTCATTCAGCTCCGGT |
| | | TCCCAACGATCAAGGCGAGTTACATGATCCCCCATGTTGTGCA |
| | | AAAAAGCGGTTAGCTCCTTCGGTCCTCCGATCGTTGTCAGAAG |
| | | TAAGTTGGCCGCAGTGTTATCACTCATGGTTATGGCAGCACTG |
| | | CATAATTCTCTTACTGTCATGCCATCCGTAAGATGCTTTTCTG |
| | | TGACTGGTGAGTACTCAACCAAGTCATTCTGAGAATAGTGTAT |
| | | GCGGCGACCGAGTTGCTCTTGCCCGGCGTCAATACGGGATAAT |
| | | ACCGCGCCACATAGCAGAACTTTAAAAGTGCTCATCATTGGAA |
| | | AACGTTCTTCGGGGCGAAAACTCTCAAGGATCTTACCGCTGTT |
| | | GAGATCCAGTTCGATGTAACCCACTCGTGCACCCAACTGATCT |
| | | TCAGCATCTTTTACTTTCACCAGCGTTTCTGGGTGAGCAAAAA |
| | | CAGGAAGGCAAAATGCCGCAAAAAAGGGAATAAGGGCGACACG |
| | | GAAATGTTGAATACTCATACTCTTCCTTTTTCAATATTATTGA |
| | | AGCATTTATCAGGGTTATTGTCTCATGAGCGGATACATATTTG |
| | | AATGTATTTAGAAAAATAAACAAATAGGGGTTCCGCGCACATT |
| | | TCCCCGAAAAGTGCCAC |
| 98 | Compound 17 | CTAAATTGTAAGCGTTAATATTTTGTTAAAATTCGCGTTAAAT |
| | (pMA-RQ) | TTTTGTTAAATCAGCTCATTTTTTAACCAATAGGCCGAAATCG |
| | | GCAAAATCCCTTATAAATCAAAAGAATAGACCGAGATAGGGTT |
| | | GAGTGGCCGCTACAGGGCGCTCCCATTCGCCATTCAGGCTGCG |
| | | CAACTGTTGGGAAGGGCGTTTCGGTGCGGGCCTCTTCGCTATT |
| | | ACGCCAGCTGGCGAAAGGGGGATGTGCTGCAAGGCGATTAAGT |
| | | TGGGTAACGCCAGGGTTTTCCCAGTCACGACGTTGTAAAACGA |
| | | CGGCCAGTGAGCGCGACGTAATACGACTCACTATAGGGCGAAT |
| | | TGGCGGAAGGCCGTCAAGGCCGCAT [symbol] ATGTTCCACGTG |
| | | TCCTTCCGGTACATCTTCGGCCTGCCTCCACTGATCCTGGTGC |
| | | TGCTGCCTGTGGCCAGCAGCGACTGTGATATCGAGGGCAAAGA |
| | | CGGCAAGCAGTACGAGAGCGTGCTGATGGTGTCCATCGACCAG |
| | | CTGCTGGACAGCATGAAGGAAATCGGCAGCAACTGCCTGAACA |
| | | ACGAGTTCAACTTCTTCAAGCGGCACATCTGCGACGCCAACAA |
| | | AGAAGGCATGTTCCTGTTCAGAGCCGCCAGAAAGCTGCGGCAG |
| | | TTCCTGAAGATGAACAGCACCGGCGACTTCGACCTGCATCTGC |
| | | TGAAAGTGTCTGAGGGCACCACCATCCTGCTGAATTGCACCGG |
| | | CCAAGTGAAGGGCAGAAAGCCTGCTGCTCTGGGAGAAGCCCAG |
| | | CCTACCAAGAGCCTGGAAGAGAACAAGTCCCTGAAAGAGCAGA |
| | | AGAAGCTGAACGACCTCTGCTTCCTGAAGCGGCTGCTGCAAGA |
| | | GATCAAGACCTGCTGGAACAAGATCCTGATGGGCACCAAAGAA |
| | | CACTGAATAGTGAGTCGTATTAACGTACCAACAAGAAGGTTCA |
| | | GCATAGTAGCTAACTTGTAGCTACTATGCTGAACCTTCTTTAT |
| | | CTTAGAGGCATATCCCTACGTACCAACAAGCGAATTACTGTGA |
| | | AAGTCAAACTTGTTGACTTTCACAGTAATTCGCTTTATCTTAG |
| | | AGGCATATCCCTACGTACCAACAAGACCAGCACACTGAGAATC |
| | | AAACTTGTTGATTCTCAGTGTGCTGGTCTTTATCTTAGAGGCA |
| | | TATCCCTTTTATCTTAGAGGCATATCCCTCTGGGCCTCATGGG |
| | | CCTTCCGCTCACTGCCCGCTTTCCAGTCGGGAAACCTGTCGTG |
| | | CCAGCTGCATTAACATGGTCATAGCTGTTTCCTTGCGTATTGG |
| | | GCGCTCTCCGCTTCCTCGCTCACTGACTCGCTGCGCTCGGTCG |
| | | TTCGGGTAAAGCCTGGGGTGCCTAATGAGCAAAAGGCCAGCAA |
| | | AAGGCCAGGAACCGTAAAAAGGCCGCGTTGCTGGCGTTTTTCC |
| | | ATAGGCTCCGCCCCCCTGACGAGCATCACAAAAATCGACGCTC |
| | | AAGTCAGAGGTGGCGAAACCCGACAGGACTATAAAGATACCAG |
| | | GCGTTTCCCCCTGGAAGCTCCCTCGTGCGCTCTCCTGTTCCGA |
| | | CCCTGCCGCTTACCGGATACCTGTCCGCCTTTCTCCCTTCGGG |
| | | AAGCGTGGCGCTTTCTCATAGCTCACGCTGTAGGTATCTCAGT |
| | | TCGGTGTAGGTCGTTCGCTCCAAGCTGGGCTGTGTGCACGAAC |
| | | CCCCCGTTCAGCCCGACCGCTGCGCCTTATCCGGTAACTATCG |
| | | TCTTGAGTCCAACCCGGTAAGACACGACTTATCGCCACTGGCA |
| | | GCAGCCACTGGTAACAGGATTAGCAGAGCGAGGTATGTAGGCG |
| | | GTGCTACAGAGTTCTTGAAGTGGTGGCCTAACTACGGCTACAC |
| | | TAGAAGAACAGTATTTGGTATCTGCGCTCTGCTGAAGCCAGTT |
| | | ACCTTCGGAAAAAGAGTTGGTAGCTCTTGATCCGGCAAACAAA |
| | | CCACCGCTGGTAGCGGTGGTTTTTTTGTTTGCAAGCAGCAGAT |
| | | TACGCGCAGAAAAAAAGGATCTCAAGAAGATCCTTTGATCTTT |
| | | TCTACGGGGTCTGACGCTCAGTGGAACGAAAACTCACGTTAAG |
| | | GGATTTTGGTCATGAGATTATCAAAAAGGATCTTCACCTAGAT |
| | | CCTTTTAAATTAAAAATGAAGTTTTAAATCAATCTAAAGTATA |
| | | TATGAGTAAACTTGGTCTGACAGTTACCAATGCTTAATCAGTG |
| | | AGGCACCTATCTCAGCGATCTGTCTATTTCGTTCATCCATAGT |
| | | TGCCTGACTCCCCGTCGTGTAGATAACTACGATACGGGAGGGC |
| | | TTACCATCTGGCCCCAGTGCTGCAATGATACCGCGAGAACCAC |
| | | GCTCACCGGCTCCAGATTTATCAGCAATAAACCAGCCAGCCGG |
| | | AAGGGCCGAGCGCAGAAGTGGTCCTGCAACTTTATCCGCCTCC |
| | | ATCCAGTCTATTAATTGTTGCCGGGAAGCTAGAGTAAGTAGTT |
| | | CGCCAGTTAATAGTTTGCGCAACGTTGTTGCCATTGCTACAGG |
| | | CATCGTGGTGTCACGCTCGTCGTTTGGTATGGCTTCATTCAGC |
| | | TCCGGTTCCCAACGATCAAGGCGAGTTACATGATCCCCCATGT |
| | | TGTGCAAAAAAGCGGTTAGCTCCTTCGGTCCTCCGATCGTTGT |
| | | CAGAAGTAAGTTGGCCGCAGTGTTATCACTCATGGTTATGGCA |
| | | GCACTGCATAATTCTCTTACTGTCATGCCATCCGTAAGATGCT |
| | | TTTCTGTGACTGGTGAGTACTCAACCAAGTCATTCTGAGAATA |
| | | GTGTATGCGGCGACCGAGTTGCTCTTGCCCGGCGTCAATACGG |
| | | GATAATACCGCGCCACATAGCAGAACTTTAAAAGTGCTCATCA |
| | | TTGGAAAACGTTCTTCGGGGCGAAAACTCTCAAGGATCTTACC |
| | | GCTGTTGAGATCCAGTTCGATGTAACCCACTCGTGCACCCAAC |
| | | TGATCTTCAGCATCTTTTACTTTCACCAGCGTTTCTGGGTGAG |
| | | CAAAAACAGGAAGGCAAAATGCCGCAAAAAAGGGAATAAGGGC |
| | | GACACGGAAATGTTGAATACTCATACTCTTCCTTTTTCAATAT |
| | | TATTGAAGCATTTATCAGGGTTATTGTCTCATGAGCGGATACA |
| | | TATTTGAATGTATTTAGAAAAATAAACAAATAGGGGTTCCGCG |
| | | CACATTTCCCCGAAAAGTGCCAC |

Bold = compound sequence
Bold and underline = compound sequence
Bold Italics = Kozak sequence
*Bolding indicates construct with modified signal peptide.

### Example 2: In Vitro Transcription of RNA Constructs and Data Analysis

PCR-based in vitro transcription is carried out using the pMA-T (Cpd.1-Cpd.4), pMK-RQ (Cpd.5) or the pMA-RQ (Cpd.6-Cpd.17) vectors encoding Cpd.1-Cpd.17 to produce mRNA. A transcription template was generated by PCR using the forward and reverse primers in Table 5. The poly(A) tail was encoded in the template resulting in a 120 bp poly(A) tail (SEQ ID NO: 153). Optimizations were made as needed to achieve specific amplification given the repetitive sequence of siRNA flanking regions. Optimizations include: 1) decreasing the amount of plasmid DNA of vector, 2) changing the DNA polymerase (Q5 hot start polymerase, New England Biolabs), 3) reducing denaturation time (30 seconds to 10 seconds) and extension time (45 seconds/kb to 10 seconds/kb) for each cycle of PCR, 4) increasing the annealing (10 seconds to 30 seconds) for each cycle of PCR, and 5) increasing the final extension time (up to 15 minutes) for each cycle of PCR. In addition, to avoid non-specific primer binding, the PCR reaction mixture was prepared on ice including thawing reagents, and the number of PCR cycles was reduced to 25.

For in vitro transcription, T7 RNA polymerase (MEGAscript kit, Thermo Fisher Scientific) was used at 37° C. for 2 hours. Synthesized RNAs were chemically modified with 100% N1-methylpseudo-UTP and co-transcriptionally capped with an anti-reverse CAP analog (ARCA; [m₂^{7,3′-O}G(5′)ppp(5′)G]) at the 5′ end (Jena Bioscience). After in vitro transcription, the mRNAs were column-purified using MEGAclear kit (Thermo Fisher Scientific) and quantified using Nanophotometer-N60 (Implen).

TABLE 5
Primers for Template Generation

| SEQ | Primer | |
|---|---|---|
| ID NO | Direction | Sequence (5′ to 3′) |
| 99 | Forward | GCTGCAAGGCGATTAAGTTG |
| 100 | Reverse | U(2′OMe)U(2′OMe)U(2′OMe)TTTTTTTTTTTTTTTTTTTTTT |
| | | TTTTTTTTTTTTTTTTTTTTTTTTTTTTTTTTTTTTTTTTTTTTTT |
| | | TTTTTTTTTTTTTTTTTTTTTTTTTTTTTTTTTTTTTTTTTTTTTT |
| | | TTTCAGCTATGACCATGTTAATGCAG |

Using in vitro transcription, Cpd.1-Cpd.17 were generated as an mRNA and tested in various in vitro models specified below for IL-2, IL-7, IL-12, and IL-15 expression and combinatorial effect of respective protein overexpression in parallel to target gene down regulation.

Determination of Molecular weight of constructs was performed as below. The molecular weight of each construct was determined from each sequence by determining the total number of each base (A, C, G, T or N1-UTP) present in each sequence and multiply the number by respective molecular weight (e.g., A: 347.2 g/mol; C 323.2 g/mol; G 363.2 g/mol; N1-UTP:338.2 g/mol). The molecular weight was determined by the sum of all weights obtained for each base and ARCA molecular weight of 817.4 g/mol. The molecular weight of each construct was used to calculate the amount of mRNA used for transfection in each well to nanomolar (nM) concentration.

Data were analyzed using GraphPad Prism 8 (San Diego, USA). For the estimation of the protein levels using ELISA in the standard or the sample, the mean absorbance value of the blank was subtracted from the mean absorbance of the standards or the samples. A standard curve was generated and plotted using a four parameters nonlinear regression according to manufacturer's protocol. To determine the concentration of proteins in each sample, the concentration of the different protein was interpolated from the standard curve. The final protein concentration of the sample was calculated by multiplication with the dilution factor. Statistical analysis was carried out using by Student's t-test or one-way ANOVA followed by Dunnet's multiple comparing tests.

### Example 3: In Vitro Transfection of HEK-293 Cells

Human embryonic kidney cells 293 (HEK-293; ATCC CRL-1573, Rockville, MD, USA) were maintained in Dulbecco's Modified Eagle's medium (DMEM, Sigma-Aldrich) supplemented with 10% (v/v) Fetal Bovine Serum (FBS, Thermofischer, Basel, Switzerland cat #10500-064). To assess the IL-2 expression the HEK-293 cells were seeded at 20,000 cell/well in a 96 well culture plate and incubated at 37° C. in a humidified atmosphere containing 5% CO₂ for 24 hours prior to transfection. Cells were then grown in DMEM growth medium containing 10% of FBS to reach confluency <80% before transfection. Thereafter, HEK-293 cells were transfected with 300 ng of specific mRNA constructs using Lipofectamine 2000 (Thermo Fisher Scientific) following the manufacturer's instructions with the mRNA to Lipofectamine ratio of 1:1 w/v. 100 μl of DMEM was removed and 50 μl of Opti-MEM (Thermo Fisher Scientific) was added to each well followed by 50 μl mRNA and Lipofectamine 2000 complex in Opti-MEM. After 5 hours of incubation, the medium was replaced by fresh growth medium and the plates were incubated for 24 hours at 37° C. in a humidified atmosphere containing 5% CO₂. Cell culture supernatant were collected to measure secreted IL-2 using ELISA (ThermoFisher Cat. #887025). Significance (**, p<0.01) was assessed by one way ANOVA followed by Dunnet's multiple comparing test using Cpd.1 as control.

IL-2 Secretion in HEK-293 Cells

Cpd.1-Cpd.4 comprising IL-2 protein coding sequence were tested for IL-2 expression and secretion from HEK-293 cells. Protein levels of secreted IL-2 were measured in the cell culture supernatant using IL-2 ELISA and are represented as fold changed referenced to Cpd.1 (containing WT IL-2 signal peptide) in FIG. 2A. The protein levels of secreted IL-2 by cells transfected with Cpd.2-Cpd.4 (containing modified IL-2 signal peptide) were about 2-fold higher than protein level of secreted IL-2 by cells transfected with Cpd.1. Taken together, the data suggest that Cpd.2-Cpd.4 with homologous modified signal peptides can facilitate enhanced cellular exit of produced IL-2 in HEK-293 cells compared to Cpd.1 with endogenous signal peptide. Data represent means±standard error of the mean of 3 replicates per Cpd. Significance (**, p<0.01) was assessed by one way ANOVA followed by Dunnet's multiple comparing test using Cpd.1 as control.

### Example 4: In Vitro Transfection of HaCaT Cells

Human keratinocytes (HaCaT; AddexBio Cat. #T0020001) were maintained in Dulbecco's Modified Eagle's medium (DMEM, Sigma-Aldrich) supplemented with 10% (v/v) Fetal Bovine Serum (FBS, Thermofischer, Basel, Switzerland cat #10500-064). To assess the IL-2 expression the HaCaT cells were seeded at 15,000 cell/well in a 96 well culture plate and incubated at 37° C. in a humidified atmosphere containing 5% CO₂ for 24 hours prior to transfection. Cells were then grown in DMEM growth medium containing 10% of FBS to reach confluency <70% before transfection. Thereafter, HaCaT cells were transfected with 300 ng of specific mRNA constructs using Lipofectamine 2000 (Invitrogen) following the manufacturer's instructions with the mRNA to Lipofectamine ratio of 1:1 w/v. 100 μl of DMEM was removed and 50 μl of Opti-MEM (Thermo Fisher Scientific) was added to each well followed by 50 μl mRNA and Lipofectamine 2000 complex in Opti-MEM. After 5 hours of incubation, the medium was replaced by fresh growth medium and the plates were incubated for 24 hours at 37° C. in a humidified atmosphere containing 5% CO₂. Cell culture supernatant were collected to measure secreted IL-2 using ELISA (ThermoFisher Cat. #887025). Significance (p<0.01) was assessed by one way ANOVA followed by Dunnet's multiple comparing test using Cpd.1 as control.

IL-2 Secretion in HaCaT Cells

Cpd.1-Cpd.4 comprising IL-2 protein coding sequence were tested for IL-2 expression and secretion from HaCaT cells. Protein levels of secreted IL-2 were measured in the cell culture supernatant using IL-2 ELISA and are represented as fold changed referenced to Cpd.1 (containing WT IL-2 signal peptide) in FIG. 2B. The protein levels of secreted IL-2 by cells transfected with Cpd.2-Cpd.4 (containing modified IL-2 signal peptide) were about 2.7-fold higher than protein level of secreted IL-2 by cells transfected with Cpd.1. Taken together, the data suggest that Cpd.2-Cpd.4 with homologous modified signal peptides can facilitate enhanced secretion of IL-2 in HaCaT cells compared to Cpd.1 with endogenous signal peptide. Data represent means±standard error of the mean of 3 replicates per Cpd. Significance (**, p<0.01) was assessed by one way ANOVA followed by Dunnet's multiple comparing test using Cpd.1 as control.

### Example 5: In Vitro Transfection of A549 Cells

Human lung epithelial carcinoma cells (A549; Sigma-Aldrich Cat. #6012804) were maintained in Dulbecco's Modified Eagle's medium high glucose (DMEM, Sigma-Aldrich) supplemented with 10% (v/v) Fetal Bovine Serum (FBS, Thermofischer, Basel, Switzerland cat #10500-064). To assess the IL-2 expression the A549 cells were seeded at 10,000 cell/well in a 96 well culture plate and incubated at 37° C. in a humidified atmosphere containing 5% CO₂ for 24 hours prior to transfection. Cells were then grown in DMEM growth medium containing 10% of FBS to reach confluency <70% before transfection. Thereafter, A549 cells were transfected with specific mRNA constructs with varying concentrations 4.4 nM-35.2 nM (0.15-1.2 μg) using Lipofectamine 2000 (Invitrogen) following the manufacturer's instructions with the mRNA to Lipofectamine ratio of 1:1 w/v. 100 μl of DMEM was removed and 50 μl of Opti-MEM (Thermo Fisher Scientific) was added to each well followed by 50 μl mRNA and Lipofectamine 2000 complex in Opti-MEM. After 5 hours of incubation, the medium was replaced by fresh growth medium and the plates were incubated for 24 hours at 37° C. in a humidified atmosphere containing 5% CO₂. Cell culture supernatant were collected to measure secreted IL-2 using ELISA (ThermoFisher Cat. #887025). Significance (**, p<0.01) was assessed by one way ANOVA followed by Dunnet's multiple comparing test using Cpd.1 as control.

IL-2 Secretion in A549 Cells

Cpd.1-Cpd.4 comprising IL-2 protein coding sequence were tested for IL-2 expression and secretion from A549 cells. Protein levels of secreted IL-2 were measured in the cell culture supernatant using IL-2 ELISA and are represented as fold changed referenced to Cpd.1 (containing WT IL-2 signal peptide) in FIG. 2C. The protein levels of secreted IL-2 by cells transfected with Cpd.2-Cpd.4 (containing modified IL-2 signal peptide) were about 1.6-fold higher than protein level of secreted IL-2 by cells transfected with Cpd.1. Taken together, the data suggest that Cpd.2-Cpd.4 with homologous modified signal peptides can facilitate enhanced secretion of IL-2 in A549 cells compared to Cpd.1 with endogenous signal peptide. Data represent means±standard error of the mean of 3 replicates per Cpd. Significance (**, p<0.01) was assessed by one way ANOVA followed by Dunnet's multiple comparing test using Cpd.1 as control.

### Example 6: Combinatorial Effect of IL-2 Secretion and VEGFA Down Regulation in A549 Cells: A VEGFA Overexpression Model

In Vitro Transfection of A549 Cells

A VEGFA overexpression model was used to evaluate simultaneous VEGFA RNA interference (RNAi) and IL-2 expression by Cpd.5 in A549 cells. The VEGFA overexpression model was established by transfecting A549 cells with 0.3 pg of VEGFA mRNA. A549 cells were co-transfected with increasing concentration 4.4 nM to 35.2 nM (0.15 to 1.2 μg) of Cpd.5 to assess dose-dependent response of Cpd.5 for VEGFA interference and IL-2 overexpression. Post transfection, the cells in a growth medium without FBS were incubated at 37° C. in a humidified atmosphere containing 5% CO₂ for 24 hours, followed by quantification of VEGFA (target mRNA to downregulate; ThermoFisher Cat. #KHG0112) and IL-2 (gene of interest to overexpress; ThermoFisher Cat. #887025) present in the same cell culture supernatant by ELISA. To assess the potency of Cpd.5 against commercially available siRNA (ThermoFisher Cat. #284703), a dose-dependent response study was performed using commercial VEGFA siRNAs and Cpd.5. A549 cells were co-transfected with VEGFA mRNA (0.3 μg/well; 9.5 nM) and either commercial VEGFA siRNAs (0.05, 0.125, 0.25, 1.25 and 2.5 mM) or Cpd.5 (4.4, 8.8, 17.6, 26.4, 35.2 and 44.02 nM corresponds to 0.15, 0.3, 0.6, 0.9, 1.2 and 1.5 pg respectively). Post transfection, the cells in a growth medium without FBS were incubated at 37° C. in a humidified atmosphere containing 5% CO₂ for 24 hours, followed by quantification of VEGFA (target mRNA to downregulate; ThermoFisher Cat. #KHG0112) and IL-2 (gene of interest to overexpress; ThermoFisher Cat. #887025) present in the same cell culture supernatant by ELISA.

Results

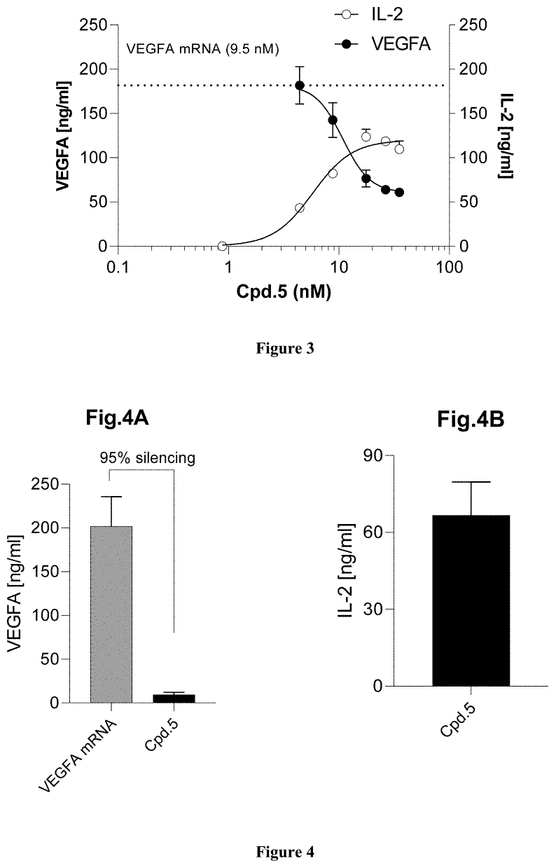

Cpd.5 comprising 3 species of VEGFA-targeting siRNA and IL-2 protein coding sequence was tested for dose-dependent VEGFA downregulation and simultaneous IL-2 expression in A549 cells by co-transfecting A549 cells with an increasing dose of Cpd.5 (4.4 nM to 35.2 nM) and constant dose of VEGFA mRNA (9.5 nM or 300 ng/well) and measuring protein levels in the cell culture supernatant by ELISA. Cpd.5 reduced VEGFA protein level (up to 70%) while increasing IL-2 protein level in a dose-dependent manner (up to above 100 ng/ml), as demonstrated in FIG. 3. Taken together, the data suggest that Cpd.5 can downregulate VEGFA without affecting IL-2 expression. Data represent means±standard error of the mean of 4 replicates.

### Example 7: Combinatorial Effect of IL-2 Secretion and VEGFA Downregulation in SCC-4 Cells: A VEGFA Overexpression Model

In Vitro Transfection of SCC-4 Cells

A VEGFA overexpression model was used to evaluate simultaneous VEGFA RNA interference (RNAi) and IL-2 expression by Cpd.5 in SCC-4 cells. The VEGFA overexpression model was established by transfecting SCC-4 cells with 9.5 nM (0.3 μg) of VEGFA mRNA. SCC-4 cells were co-transfected with increasing concertation 4.4 nM to 35.2 nM (0.15 to 1.2 μg) of Cpd.5 to assess dose-dependent response of Cpd.5 for VEGFA interference and IL-2 overexpression. Post transfection, the cells in a growth medium without FBS were incubated at 37° C. in a humidified atmosphere containing 5% CO₂ for 24 hours, followed by quantification of VEGFA (target mRNA to downregulate; ThermoFisher Cat. #KHG0112) and IL-2 (gene of interest to overexpress; ThermoFisher Cat. #887025) present in the same cell culture supernatant by ELISA. To assess the potency of Cpd.5 against VEGFA expression, SCC-4 cells were co-transfected with 9.5 nM (0.3 μg) VEGFA mRNA and Cpd.5 (4.4, 8.8, 17.6, 26.4, 35.2 and 44.02 nM corresponds to 0.15, 0.3, 0.6, 0.9, 1.2 and 1.5 μg/well). Post transfection, the cells in a growth medium without FBS were incubated at 37° C. in a humidified atmosphere containing 5% CO₂ for 24 hours, followed by quantification of VEGFA (target mRNA to downregulate; ThermoFisher Cat. #KHG0112) and IL-2 (gene of interest to overexpress; ThermoFisher Cat. #887025) present in the same cell culture supernatant by ELISA.

Results

Cpd.5, designed to have IL-2 coding sequence and 3 species of siRNA targeting VEGFA, was tested to assess the simultaneous expression of IL-2 and interference of VEGFA expression in an VEGFA overexpression model where SCC-4 cells transfected with VEGFA mRNA. Cpd.5 reduced the level of exogenously overexpressed VEGFA for up to 95% and simultaneously induced IL-2 expression (above 65 ng/ml), as demonstrated in FIG. 4A and FIG. 4B. In summary, Cpd.5 can reduce exogenously overexpressed VEGFA while simultaneously inducing IL-2 expression and secretion.

### Example 8: Combinatorial Effect of IL-2 Secretion and VEGFA Down Regulation in SCC-4 Cells: An Endogenous VEGFA Expression Model

In Vitro Transfection of SCC-4 Cells

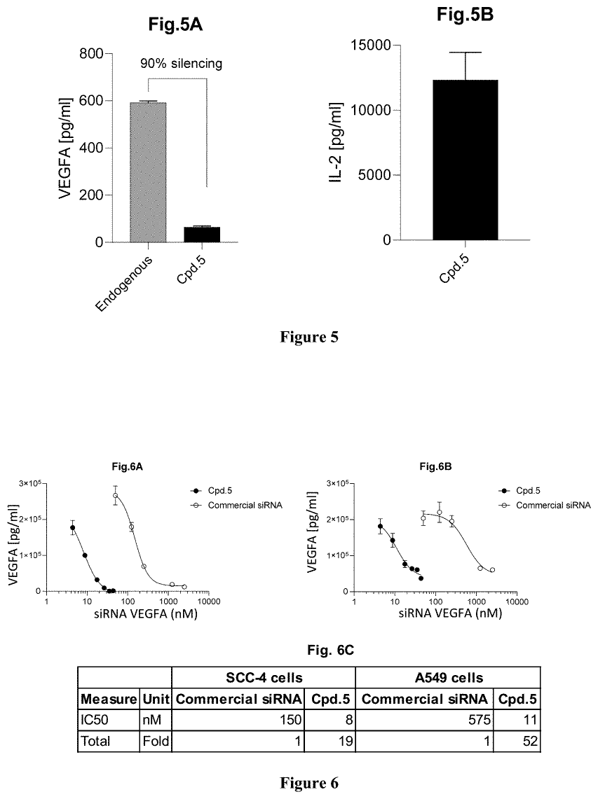

SCC-4 cells were used as an endogenous VEGFA overexpression model, as SCC-4 cells endogenously overexpress VEGFA up to 600 pg/mL in vitro (FIG. 5A), to evaluate simultaneous VEGFA RNA interference (RNAi) and IL-2 expression by Cpd.5. SCC-4 cells were transfected with 26.4 nM (0.9 μg) of Cpd.5. Cells were incubated at 37° C. in a humidified atmosphere containing 5% CO₂ for 24 hours, followed by quantification of VEGFA (ThermoFisher Cat. #KHG0112) and IL-2 (ThermoFisher Cat. #887025) present in the same cell culture supernatant by using specific ELISAs.

Results

Cpd.5, designed to have IL-2 coding sequence and 3 species of siRNA targeting VEGFA, was tested to assess the simultaneous expression of IL-2 and interference of VEGFA expression in SCC-4 cells that constitutively express VEGFA up to 600 pg/mL in vitro. Cpd.5 reduced the level of endogenous VEGFA expression for up to 90% and simultaneously induced IL-2 expression (up to 12 ng/ml), as demonstrated in FIG. 5A and FIG. 5B. Taken together Cpd.5 can reduce the level of endogenously expressed VEGFA while simultaneously inducing expression and secretion of IL-2.

### Example 9: Comparative Analysis of Cpd.5 and Commercial siRNA in VEGFA Downregulation

In Vitro Transfection of SCC-4 Cells

Human tongue squamous carcinoma cell line (SCC-4; Sigma-Aldrich, Buchs Switzerland, Cat. #89062002 CRL-1573) were maintained in Dulbecco's Modified Eagle's high glucose medium (DMEM, Sigma Aldrich) supplemented with HAM F12 (1:1)+2 mM Glutamine+10% Fetal Bovine Serum (FBS)+0.4 μg/ml hydrocortisone. Cells were seeded at 15,000 cell/well in a 96 well culture plate and incubated at 37° C. in a humidified atmosphere containing 5% CO₂ for 24 hours prior to transfection. Cells were grown in DMEM/HAM F-12 growth medium to reach confluency <70% before transfection. To assess the potency of Cpd.5 against commercially available siRNA (ThermoFisher Cat. #284703), a dose response study was performed using commercial VEGFA siRNA and Cpd.5. SCC-4 cells were co-transfected with 9.5 nM (0.3 μg) VEGFA mRNA and either commercial VEGFA siRNA (0.05, 0.125, 0.25, 1.25 and 2.5 mM) or Cpd.5 (4.4, 8.8, 17.6, 26.4, 35.2 and 44.02 nM corresponds to 0.15, 0.3, 0.6, 0.9, 1.2 and 1.5 SCC-4 cells were transfected with Cpd.5c mRNA or siRNA constructs at specified concentrations using Lipofectamine 2000 (Invitrogen) following the manufacturer's instructions with the mRNA to Lipofectamine ratio of 1:1 w/v. 100 μl of DMEM was removed and replaced with 50 μl of Opti-MEM and 50 μl mRNA and Lipofectamine 2000 complex in Opti-MEM (Thermo Fisher Scientific). After 5 hours, the medium was replaced by fresh growth medium without FBS and the plates were incubated at 37° C. in a humidified atmosphere containing 5% CO₂ for 24 hours.

Results

To calculate the inhibitory concentration of Cpd.5 against commercially available siRNA in downregulating VEGFA expression, a dose response study was performed in VEGFA overexpression model established in both SCC-4 cells and A549 cells. Both cells were co-transfected with 9.5 nM (0.3 μg) VEGFA mRNA with increasing concentration of either Cpd.5 (4.4 nM to 44.02 nM) or commercial siRNA (0.05 mM to 2.5 mM). In comparison to commercial siRNA, Cpd.5 exhibited 19-fold higher potency in SCC-4 cells and more than 52-fold higher potency in A549 cells in reducing VEGFA expression (FIG. 6A and FIG. 6B). The IC50 value of Cpd.5 in SCC-4 cells (8 nM) and in A549 cells (11 nM) are shown in FIG. 6C.

### Example 10: Combinatorial Effect of IL-2 Secretion and MICB Down Regulation in SCC-4 Cells—an Endogenous MICB Expression Models

In Vitro Transfection of SCC-4 Cells

SCC-4 cells were used an endogenous MICB expression model, as SCC-4 cells constitutively express soluble MICB (up to 40 pg/mL) and membrane bound MICB (up to 80 pg/mL) in vitro, to evaluate simultaneous MICB RNA interference (RNAi) and IL-2 expression by Cpd.6. SCC-4 cells were transfected with 35.11 nM (0.9 μg) of Cpd.6 and were incubated at 37° C. in a humidified atmosphere containing 5% CO₂ for 24 hours. MICB levels present in the cell culture supernatant and cell lysate were quantified using ELISA (ThermoFisher Cat. #BMS2303). IL-2 levels present in the same cell culture supernatant was measured using ELISA (ThermoFisher Cat. #887025).

Results

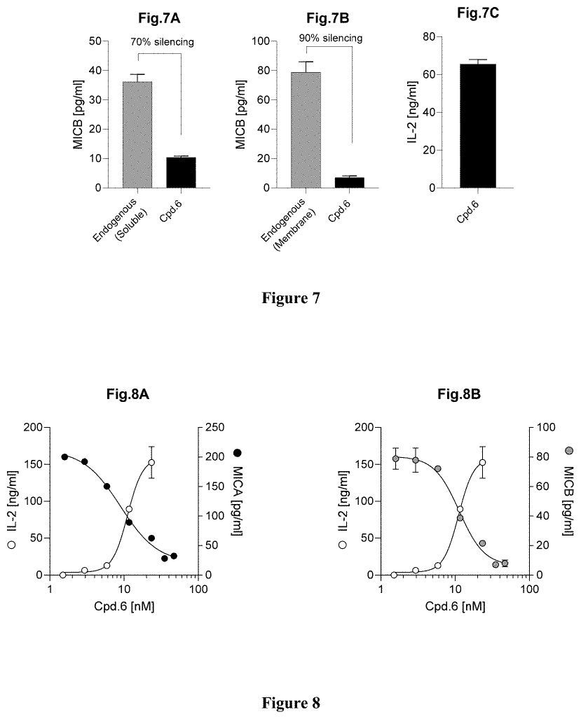

Cpd.6, designed to have IL-2 coding sequence and 3 species of siRNA targeting MICB, was tested to assess the simultaneous expression of IL-2 and interference of MICB expression in SCC-4 cells that constitutively express soluble MICB (up to 40 pg/mL) and membrane bound MICB (up to 80 pg/mL) in vitro. Cpd.6 reduced the level of endogenous expression of both soluble and membrane bound MICB for up to 70% and 90% respectively and simultaneously induced IL-2 expression (up to 65 ng/ml), as demonstrated in FIGS. 7A-7C. In brief, Cpd.6 can downregulate endogenously expressed MICB (both soluble and membrane bound) while simultaneously inducing expression and secretion of IL-2. Data represent means±standard error of the mean of four replicates.

### Example 11: Combinatorial Effect of IL-2 Secretion Together with MICA and MICB Down Regulation in SCC-4 Cells—an Endogenous MICA & MICB Expression Model

In Vitro Transfection of SCC-4 Cells

In addition to MICB, SCC-4 cells constitutively express soluble MICA (up to 200 pg/mL) in vitro, a functional analog to MICB. Due to high genomic homology between MICA and MICB (>90%), siRNAs in Cpd.6 were designed to interfere the expression of both MICA and MICB protein simultaneously. To evaluate synchronized MICA and MICB RNA interference (RNAi) with IL-2 expression and secretion by Cpd.6, SCC-4 cells were transfected with increasing doses of Cpd.6 mRNA (1.58, 2.93, 5.85, 11.7, 23.41, 35.11 and 46.81 nM) and were incubated at 37° C. in a humidified atmosphere containing 5% CO₂ for 24 hours. MICA levels present in the cell culture supernatant were quantified using ELISA (RayBioech Cat. #ELH-MICA-1). MICB levels present in the same cell culture supernatant were quantified using ELISA (ThermoFisher Cat. #BMS2303). IL-2 levels present in the same cell culture supernatant were measured using ELISA (ThermoFisher Cat. #887025).

Results

Cpd.6, designed to have IL-2 coding sequence and 3 species of siRNA targeting both MICA and MICB, was tested to assess the simultaneous expression of IL-2 and interference of MICA/MICB expression in SCC-4 cells that constitutively express soluble MICA and MICB in vitro. Cpd.6 reduced the level of endogenous expression of both soluble MICA and soluble MICB in a dose dependent manner up to 80% and simultaneously induced IL-2 expression (>150 ng/ml), as demonstrated in FIGS. 8A and 8B. In brief, Cpd.6 can downregulate endogenously expressed MICA and MICB while simultaneously inducing secretion of IL-2. Data represent means±standard error of the mean of four replicates for IL-2 level and two replicates for MICA and MICB each.

### Example 12: Bioactivity Evaluation of Cpd.3 in a Peripheral Blood Mononuclear Cells Tumour Killing Assay in a SK-OV-3 Spheroid Model

The anti-tumor activity of Cpd.3 was assessed in immune cell-mediated tumor cell killing, by using nuclear-RFP transduced SK-OV-3 tumor cell lines. For the IL-2 expression and secretion induced by Cpd.3 in spheroids, SK-OV-3-NLR cells from two dimensional (2D) culture were seeded at a single density (5000 cells/well) into an ultra-low attachment (ULA) plate and transfected with 100 ng of Cpd.3 construct using Lipofectamine 2000, then centrifuged (200× g for 10 min) to generate spheroids. Conditions were set up in quadruplicates. The supernatants were harvested at 12, 24 and 48 hours following the transfection to test for IL-2 expression by TR-FRET (PerkinElmer, Cat. #TRF1221C). For experiments with peripheral blood mononuclear cells (PBMCs), the spheroids were generated and transfected with Cpd.3 (3 ng, 10 ng, 30 ng and 100 ng) as described above and were cultured for 48 hours to allow spheroids to reach between 200-500 μm in diameter prior to PBMC addition. Following the 48 hour culture period, PBMCs from 3 healthy donors were added to wells (200,000 cells/well) in the presence of anti-CD3 antibody. Recombinant human IL-2 (2000 IU/ml) and PBMCs were added to appropriate wells as the positive control. SK-OV-3-NLR alone conditions did not receive PBMCs. Wells were imaged every 3 hours for 7 days using an IncuCyte (S3), with changes in the total nuclear localized RFP (NLR) integrated intensity measured as the readout for PBMC-mediated SK-OV-3 spheroid tumor killing. Total NLR integrated intensity was normalized to the 24 hour time point and analyzed using the spheroid module within the IncuCyte software. The graphs show data from Day 5 analyzed with an additional smoothing function using GraphPad Prism (averaging 4 values on each side and using a second order smoothing polynomial).

Results

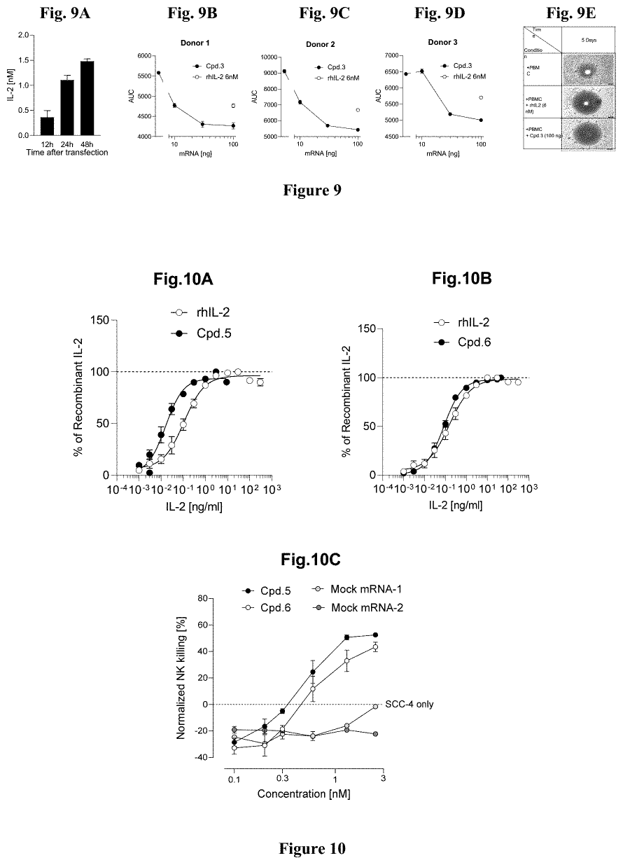

TR-FRET analysis of the supernatants collected from the spheroids which were formed from cells transfected in 3D suspension cultures with Cpd.3 (100 ng) demonstrated time dependent increase in IL-2 expression and secretion (FIG. 9A). No deficiency in spheroid formation and growth was noticed due to lipofectamine transfection. Analysis of the transfected spheroids with Cpd.3 following addition of PBMCs from 3 healthy donors demonstrated clear dose-dependent immune-mediated killing. Across all donors Cpd.3 at 30 ng and 100 ng promoted PBMC-driven tumor killing determined by the reduction in the total integrated NLR intensity measured over the period of the assay (day 6 data is presented in FIGS. 9B, 9C and 9D). The killing effect induced by Cpd.3 was substantially better than that of recombinant human IL-2 (rhIL-2) added at 6 nM concentration in all the three donors tested. FIG. 9E shows a set of representative IncuCyte images showing NLR integrity reduction after Cpd.3 treatment (100 ng) in the SK-OV-3 NLR condition compared to control at Day 5. In summary, transfection of SK-OV-3 NLR spheroids with Cpd.3 mRNA constructs enhanced PBMC-mediated tumor killing in a dose-dependent manner.

### Example 13: HEK-Blue™ hIL-2 Reporter Assay for JAK3-STATS Activation

The functional activity of Cpd.5 and Cpd.6 was tested in HEK-Blue™ IL-2 reporter cells (Invivogen, Cat. Code: hkb-il2), which are designed for studying the activation of human IL-2 receptor by monitoring the activation of JAK/STAT pathway. These cells were derived from the human embryonic kidney HEK293 cell line and engineered to express human IL-2Rα, IL-2Rβ, and IL-2Rγ genes, together with the human JAK3 and STATS genes to achieve a totally functional IL-2 signaling cascade. In addition, a STATS-inducible SEAP reporter gene was introduced. Upon IL-2 activation followed by STATS, produced SEAP can be determined in real-time with HEK-Blue™ Detection cell culture medium in cell culture supernatant. Stimulation of HEK-Blue™ IL-2 cells were achieved by recombinant human IL-2 (rhIL-2, 0.001 ng to 300 ng) or IL-2 derived from cell culture supernatant of HEK293 cells (0.001 ng-45 ng) which had been transfected with Cpd.5 or Cpd.6 (0.3 μg/well) with below details.

HEK-Blue™ hIL-2 cells were maintained in Dulbecco's Modified Eagle's medium (DMEM, Sigma Aldrich) supplemented with 10% (v/v) Fetal Bovine Serum (FBS). The antibiotic Blasticidin (10 μg/mL) and Zeocin (100 μg/mL) were added to the media to select cells containing IL-2Rα, IL-2Rβ, IL-2Rγ, JAK3, STATS and SEAP transgene plasmids. Cells were seeded at 40,000 cell/well in a 96 well culture plate and incubated at 37° C. in a humidified atmosphere containing 5% CO₂ for 24 hours prior to stimulation. Cells were grown in DMEM growth medium containing 10% of FBS to reach confluency <80% before stimulation. Defined concertation of IL-2 derived from HEK293 cell culture supernatant were collected, diluted in 20 μl of media, and added to culture media of HEK-Blue™ IL-2 cells to measure IL-2 receptor recruitment followed by JAK3-STATS pathway activation. rhIL-2 (0.001-300 ng) or IL-2 derived from Cpd.5 and Cpd.6 (0.001-45 ng) were tested in parallel. After 2 hours of incubation, SEAP activity was assessed using QUANTI-Blue™ (20 μl cell culture supernatant+180 μl QUANTI-Blue™ solution) and reading the optical density (O.D.) at 620 nm in SpectraMax i3 multi-mode plate reader (Molecular Device). Untransfected samples were used as background control and subtracted from obtained O.D. values in tested samples.

Results

Stimulation of HEK-Blue™ IL-2 cells with rhIL-2 or IL-2 derived from cell culture supernatant of HEK293 cells that had been transfected with Cpd.5 or Cpd.6 was functional as all three tested compounds induced SEAP production in a dose-dependent fashion (FIGS. 10A and 10B). In direct comparison, Cpd.5-derived IL-2 was −5× more potent (EC₅₀: 0.02 ng/ml) compared to rhIL-2 (EC₅₀: 11 ng/ml), as well as Cpd.6 being −2× more potent (EC₅₀: 0.08 ng/ml) compared to rhIL-2 (EC₅₀: 0.15 ng/ml). In summary, IL-2 derived from Cpd.5 and Cpd.6 are functional and induce IL-2 signaling cascade at least as potent as rhIL-2.

### Example 14: NK-Cell Mediated Killing Assay of Cpd.5 and Cpd.6

Natural killer cells (NK cells) have the potential to target and eliminate tumor cells and are majorly primed by IL-2 cytokine. To measure the capacity of Cpd.5 and Cpd.6 in activating NK cells through IL-2 mechanism, SCC4 cells (Sigma-Aldrich, Buchs Switzerland, Cat. #89062002 CRL-1573) and Natural killer 92 cells (NK-92, DSMZ, ACC488, Germany) were used. Dose response study (0.1 nM to 2.5 nM) was performed in SCC4 cells (10,000/well) by transfecting SCC-4 cells with Cpd.5 (IL-2 mRNA+3×VEGFA siRNA), Cpd.6 (IL-2 mRNA+3×MICA/B siRNA), mock RNA-1 (IL-4 mRNA+3×TNF-α siRNA) or mock RNA-2 (MetLuc mRNA, no siRNA) using Lipofectamine MessangerMax (ThermoFisher, Cat. #LMRNA015) in Opti-MEM. The SCC-4 cells were then incubated at 37° C. in a humidified atmosphere containing 5% CO₂ for 30 minutes in a black 96 well culture plate. NK-92 effector cells at 100,000 cell/well in Opti-MEM were added to the transfected SCC-4 target cells in Effector to Target ratio of 10:1 (E:T=10:1). After 24 hours, the black 96 well plate was sealed with a black foil on the bottom and washed 3 times with Dulbecco's Phosphate-Buffered Saline (PBS⁺⁺, BioConcept, Cat. #3-05F001) to remove NK-92 cells which were in suspension. Since SCC-4 cells are adherent in nature, 24 hours incubation led to strong adhesion of cells to the bottom of plate and only NK-92 cells were washed off The rational is that if NK cells lead to the killing of SCC-4 cells, there would be less SCC-4 cells survive and attach to the bottom of the plate after washing, which can be quantitatively measured by cell viability assay. After 3× washes, 50 μl of PBS⁺⁺ and 50 μl of CellTiter-Glo 2.0 (CTG 2.0, Promega, Cat. #G924B) reagent were added to each well and the 96 well plate was incubated at room temperature in the dark for 10 minutes. The luminescence was measured with the SpectraMax i3x (Molecular Devices) to calculate cell viability using standard settings.

Results

NK cell mediated killing assay revealed a dose dependent cell lysis of SCC-4 cells which were transfected with Cpd.5 or Cpd.6, and co-incubated with NK-92 cells. IL-2 secreted from SCC-4 cells promoted targeted killing of SCC-4 tumor cells at E:T ratio of 10:1 (>50% for Cpd.5 and >40% for Cpd.6, FIG. 10C). NK cell mediated killing was observed for SCC-4 cells transfected with both Cpd.5 and Cp.6. In brief, Cpd.5 and Cpd.6 demonstrated expected anti-tumor activity by activating NK cells in dose dependent fashion.

### Example 15: Comparative Analysis of Cpd.7 and Cpd.8 in IL-2 Expression and VEGFA Downregulation in SCC-4 Cells

SCC-4 cells were cultured and transfected as described above. To assess the potency of Cpd.7 (IL-2 mRNA+3×VEGFA siRNA) against Cpd.8 (IL-2 mRNA+5×VEGFA siRNA), a dose response study was performed using both compounds. SCC-4 cells were transfected with Cpd.7 (1.1, 2.2, 4.4, 8.8, 17.6, 26.4, 35.2 and 44.04 nM/well) or Cpd.8 (0.47, 0.94, 1.89, 3.79, 7.58, 15.15, 22.73, 30.31 and 37.88 nM/well). After 5 hours, the medium was replaced by fresh growth medium without FBS and the plates were incubated at 37° C. in a humidified atmosphere containing 5% CO₂ for 24 hours, and supernatant were collected. ELISA was performed to quantify VEGFA (ThermoFisher Cat. #KHG0112) and IL-2 (ThermoFisher Cat. #887025) levels present in the same cell culture supernatant. 80% downregulation of VEGFA was calculated using a non-linear Hill binding curve with GraphPad prism.

Results

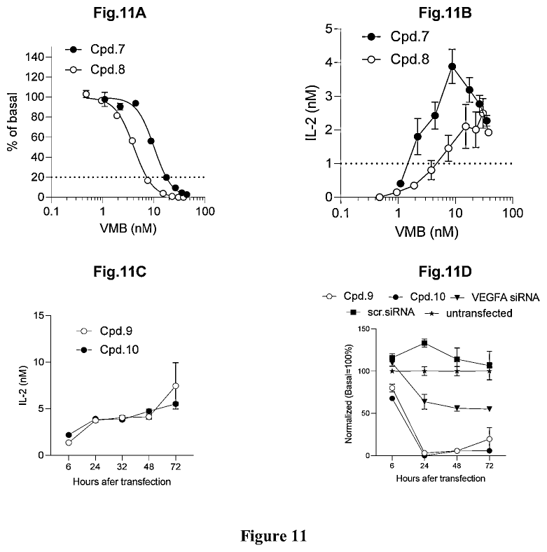

To calculate the inhibitory concentration of Cpd.7 against Cpd.8 in downregulating VEGFA expression, a dose response study was performed in SCC-4 cells transfected with Cpd.7 or Cpd.8. Cells were transfected with increasing concentrations of either Cpd.7 or Cpd.8 as described above. In comparison to Cpd.7, Cpd.8 exhibited 2.5-fold higher potency in SCC-4 cells in reducing VEGFA expression (FIG. 11A). 80% VEGF downregulation was achieved by Cpd.8 in SCC-4 cells at 8 nM whereas by Cpd.7 at 18 nM, demonstrating that increasing copy number of siRNA leads to higher level of VEGFA downregulation. However, IL-2 expression from Cpd.8 was −2 fold lower than IL-2 expression from Cpd.7 (FIG. 11B). In summary, increasing copy number of siRNA in the compounds enhances RNA interference but compromises the expression of mRNA target.

### Example 16: Time-Course Study of Cpd.9 and Cpd.10 in IL-2 Expression and VEGFA Downregulation

SCC-4 cells were cultured and transfected as described above. To assess the longitudinal potency of Cpd.9 (IL-2 mRNA+3×VEGFA siRNA, same siRNA repeated 3 times) against Cpd.10 (IL-2 mRNA+3×VEGFA siRNA, 3 different siRNAs with 30 bp in length), a time course study was performed using SCC-4 cells transfected with Cpd.9 or Cpd.10. SCC-4 cells were transfected with Cpd.9 or Cpd.10 at 30 nM/well concentration. Commercially available VEGFA siRNA (ThermoFisher Cat. #284703) were added to the experiment for comparison and scrambled siRNA (Sigma, Cat. #SIC002) was used as control. Cells were then incubated at 37° C. in a humidified atmosphere containing 5% CO₂. The samples from different wells were collected between 6 hours and 72 hours after transfection. ELISA was performed to quantify VEGFA (ThermoFisher Cat. #KHG0112) and IL-2 (ThermoFisher Cat. #887025) levels present in the same cell culture supernatant. VEGFA levels from untransfected cells at each timepoint were set to 100% and the level of VEGFA downregulation was normalized to that level at the respective time point.

Results

The time course study showed the accumulation of IL-2 over 72 hours in a similar way for both Cpd.9 and Cpd.10 (FIG. 11C). However, Cpd.10 resulted in stronger VEGFA downregulation until 72 hours as higher than 95% RNA interference level was achieved, while Cpd.9 resulted in 85% RNA interference level after 48 hours (FIG. 11D). The effect was visible even at the 6 hour time point which showed VEGFA downregulation by Cpd.10 (>30%) was higher than VEGFA downregulation by Cpd.9 (20%) as demonstrated in FIG. 11D. As observed previously, commercial VEGFA siRNA resulted in up to 45% downregulation of VEGFA. Universal scrambled siRNA did not alter the VEGFA expression throughout the experiment phase. In summary, Cpd.10 displayed long lasting VEGFA downregulation with slightly improved potency as compared to Cpd. 9.

### Example 17: Targeting Multiple Signaling Pathways in Cancer: A Combination of Multiple siRNA Targets and Immune Stimulating Cytokines in In Vitro Tumor Models

Cancer is a complex disease with multiple dysregulated signaling pathways which promote uncontrolled proliferation of cells with reduced apoptosis. The upregulation of tumor growth signals including mammalian target of rapamycin (mTOR), cyclin-dependent kinases (CDK), vascular endothelial growth factor (VEGFA), epidermal growth factor receptor (EGFR), Kirsten rat sarcoma viral oncogene (KRAS), c-Myc proto-oncogene (c-Myc) along with high expression of immune escape proteins such as MHC class I chain-related sequence A/B (MICA/B) and Programmed cell death-ligand 1 (PD-L1) are observed in tumor cells. Moreover, tumor microenvironment displays reduced level of immune stimulating cytokines such as Interleukin-2 (IL-2), Interleukin-12 (IL-12), Interleukin-15 (IL-15) and Interleukin-7 (IL-7). Therefore, downregulation of the key proteins involved in tumor growth along with upregulation of immune stimulating cytokines can be an attractive approach for cancer therapy. To measure the downregulation of multiple pro-tumor targets through RNA interference and upregulation of immune stimulating cytokines, Cpd.11, Cpd.12, Cpd.15 and Cpd.16 were designed to comprise more than one siRNA target along with an anti-tumor interleukin mRNA. The effect of these compounds in targeting multiple signaling pathways were assessed in SCC-4 cells, A549 cells and human glioblastoma cell line (U251 MG) cells.

Head and Neck Cancer In Vitro Model in SCC-4 Cells

Human tongue squamous carcinoma cell line (SCC-4) was derived from the tongue of a 55-year old male and used to simulate a head and neck cancer in vitro model in this example. SCC-4 cells were cultured and transfected as described above. To assess modulation of multiple cancer relevant targets in parallel using Cpd.11 (IL-12 mRNA+1×IDH1 siRNA+1×CDK4 siRNA+1×CDK6 siRNA), Cpd.12 (IL-12 mRNA+1×EGFR siRNA+1× mTOR siRNA+1×KRAS siRNA) and Cpd.15 (IL-15 mRNA+1×VEGFA siRNA+2× CD155 siRNA), SCC-4 cells were transfected with these compounds at 10 and 30 nM/well concentration. 5 hours after transfection, the medium was replaced by fresh growth medium without FBS and the plates were incubated at 37° C. in a humidified atmosphere containing 5% CO₂ for 24 hours, and supernatant were collected. ELISA was performed to quantify human IL-12p70 (ThermoFisher Cat. #88-7126) and human IL-15 (ThermoFisher Cat. #88-7620) levels present in the cell culture supernatant. The respective cell lysates were also processed to measure RNA abundance of siRNA target genes by relative quantification against untransfected samples by RT-qPCR using Cells-to-CT™ 1-Step Power SYBR Green kit (ThermoFisher Cat. #A25599) and primers (primer sequence details are listed in Table 6). The human 18s rRNA was used as a reference control.

Results

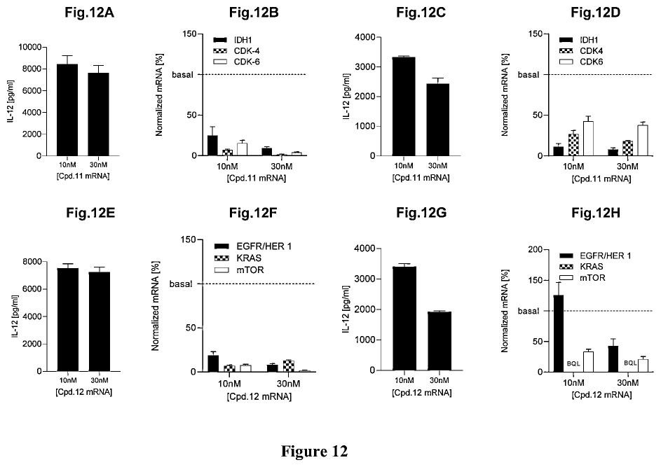

The effect of Cpd.11 comprising 1× siRNA of IDH1, CDK4 and CDK6, and IL-12 mRNA and Cpd.12 comprising 1× siRNA of EGFR, mTOR and KRAS and IL-12 mRNA was evaluated for IL-12 expression and simultaneous downregulation of target genes in SCC-4 cells transfected with two different doses (10 nM and 30 nM) of Cpd.11 or Cpd.12. The data demonstrate that both Cpd.11 and Cpd.12 lead to significant IL-12 protein expression and secretion (>7000 pg/ml) as shown in FIGS. 12A and 12E. In the same cell lysate, the RNA interference of Cpd.11 against IDH1, CDK4 and CDK6 RNA transcripts was assessed. As demonstrated in FIG. 12B, Cpd.11 downregulated endogenous IDH1 (75% for 10 nM, 90% for 30 nM), CDK4 (93% for 10 nM, 98% for 30 nM) and CDK6 (85% for 10 nM, 96% for 30 nM) levels in a dose-dependent manner. The RNA interference of Cpd.12 against EGFR, mTOR and KRAS RNA transcripts was assessed in the same cell lysate of FIG. 12E. As shown in FIG. 12F, Cpd.12 downregulated endogenous EGFR (80% for 10 nM, 92% for 30 nM), KRAS (92% for 10 nM, 83% for 30 nM) and mTOR (92% for 10 nM, 98% for 30 nM) levels in a dose-dependent manner for KRAS.

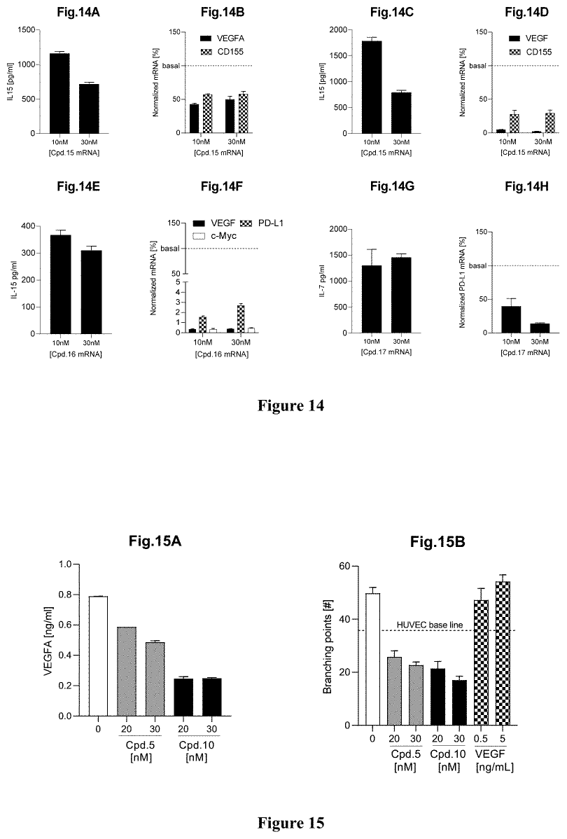

In addition, the effect of Cpd.15 comprising 1×VEGFA siRNA, 2× CD155 siRNA. and IL-15 mRNA was evaluated for IL-15 expression and simultaneous downregulation of the target genes in SCC-4 cells transfected with two different doses (10 nM and 30 nM) of Cpd.15. Results showed that Cpd.15 expresses IL-15 protein (>790 pg/ml), as shown in FIG. 14C. In the same cell lysate, the RNA interference of Cpd.15 against VEGFA and CD155

RNA transcripts was assessed using qPCR. As demonstrated in FIG. 14D, Cpd.15 downregulated endogenous VEGFA (95% for 10 nM, 98% for 30 nM), and CD155 (73% for nM, 71% for 30 nM) levels. In short, multiple signaling pathways can be targeted using Cpd.11, Cpd.12 and Cpd.15 to downregulate multiple oncology targets through siRNAs and upregulate IL-12 or IL-15 cytokine at the same time to provide anti-tumor activity either by promoting infiltration or proliferation of immune cells.

Lung Cancer In Vitro Model in A549 Cells

A549 cells are adenocarcinomic human alveolar basal epithelial cells derived from cancerous lung of a 58-years old male and were used to simulate a lung cancer in vitro model in this example. A549 cells were cultured and transfected as described above. To assess modulation of multiple cancer relevant targets in parallel using Cpd.11 (IL-12 mRNA+1× IDH1 siRNA+1×CDK4 siRNA+1×CDK6 siRNA), Cpd.12 (IL-12 mRNA+1×EGFR siRNA+1× mTOR siRNA+1×KRAS siRNA) and Cpd.15 (IL-15 mRNA+1×VEGFA siRNA+2× CD155 siRNA), A549 cells were transfected with these compounds at 10 and 30 nM/well concentration. Five hours after transfection, the medium was replaced by fresh growth medium without FBS and the plates were incubated at 37° C. in a humidified atmosphere containing 5% CO₂ for 24 hours, and supernatant were collected. ELISA was performed to quantify human IL-12p70 (ThermoFisher Cat. #88-7126) and human IL-15 (ThermoFisher Cat. #88-7620) levels present in the cell culture supernatant. The respective cell lysates were also processed to measure RNA abundance of siRNA target genes by relative quantification against untransfected samples by RT-qPCR using Cells-to-CT™ 1-Step Power SYBR Green kit (ThermoFisher Cat. #A25599) and primers (primer sequence details are listed in Table 6). The human 18s rRNA used as a reference control.

Results

The effect of Cpd.11 comprising 1× siRNA of IDH1, CDK4 and CDK6 and IL-12 mRNA and Cpd.12 comprising 1× siRNA of EGFR, mTOR KRAS, and IL-12 mRNA was evaluated for IL-12 expression and simultaneous downregulation of target genes in A549 cells transfected with two different doses (10 nM and 30 nM) of Cpd.11 or Cpd.12. The data demonstrate that both Cpd.11 and Cpd.12 lead to significant IL-12 protein expression and secretion (>1925 pg/ml) as shown in FIGS. 12C and 12G. In the same cell lysate, the RNA interference of Cpd.11 against IDH1, CDK4 and CDK6 RNA transcripts was assessed. As demonstrated in FIG. 12D, Cpd.11 downregulated endogenous IDH1 (88% for 10 nM, 92% for 30 nM), CDK4 (74% for 10 nM, 80% for 30 nM) and CDK6 (58% for 10 nM, 60% for 30 nM) levels. The RNA interference of Cpd.12 against EGFR, mTOR and KRAS RNA transcripts was assessed in same cell lysate of FIG. 12G. As shown in FIG. 12H, Cpd.12 downregulated endogenous EGFR levels (up to 58%) in SCC-4 cells transfected with 30 nM of Cpd.12. In this cell line, endogenous KRAS mRNA expression was too low to detect by KRAS qPCR assay, levels were below quantification limit even under control conditions (BQL). As shown in FIG. 12H, Cpd.12 downregulated endogenous mTOR levels in a dose-dependent manner (67% for 10 nM and 79% for 30 nM).

In addition, the effect of Cpd.15 comprising 1×VEGFA siRNA, 2× CD155 siRNA, and IL-15 mRNA was evaluated for IL-15 expression and simultaneous downregulation of target genes in A549 cells transfected with different doses (10 nM and 30 nM) of Cpd.15. As shown in FIG. 14A, Cpd.15 lead to significant IL-15 protein expression and secretion (>715 pg/ml). In the same cell lysate, the RNA interference of Cpd.15 against VEGFA and CD155 RNA transcripts was assessed using qPCR. As demonstrated in FIG. 14B, Cpd.15 downregulated endogenous VEGFA (58% for 10 nM, 51% for 30 nM) and CD155 (43% for nM, 42% for 30 nM) levels. In short, multiple signaling pathways can be targeted using Cpd.11, Cpd.12 and Cpd.15 to downregulate multiple oncology targets through siRNAs and upregulate IL-12 or IL-15 cytokine at the same time to provide anti-tumor activity either by promoting infiltration or proliferation of immune cells.

Glioblastoma Cancer In Vitro Model in U251 MG Cells

Human glioblastoma cell line (U251 MG; DSMZ, Germany, Cat. #09063001) was derived from a human malignant glioblastoma. U251 MG cells were maintained in Dulbecco's Modified Eagle's medium high glucose (DMEM, Sigma Aldrich, Cat #D0822) supplemented with 10% (v/v) Fetal Bovine Serum (FBS). Cells were seeded at 20,000 cell/well in a 96 well culture plate and incubated at 37° C. in a humidified atmosphere containing 5% CO₂ for 24 hours prior to transfection. Cells were grown in DMEM growth medium to reach confluency <70% before transfection. Thereafter, U251 MG cells were transfected with Cpd.16 (IL-15 mRNA+1×VEGFA siRNA+1× PD-L1 siRNA+1× c-Myc siRNA) at 10 nM or 30 nM concentration using Lipofectamine MessengerMax (Invitrogen) following the manufacturer's instructions with the compound to Lipofectamine ratio of 1:1 w/v. 100 μl of DMEM was removed and replaced with 90 μl of Opti-MEM (Thermo Fisher Scientific, Switzerland, Cat #31985-070) and 10 μl compound and Lipofectamine MessangerMax complex in Opti-MEM. After 5 hours, the medium was replaced by fresh growth medium without FBS and the plates were incubated at 37° C. in a humidified atmosphere containing 5% CO₂ for 24 hours. ELISA was performed to quantify human IL-15 (ThermoFisher Cat. #88-7620) levels present in the cell culture supernatant. The respective cell lysates were also processed to measure RNA abundance of siRNA target genes by relative quantification against untransfected samples by RT-qPCR using Cells-to-CT™ 1-Step Power SYBR Green kit (ThermoFisher Cat. #A25599) and primers (primer sequence details are listed in Table 6). The human 18s rRNA used as a reference control.

Results

The effect of Cpd.16 comprising 1× siRNA of VEGFA, PD-L1 and c-Myc and IL-15 mRNA was evaluated for IL-15 expression and simultaneous downregulation of target genes in U251 MG cells transfected with two different doses (10 nM and 30 nM) of Cpd.16. The data demonstrate that Cpd.16 expresses IL-15 protein (>300 pg/ml) as shown in FIG. 14E. In the same cell lysate, the RNA interference of Cpd.16 against VEGFA, PD-L1 and c-Myc RNA transcripts was assessed. As demonstrated in FIG. 14F, Cpd.16 downregulated endogenous VEGFA by 99% for 10 and 30 nM, PD-L1 by >97% for 10 and 30 nM and c-Myc by >99% for and 30 nM levels. In summary, multiple signaling pathways can be targeted using Cpd.16 to downregulate multiple oncology targets through siRNAs and to upregulate the IL-15 cytokine at the same time to provide anti-tumor activity by promoting proliferation of anti-tumor immune cells such as NK-cells and T-cells.

### Example 18: A Combination of Single siRNA Target and Immune Stimulating Cytokines in In Vitro Tumor Models

In this example, the impact of targeting a single pro-tumor gene for down regulation along with over expression of immune stimulating cytokine. The parallel modulation of cancer relevant target and cytokine secretion of Cpd.13 (IL-12 mRNA+3×EGFR siRNA), Cpd.14 (IL-12 mRNA+3× mTOR siRNA) and Cpd.17 (IL-7 mRNA+3× PD-L1 siRNA) in SCC-4 cells, A549 cells and U251MG cells was assessed. All the three cells were cultured and transfected as described above with two different doses (10 nM and 30 nM) of above compounds. 24 hours after transfection, supernatant were collected. ELISA was performed to quantify human IL-12p70 (ThermoFisher Cat. #88-7126) and human IL-7 (ThermoFisher Cat. #EHIL7) levels present in the cell culture supernatant. The respective cell lysates were also processed to measure RNA abundance of siRNA target genes by relative quantification against untransfected samples by RT-qPCR using Cells-to-CT™ 1-Step Power SYBR Green kit (ThermoFisher Cat. #A25599) and primers (primer sequence details are listed in Table 6). The human 18s rRNA used as a reference control.

Results

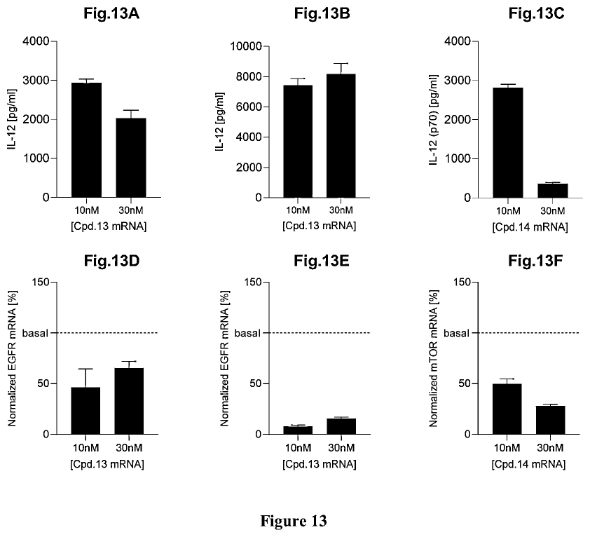

The effect of Cpd.13 comprising 3×EGFR siRNA and IL-12 mRNA was evaluated for IL-12 expression and simultaneous EGFR gene downregulation in both A549 cells and SCC-4 cells transfected with two different doses (10 nM and 30 nM) Cpd.13. As shown in FIGS. 13A and 13B, Cpd.13 expressed IL-12 protein in both A549 cells (up to 2030 pg/ml) and SCC-4 cells (up to 7420 pg/ml). In the same cell lysate, the RNA interference of Cpd.13 against EGFR RNA transcripts was assessed. As demonstrated in FIG. 13D and FIG. 13E, Cpd.13 downregulated the endogenous EGFR levels (30-40% in A549 cells and 85-92% in SCC-4 cells).

Likewise, Cpd.14 comprising 3× mTOR siRNA and IL-12 mRNA was evaluated for IL-12 expression and simultaneous mTOR gene downregulation in A549 cells transfected with two different doses (10 nM and 30 nM) of Cpd.14. As shown in FIG. 13C, Cpd.14 expressed IL-12 protein (up to 2800 pg/ml in cells transfected with 10 nM of Cpd.14 and 365 pg/ml in cells transfected with 30 nM of Cpd.14 (>7-fold lower compared to 10 nM Cpd.14)). In cells transfected with 30 nM of Cpd.14, a great level of cell death was observed as mTOR is a cell survival marker. In the same cell lysate, the RNA interference of Cpd.14 against mTOR RNA transcripts was evaluated. As demonstrated in FIG. 13F, Cpd.14 downregulated the endogenous mTOR levels (50-73% in A549 cells).

In U251 MG cells, the effect of Cpd.17 (10 nM and 30 nM concentration) comprising 3× PD-L1 siRNA and IL-7 mRNA was evaluated for IL-7 expression and simultaneous PD-L1 gene downregulation. As shown in FIG. 14G, Cpd.17 expressed IL-7 protein (up to 1300 pg/ml). In the same cell lysate, the RNA interference of Cpd.14 against PD-L1 RNA transcripts was evaluated. As demonstrated in FIG. 14H, Cpd.14 downregulated endogenous PD-L1 levels (60-87% in U251 MG cells) in a dose relevant manner.

TABLE 6
Primers used in qPCR assay

| | | | SEQ |
|---|---|---|---|
| Gene | Primer | | ID |
| Name | Direction | Sequence (5′ to 3′) | NO |
| IDH1 | Forward | GCTCTGTCTAAGGGTTGGCC | 101 |
| | Reverse | CCATGTCGTCGATGAGCCTA | 102 |
| CDK4 | Forward | GAGTCCCCAATGGAGGAGGA | 103 |
| | Reverse | TCCATCAGCCGGACAACATT | 104 |
| CDK6 | Forward | GCAGACCGGCGAGGAG | 105 |
| | Reverse | CTGTTCGTGACACTGTGCA | 106 |
| EGFR | Forward | TACCTCATCCCACAGCAGG | 107 |
| | Reverse | GCTGTCTTCCTTGATGGGAC | 108 |
| KRAS | Forward | GTACAGTGCAATGAGGGACCA | 109 |
| | Reverse | CACAAAGAAAGCCCTCCCCA | 110 |
| mTOR | Forward | CATGCATGACAACAGCCCAG | 111 |
| | Reverse | AGCTTCAGGGGCATCAAACA | 112 |
| VEGFA | Forward | TTGCCTTGCTGCTCTACCTC | 113 |
| | Reverse | GGAGGGCAGAATCATCACGA | 114 |
| CD155 | Forward | CCCAAATCACCTGGCACTCA | 115 |
| | Reverse | CTCAAAGCTCTCGTGCTCCA | 116 |
| PD-L1 | Forward | GTTGAAGGACCAGCTCTCCC | 117 |
| | Reverse | CTTGTAGTCGGCACCACCAT | 118 |
| c-Myc | Forward | ACTGTATGTGGAGCGGCTTC | 119 |
| | Reverse | CAGGTACAAGCTGGAGGTGG | 120 |
| 18s | Forward | ACCCGTTGAACCCCATTCGTGA | 121 |
| | Reverse | GCCTCACTAAACCATCCAATCGG | 122 |

### Example 19: Human Umbilical Vein Endothelial Cells (HUVEC) Tube-Formation Assay: In Vitro Angiogenesis Model

To assess the functional relevance of VEGFA downregulation potency of Cpd.5 and Cpd.10, SCC-4 cells were cultured and transfected with Cpd.5 and Cpd.10 (20 and 30 nM/well) as described above. After 5 hours, the medium was replaced by fresh growth medium without FBS and the plates were incubated at 37° C. in a humidified atmosphere containing 5% CO₂ for 24 hours to produce and secrete VEGFA into the medium, and supernatants were collected and VEGFA levels quantified by ELISA (ThermoFisher Cat. #KHG0112). The same cell culture supernatant was used to assess the functional ability of the secreted VEGF to induce angiogenesis of human umbilical vein endothelial cells (HUVECs) without treatment or 24 hours post treatment with Cpd.5 and Cpd.10. HUVECs have the ability to form three-dimensional capillary-like tubular structures (also known as pseudo-tube formation) when plated at subconfluent densities with the appropriate extracellular matrix support. The angiogenesis model was established to measure anti-angiogenesis activity of Cpd.5 and Cpd.10 in this in vitro. HUVEC cells (ATCC, Cat. #CRL-1730, # were maintained in F-12K medium (ATCC Cat. #30-2004) supplemented with 10% FBS (ATCC, #30-2020), 0.1 mg/mL heparin (Sigma, #H3393), and 30 μg/mL ECGS (Corning, #354006) at 37° C. in a humidified atmosphere containing 5% CO2 for 24 hours prior to dispensing into Matrigel coated Ibidi plates. 24 hours prior to experiment, pipet tips and μ-slide angiogenesis Ibidi plates (Ibidi, Cat. #81506) were placed at −20° C. Growth factor-reduced BD Matrigel (BD Biosciences, Cat. #354230) was thawed overnight on ice in a refrigerator. On the day of experiment, Matrigel, pipet tips and plate were kept on ice, in the laminar flow, during the Matrigel application. 10 μl of Matrigel was applied into each inner well of Ibidi plates, preventing it from flowing into the upper well. Plates coated with Matrigel were put at 37° C. for 1 hour in a humidified chamber. HUVECs were trypsinized and counted using a standard procedure, and the cells were suspended at a concentration of 5000 cells/504 in cell media either derived from SCC-4 cells supernatant (no treatment) or SCC-4 cells supernatant treated with Cpd.5 or Cpd.10 (20 nM or 30 nM) or media with recombinant VEGFA (0.5 or 5 ng/mL). Fresh HUVEC culture medium used as a baseline control. After Matrigel polymerization, 504 of cell suspension described above were loaded into each well. Ibidi plates were incubated at 37° C., 5% CO₂ for 6-hours. Cells were visualized with a microscope and images were taken (0 hour and 6 hour) and analyzed for tube formation and number of branching points.

Results

Cpd.5 and Cpd.10 designed to have IL-2 coding sequence and 3 species of siRNA targeting VEGFA, were tested to assess the interference of VEGFA expression in SCC-4 cells. Under control conditions, SCC-4 cells produced and secreted approximately 0.8 ng/ml VEGFA into the medium (FIG. 15A). Transfection with Cpd.5 reduced the VEGFA levels down to 76% and 60% at 20 and 30 nM, respectively, whereas Cpd.10 treatment reduced VEGFA more potently to 30% at both 20 and 30 nM (FIG. 15A). 50 μl of these cell culture supernatants were analyzed for their functional ability to induce branching point formation as marker of in vitro angiogenesis in HUVEC cells and compared with untreated controls or media with defined rh-VEGFA concentrations (0.5 and 5 ng/mL). FIG. 15B shows that the potency to increase branching points as measure for tube formation correlated well with medium VEGFA. SCC-4 cells under control conditions produced VEGFA to induce significant branching point formation similar to the two rh-VEGFA controls. Supernatants from both Cpd.5 and Cpd.10 strongly reduced branching points as result of reduced VEGFA levels, with Cpd.10 supernatant being slightly more potent to reduce branching point formation than Cpd.5 due to lower VEGFA levels.

The examples and embodiments described herein are for illustrative purposes only and various modifications or changes suggested to persons skilled in the art are to be included within the spirit and purview of this application and scope of the appended claims.

1.-147. (canceled)

148. A composition comprising a recombinant RNA construct or a vector encoding the recombinant RNA construct, wherein the recombinant RNA construct comprises:
(i) a first RNA sequence encoding a cytokine, and
(ii) a second RNA sequence encoding a genetic element that modulates expression of:
(a) a gene associated with tumor proliferation; or
(b) a gene associated with recognition by immune system,
wherein the first RNA sequence is linked to the second RNA sequence, and
wherein the recombinant RNA construct is a single RNA construct.

149. The composition of claim 148, wherein the first RNA sequence is linked to the second RNA sequence by a linker.

150. The composition of claim 149, wherein the linker is a tRNA linker or a linker with a sequence according to SEQ ID NO: 21.

151. The composition of claim 148, wherein the cytokine comprises interleukin-2 (IL-2), IL-12, IL-15, IL-7, or a functional fragment or functional variant thereof.

152. The composition of claim 148, wherein the cytokine comprises a signal peptide sequence, wherein the signal peptide sequences is an unmodified signal peptide sequence or a modified signal peptide sequence.

153. The composition of claim 152, wherein the signal peptide sequence is an unmodified signal peptide sequence with a sequence selected from the group consisting of SEQ ID NOs: 26 and 27-29.

154. The composition of claim 152, wherein the signal peptide sequence is a modified signal peptide sequence with an insertion, a deletion, or a substitution of at least one amino acid, wherein the modified signal peptide sequence comprising the insertion, the deletion, or the substitution of at least one amino acid comprises a sequence selected from the group consisting of SEQ ID NOs: 27-29.

155. The composition of claim 148, wherein the first RNA sequence is a messenger RNA (mRNA) sequence and the second RNA sequence is a small interfering RNA (siRNA) sequence.

156. The composition of claim 155, wherein the siRNA sequence is capable of binding to an mRNA of the gene associated with tumor proliferation or an mRNA of the gene associated with recognition by the immune system.

157. The composition of claim 156, wherein the second RNA sequence comprises 2 or more siRNA sequences, wherein each siRNA sequence of the 2 or more siRNA sequences has a different sequence that targets a different region of the same mRNA, and wherein each sequence of the 2 or more siRNA sequences is connected by a linker with a sequence according to SEQ ID NO: 22.

158. The composition of claim 156, wherein the second RNA sequence comprises 2, or more redundant siRNA sequences, wherein each sequence of the 2 or more siRNA sequences is connected by a linker with a sequence according to SEQ ID NO: 22.

159. The composition of claim 148, wherein the second RNA sequence encodes a genetic element that modulates expression of a gene associated with tumor proliferation, wherein the gene associated with tumor proliferation comprises a gene associated with angiogenesis, wherein the gene associated with angiogenesis encodes vascular endothelial growth factor (VEGF), isocitrate dehydrogenase (IDH1), cyclin-dependent kinase 4 (CDK4), CDK6, epidermal growth factor receptor (EGFR), mechanistic target of rapamycin (mTOR), Kirsten rat sarcoma viral oncogene (KRAS), cluster of differentiation (CD155), programmed cell death-ligand 1 (PD-L1), myc proto-oncogene (c-Myc), or a functional fragment or functional variant thereof.

160. The composition of claim 159, wherein the gene associated with angiogenesis encodes VEGF, wherein the VEGF comprises VEGFA, an isoform of VEGFA, placental growth factor (PIGF), or a functional fragment or a functional variant thereof.

161. The composition of claim 148, wherein the second RNA sequence encodes a genetic element that modulates expression of a gene associated with recognition by immune system, wherein the gene associated with recognition by immune system encodes MHC class I chain-related sequence A (MICA), MICB, endoplasmic reticulum protein 5 (ERp5), a disintegrin and metalloproteinase (ADAM), matrix metalloproteinase (MMP), or a functional fragment or functional variant thereof.

162. The composition of claim 148, wherein (a) the recombinant RNA construct further comprises a poly(A) tail or a 5′ cap, or (b) the vector encoding the recombinant RNA construct further comprises a Kozak sequence.

163. The composition of claim 148, wherein (a) the recombinant RNA construct comprises a sequence selected from the group consisting of SEQ ID NOs: 129-141, or (b) the vector encoding the recombinant RNA construct comprises a sequence selected from the group consisting of SEQ ID NOs: 86-98.

164. The composition of claim 148, wherein the composition further comprises a cell, wherein the cell comprises the recombinant RNA construct or the vector encoding the recombinant RNA construct, and wherein
(a) an expression level of the protein encoded by the first RNA sequence is higher in the cell compared to the expression level of the protein encoded by the first RNA sequence in a corresponding cell without the recombinant RNA construct or the vector encoding the recombinant RNA construct; and/or
(b) an expression level of a protein encoded by the gene associated with tumor proliferation or the gene associated with recognition by the immune system is lower in the cell compared to the expression level of a protein encoded by the gene associated with tumor proliferation or the gene associated with recognition by the immune system in a corresponding cell without the recombinant RNA construct or the vector encoding the recombinant RNA construct.

165. A pharmaceutical composition comprising the composition of claim 148 and a pharmaceutically acceptable excipient, carrier, or diluent.

166. A method of treating a cancer in a subject in need thereof, comprising administering to the subject a therapeutically effective amount of the pharmaceutical composition of claim 165.

167. A method of modulating two or more genes in a cell, comprising introducing to the cell a recombinant RNA construct or a vector encoding the recombinant RNA construct, wherein the recombinant RNA construct comprises:
(i) a first RNA sequence encoding a cytokine, and
(ii) a second RNA sequence encoding a genetic element that modulates expression of:
(a) a gene associated with tumor proliferation; or
(b) a gene associated with recognition by immune system,
wherein the first RNA sequence is linked to the second RNA sequence, and
wherein the recombinant RNA construct is a single RNA construct.


SEQUENCE LISTING (154 sequences)

SEQ ID NO: 1 - DNA, 468 bp, synthetic construct
  misc_feature 1..468: note = source = /note="Description of Artificial Sequence: Syntheticpolynucleotide"
  source 1..468: mol_type = other DNA
gccaccatgtacagaatgcagctgctgagctgtatcgccctgtctctggccctggtcacaaatagcgcccctaccagcagcagcaccaagaaaacacagctgcaactggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctga

SEQ ID NO: 2 - DNA, 465 bp, synthetic construct
  misc_feature 1..465: note = source = /note="Description of Artificial Sequence: Syntheticpolynucleotide"
  source 1..465: mol_type = other DNA
gccaccatgctgaaactgctgctgctcctgtgtatcgccctgtctctggccgccacaaatagcgcccctaccagcagctccaccaagaaaacacagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctga

SEQ ID NO: 3 - DNA, 468 bp, synthetic construct
  misc_feature 1..468: note = source = /note="Description of Artificial Sequence: Syntheticpolynucleotide"
  source 1..468: mol_type = other DNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctga

SEQ ID NO: 4 - DNA, 468 bp, synthetic construct
  misc_feature 1..468: note = source = /note="Description of Artificial Sequence: Syntheticpolynucleotide"
  source 1..468: mol_type = other DNA
gccaccatgttgttgctgctgctcgcctgtattgccctggcctctacagccctggtcaccaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctga

SEQ ID NO: 5 - DNA, 749 bp, synthetic construct
  misc_feature 1..749: note = source = /note="Description of Artificial Sequence: Syntheticpolynucleotide"
  source 1..749: mol_type = other DNA
gccaccatgtacagaatgcagctgctgagctgtatcgccctgtctctggccctggtcacaaatagcgcccctaccagcagcagcaccaagaaaacacagctgcaactggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaagcagaatcatcacgaagtggtacttgaccacttcgtgatgattctgctttatcttagaggcatatccctacgtaccaacaagagcttcctacagcacaacaaacttgttgttgtgctgtaggaagctctttatcttagaggcatatccctacgtaccaacaagatccgcagacgtgtaaatgtacttgacatttacacgtctgcggatctttatcttagaggcatatcccttttatcttagaggcatatccct

SEQ ID NO: 6 - DNA, 749 bp, synthetic construct
  misc_feature 1..749: note = source = /note="Description of Artificial Sequence: Syntheticpolynucleotide"
  source 1..749: mol_type = other DNA
gccaccatgtacagaatgcagctgctgagctgtatcgccctgtctctggccctggtcacaaatagcgcccctaccagcagcagcaccaagaaaacacagctgcaactggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagattagggtctgtgagatacttgatctcacagaccctaatctcctttatcttagaggcatatccctacgtaccaacaagatgccatgaagaccaagacaacttgtgtcttggtcttcatggcatctttatcttagaggcatatccctacgtaccaacaagcctgatgggaatggaacctaacttgtaggttccattcccatcaggctttatcttagaggcatatcccttttatcttagaggcatatccct

SEQ ID NO: 7 - DNA, 749 bp, synthetic construct
  misc_feature 1..749: note = source = /note="Description of Artificial Sequence: Syntheticpolynucleotide"
  source 1..749: mol_type = other DNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaagcagaatcatcacgaagtggtacttgaccacttcgtgatgattctgctttatcttagaggcatatccctacgtaccaacaagagcttcctacagcacaacaaacttgttgttgtgctgtaggaagctctttatcttagaggcatatccctacgtaccaacaagatccgcagacgtgtaaatgtacttgacatttacacgtctgcggatctttatcttagaggcatatcccttttatcttagaggcatatccct

SEQ ID NO: 8 - DNA, 911 bp, synthetic construct
  misc_feature 1..911: note = source = /note="Description of Artificial Sequence: Syntheticpolynucleotide"
  source 1..911: mol_type = other DNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaagcagaatcatcacgaagtggtacttgaccacttcgtgatgattctgctttatcttagaggcatatccctacgtaccaacaagagcttcctacagcacaacaaacttgttgttgtgctgtaggaagctctttatcttagaggcatatccctacgtaccaacaagatccgcagacgtgtaaatgtacttgacatttacacgtctgcggatctttatcttagaggcatatccctacgtaccaacaagcgcaagaaatcccggtataaacttgttataccgggatttcttgcgctttatcttagaggcatatccctacgtaccaacaaggcgaggcagcttgagttaaaacttgtttaactcaagctgcctcgcctttatcttagaggcatatcccttttatcttagaggcatatccct

SEQ ID NO: 9 - DNA, 737 bp, synthetic construct
  misc_feature 1..737: note = source = /note="Description of Artificial Sequence: Syntheticpolynucleotide"
  source 1..737: mol_type = other DNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagtaccctgatgagatcacttggatctcatcagggtactcctttatcttagaggcatatccctacgtaccaacaaggagtaccctgatgagatcacttggatctcatcagggtactcctttatcttagaggcatatccctacgtaccaacaaggagtaccctgatgagatcacttggatctcatcagggtactcctttatcttagaggcatatcccttttatcttagaggcatatccct

SEQ ID NO: 10 - DNA, 807 bp, synthetic construct
  misc_feature 1..807: note = source = /note="Description of Artificial Sequence: Syntheticpolynucleotide"
  source 1..807: mol_type = other DNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagggcagaatcatcacgaagtggtgaagtacttgacttcaccacttcgtgatgattctgccctcctttatcttagaggcatatccctacgtaccaacaagagatgagcttcctacagcacaacaaatgtgacttgcacatttgttgtgctgtaggaagctcatctctttatcttagaggcatatccctacgtaccaacaagtacaagatccgcagacgtgtaaatgttccacttgggaacatttacacgtctgcggatcttgtactttatcttagaggcatatcccttttatcttagaggcatatccct

SEQ ID NO: 11 - DNA, 1910 bp, synthetic construct
  misc_feature 1..1910: note = source = /note="Description of Artificial Sequence: Syntheticpolynucleotide"
  source 1..1910: mol_type = other DNA
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaagttccttccaaatggctctgtacttgacagagccatttggaaggaactttatcttagaggcatatccctacgtaccaacaagcatcgttcaccgagatctgaacttgtcagatctcggtgaacgatgctttatcttagaggcatatccctacgtaccaacaagaccagcagcggacaaataaaacttgtttatttgtccgctgctggtctttatcttagaggcatatcccttttatcttagaggcatatccct

SEQ ID NO: 12 - DNA, 1938 bp, synthetic construct
  misc_feature 1..1938: note = source = /note="Description of Artificial Sequence: Syntheticpolynucleotide"
  source 1..1938: mol_type = other DNA
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaaatagtgagtcgtattaacgtaccaacaagaaggagctgcccatgagaaaacttgtttctcatgggcagctccttctttatcttagaggcatatccctacgtaccaacaagtgcaatgagggaccagtacaacttgtgtactggtccctcattgcactttatcttagaggcatatccctacgtaccaacaagagctgctgaaggactcatcaacttgtgatgagtccttcagcagctctttatcttagaggcatatcccttttatcttagaggcatatccct

SEQ ID NO: 13 - DNA, 1910 bp, synthetic construct
  misc_feature 1..1910: note = source = /note="Description of Artificial Sequence: Syntheticpolynucleotide"
  source 1..1910: mol_type = other DNA
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaagaaggagctgcccatgagaaaacttgtttctcatgggcagctccttctttatcttagaggcatatccctacgtaccaacaagtccaacgaatgggcctaagaacttgtcttaggcccattcgttggactttatcttagaggcatatccctacgtaccaacaaggacagcatagacgacaccttacttgaaggtgtcgtctatgctgtcctttatcttagaggcatatcccttttatcttagaggcatatccct

SEQ ID NO: 14 - DNA, 1910 bp, synthetic construct
  misc_feature 1..1910: note = source = /note="Description of Artificial Sequence: Syntheticpolynucleotide"
  source 1..1910: mol_type = other DNA
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaagaccctgacattcgctactgtacttgacagtagcgaatgtcagggtctttatcttagaggcatatccctacgtaccaacaagagctgctgaaggactcatcaacttgtgatgagtccttcagcagctctttatcttagaggcatatccctacgtaccaacaaggccaatgacccaacatctctacttgagagatgttgggtcattggcctttatcttagaggcatatcccttttatcttagaggcatatccct

SEQ ID NO: 15 - DNA, 772 bp, synthetic construct
  misc_feature 1..772: note = source = /note="Description of Artificial Sequence: Syntheticpolynucleotide"
  source 1..772: mol_type = other DNA
gccaccatgagaatcagcaagccccacctgagatccatcagcatccagtgctacctgtgcctgctgctgaacagccactttctgacagaggccggcatccacgtgttcatcctgggctgtttttctgccggcctgcctaagaccgaggccaactgggttaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagctgaatagtgagtcgtattaacgtaccaacaaggagtaccctgatgagatcacttggatctcatcagggtactcctttatcttagaggcatatccctacgtaccaacaaggtatccatctctggctatgaacttgtcatagccagagatggatacctttatcttagaggcatatccctacgtaccaacaagtcccgtaacgccatcatcttacttgaagatgatggcgttacgggactttatcttagaggcatatcccttttatcttagaggcatatccct

SEQ ID NO: 16 - DNA, 772 bp, synthetic construct
  misc_feature 1..772: note = source = /note="Description of Artificial Sequence: Syntheticpolynucleotide"
  source 1..772: mol_type = other DNA
gccaccatgagaatcagcaagccccacctgagatccatcagcatccagtgctacctgtgcctgctgctgaacagccactttctgacagaggccggcatccacgtgttcatcctgggctgtttttctgccggcctgcctaagaccgaggccaactgggttaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagctgaatagtgagtcgtattaacgtaccaacaaggagtaccctgatgagatcacttggatctcatcagggtactcctttatcttagaggcatatccctacgtaccaacaagaaggttcagcatagtagctaacttgtagctactatgctgaaccttctttatcttagaggcatatccctacgtaccaacaaggacgacgagaccttcatcaaacttgttgatgaaggtctcgtcgtcctttatcttagaggcatatcccttttatcttagaggcatatccct

SEQ ID NO: 17 - DNA, 821 bp, synthetic construct
  misc_feature 1..821: note = source = /note="Description of Artificial Sequence: Syntheticpolynucleotide"
  source 1..821: mol_type = other DNA
gccaccatgttccacgtgtccttccggtacatcttcggcctgcctccactgatcctggtgctgctgcctgtggccagcagcgactgtgatatcgagggcaaagacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtctgagggcaccaccatcctgctgaattgcaccggccaagtgaagggcagaaagcctgctgctctgggagaagcccagcctaccaagagcctggaagagaacaagtccctgaaagagcagaagaagctgaacgacctctgcttcctgaagcggctgctgcaagagatcaagacctgctggaacaagatcctgatgggcaccaaagaacactgaatagtgagtcgtattaacgtaccaacaagaaggttcagcatagtagctaacttgtagctactatgctgaaccttctttatcttagaggcatatccctacgtaccaacaagcgaattactgtgaaagtcaaacttgttgactttcacagtaattcgctttatcttagaggcatatccctacgtaccaacaagaccagcacactgagaatcaaacttgttgattctcagtgtgctggtctttatcttagaggcatatcccttttatcttagaggcatatccct

SEQ ID NO: 18 - DNA, 17 bp, synthetic construct
  misc_feature 1..17: note = source = /note="Description of Artificial Sequence: Syntheticoligonucleotide"
  source 1..17: mol_type = other DNA
taatacgactcactata

SEQ ID NO: 19 - skipped

SEQ ID NO: 20 - DNA, 77 bp, synthetic construct
  source 1..77: mol_type = other DNA
aacaaagcaccagtggtctagtggtagaatagtaccctgccacggtacagacccgggttcgattcccggctggtgca

SEQ ID NO: 21 - DNA, 28 bp, synthetic construct
  misc_feature 1..28: note = source = /note="Description of Artificial Sequence: Syntheticoligonucleotide"
  source 1..28: mol_type = other DNA
atagtgagtcgtattaacgtaccaacaa

SEQ ID NO: 22 - DNA, 34 bp, synthetic construct
  misc_feature 1..34: note = source = /note="Description of Artificial Sequence: Syntheticoligonucleotide"
  source 1..34: mol_type = other DNA
tttatcttagaggcatatccctacgtaccaacaa

SEQ ID NO: 23 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 24 - protein, 133 aa, Homo sapiens [sequence omitted]

SEQ ID NO: 25 - DNA, 822 bp, Homo sapiens
  source 1..822: mol_type = unassigned DNA
agttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 26 - protein, 20 aa, Homo sapiens
MYRMQLLSCIALSLALVTNS

SEQ ID NO: 27 - protein, 19 aa, synthetic construct
  REGION 1..19: note = source = /note="Description of Artificial Sequence: Syntheticpeptide"
MLKLLLLLCIALSLAATNS

SEQ ID NO: 28 - protein, 20 aa, synthetic construct
  REGION 1..20: note = source = /note="Description of Artificial Sequence: Syntheticpeptide"
MLLLLLACIALASTAAATNS

SEQ ID NO: 29 - protein, 20 aa, synthetic construct
  REGION 1..20: note = source = /note="Description of Artificial Sequence: Syntheticpeptide"
MLLLLLACIALASTALVTNS

SEQ ID NO: 30 - DNA, 60 bp, Homo sapiens
  source 1..60: mol_type = unassigned DNA
atgtacagaatgcagctgctgagctgtatcgccctgtctctggccctggtcacaaatagc

SEQ ID NO: 31 - DNA, 57 bp, synthetic construct
  misc_feature 1..57: note = source = /note="Description of Artificial Sequence: Syntheticoligonucleotide"
  source 1..57: mol_type = other DNA
atgctgaaactgctgctgctcctgtgtatcgccctgtctctggccgccacaaatagc

SEQ ID NO: 32 - DNA, 60 bp, synthetic construct
  misc_feature 1..60: note = source = /note="Description of Artificial Sequence: Syntheticoligonucleotide"
  source 1..60: mol_type = other DNA
atgttgttgctgctgctcgcctgtattgccctggcctctacagccgccgctacaaattct

SEQ ID NO: 33 - DNA, 60 bp, synthetic construct
  misc_feature 1..60: note = source = /note="Description of Artificial Sequence: Syntheticoligonucleotide"
  source 1..60: mol_type = other DNA
atgttgttgctgctgctcgcctgtattgccctggcctctacagccctggtcaccaattct

SEQ ID NO: 34 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 35 - DNA, 699 bp, Homo sapiens
  source 1..699: mol_type = unassigned DNA
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 36 - RNA, 699 nt, Homo sapiens
  source 1..699: mol_type = mRNA
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 37 - protein, 383 aa, Homo sapiens
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 38 - DNA, 1152 bp, Homo sapiens
  source 1..1152: mol_type = unassigned DNA
atggggctgggcccggtcttcctgcttctggctggcatcttcccttttgcacctccgggagctgctgctgagccccacagtcttcgttataacctcacggtgctgtcctgggatggatctgtgcagtcagggtttctcactgaggtacatctggatggtcagcccttcctgcgctgtgacaggcagaaatgcagggcaaagccccagggacagtgggcagaagatgtcctgggaaataagacatgggacagagagaccagagacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactaaggaatggacaatgccccagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagtgccccccatggtgaatgtcacccgcagcgaggcctcagagggcaacattaccgtgacatgcagggcttctggcttctatccctggaatatcacactgagctggcgtcaggatggggtatctttgagccacgacacccagcagtggggggatgtcctgcctgatgggaatggaacctaccagacctgggtggccaccaggatttgccaaggagaggagcagaggttcacctgctacatggaacacagcgggaatcacagcactcaccctgtgccctctgggaaagtgctggtgcttcagagtcattggcagacattccatgtttctgctgttgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctag

SEQ ID NO: 39 - RNA, 1152 nt, Homo sapiens
  source 1..1152: mol_type = mRNA
atggggctgggcccggtcttcctgcttctggctggcatcttcccttttgcacctccgggagctgctgctgagccccacagtcttcgttataacctcacggtgctgtcctgggatggatctgtgcagtcagggtttctcactgaggtacatctggatggtcagcccttcctgcgctgtgacaggcagaaatgcagggcaaagccccagggacagtgggcagaagatgtcctgggaaataagacatgggacagagagaccagagacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactaaggaatggacaatgccccagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagtgccccccatggtgaatgtcacccgcagcgaggcctcagagggcaacattaccgtgacatgcagggcttctggcttctatccctggaatatcacactgagctggcgtcaggatggggtatctttgagccacgacacccagcagtggggggatgtcctgcctgatgggaatggaacctaccagacctgggtggccaccaggatttgccaaggagaggagcagaggttcacctgctacatggaacacagcgggaatcacagcactcaccctgtgccctctgggaaagtgctggtgcttcagagtcattggcagacattccatgtttctgctgttgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctag

SEQ ID NO: 40 - protein, 383 aa, Homo sapiens
MGLGRVLLFLAVAFPFAPPAAAAEPHSLRYNLMVLSQDGSVQSGFLAEGHLDGQPFLRYDRQKRRAKPQGQWAENVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGPELVSLQVLDQHPVGTGDHRDAAQLGFQPLMSATGSTGSTEGT

SEQ ID NO: 41 - DNA, 1152 bp, Homo sapiens
  source 1..1152: mol_type = unassigned DNA
atggggctgggccgggtcctgctgtttctggccgtcgccttcccttttgcacccccggcagccgccgctgagccccacagtcttcgttacaacctcatggtgctgtcccaggatggatctgtgcagtcagggtttctcgctgagggacatctggatggtcagcccttcctgcgctatgacaggcagaaacgcagggcaaagccccagggacagtgggcagaaaatgtcctgggagctaagacctgggacacagagaccgaggacttgacagagaatgggcaagacctcaggaggaccctgactcatatcaaggaccagaaaggaggcttgcattccctccaggagattagggtctgtgagatccatgaagacagcagcaccaggggctcccggcatttctactacgatggggagctcttcctctcccaaaacctggagactcaagaatcgacagtgccccagtcctccagagctcagaccttggctatgaacgtcacaaatttctggaaggaagatgccatgaagaccaagacacactatcgcgctatgcaggcagactgcctgcagaaactacagcgatatctgaaatccggggtggccatcaggagaacagtgccccccatggtgaatgtcacctgcagcgaggtctcagagggcaacatcaccgtgacatgcagggcttccagcttctatccccggaatatcacactgacctggcgtcaggatggggtatctttgagccacaacacccagcagtggggggatgtcctgcctgatgggaatggaacctaccagacctgggtggccaccaggattcgccaaggagaggagcagaggttcacctgctacatggaacacagcgggaatcacggcactcaccctgtgccctctgggaaggcgctggtgcttcagagtcaacggacagactttccatatgtttctgctgctatgccatgttttgttattattattattctctgtgtcccttgttgcaagaagaaaacatcagcggcagagggtccagagcttgtgagcctgcaggtcctggatcaacacccagttgggacaggagaccacagggatgcagcacagctgggatttcagcctctgatgtcagctactgggtccactggttccactgagggcacctag

SEQ ID NO: 42 - RNA, 1152 nt, Homo sapiens
  source 1..1152: mol_type = mRNA
atggggctgggccgggtcctgctgtttctggccgtcgccttcccttttgcacccccggcagccgccgctgagccccacagtcttcgttacaacctcatggtgctgtcccaggatggatctgtgcagtcagggtttctcgctgagggacatctggatggtcagcccttcctgcgctatgacaggcagaaacgcagggcaaagccccagggacagtgggcagaaaatgtcctgggagctaagacctgggacacagagaccgaggacttgacagagaatgggcaagacctcaggaggaccctgactcatatcaaggaccagaaaggaggcttgcattccctccaggagattagggtctgtgagatccatgaagacagcagcaccaggggctcccggcatttctactacgatggggagctcttcctctcccaaaacctggagactcaagaatcgacagtgccccagtcctccagagctcagaccttggctatgaacgtcacaaatttctggaaggaagatgccatgaagaccaagacacactatcgcgctatgcaggcagactgcctgcagaaactacagcgatatctgaaatccggggtggccatcaggagaacagtgccccccatggtgaatgtcacctgcagcgaggtctcagagggcaacatcaccgtgacatgcagggcttccagcttctatccccggaatatcacactgacctggcgtcaggatggggtatctttgagccacaacacccagcagtggggggatgtcctgcctgatgggaatggaacctaccagacctgggtggccaccaggattcgccaaggagaggagcagaggttcacctgctacatggaacacagcgggaatcacggcactcaccctgtgccctctgggaaggcgctggtgcttcagagtcaacggacagactttccatatgtttctgctgctatgccatgttttgttattattattattctctgtgtcccttgttgcaagaagaaaacatcagcggcagagggtccagagcttgtgagcctgcaggtcctggatcaacacccagttgggacaggagaccacagggatgcagcacagctgggatttcagcctctgatgtcagctactgggtccactggttccactgagggcacctag

SEQ ID NO: 43 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 44 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 45 - DNA, 1230 bp, Homo sapiens
  source 1..1230: mol_type = unassigned DNA
atttcgctttcattttgggccgagctggaggcggcggggccgtcccggaacggctgcggccgggcaccccgggagttaatccgaaagcgccgcaagccccgcgggccggccgcaccgcacgtgtcaccgagaagctgatgtagagagagacacagaaggagacagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtgtttttcattcactttaatagaaggg

SEQ ID NO: 46 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 47 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 48 - DNA, 1410 bp, Homo sapiens
  source 1..1410: mol_type = unassigned DNA
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaat

SEQ ID NO: 49 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 50 - DNA, 1245 bp, Homo sapiens
  source 1..1245: mol_type = unassigned DNA
atgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaa

SEQ ID NO: 51 - RNA, 1245 nt, Homo sapiens
  source 1..1245: mol_type = mRNA
atgtccaaaaaaatcagtggcggttctgtggtagagatgcaaggagatgaaatgacacgaatcatttgggaattgattaaagagaaactcatttttccctacgtggaattggatctacatagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaatacagagcaactgattttgttgttcctgggcctggaaaagtagagataacctacacaccaagtgacggaacccaaaaggtgacatacctggtacataactttgaagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattgcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaatgtgcaacgttctgactacttgaatacatttgagttcatggataaacttggagaaaacttgaagatcaaactagctcaggccaaactttaa

SEQ ID NO: 52 - protein, 303 aa, Homo sapiens
MATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 53 - DNA, 912 bp, Homo sapiens
  source 1..912: mol_type = unassigned DNA
atggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtga

SEQ ID NO: 54 - RNA, 912 nt, Homo sapiens
  source 1..912: mol_type = mRNA
atggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtga

SEQ ID NO: 55 - protein, 326 aa, Homo sapiens
MEKDGLCRADQQYECVAEIGEGAYGKVFKARDLKNGGRFVALKRVRVQTGEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDVIGLPGEEDWPRDVALPRQAFHSKSAQPIEKFVTDIDELGKDLLLKCLTFNPAKRISAYSALSHPYFQDLERCKENLDSHLPPSQNTSELNTA

SEQ ID NO: 56 - DNA, 981 bp, Homo sapiens
  source 1..981: mol_type = unassigned DNA
atggagaaggacggcctgtgccgcgctgaccagcagtacgaatgcgtggcggagatcggggagggcgcctatgggaaggtgttcaaggcccgcgacttgaagaacggaggccgtttcgtggcgttgaagcgcgtgcgggtgcagaccggcgaggagggcatgccgctctccaccatccgcgaggtggcggtgctgaggcacctggagaccttcgagcaccccaacgtggtcaggttgtttgatgtgtgcacagtgtcacgaacagacagagaaaccaaactaactttagtgtttgaacatgtcgatcaagacttgaccacttacttggataaagttccagagcctggagtgcccactgaaaccataaaggatatgatgtttcagcttctccgaggtctggactttcttcattcacaccgagtagtgcatcgcgatctaaaaccacagaacattctggtgaccagcagcggacaaataaaactcgctgacttcggccttgcccgcatctatagtttccagatggctctaacctcagtggtcgtcacgctgtggtacagagcacccgaagtcttgctccagtccagctacgccacccccgtggatctctggagtgttggctgcatatttgcagaaatgtttcgtagaaagcctctttttcgtggaagttcagatgttgatcaactaggaaaaatcttggacgtgattggactcccaggagaagaagactggcctagagatgttgcccttcccaggcaggcttttcattcaaaatctgcccaaccaattgagaagtttgtaacagatatcgatgaactaggcaaagacctacttctgaagtgtttgacatttaacccagccaaaagaatatctgcctacagtgccctgtctcacccatacttccaggacctggaaaggtgcaaagaaaacctggattcccacctgccgcccagccagaacacctcggagctgaatacagcctga

SEQ ID NO: 57 - RNA, 981 nt, Homo sapiens
  source 1..981: mol_type = mRNA
atggagaaggacggcctgtgccgcgctgaccagcagtacgaatgcgtggcggagatcggggagggcgcctatgggaaggtgttcaaggcccgcgacttgaagaacggaggccgtttcgtggcgttgaagcgcgtgcgggtgcagaccggcgaggagggcatgccgctctccaccatccgcgaggtggcggtgctgaggcacctggagaccttcgagcaccccaacgtggtcaggttgtttgatgtgtgcacagtgtcacgaacagacagagaaaccaaactaactttagtgtttgaacatgtcgatcaagacttgaccacttacttggataaagttccagagcctggagtgcccactgaaaccataaaggatatgatgtttcagcttctccgaggtctggactttcttcattcacaccgagtagtgcatcgcgatctaaaaccacagaacattctggtgaccagcagcggacaaataaaactcgctgacttcggccttgcccgcatctatagtttccagatggctctaacctcagtggtcgtcacgctgtggtacagagcacccgaagtcttgctccagtccagctacgccacccccgtggatctctggagtgttggctgcatatttgcagaaatgtttcgtagaaagcctctttttcgtggaagttcagatgttgatcaactaggaaaaatcttggacgtgattggactcccaggagaagaagactggcctagagatgttgcccttcccaggcaggcttttcattcaaaatctgcccaaccaattgagaagtttgtaacagatatcgatgaactaggcaaagacctacttctgaagtgtttgacatttaacccagccaaaagaatatctgcctacagtgccctgtctcacccatacttccaggacctggaaaggtgcaaagaaaacctggattcccacctgccgcccagccagaacacctcggagctgaatacagcctga

SEQ ID NO: 58 - protein, 1210 aa, Homo sapiens [sequence omitted]

SEQ ID NO: 59 - DNA, 3633 bp, Homo sapiens
  source 1..3633: mol_type = unassigned DNA
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 60 - RNA, 3633 nt, Homo sapiens
  source 1..3633: mol_type = mRNA
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 61 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 62 - DNA, 7650 bp, Homo sapiens
  source 1..7650: mol_type = unassigned DNA
atgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaa

SEQ ID NO: 63 - RNA, 7650 nt, Homo sapiens
  source 1..7650: mol_type = mRNA
atgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaa

SEQ ID NO: 64 - protein, 188 aa, Homo sapiens [sequence omitted]

SEQ ID NO: 65 - DNA, 567 bp, Homo sapiens
  source 1..567: mol_type = unassigned DNA
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 66 - RNA, 567 nt, Homo sapiens
  source 1..567: mol_type = mRNA
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 67 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 68 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 69 - DNA, 858 bp, Homo sapiens
  source 1..858: mol_type = unassigned DNA
atgttccatcatgttccatgctgctgacgtcacatggagcacagaaatcaatgttagcagatagccagcccatacaagatcgtattgtattgtaggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagt

SEQ ID NO: 70 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGMSRNAIIFLVLGILVFLILLGIGIYFYWSKCSREVLWHCHLCPSSTEHASASANGHVSYSAVSRENSSSQDPQTEGTR

SEQ ID NO: 71 - DNA, 1254 bp, Homo sapiens
  source 1..1254: mol_type = unassigned DNA
atggcccgagccatggccgccgcgtggccgctgctgctggtggcgctactggtgctgtcctggccacccccaggaaccggggacgtcgtcgtgcaggcgcccacccaggtgcccggcttcttgggcgactccgtgacgctgccctgctacctacaggtgcccaacatggaggtgacgcatgtgtcacagctgacttgggcgcggcatggtgaatctggcagcatggccgtcttccaccaaacgcagggccccagctattcggagtccaaacggctggaattcgtggcagccagactgggcgcggagctgcggaatgcctcgctgaggatgttcgggttgcgcgtagaggatgaaggcaactacacctgcctgttcgtcacgttcccgcagggcagcaggagcgtggatatctggctccgagtgcttgccaagccccagaacacagctgaggttcagaaggtccagctcactggagagccagtgcccatggcccgctgcgtctccacagggggtcgcccgccagcccaaatcacctggcactcagacctgggcgggatgcccaatacgagccaggtgccagggttcctgtctggcacagtcactgtcaccagcctctggatattggtgccctcaagccaggtggacggcaagaatgtgacctgcaaggtggagcacgagagctttgagaagcctcagctgctgactgtgaacctcaccgtgtactaccccccagaggtatccatctctggctatgataacaactggtaccttggccagaatgaggccaccctgacctgcgatgctcgcagcaacccagagcccacaggctataattggagcacgaccatgggtcccctgccaccctttgctgtggcccagggcgcccagctcctgatccgtcctgtggacaaaccaatcaacacaactttaatctgcaacgtcaccaatgccctaggagctcgccaggcagaactgaccgtccaggtcaaagagggacctcccagtgagcactcaggcatgtcccgtaacgccatcatcttcctggttctgggaatcctggtttttctgatcctgctggggatcgggatttatttctattggtccaaatgttcccgtgaggtcctttggcactgtcatctgtgtccctcgagtacagagcatgccagcgcctcagctaatgggcatgtctcctattcagctgtgagcagagagaacagctcttcccaggatccacagacagagggcacaaggtga

SEQ ID NO: 72 - RNA, 1254 nt, Homo sapiens
  source 1..1254: mol_type = mRNA
atggcccgagccatggccgccgcgtggccgctgctgctggtggcgctactggtgctgtcctggccacccccaggaaccggggacgtcgtcgtgcaggcgcccacccaggtgcccggcttcttgggcgactccgtgacgctgccctgctacctacaggtgcccaacatggaggtgacgcatgtgtcacagctgacttgggcgcggcatggtgaatctggcagcatggccgtcttccaccaaacgcagggccccagctattcggagtccaaacggctggaattcgtggcagccagactgggcgcggagctgcggaatgcctcgctgaggatgttcgggttgcgcgtagaggatgaaggcaactacacctgcctgttcgtcacgttcccgcagggcagcaggagcgtggatatctggctccgagtgcttgccaagccccagaacacagctgaggttcagaaggtccagctcactggagagccagtgcccatggcccgctgcgtctccacagggggtcgcccgccagcccaaatcacctggcactcagacctgggcgggatgcccaatacgagccaggtgccagggttcctgtctggcacagtcactgtcaccagcctctggatattggtgccctcaagccaggtggacggcaagaatgtgacctgcaaggtggagcacgagagctttgagaagcctcagctgctgactgtgaacctcaccgtgtactaccccccagaggtatccatctctggctatgataacaactggtaccttggccagaatgaggccaccctgacctgcgatgctcgcagcaacccagagcccacaggctataattggagcacgaccatgggtcccctgccaccctttgctgtggcccagggcgcccagctcctgatccgtcctgtggacaaaccaatcaacacaactttaatctgcaacgtcaccaatgccctaggagctcgccaggcagaactgaccgtccaggtcaaagagggacctcccagtgagcactcaggcatgtcccgtaacgccatcatcttcctggttctgggaatcctggtttttctgatcctgctggggatcgggatttatttctattggtccaaatgttcccgtgaggtcctttggcactgtcatctgtgtccctcgagtacagagcatgccagcgcctcagctaatgggcatgtctcctattcagctgtgagcagagagaacagctcttcccaggatccacagacagagggcacaaggtga

SEQ ID NO: 73 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 74 - DNA, 873 bp, Homo sapiens
  source 1..873: mol_type = unassigned DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 75 - RNA, 873 nt, Homo sapiens
  source 1..873: mol_type = mRNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 76 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 77 - DNA, 1365 bp, Homo sapiens
  source 1..1365: mol_type = unassigned DNA
atggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 78 - RNA, 1365 nt, Homo sapiens
  source 1..1365: mol_type = mRNA
atggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 79 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 80 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 81 - DNA, 534 bp, Homo sapiens
  source 1..534: mol_type = unassigned DNA
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 82 - DNA, 2842 bp, synthetic construct
  misc_feature 1..2842: note = source = /note="Description of Artificial Sequence: Syntheticpolynucleotide"
  source 1..2842: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgtacagaatgcagctgctgagctgtatcgccctgtctctggccctggtcacaaatagcgcccctaccagcagcagcaccaagaaaacacagctgcaactggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 83 - DNA, 2839 bp, synthetic construct
  misc_feature 1..2839: note = source = /note="Description of Artificial Sequence: Syntheticpolynucleotide"
  source 1..2839: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgctgaaactgctgctgctcctgtgtatcgccctgtctctggccgccacaaatagcgcccctaccagcagctccaccaagaaaacacagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 84 - DNA, 2842 bp, synthetic construct
  misc_feature 1..2842: note = source = /note="Description of Artificial Sequence: Syntheticpolynucleotide"
  source 1..2842: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 85 - DNA, 2842 bp, synthetic construct
  misc_feature 1..2842: note = source = /note="Description of Artificial Sequence: Syntheticpolynucleotide"
  source 1..2842: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccacgtgtcttgtccagagctcgccaccatgttgttgctgctgctcgcctgtattgccctggcctctacagccctggtcaccaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaggtacctggagcacaagactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 86 - DNA, 3027 bp, synthetic construct
  misc_feature 1..3027: note = source = /note="Description of Artificial Sequence: Syntheticpolynucleotide"
  source 1..3027: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgccaccatgtacagaatgcagctgctgagctgtatcgccctgtctctggccctggtcacaaatagcgcccctaccagcagcagcaccaagaaaacacagctgcaactggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaagcagaatcatcacgaagtggtacttgaccacttcgtgatgattctgctttatcttagaggcatatccctacgtaccaacaagagcttcctacagcacaacaaacttgttgttgtgctgtaggaagctctttatcttagaggcatatccctacgtaccaacaagatccgcagacgtgtaaatgtacttgacatttacacgtctgcggatctttatcttagaggcatatcccttttatcttagaggcatatccctctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 87 - DNA, 3090 bp, synthetic construct
  misc_feature 1..3090: note = source = /note="Description of Artificial Sequence: Syntheticpolynucleotide"
  source 1..3090: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgtacagaatgcagctgctgagctgtatcgccctgtctctggccctggtcacaaatagcgcccctaccagcagcagcaccaagaaaacacagctgcaactggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagattagggtctgtgagatacttgatctcacagaccctaatctcctttatcttagaggcatatccctacgtaccaacaagatgccatgaagaccaagacaacttgtgtcttggtcttcatggcatctttatcttagaggcatatccctacgtaccaacaagcctgatgggaatggaacctaacttgtaggttccattcccatcaggctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 88 - DNA, 3090 bp, synthetic construct
  misc_feature 1..3090: note = source = /note="Description of Artificial Sequence: Syntheticpolynucleotide"
  source 1..3090: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaagcagaatcatcacgaagtggtacttgaccacttcgtgatgattctgctttatcttagaggcatatccctacgtaccaacaagagcttcctacagcacaacaaacttgttgttgtgctgtaggaagctctttatcttagaggcatatccctacgtaccaacaagatccgcagacgtgtaaatgtacttgacatttacacgtctgcggatctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 89 - DNA, 3252 bp, synthetic construct
  misc_feature 1..3252: note = source = /note="Description of Artificial Sequence: Syntheticpolynucleotide"
  source 1..3252: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaagcagaatcatcacgaagtggtacttgaccacttcgtgatgattctgctttatcttagaggcatatccctacgtaccaacaagagcttcctacagcacaacaaacttgttgttgtgctgtaggaagctctttatcttagaggcatatccctacgtaccaacaagatccgcagacgtgtaaatgtacttgacatttacacgtctgcggatctttatcttagaggcatatccctacgtaccaacaagcgcaagaaatcccggtataaacttgttataccgggatttcttgcgctttatcttagaggcatatccctacgtaccaacaaggcgaggcagcttgagttaaaacttgtttaactcaagctgcctcgcctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 90 - DNA, 3015 bp, synthetic construct
  misc_feature 1..3015: note = source = /note="Description of Artificial Sequence: Syntheticpolynucleotide"
  source 1..3015: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagtaccctgatgagatcacttggatctcatcagggtactcctttatcttagaggcatatccctacgtaccaacaaggagtaccctgatgagatcacttggatctcatcagggtactcctttatcttagaggcatatccctacgtaccaacaaggagtaccctgatgagatcacttggatctcatcagggtactcctttatcttagaggcatatcccttttatcttagaggcatatccctctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 91 - DNA, 3085 bp, synthetic construct
  misc_feature 1..3085: note = source = /note="Description of Artificial Sequence: Syntheticpolynucleotide"
  source 1..3085: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagggcagaatcatcacgaagtggtgaagtacttgacttcaccacttcgtgatgattctgccctcctttatcttagaggcatatccctacgtaccaacaagagatgagcttcctacagcacaacaaatgtgacttgcacatttgttgtgctgtaggaagctcatctctttatcttagaggcatatccctacgtaccaacaagtacaagatccgcagacgtgtaaatgttccacttgggaacatttacacgtctgcggatcttgtactttatcttagaggcatatcccttttatcttagaggcatatccctctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 92 - DNA, 4251 bp, synthetic construct
  misc_feature 1..4251: note = source = /note="Description of Artificial Sequence: Syntheticpolynucleotide"
  source 1..4251: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaagttccttccaaatggctctgtacttgacagagccatttggaaggaactttatcttagaggcatatccctacgtaccaacaagcatcgttcaccgagatctgaacttgtcagatctcggtgaacgatgctttatcttagaggcatatccctacgtaccaacaagaccagcagcggacaaataaaacttgtttatttgtccgctgctggtctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 93 - DNA, 4251 bp, synthetic construct
  misc_feature 1..4251: note = source = /note="Description of Artificial Sequence: Syntheticpolynucleotide"
  source 1..4251: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaagaaggagctgcccatgagaaaacttgtttctcatgggcagctccttctttatcttagaggcatatccctacgtaccaacaagtgcaatgagggaccagtacaacttgtgtactggtccctcattgcactttatcttagaggcatatccctacgtaccaacaagagctgctgaaggactcatcaacttgtgatgagtccttcagcagctctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 94 - DNA, 4251 bp, synthetic construct
  misc_feature 1..4251: note = source = /note="Description of Artificial Sequence: Syntheticpolynucleotide"
  source 1..4251: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaagaaggagctgcccatgagaaaacttgtttctcatgggcagctccttctttatcttagaggcatatccctacgtaccaacaagtccaacgaatgggcctaagaacttgtcttaggcccattcgttggactttatcttagaggcatatccctacgtaccaacaaggacagcatagacgacaccttacttgaaggtgtcgtctatgctgtcctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 95 - DNA, 4251 bp, synthetic construct
  misc_feature 1..4251: note = source = /note="Description of Artificial Sequence: Syntheticpolynucleotide"
  source 1..4251: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaagaccctgacattcgctactgtacttgacagtagcgaatgtcagggtctttatcttagaggcatatccctacgtaccaacaagagctgctgaaggactcatcaacttgtgatgagtccttcagcagctctttatcttagaggcatatccctacgtaccaacaaggccaatgacccaacatctctacttgagagatgttgggtcattggcctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 96 - DNA, 3113 bp, synthetic construct
  misc_feature 1..3113: note = source = /note="Description of Artificial Sequence: Syntheticpolynucleotide"
  source 1..3113: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgagaatcagcaagccccacctgagatccatcagcatccagtgctacctgtgcctgctgctgaacagccactttctgacagaggccggcatccacgtgttcatcctgggctgtttttctgccggcctgcctaagaccgaggccaactgggttaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagctgaatagtgagtcgtattaacgtaccaacaaggagtaccctgatgagatcacttggatctcatcagggtactcctttatcttagaggcatatccctacgtaccaacaaggtatccatctctggctatgaacttgtcatagccagagatggatacctttatcttagaggcatatccctacgtaccaacaagtcccgtaacgccatcatcttacttgaagatgatggcgttacgggactttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 97 - DNA, 3113 bp, synthetic construct
  misc_feature 1..3113: note = source = /note="Description of Artificial Sequence: Syntheticpolynucleotide"
  source 1..3113: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgagaatcagcaagccccacctgagatccatcagcatccagtgctacctgtgcctgctgctgaacagccactttctgacagaggccggcatccacgtgttcatcctgggctgtttttctgccggcctgcctaagaccgaggccaactgggttaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagctgaatagtgagtcgtattaacgtaccaacaaggagtaccctgatgagatcacttggatctcatcagggtactcctttatcttagaggcatatccctacgtaccaacaagaaggttcagcatagtagctaacttgtagctactatgctgaaccttctttatcttagaggcatatccctacgtaccaacaaggacgacgagaccttcatcaaacttgttgatgaaggtctcgtcgtcctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 98 - DNA, 3162 bp, synthetic construct
  misc_feature 1..3162: note = source = /note="Description of Artificial Sequence: Syntheticpolynucleotide"
  source 1..3162: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatgccaccatgttccacgtgtccttccggtacatcttcggcctgcctccactgatcctggtgctgctgcctgtggccagcagcgactgtgatatcgagggcaaagacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtctgagggcaccaccatcctgctgaattgcaccggccaagtgaagggcagaaagcctgctgctctgggagaagcccagcctaccaagagcctggaagagaacaagtccctgaaagagcagaagaagctgaacgacctctgcttcctgaagcggctgctgcaagagatcaagacctgctggaacaagatcctgatgggcaccaaagaacactgaatagtgagtcgtattaacgtaccaacaagaaggttcagcatagtagctaacttgtagctactatgctgaaccttctttatcttagaggcatatccctacgtaccaacaagcgaattactgtgaaagtcaaacttgttgactttcacagtaattcgctttatcttagaggcatatccctacgtaccaacaagaccagcacactgagaatcaaacttgttgattctcagtgtgctggtctttatcttagaggcatatcccttttatcttagaggcatatccctctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 99 - DNA, 20 bp, synthetic construct
  misc_feature 1..20: note = source = /note="Description of Artificial Sequence: Syntheticprimer"
  source 1..20: mol_type = other DNA
gctgcaaggcgattaagttg

SEQ ID NO: 100 - DNA, 143 bp, synthetic construct
  misc_feature 1..143: note = source = /note="Description of Artificial Sequence: Synthetic primer"
  source 1..143: mol_type = other DNA
  misc_feature 1..143: note = Combined DNA/RNA Molecule: Synthetic primer
  misc_feature 1..3: note = RNA fragment
  modified_base 1..3: mod_base = um
  misc_feature 4..143: note = DNA fragment
ttttttttttttttttttttttttttttttttttttttttttttttttttttttttttttttttttttttttttttttttttttttttttttttttttttttttttttttttttttttttcagctatgaccatgttaatgcag

SEQ ID NO: 101 - DNA, 20 bp, synthetic construct
  misc_feature 1..20: note = source = /note="Description of Artificial Sequence: Syntheticprimer"
  source 1..20: mol_type = other DNA
gctctgtctaagggttggcc

SEQ ID NO: 102 - DNA, 20 bp, synthetic construct
  misc_feature 1..20: note = source = /note="Description of Artificial Sequence: Syntheticprimer"
  source 1..20: mol_type = other DNA
ccatgtcgtcgatgagccta

SEQ ID NO: 103 - DNA, 20 bp, synthetic construct
  misc_feature 1..20: note = source = /note="Description of Artificial Sequence: Syntheticprimer"
  source 1..20: mol_type = other DNA
gagtccccaatggaggagga

SEQ ID NO: 104 - DNA, 20 bp, synthetic construct
  misc_feature 1..20: note = source = /note="Description of Artificial Sequence: Syntheticprimer"
  source 1..20: mol_type = other DNA
tccatcagccggacaacatt

SEQ ID NO: 105 - DNA, 16 bp, synthetic construct
  misc_feature 1..16: note = source = /note="Description of Artificial Sequence: Syntheticprimer"
  source 1..16: mol_type = other DNA
gcagaccggcgaggag

SEQ ID NO: 106 - DNA, 19 bp, synthetic construct
  misc_feature 1..19: note = source = /note="Description of Artificial Sequence: Syntheticprimer"
  source 1..19: mol_type = other DNA
ctgttcgtgacactgtgca

SEQ ID NO: 107 - DNA, 19 bp, synthetic construct
  misc_feature 1..19: note = source = /note="Description of Artificial Sequence: Syntheticprimer"
  source 1..19: mol_type = other DNA
tacctcatcccacagcagg

SEQ ID NO: 108 - DNA, 20 bp, synthetic construct
  misc_feature 1..20: note = source = /note="Description of Artificial Sequence: Syntheticprimer"
  source 1..20: mol_type = other DNA
gctgtcttccttgatgggac

SEQ ID NO: 109 - DNA, 21 bp, synthetic construct
  misc_feature 1..21: note = source = /note="Description of Artificial Sequence: Syntheticprimer"
  source 1..21: mol_type = other DNA
gtacagtgcaatgagggacca

SEQ ID NO: 110 - DNA, 20 bp, synthetic construct
  misc_feature 1..20: note = source = /note="Description of Artificial Sequence: Syntheticprimer"
  source 1..20: mol_type = other DNA
cacaaagaaagccctcccca

SEQ ID NO: 111 - DNA, 20 bp, synthetic construct
  misc_feature 1..20: note = source = /note="Description of Artificial Sequence: Syntheticprimer"
  source 1..20: mol_type = other DNA
catgcatgacaacagcccag

SEQ ID NO: 112 - DNA, 20 bp, synthetic construct
  misc_feature 1..20: note = source = /note="Description of Artificial Sequence: Syntheticprimer"
  source 1..20: mol_type = other DNA
agcttcaggggcatcaaaca

SEQ ID NO: 113 - DNA, 20 bp, synthetic construct
  misc_feature 1..20: note = source = /note="Description of Artificial Sequence: Syntheticprimer"
  source 1..20: mol_type = other DNA
ttgccttgctgctctacctc

SEQ ID NO: 114 - DNA, 20 bp, synthetic construct
  misc_feature 1..20: note = source = /note="Description of Artificial Sequence: Syntheticprimer"
  source 1..20: mol_type = other DNA
ggagggcagaatcatcacga

SEQ ID NO: 115 - DNA, 20 bp, synthetic construct
  misc_feature 1..20: note = source = /note="Description of Artificial Sequence: Syntheticprimer"
  source 1..20: mol_type = other DNA
cccaaatcacctggcactca

SEQ ID NO: 116 - DNA, 20 bp, synthetic construct
  misc_feature 1..20: note = source = /note="Description of Artificial Sequence: Syntheticprimer"
  source 1..20: mol_type = other DNA
ctcaaagctctcgtgctcca

SEQ ID NO: 117 - DNA, 20 bp, synthetic construct
  misc_feature 1..20: note = source = /note="Description of Artificial Sequence: Syntheticprimer"
  source 1..20: mol_type = other DNA
gttgaaggaccagctctccc

SEQ ID NO: 118 - DNA, 20 bp, synthetic construct
  misc_feature 1..20: note = source = /note="Description of Artificial Sequence: Syntheticprimer"
  source 1..20: mol_type = other DNA
cttgtagtcggcaccaccat

SEQ ID NO: 119 - DNA, 20 bp, synthetic construct
  misc_feature 1..20: note = source = /note="Description of Artificial Sequence: Syntheticprimer"
  source 1..20: mol_type = other DNA
actgtatgtggagcggcttc

SEQ ID NO: 120 - DNA, 20 bp, synthetic construct
  misc_feature 1..20: note = source = /note="Description of Artificial Sequence: Syntheticprimer"
  source 1..20: mol_type = other DNA
caggtacaagctggaggtgg

SEQ ID NO: 121 - DNA, 22 bp, synthetic construct
  misc_feature 1..22: note = source = /note="Description of Artificial Sequence: Syntheticprimer"
  source 1..22: mol_type = other DNA
acccgttgaaccccattcgtga

SEQ ID NO: 122 - DNA, 23 bp, synthetic construct
  misc_feature 1..23: note = source = /note="Description of Artificial Sequence: Syntheticprimer"
  source 1..23: mol_type = other DNA
gcctcactaaaccatccaatcgg

SEQ ID NO: 123 - DNA, 1848 bp, Homo sapiens
  source 1..1848: mol_type = unassigned DNA
cctcgcacgcactgcgggctccggcgctgcgggctggccgggcgctgcgggctgaccgggcgctccgggaactcggctcgggaacctcgtctgcggtgggcggggccggcccggagccccgccccggctcagtccctgaaacccaggcgcggaccggctgcagtctcagaagggagctgctgtctgcggaggaaactgcatcgacggacggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggatcctgctggggccgccacggcctggctggtgggaaggccggcagcgggcggaggggatccagccacttccccctcttcttctgaagatcagaacattcagctctggagaacagtggttgcctgggggcttttgccactccttgtcccccgtgatctcccctcacactttgccatttgcttgtactgggacattgttctttccggccaaggtgccaccaccctgccccccctaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaacgct

SEQ ID NO: 124 - DNA, 4391 bp, Homo sapiens
  source 1..4391: mol_type = unassigned DNA
agcggcggccggaagctggctgagccggcctttggtaacgccacctgcacttctgggggcgtcgagcctggcggtagaatcttcccagtaggcggcgcgggagggaaaagaggattgaggggctaggccgggcggatcccgtcctcccccgatgtgagcagttttccgaaaccccgtcaggcgaaggctgcccagagaggtggagtcggtagcggggccgggaacatgaggcagtctctcctattcctgaccagcgtggttcctttcgtgctggcgccgcgacctccggatgacccgggcttcggcccccaccagagactcgagaagcttgattctttgctctcagactacgatattctctctttatctaatatccagcagcattcggtaagaaaaagagatctacagacttcaacacatgtagaaacactactaactttttcagctttgaaaaggcattttaaattatacctgacatcaagtactgaacgtttttcacaaaatttcaaggtcgtggtggtggatggtaaaaacgaaagcgagtacactgtaaaatggcaggacttcttcactggacacgtggttggtgagcctgactctagggttctagcccacataagagatgatgatgttataatcagaatcaacacagatggggccgaatataacatagagccactttggagatttgttaatgataccaaagacaaaagaatgttagtttataaatctgaagatatcaagaatgtttcacgtttgcagtctccaaaagtgtgtggttatttaaaagtggataatgaagagttgctcccaaaagggttagtagacagagaaccacctgaagagcttgttcatcgagtgaaaagaagagctgacccagatcccatgaagaacacgtgtaaattattggtggtagcagatcatcgcttctacagatacatgggcagaggggaagagagtacaactacaaattacttaatagagctaattgacagagttgatgacatctatcggaacacttcatgggataatgcaggttttaaaggctatggaatacagatagagcagattcgcattctcaagtctccacaagaggtaaaacctggtgaaaagcactacaacatggcaaaaagttacccaaatgaagaaaaggatgcttgggatgtgaagatgttgctagagcaatttagctttgatatagctgaggaagcatctaaagtttgcttggcacaccttttcacataccaagattttgatatgggaactcttggattagcttatgttggctctcccagagcaaacagccatggaggtgtttgtccaaaggcttattatagcccagttgggaagaaaaatatctatttgaatagtggtttgacgagcacaaagaattatggtaaaaccatccttacaaaggaagctgacctggttacaactcatgaattgggacataattttggagcagaacatgatccggatggtctagcagaatgtgccccgaatgaggaccagggagggaaatatgtcatgtatcccatagctgtgagtggcgatcacgagaacaataagatgttttcaaactgcagtaaacaatcaatctataagaccattgaaagtaaggcccaggagtgttttcaagaacgcagcaataaagtttgtgggaactcgagggtggatgaaggagaagagtgtgatcctggcatcatgtatctgaacaacgacacctgctgcaacagcgactgcacgttgaaggaaggtgtccagtgcagtgacaggaacagtccttgctgtaaaaactgtcagtttgagactgcccagaagaagtgccaggaggcgattaatgctacttgcaaaggcgtgtcctactgcacaggtaatagcagtgagtgcccgcctccaggaaatgctgaagatgacactgtttgcttggatcttggcaagtgtaaggatgggaaatgcatccctttctgcgagagggaacagcagctggagtcctgtgcatgtaatgaaactgacaactcctgcaaggtgtgctgcagggacctttctggccgctgtgtgccctatgtcgatgctgaacaaaagaacttatttttgaggaaaggaaagccctgtacagtaggattttgtgacatgaatggcaaatgtgagaaacgagtacaggatgtaattgaacgattttgggatttcattgaccagctgagcatcaatacttttggaaagtttttagcagacaacatcgttgggtctgtcctggttttctccttgatattttggattcctttcagcattcttgtccattgtgtggataagaaattggataaacagtatgaatctctgtctctgtttcaccccagtaacgtcgaaatgctgagcagcatggattctgcatcggttcgcattatcaaaccctttcctgcgccccagactccaggccgcctgcagcctgcccctgtgatcccttcggcgccagcagctccaaaactggaccaccagagaatggacaccatccaggaagaccccagcacagactcacatatggacgaggatgggtttgagaaggaccccttcccaaatagcagcacagctgccaagtcatttgaggatctcacggaccatccggtcaccagaagtgaaaaggctgcctcctttaaactgcagcgtcagaatcgtgttgacagcaaagaaacagagtgctaatttagttctcagctcttctgacttaagtgtgcaaaatatttttatagatttgacctacaaatcaatcacagcttgtattttgtgaagactgggaagtgacttagcagatgctggtcatgtgtttgaacttcctgcaggtaaacagttcttgtgtggtttggcccttctccttttgaaaaggtaaggtgaaggtgaatctagcttattttgaggctttcaggttttagtttttaaaatatcttttgacctgtggtgcaaaagcagaaaatacagctggattgggttatgaatatttacgtttttgtaaattaatcttttatattgataacagcactgactagggaaatgatcagtttttttttatacactgtaatgaaccgctgaatatgaggcatttggcatttatttgtgatgacaactggaatagttttttttttttttttttttttttgccttcaactaaaaacaaaggagataaatctagtatacattgtctctaaattgtgggtctatttctagttattacccagagtttttatgtagcagggaaaatatatatctaaatttagaaatcatttgggttaatatggctcttcataattctaagactaatgctctctagaaacctaaccacctaccttacagtgagggctatacatggtagccagttgaatttatggaatctaccaactgtttagggccctgatttgctgggcagtttttctgtattttataagtatcttcatgtatccctgttactgatagggatacatgctcttagaaaattcactattggctgggagtggtggctcatgcctgtaatcccagcacttggagaggctgaggttgcgccactacactccagcctgggtgacagagtgagactctgcctcaaaaaaaaaaaaaaaaaaaaaaaattcactatctacaaacctagaatatttaaaatacaaagattgcctgttttcaaacactattgaataagagggtgagatatttcttaacaacaacaacaacaaaaaaaacaggttgttttgaatgtgatgagccagccaggagatagaatactacctgcccttagggttgggggctgtccccacaagacttgatacttcagaaaccctttttattgacccacaagcagatatttgaattacttcttactttattgctccaggattctggatgggctgcatttactgtgtgaaggataaaaatcattagcctggattctgatttctataaattgccattaaaagctttttttcccctaagaactgaaatgtgctcaccagccaaaacattttaacttgtaaattttgagggcagttaaccaaacctgtgactaatcatatctcctcctaccccccatttccaaggacatttgttactcagatacttgttatactaatacttgaacttgtaccttatggtatttgctatcttttaactagtcatgatattcttatactttagttacacttttggaatttgatacaaggttgagtggggtgtgtgggtgtatgtatgagtgaaacagttctcaaaagaatgtaagaaaaaccatttttataaaattgtgactttttaaaaacatagtctttgtcatttatagaattaacaagctgctcagggtatattttatagctgtagcactgatatctgcattaataaatactgtcgaaacacaa

SEQ ID NO: 125 - RNA, 468 nt, synthetic construct
  misc_feature 1..468: note = source = /note="Description of Artificial Sequence: Syntheticpolynucleotide"
  source 1..468: mol_type = other RNA
gccaccatgtacagaatgcagctgctgagctgtatcgccctgtctctggccctggtcacaaatagcgcccctaccagcagcagcaccaagaaaacacagctgcaactggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctga

SEQ ID NO: 126 - RNA, 465 nt, synthetic construct
  misc_feature 1..465: note = source = /note="Description of Artificial Sequence: Syntheticpolynucleotide"
  source 1..465: mol_type = other RNA
gccaccatgctgaaactgctgctgctcctgtgtatcgccctgtctctggccgccacaaatagcgcccctaccagcagctccaccaagaaaacacagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctga

SEQ ID NO: 127 - RNA, 468 nt, synthetic construct
  misc_feature 1..468: note = source = /note="Description of Artificial Sequence: Syntheticpolynucleotide"
  source 1..468: mol_type = other RNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctga

SEQ ID NO: 128 - RNA, 468 nt, synthetic construct
  misc_feature 1..468: note = source = /note="Description of Artificial Sequence: Syntheticpolynucleotide"
  source 1..468: mol_type = other RNA
gccaccatgttgttgctgctgctcgcctgtattgccctggcctctacagccctggtcaccaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctga

SEQ ID NO: 129 - RNA, 749 nt, synthetic construct
  misc_feature 1..749: note = source = /note="Description of Artificial Sequence: Syntheticpolynucleotide"
  source 1..749: mol_type = other RNA
gccaccatgtacagaatgcagctgctgagctgtatcgccctgtctctggccctggtcacaaatagcgcccctaccagcagcagcaccaagaaaacacagctgcaactggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaagcagaatcatcacgaagtggtacttgaccacttcgtgatgattctgctttatcttagaggcatatccctacgtaccaacaagagcttcctacagcacaacaaacttgttgttgtgctgtaggaagctctttatcttagaggcatatccctacgtaccaacaagatccgcagacgtgtaaatgtacttgacatttacacgtctgcggatctttatcttagaggcatatcccttttatcttagaggcatatccct

SEQ ID NO: 130 - RNA, 749 nt, synthetic construct
  misc_feature 1..749: note = source = /note="Description of Artificial Sequence: Syntheticpolynucleotide"
  source 1..749: mol_type = other RNA
gccaccatgtacagaatgcagctgctgagctgtatcgccctgtctctggccctggtcacaaatagcgcccctaccagcagcagcaccaagaaaacacagctgcaactggaacacctcctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagattagggtctgtgagatacttgatctcacagaccctaatctcctttatcttagaggcatatccctacgtaccaacaagatgccatgaagaccaagacaacttgtgtcttggtcttcatggcatctttatcttagaggcatatccctacgtaccaacaagcctgatgggaatggaacctaacttgtaggttccattcccatcaggctttatcttagaggcatatcccttttatcttagaggcatatccct

SEQ ID NO: 131 - RNA, 749 nt, synthetic construct
  misc_feature 1..749: note = source = /note="Description of Artificial Sequence: Syntheticpolynucleotide"
  source 1..749: mol_type = other RNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaagcagaatcatcacgaagtggtacttgaccacttcgtgatgattctgctttatcttagaggcatatccctacgtaccaacaagagcttcctacagcacaacaaacttgttgttgtgctgtaggaagctctttatcttagaggcatatccctacgtaccaacaagatccgcagacgtgtaaatgtacttgacatttacacgtctgcggatctttatcttagaggcatatcccttttatcttagaggcatatccct

SEQ ID NO: 132 - RNA, 911 nt, synthetic construct
  misc_feature 1..911: note = source = /note="Description of Artificial Sequence: Syntheticpolynucleotide"
  source 1..911: mol_type = other RNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaagcagaatcatcacgaagtggtacttgaccacttcgtgatgattctgctttatcttagaggcatatccctacgtaccaacaagagcttcctacagcacaacaaacttgttgttgtgctgtaggaagctctttatcttagaggcatatccctacgtaccaacaagatccgcagacgtgtaaatgtacttgacatttacacgtctgcggatctttatcttagaggcatatccctacgtaccaacaagcgcaagaaatcccggtataaacttgttataccgggatttcttgcgctttatcttagaggcatatccctacgtaccaacaaggcgaggcagcttgagttaaaacttgtttaactcaagctgcctcgcctttatcttagaggcatatcccttttatcttagaggcatatccct

SEQ ID NO: 133 - RNA, 737 nt, synthetic construct
  misc_feature 1..737: note = source = /note="Description of Artificial Sequence: Syntheticpolynucleotide"
  source 1..737: mol_type = other RNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagtaccctgatgagatcacttggatctcatcagggtactcctttatcttagaggcatatccctacgtaccaacaaggagtaccctgatgagatcacttggatctcatcagggtactcctttatcttagaggcatatccctacgtaccaacaaggagtaccctgatgagatcacttggatctcatcagggtactcctttatcttagaggcatatcccttttatcttagaggcatatccct

SEQ ID NO: 134 - RNA, 807 nt, synthetic construct
  misc_feature 1..807: note = source = /note="Description of Artificial Sequence: Syntheticpolynucleotide"
  source 1..807: mol_type = other RNA
gccaccatgttgttgctgctgctcgcctgtattgccctggcctctacagccgccgctacaaattctgcccctaccagcagctccaccaagaaaacccagctgcaactggaacatctgctgctggacctgcagatgatcctgaacggcatcaacaactacaagaaccccaagctgacccggatgctgaccttcaagttctacatgcccaagaaggccaccgagctgaagcacctccagtgcctggaagaggaactgaagcccctggaagaagtgctgaatctggcccagagcaagaacttccacctgaggcctagggacctgatcagcaacatcaacgtgatcgtgctggaactgaaaggcagcgagacaaccttcatgtgcgagtacgccgacgagacagctaccatcgtggaatttctgaaccggtggatcaccttctgccagagcatcatcagcaccctgacctgaatagtgagtcgtattaacgtaccaacaaggagggcagaatcatcacgaagtggtgaagtacttgacttcaccacttcgtgatgattctgccctcctttatcttagaggcatatccctacgtaccaacaagagatgagcttcctacagcacaacaaatgtgacttgcacatttgttgtgctgtaggaagctcatctctttatcttagaggcatatccctacgtaccaacaagtacaagatccgcagacgtgtaaatgttccacttgggaacatttacacgtctgcggatcttgtactttatcttagaggcatatcccttttatcttagaggcatatccct

SEQ ID NO: 135 - RNA, 1910 nt, synthetic construct
  misc_feature 1..1910: note = source = /note="Description of Artificial Sequence: Syntheticpolynucleotide"
  source 1..1910: mol_type = other RNA
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaagttccttccaaatggctctgtacttgacagagccatttggaaggaactttatcttagaggcatatccctacgtaccaacaagcatcgttcaccgagatctgaacttgtcagatctcggtgaacgatgctttatcttagaggcatatccctacgtaccaacaagaccagcagcggacaaataaaacttgtttatttgtccgctgctggtctttatcttagaggcatatcccttttatcttagaggcatatccct

SEQ ID NO: 136 - RNA, 1938 nt, synthetic construct
  misc_feature 1..1938: note = source = /note="Description of Artificial Sequence: Syntheticpolynucleotide"
  source 1..1938: mol_type = other RNA
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaaatagtgagtcgtattaacgtaccaacaagaaggagctgcccatgagaaaacttgtttctcatgggcagctccttctttatcttagaggcatatccctacgtaccaacaagtgcaatgagggaccagtacaacttgtgtactggtccctcattgcactttatcttagaggcatatccctacgtaccaacaagagctgctgaaggactcatcaacttgtgatgagtccttcagcagctctttatcttagaggcatatcccttttatcttagaggcatatccct

SEQ ID NO: 137 - RNA, 1910 nt, synthetic construct
  misc_feature 1..1910: note = source = /note="Description of Artificial Sequence: Syntheticpolynucleotide"
  source 1..1910: mol_type = other RNA
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaagaaggagctgcccatgagaaaacttgtttctcatgggcagctccttctttatcttagaggcatatccctacgtaccaacaagtccaacgaatgggcctaagaacttgtcttaggcccattcgttggactttatcttagaggcatatccctacgtaccaacaaggacagcatagacgacaccttacttgaaggtgtcgtctatgctgtcctttatcttagaggcatatcccttttatcttagaggcatatccct

SEQ ID NO: 138 - RNA, 1910 nt, synthetic construct
  misc_feature 1..1910: note = source = /note="Description of Artificial Sequence: Syntheticpolynucleotide"
  source 1..1910: mol_type = other RNA
gccaccatgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggatctggcggaggtggaagcggcggaggcggatctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaatagtgagtcgtattaacgtaccaacaagaccctgacattcgctactgtacttgacagtagcgaatgtcagggtctttatcttagaggcatatccctacgtaccaacaagagctgctgaaggactcatcaacttgtgatgagtccttcagcagctctttatcttagaggcatatccctacgtaccaacaaggccaatgacccaacatctctacttgagagatgttgggtcattggcctttatcttagaggcatatcccttttatcttagaggcatatccct

SEQ ID NO: 139 - RNA, 772 nt, synthetic construct
  misc_feature 1..772: note = source = /note="Description of Artificial Sequence: Syntheticpolynucleotide"
  source 1..772: mol_type = other RNA
gccaccatgagaatcagcaagccccacctgagatccatcagcatccagtgctacctgtgcctgctgctgaacagccactttctgacagaggccggcatccacgtgttcatcctgggctgtttttctgccggcctgcctaagaccgaggccaactgggttaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagctgaatagtgagtcgtattaacgtaccaacaaggagtaccctgatgagatcacttggatctcatcagggtactcctttatcttagaggcatatccctacgtaccaacaaggtatccatctctggctatgaacttgtcatagccagagatggatacctttatcttagaggcatatccctacgtaccaacaagtcccgtaacgccatcatcttacttgaagatgatggcgttacgggactttatcttagaggcatatcccttttatcttagaggcatatccct

SEQ ID NO: 140 - RNA, 772 nt, synthetic construct
  misc_feature 1..772: note = source = /note="Description of Artificial Sequence: Syntheticpolynucleotide"
  source 1..772: mol_type = other RNA
gccaccatgagaatcagcaagccccacctgagatccatcagcatccagtgctacctgtgcctgctgctgaacagccactttctgacagaggccggcatccacgtgttcatcctgggctgtttttctgccggcctgcctaagaccgaggccaactgggttaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagctgaatagtgagtcgtattaacgtaccaacaaggagtaccctgatgagatcacttggatctcatcagggtactcctttatcttagaggcatatccctacgtaccaacaagaaggttcagcatagtagctaacttgtagctactatgctgaaccttctttatcttagaggcatatccctacgtaccaacaaggacgacgagaccttcatcaaacttgttgatgaaggtctcgtcgtcctttatcttagaggcatatcccttttatcttagaggcatatccct

SEQ ID NO: 141 - RNA, 821 nt, synthetic construct
  misc_feature 1..821: note = source = /note="Description of Artificial Sequence: Syntheticpolynucleotide"
  source 1..821: mol_type = other RNA
gccaccatgttccacgtgtccttccggtacatcttcggcctgcctccactgatcctggtgctgctgcctgtggccagcagcgactgtgatatcgagggcaaagacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtctgagggcaccaccatcctgctgaattgcaccggccaagtgaagggcagaaagcctgctgctctgggagaagcccagcctaccaagagcctggaagagaacaagtccctgaaagagcagaagaagctgaacgacctctgcttcctgaagcggctgctgcaagagatcaagacctgctggaacaagatcctgatgggcaccaaagaacactgaatagtgagtcgtattaacgtaccaacaagaaggttcagcatagtagctaacttgtagctactatgctgaaccttctttatcttagaggcatatccctacgtaccaacaagcgaattactgtgaaagtcaaacttgttgactttcacagtaattcgctttatcttagaggcatatccctacgtaccaacaagaccagcacactgagaatcaaacttgttgattctcagtgtgctggtctttatcttagaggcatatcccttttatcttagaggcatatccct

SEQ ID NO: 142 - protein, 22 aa, Homo sapiens
MCPARSLLLVATLVLLDHLSLA

SEQ ID NO: 143 - protein, 22 aa, Homo sapiens
MCHQQLVISWFSLVFLASPLVA

SEQ ID NO: 144 - protein, 29 aa, Homo sapiens
MRISKPHLRSISIQCYLCLLLNSHFLTEA

SEQ ID NO: 145 - protein, 25 aa, Homo sapiens
MFHVSFRYIFGLPPLILVLLPVASS

SEQ ID NO: 146 - DNA, 66 bp, Homo sapiens
  source 1..66: mol_type = unassigned DNA
atgtgtccagcgcgcagcctcctccttgtggctaccctggtcctcctggaccacctcagtttggcc

SEQ ID NO: 147 - DNA, 66 bp, Homo sapiens
  source 1..66: mol_type = unassigned DNA
atgtgtcaccagcagttggtcatctcttggttttccctggtttttctggcatctcccctcgtggcc

SEQ ID NO: 148 - DNA, 87 bp, Homo sapiens
  source 1..87: mol_type = unassigned DNA
atgagaatttcgaaaccacatttgagaagtatttccatccagtgctacttgtgtttacttctaaacagtcattttctaactgaagct

SEQ ID NO: 149 - DNA, 75 bp, Homo sapiens
  source 1..75: mol_type = unassigned DNA
atgttccacgtgtccttccggtacatcttcggcctgcctccactgatcctggtgctgctgcctgtggccagcagc

SEQ ID NO: 150 - RNA, 220 nt, synthetic construct
  misc_feature 1..220: note = /note="This sequence may encompass 1-220 nucleotides"
  misc_feature 1..220: note = source = /note="Description of Artificial Sequence: Syntheticpolynucleotide"
  misc_feature 1..220: note = source = /note="See specification as filed for detailed description ofsubstitutions and preferred embodiments"
  source 1..220: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 151 - RNA, 200 nt, synthetic construct
  misc_feature 1..200: note = /note="This sequence may encompass 1-200 nucleotides"
  misc_feature 1..200: note = source = /note="Description of Artificial Sequence: Syntheticpolynucleotide"
  misc_feature 1..200: note = source = /note="See specification as filed for detailed description ofsubstitutions and preferred embodiments"
  source 1..200: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 152 - RNA, 220 nt, synthetic construct; identical to SEQ ID NO: 150
  misc_feature 1..220: note = /note="This sequence may encompass 20-220 nucleotides"
  misc_feature 1..220: note = source = /note="Description of Artificial Sequence: Syntheticpolynucleotide"
  misc_feature 1..220: note = source = /note="See specification as filed for detailed description ofsubstitutions and preferred embodiments"
  source 1..220: mol_type = other RNA

SEQ ID NO: 153 - RNA, 120 nt, synthetic construct
  misc_feature 1..120: note = source = /note="Description of Artificial Sequence: Syntheticpolynucleotide"
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 154 - DNA, 120 bp, synthetic construct
  misc_feature 1..120: note = source = /note="Description of Artificial Sequence: Syntheticpolynucleotide"
  source 1..120: mol_type = other DNA
tttttttttttttttttttttttttttttttttttttttttttttttttttttttttttttttttttttttttttttttttttttttttttttttttttttttttttttttttttttttt